(19) United States
(12) Patent Application Publication
(10) Pub. No.: US 2009/0137601 A1
(43) Pub. Date: May 28, 2009

(54) L-Phenylalanine Derivatives

(75) Inventors: Simon Thomas Barry, Macclesfield (GB); Jason Grant Kettle, Macclesfield (GB); David Alan Rudge, Macclesfield (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/094,367

(22) PCT Filed: Nov. 22, 2006
(86) PCT No.: PCT/GB2006/004337

§ 371 Date: May 20, 2008

Related U.S. Application Data
(60) Provisional application No. 60/739,456, filed on Nov. 23, 2005.

(51) Int. Cl. A61K 31/502; C07D 211/96; A61K 31/445; A61K 31/351; A61P 35/00; A61P 9/10; A61P 31/00; C07D 309/06; C07D 401/06

(52) U.S. Cl. 514/252.03; 546/234; 514/331; 544/238; 549/426; 514/459

(57) ABSTRACT

The present invention relates to compounds that inhibit of a5b1 function, processes for their preparation, pharmaceutical compositions containing them as the active ingredient, to their use as medicaments and to their use in the manufacture of medicaments for use in the treatment in warm blooded animals such as humans of diseases that have a significant angiogenesis or vascular component such as for treatment of solid tumours. The present invention also relates to compounds that inhibit a5b1, and also that exhibit appropriate selectivity profile(s) against other integrins.

### BACKGROUND OF THE INVENTION

Many physiological and disease processes require cells to contact other cells and/or extracellular matrix. These adhesion events may be required for a variety of functions such as proliferation, migration, differentiation or survival. Cell adhesion interactions are mediated through several different protein families including selecting, cadherins, immunoglobulins and integrins. Because such adhesion events often play an essential role in diseases, pharmacological disruption of cell adhesion molecules may provide an effective therapeutic strategy. The integrin superfamily of adhesion molecules is believed to play a particularly important role in diverse acute and chronic disease states such as cancer, inflammatory diseases, stroke and neurodegenerative disorders.

The integrin superfamily is made up of structurally and functionally related surface glycoproteins that consist of non-covalently linked heterodimers consisting of α and β subunits. To-date, 18 different α and β subunits have been identified in mammals, which are known to form at least 24 different receptors. Each individual integrin molecule is able to specifically interact with multiple extracellular ligands, and there are a large number of such ligands such as collagens, fibronectins, fibrinogens vitronectins, and others. Thus, integrins represent a very complex biological area.

The integrin α5β1 (hereinafter a5b1) is composed of an α5 (hereinafter a5) and β1 (hereinafter b1) subunit. Only the b1 subunit can dimerise with a5. The a5b1 integrin is widely expressed in most tissues, although it is important for mediating cell adhesion to specific matrix proteins containing a short arginine-glycine-aspartate (RGD) motif. This motif is found in a variety of provisional extracellular matrix components such as fibronectin, fibrin and vitronectin. However, a5b1 is generally more selective towards fibronectin.

There is compelling evidence that a5b1 interaction with fibronectin plays an important role in physiopathological angiogenesis and vascular integrity. Endothelial cells express a variety of integrins, although a5b1 is particularly important for adhesion of endothelial cells to fibronectin of the provisional matrix. Fibronectin is upregulated in tumour tissue and wound-healing, and the ED-B splice variant of fibronectin is preferentially expressed on blood vessels of tumour tissues. Furthermore, immunohistochemical analysis has shown that a5b1 expression is upregulated in tumour vasculature. Transgenic studies show that a5 and b1 null mice are embryonic lethal and display defects in development of early vascular systems, revealing an important functional role. Moreover, functional studies using agents such as blocking RGD peptides or neutralising antibodies have shown that disruption of a5b1 interaction with its cognate ligands has anti-angiogenic effects.

In addition to a5b1, other integrin family members such as avb3 and aiibb3 can also interact with RGD-containing ligands. Other integrins can bind to ligands via non-RGD binding domains. An example of particular importance and relevance is a4b1 which binds via a leucine-aspartate-valine (LDV) motif to ligands that include the connecting segment-1 region of fibronectin. Since there are a variety of integrins that share the same ligand or binding-domain with a5b1, it will be important to develop therapeutic agents that are selective towards a5b1 activity, and thus reduce any potential adverse pharmacological affects that result from inhibition of other integrin types. However, since other endothelial integrins such as avb3, avb5 and a4b1 are also involved in possible pathological events, it is possible that agents which target such integrins in addition to a5b1, may have additional therapeutic activity.

Taken together, the expression and functional data suggest that selective inhibition of a5b1 function provides an attractive therapeutic strategy to combat diseases that have a significant angiogenesis or vascular component such as for treatment of solid tumours. There is thus a clear need to develop compounds that inhibit a5b1 with appropriate pharmacokinetic and pharmacodynamic drug properties, and also that exhibit appropriate selectivity profile(s) against other integrins.

### SUMMARY OF THE INVENTION

These and other needs are met by the present invention which is directed to a compound of the formula I:

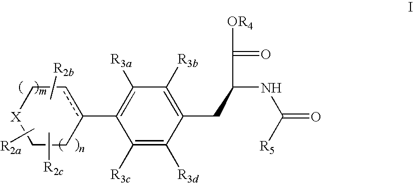

or a pharmaceutical acceptable salt, prodrug, or hydrate thereof, wherein:
X is O, N—R₁, or S(O)ₓ, wherein x is 0, 1, or 2;
m and n are each independently 0, 1, or 2;
“ [symbol] ” is a bond or is absent;
R₁ is (a) H, or an optionally substituted group selected from (C₁-C₆)alkyl, (C₃-C₆)cycloalkyl, heterocycloalkyl, (C₃-C₆)cycloalkyl(C₁-C₆)alkyl, heterocycloalkyl(C₁-C₆)alkyl, aryl, heteroaryl, aralkyl, or heteroaralkyl; or R₁ is
   (b)

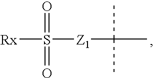

wherein

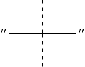

indicates the point of attachment and Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl; or R₁ is
(c)

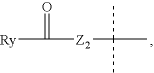

wherein

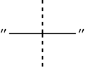

indicates the point of attachment and Z₂ is optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent and R^{y} is an optionally substituted group selected from (C₁-C₆)alkyl, (C₁-C₆)alkoxy, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or NR′R″, wherein R′ and R″ are each independently H or (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring; or R₁ is
(d) R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring;
R₂ₐ, R_{2b}, and R_{2c} are each independently H, halo, hydroxy, (C₁-C₃)alkyl, or (C₁-C₃)alkoxy, or if two of R₂ₐ, R_{2b}, and R_{2c} are attached to the same carbon, they may form oxo;
R₃ₐ, R_{3b}, R_{3c}, and R_{3d} are each independently H, halo, (C₁-C₃)alkyl, or (C₁-C₃)alkoxy;
R₄ is H, (C₁-C₆)alkyl, aryl, heteroaryl, aralkyl, heteroaralkyl; and
R₅ is aryl which is ortho-substituted with at least one group selected from (C₁-C₃)alkyl, (C₁-C₃)alkoxy or halogen, and which is optionally additionally substituted with 1 or 2 groups selected from (C₁-C₃)alkyl, (C₁-C₃)alkoxy, halogen, cyano or heterocycloalkyl, provided that when X is N—S(O)₂Me, R₅ is

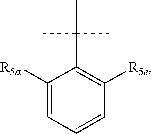

wherein R₅ₐ and R₅ₑ are each independently halo or (C₁-C₃)alkyl.

Also provided is a compound of the formula I as hereinbefore defined, or a pharmaceutically acceptable salt, prodrug, or hydrate thereof, wherein X, Y, m, n, R₂ₐ, R_{2b}, R_{2c}, R₃ₐ, R_{3b}, R_{3c}, R_{3d} and R₄ are as hereinbefore defined; and

R₅ is aryl which is ortho-substituted with at least one group selected from (C₁-C₃)alkyl or halogen, and which is optionally additionally substituted with 1 or 2 groups selected from (C₁-C₃)alkyl, (C₁-C₃)alkoxy or halogen, provided that when X is N—S(O)₂Me, R₅ is

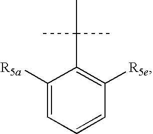

wherein R₅ₐ and R₅ₑ are each independently halo or (C₁-C₃)alkyl.

What is also provided is a compound of formula I, which is a compound of the formula IA:

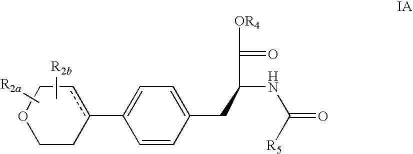

or a pharmaceutically acceptable salt, prodrug, or hydrate thereof, wherein R₂ₐ, R_{2b}, R₄, and R₅ are as defined for a compound of formula I.

What is also provided is a compound of formula I, which is a compound of the formula IB:

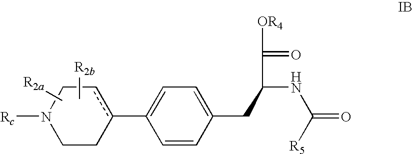

or a pharmaceutically acceptable salt, prodrug, or hydrate thereof, wherein R₂ₐ, R_{2b}, R₄, and R₅ are as defined for a compound of formula I and wherein R_{c} is an optionally substituted group selected from aralkyl, aryl, or heteroaryl.

What is also provided is a compound of formula I, which is a compound formula IC:

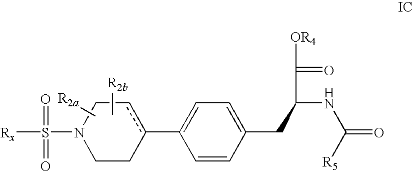

or a pharmaceutically acceptable salt, prodrug, or hydrate thereof, wherein R₂ₐ, R_{2b}, R₄, and R₅ are as defined for a compound of formula I and wherein Rₓ is an optionally substituted group selected from (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl.

What is also provided is a compound of formula I, which is a compound of formula ID:

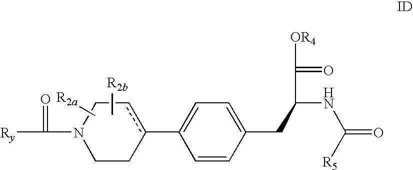

or a pharmaceutically acceptable salt, prodrug, or hydrate thereof, wherein R₂ₐ, R_{2b}, R₄, and R₅ are as defined for a compound of formula I and wherein R_{y} is an optionally substituted group selected from (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or NR′R″, wherein R′ and R″ are each independently H or (C₁-C₆)alkyl, aryl, heteroaryl, aralkyl, or heteroaralkyl, (C₁-C₆)alkoxy, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl or taken together with the nitrogen to which they are attached form an optionally substituted 3, 4, 5, 6, or 7-membered ring.

What is also provided is a compound of formula I which is selected from:

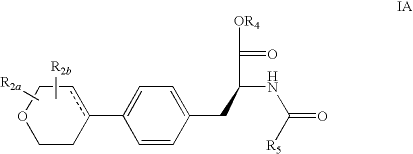

N-(2,6-dichlorobenzoyl)-4-(3,6-dihydro-2H-pyran-4-yl)-L-phenylalanine;
N-(2,6-dichlorobenzoyl)-4-(tetrahydro-2H-pyran-4-yl)-L-phenylalanine;
4-(3,6-dihydro-2H-pyran-4-yl)-N-(2,6-dimethylbenzoyl)-L-phenylalanine;

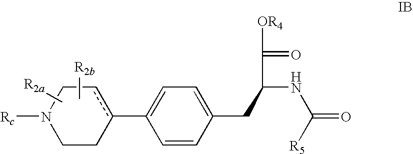

4-(1-benzylpiperidin-4-yl)-N-(2,6-dichlorobenzoyl)-L-phenylalanine;
N-(2,6-dichlorobenzoyl)-4-[1-(2-fluorobenzyl)piperidin-4-yl]-L-phenylalanine;
N-(2,6-dichlorobenzoyl)-4-[1-(3-fluorobenzyl)piperidin-4-yl]-L-phenylalanine;
N-(2,6-dichlorobenzoyl)-4-[1-(4-fluorobenzyl)piperidin-4-yl]-L-phenylalanine;
4-[1-(2-aminobenzyl)piperidin-4-yl]-N-(2,6-dichlorobenzoyl)-L-phenylalanine;
4-[1-(3-cyanobenzyl)piperidin-4-yl]-N-(2,6-dichlorobenzoyl)-L-phenylalanine;
N-(2,6-dichlorobenzoyl)-4-[1-(3-methoxybenzyl)piperidin-4-yl]-L-phenylalanine;
N-(2,6-dichlorobenzoyl)-4-[1-(4-methoxybenzyl)piperidin-4-yl]-L-phenylalanine;
N-(2,6-dichlorobenzoyl)-4-[1-(2-methoxybenzyl)piperidin-4-yl]-L-phenylalanine;
4-[1-(2-chlorobenzyl)piperidin-4-yl]-N-(2,6-dichlorobenzoyl)-L-phenylalanine;
4-[1-(3-chlorobenzyl)piperidin-4-yl]-N-(2,6-dichlorobenzoyl)-L-phenylalanine;
4-[1-(4-chlorobenzyl)piperidin-4-yl]-N-(2,6-dichlorobenzoyl)-L-phenylalanine;
N-(2,6-dichlorobenzoyl)-4-[1-(2-hydroxyethyl)-1,2,3,6-tetrahydropyridin-4-yl]-L-phenylalanine;
4-(1-benzyl-1,2,3,6-tetrahydropyridin-4-yl)-N-(2,6-dichlorobenzoyl)-L-phenylalanine;
N-(2,6-dichlorobenzoyl)-4-(1-isobutyl-1,2,3,6-tetrahydropyridin-4-yl)-L-phenylalanine;
4-[1-(2-amino-2-oxoethyl)-1,2,3,6-tetrahydropyridin-4-yl]-N-(2,6-dichlorobenzoyl)-L-phenylalanine;
N-(2,6-dichlorobenzoyl)-4-[1-(2-methylbenzyl)-1,2,3,6-tetrahydropyridin-4-yl]-L-phenylalanine;
N-(2,6-dichlorobenzoyl)-4-[1-(2-fluorobenzyl)-1,2,3,6-tetrahydropyridin-4-yl]-L-phenylalanine;
N-(2,6-dichlorobenzoyl)-4-[1-(2-chlorobenzyl)-1,2,3,6-tetrahydropyridin-4-yl]-L-phenylalanine;
N-(2,6-dichlorobenzoyl)-4-[1-(3-fluorobenzyl)-1,2,3,6-tetrahydropyridin-4-yl]-L-phenylalanine;
N-(2,6-dichlorobenzoyl)-4-[1-(3-chlorobenzyl)-1,2,3,6-tetrahydropyridin-4-yl]-L-phenylalanine;
N-(2,6-dichlorobenzoyl)-4-[1-(3-methoxybenzyl)-1,2,3,6-tetrahydropyridin-4-yl]-L-phenylalanine;
N-(2,6-dichlorobenzoyl)-4-[1-(3-methylbenzyl)-1,2,3,6-tetrahydropyridin-4-yl]-L-phenylalanine;
N-(2,6-dichlorobenzoyl)-4-[1-(4-fluorobenzyl)-1,2,3,6-tetrahydropyridin-4-yl]-L-phenylalanine;
N-(2,6-dichlorobenzoyl)-4-[1-(4-chlorobenzyl)-1,2,3,6-tetrahydropyridin-4-yl]-L-phenylalanine;
N-(2,6-dichlorobenzoyl)-4-[1-(2-methoxybenzyl)-1,2,3,6-tetrahydropyridin-4-yl]-L-phenylalanine;
N-(2,6-dichlorobenzoyl)-4-[1-(4-hydroxybenzyl)-1,2,3,6-tetrahydropyridin-4-yl]-L-phenylalanine;
N-(2,6-dichlorobenzoyl)-4-[1-(3,5-dimethoxybenzyl)-1,2,3,6-tetrahydropyridin-4-yl]-L-phenylalanine;
N-(2,6-dichlorobenzoyl)-4-[1-(2-hydroxybenzyl)-1,2,3,6-tetrahydropyridin-4-yl]-L-phenylalanine;
N-(2,6-dichlorobenzoyl)-4-[1-(4-cyanobenzyl)-1,2,3,6-tetrahydropyridin-4-yl]-L-phenylalanine;
N-(2,6-dichlorobenzoyl)-4-{1-[(5-methylisoxazol-3-yl)methyl]-1,2,3,6-tetrahydropyridin-4-yl}-L-phenylalanine;
N-(2,6-dichlorobenzoyl)-4-{1-[(3,5-dimethylisoxazol-4-yl)methyl]-1,2,3,6-tetrahydropyridin-4-yl}-L-phenylalanine;
N-(2,6-dichlorobenzoyl)-4-[1-(3-cyanobenzyl)-1,2,3,6-tetrahydropyridin-4-yl]-L-phenylalanine;
N-(2,6-dichlorobenzoyl)-4-[1-(3-chloro-4-fluorobenzyl)-1,2,3,6-tetrahydropyridin-4-yl]-L-phenylalanine;
N-(2,6-dichlorobenzoyl)-4-[1-(2,5-difluorobenzyl)-1,2,3,6-tetrahydropyridin-4-yl]-L-phenylalanine;
N-(2,6-dichlorobenzoyl)-4-[1-(2,4-difluorobenzyl)-1,2,3,6-tetrahydropyridin-4-yl]-L-phenylalanine;
N-(2,6-dichlorobenzoyl)-4-[1-(2,3-difluorobenzyl)-1,2,3,6-tetrahydropyridin-4-yl]-L-phenylalanine;
4-[1-(1,3-benzodioxol-5-ylmethyl)-1,2,3,6-tetrahydropyridin-4-yl]-N-(2,6-dichlorobenzoyl)-L-phenylalanine;
N-(2,6-dichlorobenzoyl)-4-[1-(3,4-difluorobenzyl)-1,2,3,6-tetrahydropyridin-4-yl]-L-phenylalanine;
4-{1-[4-(acetylamino)benzyl]-1,2,3,6-tetrahydropyridin-4-yl}-N-(2,6-dichlorobenzoyl)-L-phenylalanine;
N-(2,6-dichlorobenzoyl)-4-[1-(2,5-dimethoxybenzyl)-1,2,3,6-tetrahydropyridin-4-yl]-L-phenylalanine;
N-(2,6-dichlorobenzoyl)-4-{1-[4-(methylsulfonyl)benzyl]-1,2,3,6-tetrahydropyridin-4-yl}-L-phenylalanine;
N-(2,6-dichlorobenzoyl)-4-(1-phenyl-1,2,3,6-tetrahydropyridin-4-yl)-L-phenylalanine;

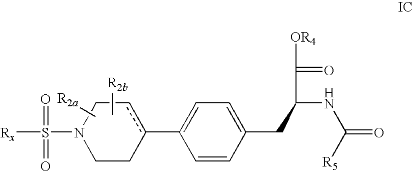

N-(2,6-dimethylbenzoyl)-4-[1-(methylsulfonyl)-1,2,3,6-tetrahydropyridin-4-yl]-L-phenylalanine;
N-(2,6-dichlorobenzoyl)-4-[1-(methylsulfonyl)-1,2,3,6-tetrahydropyridin-4-yl]-L-phenylalanine;
N-(2-chloro-4-fluorobenzoyl)-4-[1-(methylsulfonyl)-1,2,3,6-tetrahydropyridin-4-yl]-L-phenylalanine;
N-(4-bromo-2-chlorobenzoyl)-4-[1-(methylsulfonyl)-1,2,3,6-tetrahydropyridin-4-yl]-L-phenylalanine;
N-(2-chloro-4-pyrrolidin-1-ylbenzoyl)-4-[1-(methylsulfonyl)-1,2,3,6-tetrahydropyridin-4-yl]-L-phenylalanine;
N-(2,6-dichlorobenzoyl)-4-[1-(methylsulfonyl)piperidin-4-yl]-L-phenylalanine;
N-(2-chlorobenzoyl)-4-[1-(methylsulfonyl)piperidin-4-yl]-L-phenylalanine;
N-(2-chloro-6-methylbenzoyl)-4-[1-(pyridin-3-ylsulfonyl)-1,2,3,6-tetrahydropyridin-4-yl]-L-phenylalanine;
4-[1-(butylsulfonyl)piperidin-4-yl]-N-(2,6-dichlorobenzoyl)-L-phenylalanine;
N-(2,6-dichlorobenzoyl)-4-[1-(pyridin-3-ylsulfonyl)piperidin-4-yl]-L-phenylalanine;
N-(2,6-dichlorobenzoyl)-4-[1-(2-thienylsulfonyl)piperidin-4-yl]-L-phenylalanine;
N-(2,6-dichlorobenzoyl)-4-[1-(phenylsulfonyl)-1,2,3,6-tetrahydropyridin-4-yl]-L-phenylalanine;
N-(2,6-dichlorobenzoyl)-4-{1-[(4-fluorophenyl)sulfonyl]-1,2,3,6-tetrahydropyridin-4-yl}-L-phenylalanine;
N-(2,6-dichlorobenzoyl)-4-[1-(propylsulfonyl)-1,2,3,6-tetrahydropyridin-4-yl]-L-phenylalanine;
N-(2,6-dichlorobenzoyl)-4-{1-[(3-fluorophenyl)sulfonyl]-1,2,3,6-tetrahydropyridin-4-yl}-L-phenylalanine;
N-(2,6-dichlorobenzoyl)-4-{1-[(1,3,5-trimethyl-1H-pyrazol-4-yl)sulfonyl]-1,2,3,6-tetrahydropyridin-4-yl}-L-phenylalanine;
N-(2,6-dichlorobenzoyl)-4-[1-(2-thienylsulfonyl)-1,2,3,6-tetrahydropyridin-4-yl}-L-phenylalanine;
N-(2,6-dichlorobenzoyl)-4-{1-[(1,2-dimethyl-1H-imidazol-4-yl)sulfonyl]-1,2,3,6-tetrahydropyridin-4-yl}-L-phenylalanine;
N-(2,6-dichlorobenzoyl)-4-{1-[(3-chlorophenyl)sulfonyl]-1,2,3,6-tetrahydropyridin-4-yl}-L-phenylalanine;
N-(2,6-dichlorobenzoyl)-4-{1-[(4-methoxyphenyl)sulfonyl]-1,2,3,6-tetrahydropyridin-4-yl}-L-phenylalanine;
4-[1-(cyclopropylsulfonyl)-1,2,3,6-tetrahydropyridin-4-yl]-N-(2,6-dichlorobenzoyl)-L-phenylalanine;
N-(2,6-dichlorobenzoyl)-4-{1-[(2,4-dimethyl-1,3-thiazol-5-yl)sulfonyl]-1,2,3,6-tetrahydropyridin-4-yl}-L-phenylalanine;

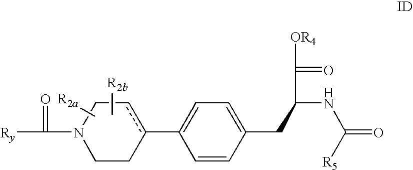

N-(2-chloro-4,5-dimethoxybenzoyl)-4-[1-(4-fluorobenzoyl)-1,2,3,6-tetrahydropyridin-4-yl]-L-phenylalanine;
N-(2-chloro-4,5-dimethoxybenzoyl)-4-[1-(3-fluorobenzoyl)-1,2,3,6-tetrahydropyridin-4-yl]-L-phenylalanine;
N-(2-chloro-6-methylbenzoyl)-4-[1-(3-fluorobenzoyl)-1,2,3,6-tetrahydropyridin-4-yl]-L-phenylalanine;
4-[1-(4-cyanobenzoyl)-1,2,3,6-tetrahydropyridin-4-yl]-N-(2,6-dimethylbenzoyl)-L-phenylalanine;
4-[1-(1H-benzimidazol-2-ylcarbonyl)-1,2,3,6-tetrahydropyridin-4-yl]-N-(2-chloro-3-fluorobenzoyl)-L-phenylalanine;
N-(2,6-dimethylbenzoyl)-4-[1-(3-methoxybenzoyl)-1,2,3,6-tetrahydropyridin-4-yl]-L-phenylalanine;
N-(2-chloro-4,5-dimethoxybenzoyl)-4-[1-(3-methoxybenzoyl)-1,2,3,6-tetrahydropyridin-4-yl]-L-phenylalanine;
N-(2-chloro-6-methylbenzoyl)-4-[1-(3-methoxybenzoyl)-1,2,3,6-tetrahydropyridin-4-yl]-L-phenylalanine;
N-(2,6-dimethylbenzoyl)-4-{1-[(2-methyl-1,3-thiazol-4-yl)carbonyl]-1,2,3,6-tetrahydropyridin-4-yl}-L-phenylalanine;
4-[1-(1H-benzimidazol-2-ylcarbonyl)-1,2,3,6-tetrahydropyridin-4-yl]-N-(4-cyano-2-methoxybenzoyl)-L-phenylalanine;
4-[1-(1H-benzimidazol-2-ylcarbonyl)-1,2,3,6-tetrahydropyridin-4-yl]-N-(2-chloro-4,5-dimethoxybenzoyl)-L-phenylalanine;
N-(2-chloro-4,5-dimethoxybenzoyl)-4-{1-[(2-methyl-1,3-thiazol-4-yl)carbonyl]-1,2,3,6-tetrahydropyridin-4-yl}-L-phenylalanine;
N-(2-chloro-6-methylbenzoyl)-4-[1-(3-cyanobenzoyl)-1,2,3,6-tetrahydropyridin-4-yl]-L-phenylalanine;
4-[1-(1H-benzimidazol-2-ylcarbonyl)-1,2,3,6-tetrahydropyridin-4-yl]-N-(2-chloro-6-methylbenzoyl)-L-phenylalanine;
N-(2-chloro-6-methylbenzoyl)-4-[1-(4-fluorobenzyl)-1,2,3,6-tetrahydropyridin-4-yl]-L-phenylalanine;
N-(2-chloro-3-fluorobenzoyl)-4-[1-(4-fluorobenzyl)-1,2,3,6-tetrahydropyridin-4-yl]-L-phenylalanine;
N-(2,6-dimethylbenzoyl)-4-[1-(4-fluorobenzyl)-1,2,3,6-tetrahydropyridin-4-yl]-L-phenylalanine;
4-[1-(cinnolin-4-ylcarbonyl)-1,2,3,6-tetrahydropyridin-4-yl]-N-(2,6-dimethylbenzoyl)-L-phenylalanine;
4-(1-acetyl-1,2,3,6-tetrahydropyridin-4-yl)-N-(2-chloro-6-methylbenzoyl)-L-phenylalanine;
N-(2-chloro-6-methylbenzoyl)-4-[1-(cyclopropylcarbonyl)-1,2,3,6-tetrahydropyridin-4-yl]-L-phenylalanine;
N-(2-chloro-6-methylbenzoyl)-4-[1-(pyridin-2-ylcarbonyl)-1,2,3,6-tetrahydropyridin-4-yl]-L-phenylalanine;
N-(2-chloro-4-fluorobenzoyl)-4-[1-(cinnolin-4-ylcarbonyl)-1,2,3,6-tetrahydropyridin-4-yl]-L-phenylalanine;
N-(2-chloro-4-fluorobenzoyl)-4-[1-(4-fluorobenzoyl)-1,2,3,6-tetrahydropyridin-4-yl]-L-phenylalanine;
N-(2,6-dimethylphenyl)-4-[1-(4-fluorobenzyl)-1,2,3,6-tetrahydropyridin-4-yl]-L-phenylalanine;
N-(2,6-dichlorobenzoyl)-4-[1-(pyrazin-2-ylcarbonyl)piperidin-4-yl]-L-phenylalanine;
N-(2,6-dichlorobenzoyl)-4-[1-(1,3-thiazol-4-ylcarbonyl)piperidin-4-yl]-L-phenylalanine;
N-(2,6-dichlorobenzoyl)-4-[1-(1,3-thiazol-5-ylcarbonyl)piperidin-4-yl]-L-phenylalanine;
N-(2,6-dichlorobenzoyl)-4-(1-isonicotinoylpiperidin-4-yl)-L-phenylalanine;
N-(2,6-dichlorobenzoyl)-4-[1-(1,2,5-thiadiazol-3-ylcarbonyl)piperidin-4-yl]-L-phenylalanine;
4-[1-(cinnolin-4-ylcarbonyl)piperidin-4-yl]-N-(2,6-dichlorobenzoyl)-L-phenylalanine;
N-(2,6-dichlorobenzoyl)-4-{1-[(1-methyl-1H-pyrrol-2-yl)carbonyl]piperidin-4-yl}-L-phenylalanine;
4-[1-(1H-benzimidazol-2-ylcarbonyl)piperidin-4-yl]-N-(2,6-dichlorobenzoyl)-L-phenylalanine;
N-(2,6-dichlorobenzoyl)-4-[1-(1H-indazol-3-ylcarbonyl)piperidin-4-yl]-L-phenylalanine;
N-(2,6-dichlorobenzoyl)-4-[1-(methoxyacetyl)piperidin-4-yl]-L-phenylalanine;
N-(2,6-dichlorobenzoyl)-4-[1-(3-methylbutanoyl)piperidin-4-yl]-L-phenylalanine;
4-{1-[(1-cyanocyclopropyl)carbonyl]piperidin-4-yl}-N-(2,6-dichlorobenzoyl)-L-phenylalanine;
N-(2,6-dichlorobenzoyl)-4-[1-(2-fluorobenzoyl)piperidin-4-yl]-L-phenylalanine;
N-(2,6-dichlorobenzoyl)-4-[1-(3-fluorobenzoyl)piperidin-4-yl]-L-phenylalanine;
N-(2,6-dichlorobenzoyl)-4-[1-(4-fluorobenzoyl)piperidin-4-yl]-L-phenylalanine;
4-[1-(3-cyanobenzoyl)piperidin-4-yl]-N-(2,6-dichlorobenzoyl)-L-phenylalanine;
4-[1-(4-cyanobenzoyl)piperidin-4-yl]-N-(2,6-dichlorobenzoyl)-L-phenylalanine;
N-(2,6-dichlorobenzoyl)-4-[1-(2-methoxybenzoyl)piperidin-4-yl]-L-phenylalanine;
N-(2,6-dichlorobenzoyl)-4-[1-(3-methoxybenzoyl)piperidin-4-yl]-L-phenylalanine;
N-(2,6-dichlorobenzoyl)-4-[1-(4-methoxybenzoyl)piperidin-4-yl]-L-phenylalanine;
4-[1-(2-chlorobenzoyl)piperidin-4-yl]-N-(2,6-dichlorobenzoyl)-L-phenylalanine;
4-[1-(4-chlorobenzoyl)piperidin-4-yl]-N-(2,6-dichlorobenzoyl)-L-phenylalanine;
4-[1-(1,3-benzodioxol-4-ylcarbonyl)piperidin-4-yl]-N-(2,6-dichlorobenzoyl)-L-phenylalanine;
N-(2,6-dichlorobenzoyl)-4-[1-(3-phenylpropanoyl)-1,2,3,6-tetrahydropyridin-4-yl]-L-phenylalanine;
4-(1-acetyl-1,2,3,6-tetrahydropyridin-4-yl)-N-(2,6-dichlorobenzoyl)-L-phenylalanine;
N-(2,6-dichlorobenzoyl)-4-[1-(benzoyl)-1,2,3,6-tetrahydropyridin-4-yl]-L-phenylalanine;
N-(2,6-dichlorobenzoyl)-4-[1-(2-methylbenzoyl)-1,2,3,6-tetrahydropyridin-4-yl]-L-phenylalanine;
N-(2,6-dichlorobenzoyl)-4-[1-(3-cyanobenzoyl)-1,2,3,6-tetrahydropyridin-4-yl]-L-phenylalanine;
N-(2,6-dichlorobenzoyl)-4-[1-(4-cyanobenzoyl)-1,2,3,6-tetrahydropyridin-4-yl]-L-phenylalanine;
N-(2,6-dichlorobenzoyl)-4-[1-(4-methylbenzoyl)-1,2,3,6-tetrahydropyridin-4-yl]-L-phenylalanine;
N-(2,6-dichlorobenzoyl)-4-[1-(2-fluorobenzoyl)-1,2,3,6-tetrahydropyridin-4-yl]-L-phenylalanine;
4-[1-(cyclopropylcarbonyl)-1,2,3,6-tetrahydropyridin-4-yl]-N-(2,6-dichlorobenzoyl)-L-phenylalanine;
N-(2,6-dichlorobenzoyl)-4-[1-(3-fluorobenzoyl)-1,2,3,6-tetrahydropyridin-4-yl]-L-phenylalanine;
N-(2,6-dichlorobenzoyl)-4-[1-(3-methoxybenzoyl)-1,2,3,6-tetrahydropyridin-4-yl]-L-phenylalanine;
N-(2,6-dichlorobenzoyl)-4-[1-(hydroxyacetyl)-1,2,3,6-tetrahydropyridin-4-yl]-L-phenylalanine;
N-(2,6-dichlorobenzoyl)-4-[1-(2-cyanobenzoyl)-1,2,3,6-tetrahydropyridin-4-yl]-L-phenylalanine;
N-(2,6-dichlorobenzoyl)-4-{1-[(1-methyl-1H-imidazol-2-yl)carbonyl]-1,2,3,6-tetrahydropyridin-4-yl}-L-phenylalanine;
N-(2,6-dichlorobenzoyl)-4-{1-[(1,5-dimethyl-1H-pyrrol-2-yl)carbonyl]-1,2,3,6-tetrahydropyridin-4-yl}-L-phenylalanine;
N-(2,6-dichlorobenzoyl)-4-{1-[(1-methyl-1H-pyrazol-3-yl)carbonyl]-1,2,3,6-tetrahydropyridin-4-yl}-L-phenylalanine;
N-(2,6-dichlorobenzoyl)-4-{1-[(5-methylisoxazol-3-yl)carbonyl]-1,2,3,6-tetrahydropyridin-4-yl}-L-phenylalanine;
N-(2,6-dichlorobenzoyl)-4-{1-[(5-methylpyridin-3-yl)carbonyl]-1,2,3,6-tetrahydropyridin-4-yl}-L-phenylalanine;
N-(2,6-dichlorobenzoyl)-4-[1-(1,3-thiazol-4-ylcarbonyl)-1,2,3,6-tetrahydropyridin-4-yl]-L-phenylalanine;
N-(2,6-dichlorobenzoyl)-4-{1-[(4-methyl-1,3-thiazol-5-yl)carbonyl]-1,2,3,6-tetrahydropyridin-4-yl}-L-phenylalanine;
N-(2,6-dichlorobenzoyl)-4-[1-(5-methyl-2-furoyl)-1,2,3,6-tetrahydropyridin-4-yl]-L-phenylalanine;
N-(2,6-dichlorobenzoyl)-4-{1-[(3-methylisoxazol-4-yl)carbonyl]-1,2,3,6-tetrahydropyridin-4-yl}-L-phenylalanine;
4-[1-(1-benzofuran-2-ylcarbonyl)-1,2,3,6-tetrahydropyridin-4-yl]-N-(2,6-dichlorobenzoyl)-L-phenylalanine;
4-[1-(cinnolin-4-ylcarbonyl)-1,2,3,6-tetrahydropyridin-4-yl]-N-(2,6-dichlorobenzoyl)-L-phenylalanine;
N-(2,6-dichlorobenzoyl)-4-[1-(isoquinolin-1-ylcarbonyl)-1,2,3,6-tetrahydropyridin-4-yl]-L-phenylalanine;
N-(2,6-dichlorobenzoyl)-4-{1-[(1,5-dimethyl-1H-pyrazol-3-yl)carbonyl]-1,2,3,6-tetrahydropyridin-4-yl}-L-phenylalanine;
N-(2,6-dichlorobenzoyl)-4-[1-(1H-indazol-3-ylcarbonyl)-1,2,3,6-tetrahydropyridin-4-yl]-L-phenylalanine;
N-(2,6-dichlorobenzoyl)-4-[1-(3-thienylcarbonyl)-1,2,3,6-tetrahydropyridin-4-yl]-L-phenylalanine;
N-(2,6-dichlorobenzoyl)-4-[1-(pyridin-2-ylcarbonyl)-1,2,3,6-tetrahydropyridin-4-yl]-L-phenylalanine;
N-(2,6-dichlorobenzoyl)-4-[1-(2-thienylcarbonyl)-1,2,3,6-tetrahydropyridin-4-yl]-L-phenylalanine;
N-(2,6-dichlorobenzoyl)-4-[1-(quinolin-4-ylcarbonyl)-1,2,3,6-tetrahydropyridin-4-yl]-L-phenylalanine;
N-(2,6-dichlorobenzoyl)-4-{1-[(5-methylpyrazin-2-yl)carbonyl]-1,2,3,6-tetrahydropyridin-4-yl}-L-phenylalanine;
4-[1-(1H-benzimidazol-2-ylcarbonyl)-1,2,3,6-tetrahydropyridin-4-yl]-N-(2,6-dichlorobenzoyl)-L-phenylalanine;
N-(2,6-dichlorobenzoyl)-4-{1-[4-(trifluoromethyl)benzoyl]-1,2,3,6-tetrahydropyridin-4-yl}-L-phenylalanine;
N-(2,6-dichlorobenzoyl)-4-{1-[3-(trifluoromethyl)benzoyl]-1,2,3,6-tetrahydropyridin-4-yl}-L-phenylalanine;
N-(2,6-dichlorobenzoyl)-4-{1-[2-chlorobenzoyl]-1,2,3,6-tetrahydropyridin-4-yl}-L-phenylalanine;
N-(2,6-dichlorobenzoyl)-4-{1-[3-chlorobenzoyl]-1,2,3,6-tetrahydropyridin-4-yl}-L-phenylalanine;
N-(2,6-dichlorobenzoyl)-4-{1-[4-(hydroxymethyl)benzoyl]-1,2,3,6-tetrahydropyridin-4-yl}-L-phenylalanine;
N-(2,6-dichlorobenzoyl)-4-{1-[2-(trifluoromethyl)benzoyl]-1,2,3,6-tetrahydropyridin-4-yl}-L-phenylalanine;
N-(2,6-dichlorobenzoyl)-4-{1-[2-methoxybenzoyl]-1,2,3,6-tetrahydropyridin-4-yl}-L-phenylalanine;
N-(2,6-dichlorobenzoyl)-4-{1-[(1,1-dioxidotetrahydro-2H-thiopyran-4-yl)carbonyl]-1,2,3,6-tetrahydropyridin-4-yl}-L-phenylalanine;
N-(2,6-dichlorobenzoyl)-4-{1-[(3,5-dimethyl-1H-pyrazol-1-yl)acetyl]-1,2,3,6-tetrahydropyridin-4-yl}-L-phenylalanine;
N-(2,6-dichlorobenzoyl)-4-{1-[(2,5-dimethyl-1,3-thiazol-4-yl)acetyl]-1,2,3,6-tetrahydropyridin-4-yl}-L-phenylalanine;
4-[1-(2,1,3-benzoxadiazol-5-ylcarbonyl)-1,2,3,6-tetrahydropyridin-4-yl]-N-(2,6-dichlorobenzoyl)-L-phenylalanine;
N-(2,6-dichlorobenzoyl)-4-[1-(3-hydroxy-2,2-dimethylpropanoyl)-1,2,3,6-tetrahydropyridin-4-yl]-L-phenylalanine;
N-(2,6-dichlorobenzoyl)-4-{1-[(2R)-3,3,3-trifluoro-2-hydroxy-2-methylpropanoyl]-1,2,3,6-tetrahydropyridin-4-yl}-L-phenylalanine;
4-[1-(1,2-benzisoxazol-3-ylacetyl)-1,2,3,6-tetrahydropyridin-4-yl]-N-(2,6-dichlorobenzoyl)-L-phenylalanine;
N-(2,6-dichlorobenzoyl)-4-[1-(1H-1,2,4-triazol-1-ylacetyl)-1,2,3,6-tetrahydropyridin-4-yl]-L-phenylalanine;
N-(2,6-dichlorobenzoyl)-4-[1-(tetrahydro-2H-pyran-4-ylacetyl)-1,2,3,6-tetrahydropyridin-4-yl]-L-phenylalanine;
N-(2,6-dichlorobenzoyl)-4-[1-(tetrahydro-2H-pyran-4-ylcarbonyl)-1,2,3,6-tetrahydropyridin-4-yl]-L-phenylalanine;
4-[1-(N-acetylglycyl)-1,2,3,6-tetrahydropyridin-4-yl]-N-(2,6-dichlorobenzoyl)-L-phenylalanine;
N-(2,6-dichlorobenzoyl)-4-{1-[(3-methoxyphenyl)acetyl]-1,2,3,6-tetrahydropyridin-4-yl}-L-phenylalanine;
4-[1-(cyclohexylacetyl)-1,2,3,6-tetrahydropyridin-4-yl]-N-(2,6-dichlorobenzoyl)-L-phenylalanine;
4-[1-(cyclobutylcarbonyl)-1,2,3,6-tetrahydropyridin-4-yl]-N-(2,6-dichlorobenzoyl)-L-phenylalanine;
4-[1-(cyclohexylcarbonyl)-1,2,3,6-tetrahydropyridin-4-yl]-N-(2,6-dichlorobenzoyl)-L-phenylalanine;
N-(2,6-dichlorobenzoyl)-4-[1-(pyridin-3-ylacetyl)-1,2,3,6-tetrahydropyridin-4-yl]-L-phenylalanine;
N-(2,6-dichlorobenzoyl)-4-[1-(3-pyrrolidin-1-ylbenzoyl)-1,2,3,6-tetrahydropyridin-4-yl]-L-phenylalanine;
4-[1-(4-cyano-2-methoxybenzoyl)-1,2,3,6-tetrahydropyridin-4-yl]-N-(2,6-dichlorobenzoyl)-L-phenylalanine;
N-(2,6-dichlorobenzoyl)-4-[1-(3-fluoro-2-methoxybenzoyl)-1,2,3,6-tetrahydropyridin-4-yl]-L-phenylalanine;
4-[1-(4-chloro-2-methoxybenzoyl)-1,2,3,6-tetrahydropyridin-4-yl]-N-(2,6-dichlorobenzoyl)-L-phenylalanine;
4-{1-[3-(acetylamino)benzoyl]-1,2,3,6-tetrahydropyridin-4-yl}-N-(2,6-dichlorobenzoyl)-L-phenylalanine;
N-(2,6-dichlorobenzoyl)-4-[1-(4-fluoro-3-methylbenzoyl)-1,2,3,6-tetrahydropyridin-4-yl]-L-phenylalanine;
4-[1-(2-chloro-5-fluorobenzoyl)-1,2,3,6-tetrahydropyridin-4-yl]-N-(2,6-dichlorobenzoyl)-L-phenylalanine;
N-(2,6-dichlorobenzoyl)-4-{1-[(3-methylisoxazol-5-yl)acetyl]-1,2,3,6-tetrahydropyridin-4-yl}-L-phenylalanine;
N-(2,6-dichlorobenzoyl)-4-[1-(2,3-dihydro-1H-inden-2-ylacetyl)-1,2,3,6-tetrahydropyridin-4-yl]-L-phenylalanine;
N-(2,6-dichlorobenzoyl)-4-{1-[(1,1-dioxidotetrahydro-3-thienyl)acetyl]-1,2,3,6-tetrahydropyridin-4-yl}-L-phenylalanine;
N-(2,6-dichlorobenzoyl)-4-[1-(1,2,5-thiadiazol-3-ylcarbonyl)-1,2,3,6-tetrahydropyridin-4-yl]-L-phenylalanine;
N-(2,6-dichlorobenzoyl)-4-{1-[(4-isopropyl-1,2,3-thiadiazol-5-yl)carbonyl]-1,2,3,6-tetrahydropyridin-4-yl}-L-phenylalanine;
4-[1-(2,1-benzisoxazol-3-ylcarbonyl)-1,2,3,6-tetrahydropyridin-4-yl]-N-(2,6-dichlorobenzoyl)-L-phenylalanine;
N-(2,6-dichlorobenzoyl)-4-[1-(quinolin-3-ylcarbonyl)-1,2,3,6-tetrahydropyridin-4-yl]-L-phenylalanine;
N-(2,6-dichlorobenzoyl)-4-[1-(1-methylpiperidin-4-ylcarbonyl)-1,2,3,6-tetrahydropyridin-4-yl]-L-phenylalanine;
N-(2,6-dichlorobenzoyl)-4-(1-{[(2-fluorophenyl)amino]carbonyl}-1,2,3,6-tetrahydropyridin-4-yl)-L-phenylalanine;
N-(2,6-dichlorobenzoyl)-4-(1-{[(2-methoxyphenyl)amino]carbonyl}-1,2,3,6-tetrahydropyridin-4-yl)-L-phenylalanine;
N-(2,6-dichlorobenzoyl)-4-{1-[(propylamino)carbonyl]-1,2,3,6-tetrahydropyridin-4-yl}-L-phenylalanine;
4-{1-[(benzylamino)carbonyl]-1,2,3,6-tetrahydropyridin-4-yl}-N-(2,6-dichlorobenzoyl)-L-phenylalanine;
N-(2,6-dichlorobenzoyl)-4-(1-{[(2-fluorobenzyl)amino]carbonyl}-1,2,3,6-tetrahydropyridin-4-yl)-L-phenylalanine;
4-[1-(aminocarbonyl)-1,2,3,6-tetrahydropyridin-4-yl]-N-(2,6-dichlorobenzoyl)-L-phenylalanine;
N-(2,6-dichlorobenzoyl)-4-{1-[(1-methyl-1H-indol-2-yl)carbonyl]-1,2,3,6-tetrahydropyridin-4-yl}-L-phenylalanine; and
4-[1-(1H-benzimidazol-1-ylacetyl)-1,2,3,6-tetrahydropyridin-4-yl]-N-(2,6-dichlorobenzoyl)-L-phenylalanine;

or a pharmaceutically acceptable salt, prodrug, or hydrate thereof.

What is also provided is a compound of formula I, IA, IB, IC, or ID or a pharmaceutically acceptable salt, prodrug, or solvate thereof in association with a pharmaceutically acceptable carrier, diluent, or excipient.

What is also provided is a compound of formula I, IA, IB, IC, or ID or a pharmaceutically acceptable salt, prodrug, or solvate thereof, which is an integrin inhibitor useful for controlling pathologically angiogenic diseases, thrombosis, cardiac infarction, coronary heart diseases, arteriosclerosis, tumours, osteoporosis, inflammations or infections.

What is also provided is a method of treating a disease or condition mediated by a5b1 which comprises administering to a patient in need of such treatment a compound of formula compound of formula I, IA, IB, IC, or ID or a pharmaceutically acceptable salt, prodrug, or solvate thereof.

What is also provided is a process for the preparation of a compound of formula I as summarized in Schemes 1-4 infra.

### DETAILED DESCRIPTION OF THE INVENTION

Unless otherwise stated, the following terms used in the specification and claims have the following meanings.

### DEFINITIONS

“Halo” means fluoro, chloro, bromo or iodo.

“(C₁-C₆)Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, tert-butyl, sec-butyl, n-pentyl, n-hexyl, and the like. Examples of optional substituents that may be present on a (C₁-C₆)alkyl group include one or more substituents selected from (C₁-C₃)alkyl, aryl (for example phenyl), heteroaryl (for example a monocyclic heteroaryl group as defined hereinafter), (C₁-C₃)haloalkyl, (C₁-C₃)alkoxy, (C₁-C₃)alkylthio, —O(CH₂)₁₋₅CF₃, halo, nitro, cyano, ═O, ═S, —OH, —SH, —CF₃, —OCF₃, —C(O)OR₆ (for example —C(O)OH and —C(O)O(C₁-C₆)alkyl), —OC(O)R₆, —NR₆R₇ (for example, —NH₂, —NH(C₁-C₆)alkyl or —N[(C₁-C₆)alkyl)₂], —C(O)NR⁶R⁷, —NHC(O)R⁶, —N[(C₁-C₆)alkyl]C(O)R₆, —C(O)R₆, —SR₆, —SOR₆, —SO₂R₆, —SO₂NR₆R₇ hydroxy-(C₁-C₃)alkyl, (C₁-C₃)alkoxy-(C₁-C₃)alkyl and NR₆R₇—(C₁-C₃)alkyl-; wherein R₆ and R₇ are independently hydrogen, alkyl (for example (C₁-C₆)alkyl, particularly (C₁-C₄)alkyl), heteroaryl (for example a monocyclic heteroaryl group as defined hereinafter) or aryl (for example phenyl) or R₆ and R₇ together with the nitrogen to which they are attached form a 4- to 7-membered ring (for example a 4- to 7-membered nitrogen containing heterocycloalkyl group as defined herein, such as a monocyclic nitrogen containing heterocycloalkyl group, for example azetidinyl, pyrrolidinyl, piperidinyl, piperazinyl or morpholinyl). Particularly, R⁶ and R⁷ are independently selected from hydrogen, (C₁-C₄)alkyl, phenyl or R⁶ and R⁷ together with the nitrogen to which they are attached form a 4- to 7-membered heterocycloalkyl group, for example pyrrolidinyl, piperidinyl, piperazinyl or morpholinyl.

An “alkylene,” “alkenylene,” or “alkynylene” group is an alkyl, alkenyl, or alkynyl group that is positioned between and serves to connect two other chemical groups. Thus, “(C₁-C₆)alkylene” means a linear saturated divalent hydrocarbon radical of one to six carbon atoms or a branched saturated divalent hydrocarbon radical of three to six carbon atoms, e.g., methylene, ethylene, propylene, 2-methylpropylene, pentylene, and the like. (C₁-C₆)alkylene may be substituted with one or more of the substituents selected from those provided for (C₁-C₆)alkyl.

“(C₂-C₆)Alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like. (C₁-C₆)Alkenylene may be substituted with one or more of the substituents selected from those provided for (C₁-C₆)alkyl.

“(C₂-C₆)Alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like. (C₁-C₆)Alkynylene may be substituted with one or more of the substituents selected from those provided for (C₁-C₆)alkyl.

“(C₃-C₆)Cycloalkyl” means a hydrocarbon ring containing from 3 to 6 carbon atoms, for example, cyclopropyl, cyclobutyl, cyclopentyl, cyclohexyl. Where possible, the cycloalkyl group may contain double bonds, for example, 3-cyclohexen-1-yl. The cycloalkyl ring may be optionally substituted as provided for (C₁-C₆)alkyl, or two adjacent substituents on a (C₃-C₆)cycloalkyl group together with the carbon atoms to which they are attached form a phenyl ring which is fused to the (C₃-C₆)cycloalkyl group, for example two adjacent substituents on a cyclopentyl ring together with the carbon atoms to which they are attached form a phenyl ring to give a 2,3-dihydro-1H-inden-2-yl group. For example, a (C₃-C₆)cycloalkyl group may be unsubstituted or substituted by 1 to 3 substituents selected from (C₁-C₃)alkyl, (C₁-C₃)haloalkyl, (C₁-C₃)alkoxy, hydroxy, thiol, nitro, halogen, amino, (C₁-C₃)alkylamino and di-[(C₁-C₃)]alkyl]amino, formyl, carboxyl, —CN, —NHCOR₆, —CONHR₆, —CO₂R₆, —COR₆, aryl, or heteroaryl, wherein R₆, alkyl, aryl, and heteroaryl are as defined herein. Examples of substituted (C₃-C₆)cycloalkyl groups include 1-cyanocyclopropyl, 1-fluorocyclopropyl, 2-iodocyclobutyl, 2,3-dimethylcyclopentyl, 2,2-dimethoxycyclohexyl or 3-phenylcyclopentyl.

“(C₃-C₆)Cycloalkyl(C₁-C₆)alkylene” means a (C₃-C₆)cycloalkyl group covalently attached to a (C₁-C₆)alkylene group, both of which are defined herein, for example cyclopropylmethyl, cyclobutylmethyl, cyclopentylmethyl or cyclohexylmethyl. (C₃-C₆)Cycloalkyl(C₁-C₆)alkylene may be optionally substituted as provided for (C₁-C₆)alkyl.

“(C₁-C₆)alkoxy” includes for example methoxy, ethoxy, propoxy and isopropoxy. (C₁-C₆)alkoxy may be optionally substituted as provided for (C₁-C₆)alkyl.

The term “heterocycloalkyl” means non-aromatic, monocyclic, fused, bridged, or spiro bicyclic saturated or partially saturated heterocyclic ring system(s) which optionally may be substituted with up to 4 groups selected from those recited above as substituents for alkyl. Monocyclic heterocyclic rings contain from about 3 to 12 ring atoms, with from 1 to 5 heteroatoms selected from N, O, and S, and preferably from 3 to 7 member atoms, in the ring. Bicyclic heterocycles contain from 7 to 17 member atoms, preferably 7 to 12 member atoms, in the ring. Bicyclic heterocycles contain from about 7 to about 17 ring atoms, preferably from 7 to 12 ring atoms. Bicyclic heterocyclic(s) rings may be fused, spiro, or bridged ring systems. Partially saturated heterocycles are heterocyclic ring systems that are not completely saturated and include partially aromatic ring systems in the sense that one ring of a fused ring system may be aromatic and the other non-aromatic, for example indoline Examples of heterocyclic groups include cyclic ethers (oxiranes) such as ethyleneoxide, tetrahydrofuran, tetrahydropyran, dioxane, and substituted cyclic ethers, wherein the substituents are those described above for the alkyl and cycloalkyl groups. Typical substituted cyclic ethers include propyleneoxide, phenyloxirane (styrene oxide), cis-2-butene-oxide (2,3-dimethyloxirane), 3-chlorotetrahydrofuran, 2,6-dimethyl-1,4-dioxane, and the like. Heterocycles containing nitrogen are groups such as pyrrolidine, piperidine, piperazine, tetrahydrotriazine, tetrahydropyrazole, and substituted groups such as 3-aminopyrrolidine, 4-methylpiperazin-1-yl, and the like. Typical sulfur containing heterocycles include tetrahydrothiophene, dihydro-1,3-dithiol-2-yl, and hexahydrothiepin-4-yl. Other commonly employed heterocycles include dihydro-oxathiol-4-yl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene and tetrahydrothiopyran.

“Heterocycloalkyl(C₁-C₆)alkylene” means a heterocycloalkyl group covalently attached to a (C₁-C₆)alkylene group, both of which are defined herein, for example pyrrolidinylmethyl, piperidinylmethyl, morpholinylmethyl and piperazinylmethyl. (C₃-C₆)Heterocycloalkyl(C₁-C₆)alkylene may be optionally substituted as provided for (C₁-C₆)alkyl.

The term “aryl” means a cyclic or polycyclic aromatic ring having from 5 to 12 carbon atoms. Aryl may be unsubstituted or substituted with up to 4 groups selected from those recited above as substituents for (C₁-C₆)alkyl; or two substituents on the aryl ring form a (C₁-C₄)alkylenedioxy group (for example two adjacent substituents form a methylenedioxy or ethylenedioxy group); or two substituents on the aryl ring form a (C₃-C₆)cycloalkyl group (for example two adjacent substituents on a phenyl ring, together with the phenyl ring to which they are attached form a 2,3-dihydroindenyl group). The term aryl includes both monovalent species and divalent species. Examples of aryl groups include, but are not limited to, phenyl, biphenyl, naphthyl, each of which may be optionally substituted with 1 or more (for example 1 to 4) substituents as defined above as substituents for (C₁-C₆)alkyl, examples of substituted aryl include 2-chlorophenyl, 3-chlorophenyl, 4-chlorophenyl, 2-fluororophenyl, 3-fluorophenyl, 4-fluorophenyl, 2-methylphenyl, 3-methylphenyl, 4-methylphenyl, 2-hydroxyphenyl, 3-hydroxyphenyl, 4-hydroxyphenyl, 2-methoxyphenyl, 3-methoxyphenyl, 4-methoxyphenyl, 2-aminophenyl, 2-cyanophenyl, 3-cyanophenyl, 4-cyanophenyl, 4-methylsulfonylphenyl, 4-acetylaminophenyl, 3-pyrrolidinylphenyl, 4-hydroxymethylphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, 2-trifluoromethylphenyl, 3-trifluoromethylphenyl, 4-trifluoromethylphenyl and the like.

Aralkyl means an aryl group covalently attached to a (C₁-C₆)alkylene group, both of which are defined herein. Aralkyl may be optionally substituted as provided for (C₁-C₆)alkyl. Examples of aralkyl groups include benzyl, phenylethyl, 3-(3-chlorophenyl)-2-methylpentyl, 2-chlorobenzyl, 3-chlorobenzyl, 4-chlorobenzyl, 2-fluorobenzyl, 3-fluorobenzyl, 4-fluorobenzyl, 2-methylbenzyl, 3-methylbenzyl, 4-methylbenzyl, 2-hydroxybenzyl, 3-hydroxybenzyl, 4-hydroxybenzyl, 2-methoxybenzyl, 3-methoxybenzyl, 4-methoxybenzyl, 2-aminobenzyl, 2-cyanobenzyl, 3-cyanobenzyl, 4-cyanobenzyl, 4-methylsulfonylbenzyl, 4-acetylaminobenzyl, 2-chloro-3-methylbenzyl, 2-chloro-4-methylbenzyl, 2-chloro-5-methylbenzyl, 3-chloro-2-methylbenzyl, 3-chloro-4-methylbenzyl, 4-chloro-2-methylbenzyl, 4-chloro-3-methylbenzyl, 5-chloro-2-methylbenzyl, 2,3-dichlorobenzyl, 2,5-dichlorobenzyl, 3,4-dichlorobenzyl, 2,3-dimethylbenzyl, 3,4-dimethylbenzyl, and the like.

The term “heteroaryl” means an aromatic mono-, bi- or polycyclic ring incorporating one or more (for example 1 to 4) heteroatoms selected from N, O and S. Heteroaryl may be unsubstituted or substituted with up to 4 groups selected from those recited above as substituents for (C₁-C₆)alkyl. The term heteroaryl includes both monovalent species and divalent species. Examples of monocyclic heteroaryl include, but are not limited to substituted or unsubstituted thienyl, furanyl, pyrrolyl, imidazolyl, pyrazolyl, thiazolyl, isothiazolyl, isoxazolyl, oxazolyl, thiadiazolyl, triazolyl, tetrazolyl, pyridinyl, pyrazinyl or pyrimidinyl. Monocyclic diheteroaryl groups (monocyclic heteroaromatic groups with 2 heteroatoms) include, but are not limited to 1-, 2-, 4- or 5-imidazolyl, 1-, 3-, 4- or 5-pyrazolyl, 3-, 4- or 5-isothiazolyl, 3-, 4- or 5-isoxazolyl, 2-pyrazinyl, 2-, 4- or 5-pyrimidinyl. Examples of monocyclic heteroaromatic groups with 3 or more heteroatoms include, but are not limited to, 1-, 3- or 5-triazolyl, 1-, 2- or 3-tetrazolyl, 1,2,5-thiadiazol-3-yl or 1,2,3-thiadiazol-5-yl). Examples of bicyclic and polycyclic heteroaryl groups include but are not limited to 1-, 2-, 3-, 5-, 6-, 7- or 8-indolizinyl, 1-, 3-, 4-, 5-, 6- or 7-isoindolyl, 2-, 3-, 4-, 5-, 6- or 7-indolyl, 2-, 3-, 4-, 5-, 6- or 7-indazolyl, 2-, 4-, 5-, 6-, 7- or 8-purinyl, 1-, 2-, 3-, 4-, 6-, 7-, 8- or 9-quinolizinyl, 2-, 3-, 4-, 5-, 6-, 7- or 8-quinolinyl, 1-, 3-, 4-, 5-, 6-, 7- or 8-isoquinolinyl, 1-, 4-, 5-, 6-, 7- or 8-phthalazinyl, 2-, 3-, 4-, 5- or 6-naphthyridinyl, 2-, 3-, 5-, 6-, 7- or 8-quinazolinyl, 3-, 4-, 5-, 6-, 7- or 8-cinnolinyl, 2-, 4-, 6- or 7-pteridinyl, 1-, 2-, 3-, 4-, 5-, 6-, 7- or 8-4-aH carbazolyl, 1-, 2-, 3-, 4-, 5-, 6-, 7- or 8-carbazolyl, 1-, 3-, 4-, 5-, 6-, 7-, 8- or 9-carbolinyl, 1-, 2-, 3-, 4-, 6-, 7-, 8-, 9- or 10-phenanthridinyl, 1-, 2-, 3-, 4-, 5-, 6-, 7-, 8- or 9-acridinyl, 1-, 2-, 4-, 5-, 6-, 7-, 8- or 9-perimidinyl, 2-, 3-, 4-, 5-, 6-, 8-, 9- or 10-phenathrolinyl, 1-, 2-, 3-, 4-, 6-, 7-, 8- or 9-phenazinyl, 1-, 2-, 3-, 4-, 6-, 7-, 8-, 9- or 10-phenothiazinyl, 1-, 2-, 3-, 4-, 6-, 7-, 8-, 9- or 10-phenoxazinyl, 2-, 3-, 4-, 5-, 6- or 1-, 3-, 4-, 5-, 6-, 7-, 8-, 9- or 10-benzisoquinolinyl, 2-, 3-, 4 or thieno[2,3-b]furanyl, 2-, 3-, 5-, 6-, 7-, 8-, 9-, 10- or 11-7H-pyrazino[2,3-c]carbazolyl, 2-, 3-, 5-, 6- or 7-2H-furo[3,2-b]-pyranyl, 2-, 3-, 4-, 5-, 7- or 8-5H-pyrido[2,3-d]-o-oxazinyl, 1-, 3- or 5-1H-pyrazolo[4,3-d]-oxazolyl, 2-, 4- or 5-4H-imidazo[4,5-d]thiazolyl, 3-, 5- or 8-pyrazino[2,3-d]pyridazinyl, 2-, 3-, 5- or 6-imidazo[2,1-b]thiazolyl, 1-, 3-, 6-, 7-, 8- or 9-furo[3,4-c]cinnolinyl, 1-, 2-, 3-, 4-, 5-, 6-, 7-, 8-, 9-, 10 or 11-4H-pyrido[2,3-c]carbazolyl, 2-, 3-, 6- or 7-imidazo[1,2-b][1,2,4]triazinyl, 7-benzo[b]thienyl, 2-, 4-, 5-, 6- or 7-benzoxazolyl, 3-, 4-, 5-, 6- or 7-benzisoxazolyl, 4- or 5-(2,1,3-benzisoxadiazolyl), 2-, 4-, 5-, 6- or 7-benzimidazolyl, 2-, 4-, 5-, 6- or 7-benzothiazolyl, 2-, 3-, 4-, 5-, 6- or 7-benzo[b]furanyl, 1-, 2-, 4-, 5-, 6-, 7-, 8- or 9-benzoxapinyl, 2-, 4-, 5-, 6-, 7- or 8-benzoxazinyl, 1-, 2-, 3-, 5-, 6-, 7-, 8-, 9-, 10- or 11-1H-pyrrolo[1,2-b][2]benzazapinyl. Typical fused heteroaryl groups include, but are not limited to 2-, 3-, 4-, 5-, 6-, 7- or 8-quinolinyl, 1-, 3-, 4-, 5-, 6-, 7- or 8-isoquinolinyl, 2-, 3-, 4-, 5-, 6- or 7-indolyl, 2-, 3-, 4-, 5-, 6- or 7-benzo[b]thienyl, 2-, 4-, 5-, 6- or 7-benzoxazolyl, 2-, 4-5-, 6- or 7-benzimidazolyl, 2-, 4-, 5-, 6- or 7-benzothiazolyl.

“Heteroaralkyl” means an heteroaryl group covalently attached to a (C₁-C₆)alkylene group, both of which are defined herein. Heteroaralkyl may be optionally substituted as provided for (C₁-C₆)alkyl. Examples of heteroaralkyl groups include pyridin-3-ylmethyl, 3-(benzofuran-2-yl)propyl, 1,3-thiazolylmethyl, isoxazolylmethyl, 1,2,4-triazolylmethyl, pyridinylmethyl, pyrimidinylmethyl or pyrazinylmethyl and the like.

“Haloalkyl” means alkyl substituted with one or more same or different halo atoms, e.g., —CH₂Cl, —CF₃, —CH₂CF₃, —CH₂CCl₃, and the like.

“Optionally substituted” means that the group at issue is optionally substituted as provided herein.

1. Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. For example, if the R₂ₐ and R_{2c} substituents in a compound of Formula (I) are attached to the same carbon and are different, then the carbon to which they are attached is an asymmetric center and the compound of Formula (I) can exist as an (R)- or (S)-stereoisomer relative to that carbon. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001).

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the specification and claims includes both one and more than one such excipient.

A “pharmaceutically acceptable counterion” means an ion having a charge opposite to that of the substance with which it is associated and that is pharmaceutically acceptable. Representative examples include, but are not limited to, chloride, bromide, iodide, methanesulfonate, p-tolylsulfonate, trifluoroacetate, acetate, and the like.
1. A “pharmaceutically acceptable salt” of a compound means a salt that is pharmaceutically acceptable and that possesses the desired pharmacological activity of the parent compound. Such salts include:
   acid addition salts, formed with inorganic acids such as hydrochloric acid, hydrobromic acid, sulfuric acid, nitric acid, phosphoric acid, and the like; or
   formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynapthoic acid, salicylic acid, stearic acid, muconic acid, and the like; or
2. salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Leaving group” has the meaning conventionally associated with it in synthetic organic chemistry i.e., an atom or group capable of being displaced by a nucleophile and includes halogen (such as chloro, bromo, iodo), alkanesulfonyloxy (such as mesyloxy or trifluoromethylsulfonyloxy) or arenesulfonyloxy (such as tosyloxy), and the like. Leaving Groups are well known in the art and are catalogued in “Protective Groups in Organic Synthesis 3^{rd} Ed.”, edited by Theodora Green and Peter Wuts (John Wiley, 1999).

The compounds of Formula (I) may be administered in the form of a pro-drug which is broken down in the human or animal body to give a compound of the Formula (I). A “Pro-drug” is any compound which releases an active parent drug according to Formula (I) in vivo when such prodrug is administered to a mammalian subject. Prodrugs of a compound of Formula (I) are prepared by modifying functional groups present in the compound of Formula (I) in such a way that the modifications may be cleaved in vivo to release the parent compound. Examples of prodrugs include, but are not limited to esters (e.g., acetate, formate, and benzoate derivatives), carbamates (e.g., N,N-dimethylaminocarbonyl) of hydroxy functional groups in compounds of Formula (I); or esters of carboxy functional groups in compounds of formula I; and the like.

Various forms of pro-drugs are known in the art. For examples of such pro-drug derivatives, see:
1. Design of Prodrugs, edited by H. Bundgaard, (Elsevier, 1985) and Methods in Enzymology, Vol. 42, p. 309-396, edited by K. Widder, et al. (Academic Press, 1985);
2. A Textbook of Drug Design and Development, edited by Krogsgaard-Larsen and H. Bundgaard, Chapter 5 “Design and Application of Prodrugs”, by H. Bundgaard p. 113-191 (1991);
3. H. Bundgaard, Advanced Drug Delivery Reviews, 8, 1-38 (1992);
4. H. Bundgaard, et al., Journal of Pharmaceutical Sciences, 77, 285 (1988);
5. N. Kakeya, et al., Chem Pharm Bull, 32, 692 (1984);
6. K. Beaumont et. al., Current Drug Metabolism, 4, 461 (2003).

An in-vivo hydrolysable ester of a compound of the Formula (I) containing a carboxy or a hydroxy group is, for example, a pharmaceutically-acceptable ester which is hydrolysed in the human or animal body to produce the parent acid or alcohol. Suitable pharmaceutically-acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters, for example 5-methyl-1,3-dioxolen-2-onylmethyl; and C₁₋₆alkoxycarbonyloxyethyl esters.

An in-vivo hydrolysable ester of a compound of the formula I containing a carboxy or a hydroxy group is, for example, a pharmaceutically-acceptable ester which is hydrolysed in the human or animal body to produce the parent acid or alcohol. Suitable pharmaceutically-acceptable esters for carboxy include (C₁-C₆)alkyl esters, for example ethyl or isopropyl esters; (C₁-C₆)alkoxymethyl esters for example methoxymethyl, (C₁-C₆)alkanoyloxymethyl esters for example pivaloyloxymethyl, phthalidyl esters, (C₃-C₈)cycloalkoxycarbonyloxy(C₁-C₆)alkyl esters for example 1-cyclohexylcarbonyloxyethyl; 1,3-dioxolen-2-onylmethyl esters, for example 5-methyl-1,3-dioxolen-2-onylmethyl; and C₁₋₆alkoxycarbonyloxyethyl esters.

“Treating” or “treatment” of a disease includes:
1. preventing the disease, i.e. causing the clinical symptoms of the disease not to develop in a mammal that may be exposed to or predisposed to the disease but does not yet experience or display symptoms of the disease;
2. inhibiting the disease, i.e., arresting or reducing the development of the disease or its clinical symptoms; or
3. relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The phrase “compound of the invention” means those compounds which are disclosed herein, both generically and specifically, except for the following:
N-(2-chloro-4-fluorobenzoyl)-4-{1-[(2R)-2-hydroxypropanoyl]-1,2,3,6-tetrahydropyridin-4-yl}-L-phenylalanine;
4-(1-acetyl-1,2,3,6-tetrahydropyridin-4-yl)-N-(4-chloro-2,5-difluorobenzoyl)-L-phenylalanine;
N-(2-chloro-3-fluorobenzoyl)-4-[1-(propylsulfonyl)-1,2,3,6-tetrahydropyridin-4-yl]-L-phenylalanine;
4-[1-(propylsulfonyl)-1,2,3,6-tetrahydropyridin-4-yl]-N-(2,3,5-trifluorobenzoyl)-L-phenylalanine;
4-(1-acetyl-1,2,3,6-tetrahydropyridin-4-yl)-N-(2-chloro-3-fluorobenzoyl)-L-phenylalanine; and
N-(2-chloro-3-fluorobenzoyl)-4-[1-(pyridin-2-ylcarbonyl)-1,2,3,6-tetrahydropyridin-4-yl]-L-phenylalanine.

It is to be understood that certain compounds of the formula I may exist in solvated as well as unsolvated forms such as, for example, hydrated forms. It is to be understood that the invention encompasses all such solvated forms which exhibit an inhibitory effect on a5b1, for example an antiangiogenic effect.

It is also to be understood that certain compounds of the formula I may exhibit polymorphism, and that the invention encompasses all such forms which exhibit an inhibitory effect on a5b1, for example an antiangiogenic effect.

It is also to be understood that the invention relates to all tautomeric forms of the compounds of the formula I which exhibit an inhibitory effect on a5b1, for example an antiangiogenic effect.

### Invention Compounds

We turn now to a compound of formula I.

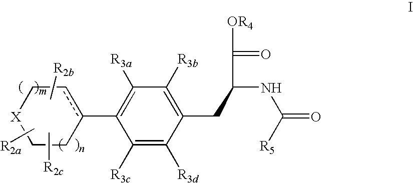

In one embodiment of the invention, in a compound of formula I, X is O or N—R₁.

In another embodiment “-----” is a bond.

In another embodiment “-----” is absent.

In another embodiment of the invention, X is O.

In another embodiment of the invention, X is NH.

In another embodiment “-----” is a bond and X is NR₁ wherein R₁ has any of the values defined herein.

In another embodiment “-----” is absent and X is NR₁ wherein R₁ has any of the values defined herein.

In another embodiment X is NR₁ and R₁ is selected from:
(a) an optionally substituted group selected from (C₁-C₆)alkyl, (C₃-C₆)cycloalkyl, heterocycloalkyl, (C₃-C₆)cycloalkyl(C₁-C₆)alkyl, heterocycloalkyl(C₁-C₆)alkyl, aryl, heteroaryl, aralkyl, or heteroaralkyl; or R₁ is
(b)

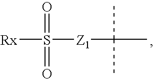

wherein

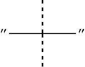

indicates the point of attachment and Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl; or R₁ is
(c)

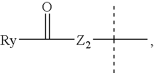

wherein

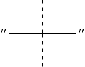

indicates the point of attachment and Z₂ is optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent and R_{y} is an optionally substituted group selected from (C₁-C₆)alkyl, (C₁-C₆)alkoxy, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or NR′R″, wherein R′ and R″ are each independently H or (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring; or R₁ is
(d) R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring.

In another embodiment of the invention X is NR₁ and R₁ is selected from optionally substituted aralkyl,

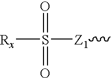

and

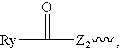

wherein [symbol] indicates the point of attachment; and Rₓ, R_{y}, Z₁ and Z₂ are as hereinbefore defined.

In another embodiment of the invention X is NR₁ and R₁ is selected from optionally substituted (C₁-C₆)alkyl, aralkyl (for example optionally substituted benzyl or phenylethyl) or heteroaralkyl; or R₁ is

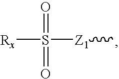

wherein [symbol] indicates the point of attachment, Z₁ is absent and Rₓ is an optionally substituted group selected from (C₁-C₆)alkyl, (C₃-C₆)cycloalkyl, (C₃-C₆)cycloalkyl(C₁-C₃)alkylene, aryl, heteroaryl, benzyl and heteroaryl(C₁-C₃)alkyl; or R₁ is

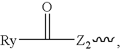

wherein [symbol] indicates the point of attachment, Z₂ is absent, R_{y} is an optionally substituted group selected from (C₁-C₆)alkyl, (C₁-C₆)alkoxy, (C₃-C₆)cycloalkyl, (C₃-C₆)cycloalkyl(C₁-C₃)alkylene, aryl, heterocycloalkyl, heteroaryl, benzyl, heteroaryl(C₁-C₃)alkyl, heterocycloalkyl(C₁-C₃)alkylene and NR′R″, wherein R′ and R″ are each independently H or optionally substituted (C₁-C₆)alkyl, phenyl or benzyl or R′ and R″ taken together with the nitrogen to which they are attached form an optionally substituted 4, 5 or 6-membered ring;

and wherein the optional substituents that may be present on R₁ are independently selected from (C₁-C₃)alkyl, (C₁-C₃)alkoxy, halo, cyano, —OH, —CF₃, —OCF₃, —NR⁶R⁷ (for example, —NH₂, —NH(C₁-C₆)alkyl or —N[(C₁-C₆)alkyl)]₂), —NHCOR⁶, —N[(C₁-C₆)alkyl]C(O)R⁶, —C(O)NR⁶R⁷, —C(O)(C₁-C₄)alkyl, —SO₂(C₁-C₄)alkyl and —SO₂NR⁶R⁷; wherein R⁶ and R⁷ are independently selected from hydrogen and (C₁-C₄)alkyl, or R⁶ and R₇ together with the nitrogen to which they are attached form a 4- to 6-membered heterocycloalkyl group, for example an azetidinyl, pyrrolidinyl, piperidinyl, piperazinyl or morpholinyl ring;

or two adjacent substituents on an aryl group within an R₁ group form a (C₁-C₄)alkylenedioxy group such as methylenedioxy.

In another embodiment of the invention X is NR₁ and R₁ is selected from one of the groups (1) to (5) below:
(1) optionally substituted (C₁-C₄)alkyl; or
(2) optionally substituted aralkyl selected from optionally substituted benzyl or phenylethyl; or
(3) optionally substituted heteroaryl(C₁-C₃)alkyl (for example, optionally substituted 1H-pyrazolyl(C₁-C₃)alkyl, 1,3-thiazolyl(C₁-C₃)alkyl, isoxazolyl(C₁-C₃)alkyl, 1H-1,2,4-triazolyl(C₁-C₃)alkyl, pyridinyl(C₁-C₃)alkyl, benzisoxazolyl(C₁-C₃)alkyl or benzimidazolyl(C₁-C₃)alkyl); or
(4)

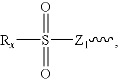

wherein [symbol] indicates the point of attachment, Z₁ is absent, and Rₓ is an optionally substituted group selected from (i) to (vii):
   (i) optionally substituted (C₁-C₄)alkyl;
   (ii) optionally substituted (C₃-C₆)cycloalkyl (for example cyclopropyl, cyclobutyl, cyclopentyl or cyclohexyl);
   (iii) optionally substituted (C₃-C₆)cycloalkyl(C₁-C₃)alkylene (for example optionally substituted cyclopropylmethyl, cyclobutylmethyl, cyclopentylmethyl or cyclohexylmethyl);
   (iv) optionally substituted phenyl;
   (v) optionally substituted heteroaryl (for example an optionally substituted 5 or 6 membered heteroaryl such as optionally substituted thienyl, 1H-pyrazolyl, 1H-imidazolyl, 1,3-thiazolyl, isoxazolyl, 1,2,5-thiadiazolyl, 1,2,3-thiadiazolyl, furanyl, pyrrolyl, pyridinyl or pyrazinyl; or an optionally substituted bicyclic heteroaryl group such as optionally substituted isoquinolinyl, quinolinyl, cinnolinyl, 1H-benzimidazolyl, benzofuranyl, 2,1,3-benzoxadiazolyl, 2,1-benzisoxazolyl, 1H-indazolyl or 1H-indolyl);
   (vi) optionally substituted benzyl; and
   (vii) optionally substituted heteroaryl(C₁-C₃)alkylene (for example optionally substituted 1H-pyrazolyl(C₁-C₃)alkyl, 1,3-thiazolyl(C₁-C₃)alkyl, isoxazolyl(C₁-C₃)alkyl, 1H-1,2,4-triazolyl(C₁-C₃)alkyl, pyridinyl(C₁-C₃)alkyl, benzisoxazolyl(C₁-C₃)alkyl or benzimidazolyl(C₁-C₃)alkyl); or
(5)

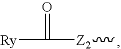

wherein [symbol] indicates the point of attachment, Z₂ is absent, and R_{y} is an optionally substituted group selected from (i) to (x):
   (i) optionally substituted (C₁-C₄)alkyl;
   (ii) optionally substituted (C₃-C₆)cycloalkyl (for example optionally substituted cyclopropyl, cyclobutyl, cyclopentyl or cyclohexyl);
   (iii) optionally substituted (C₃-C₆)cycloalkyl(C₁-C₃)alkylene (for example optionally substituted cyclopropylmethyl, cyclobutylmethyl, cyclopentylmethyl or cyclohexylmethyl);
   (iv) optionally substituted heterocycloalkyl (for example optionally substituted 1,1-dioxotetrahydrothiopyranyl, tetrahydropyranyl, 1,1-dioxotetrahydrothienyl or piperidinyl);
   (v) optionally substituted phenyl;
   (vi) optionally substituted heteroaryl (for example an optionally substituted 5 or 6 membered heteroaryl such as optionally substituted thienyl, 1H-pyrazolyl, 1H-imidazolyl, 1,3-thiazolyl, isoxazolyl, 1,2,5-thiadiazolyl, 1,2,3-thiadiazolyl, furanyl, pyrrolyl, pyridinyl or pyrazinyl; or an optionally substituted bicyclic heteroaryl group such as optionally substituted isoquinolinyl, quinolinyl, cinnolinyl, 1H-benzimidazolyl, benzofuranyl, 2,1,3-benzoxadiazolyl, 2,1-benzisoxazolyl, 1H-indazolyl or 1H-indolyl);
   (vii) optionally substituted benzyl;
   (viii) optionally substituted heteroaryl(C₁-C₃)alkyl (for example optionally substituted 1H-pyrazolyl(C₁-C₃)alkyl, 1,3-thiazolyl(C₁-C₃)alkyl, isoxazolyl(C₁-C₃)alkyl, 1H-1,2,4-triazolyl(C₁-C₃)alkyl, pyridinyl(C₁-C₃)alkyl, benzisoxazolyl(C₁-C₃)alkyl or benzimidazolyl(C₁-C₃)alkyl);
   (ix) optionally substituted heterocycloalkyl(C₁-C₃)alkyl (for example optionally substituted 1,1-dioxotetrahydrothiopyranylmethyl, 1,1-dioxotetrahydrothienylmethyl or tetrahydropyranylmethyl); and
   (x) NR′R″, wherein R′ and R″ are each independently H or optionally substituted (C₁-C₄)alkyl, phenyl or benzyl or R′ and R″ taken together with the nitrogen to which they are attached form an optionally substituted azetidinyl, pyrrolidinyl, piperidinyl or morpholinyl ring;
   and wherein the optional substituents that may be present on R₁ are as hereinbefore defined, for example the optional substituents on any alkyl, cycloalkyl, cycloalkyl-alkylene, heterocycloalkyl or heterocycloalkyl(C₁-C₃)alkyl group in R₁ are independently selected from 1 or more (for example 1, 2 or 3) substituents selected from hydroxy, cyano, —CF₃, (C₁-C₃)alkoxy, —NR₆R₇ (for example, —NH₂, —NH(C₁-C₃)alkyl or —N[(C₁-C₃)alkyl)₂), —C(O)NR⁶R⁷, —NHC(O)R⁶ or —N[(C₁-C₆)alkyl]C(O)R₆; and wherein the optional substituents which may be present on any phenyl, benzyl, heteroaryl or heteroaryl(C₁-C₃)alkyl group in R₁ are independently selected from 1 or more (for example 1, 2 or 3) substituents selected from (C₁-C₃)alkyl, (C₁-C₃)haloalkyl (such as CF₃), (C₁-C₃)alkoxy, (C₁-C₃)alkylthio, halo, nitro, cyano, hydroxy, —C(O)OR₆ (for example —C(O)OH and —C(O)O(C₁-C₆)alkyl), —NR₆R₇ (for example, —NH₂, —NH(C₁-C₆)alkyl or —N[(C₁-C₆)alkyl)₂), —C(O)NR⁶R⁷, —NHC(O)R⁶, —N[(C₁-C₆)alkyl]C(O)R₆, —SO₂R₆, hydroxy-(C₁-C₃)alkyl-, (C₁-C₃)alkoxy-(C₁-C₃)alkyl- and NR₆R₇—(C₁-C₃)alkyl-; wherein R₆ and R₇ are independently hydrogen or (C₁-C₄)alkyl, or R₆ and R₇ together with the nitrogen to which they are attached form a 4- to 6-membered ring (for example a monocyclic nitrogen containing heterocycloalkyl group, such as azetidinyl, pyrrolidinyl, piperidinyl, piperazinyl or morpholinyl); or two adjacent substituents on a phenyl ring in R₁ form a methylenedioxy or ethylenedioxy group.

In another embodiment of the invention X is NR₁ and R₁ is selected from optionally substituted aralkyl (for example benzyl); or R₁ is

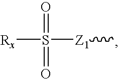

wherein [symbol] indicates the point of attachment, Z₁ is absent and Rₓ is an optionally substituted group selected from (C₁-C₆)alkyl, (C₃-C₆)cycloalkyl, (C₃-C₆)cycloalkyl(C₁-C₆)alkylene, aryl, heteroaryl benzyl and heteroarylmethyl; or R₁ is

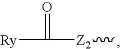

wherein [symbol] indicates the point of attachment, Z₂ is absent, R_{y} is an optionally substituted group selected from (C₁-C₆)alkyl, (C₁-C₆)alkoxy, (C₃-C₆)cycloalkyl, (C₃-C₆)cycloalkyl(C₁-C₆)alkylene, aryl, heteroaryl, benzyl and heteroarylmethyl;

and wherein the optional substituents that may be present on R₁ are independently selected from (C₁-C₃)alkyl, (C₁-C₃)alkoxy, phenyl, halo, cyano, —OH, —CF₃, —OCF₃, —NR⁶R⁷ (for example, —NH₂, —NH(C₁-C₆)alkyl or —N[(C₁-C₆)alkyl)]₂), —NHCOR⁶, —N[(C₁-C₆)alkyl]C(O)R⁶, —C(O)NR⁶R⁷, —C(O)(C₁-C₄)alkyl, —SO₂(C₁-C₄)alkyl and —SO₂NR⁶R⁷; wherein R⁶ and R⁷ are independently selected from hydrogen and (C₁-C₄)alkyl;

or two adjacent substituents on an aryl group within an R₁ group form a (C₁-C₄)alkylenedioxy group such as methylenedioxy.

In another embodiment of the invention X is NR₁ and R₁ is selected from one of the groups (1) to (4) below:
(1) optionally substituted benzyl; or
(2) optionally substituted heteroarylmethyl (for example, optionally substituted isoxazolylmethyl);
(3)

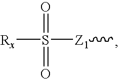

wherein [symbol] indicates the point of attachment, Z₁ is absent, and Rₓ is an optionally substituted group selected from (i) to (iii):
   (i) optionally substituted (C₁-C₄)alkyl (for example optionally substituted methyl, ethyl, propyl, isopropyl, butyl isobutyl or tert butyl);
   (ii) optionally substituted phenyl; or
   (iii) optionally substituted heteroaryl (for example an optionally substituted 5 or 6 membered heteroaryl such as optionally substituted thienyl, 1H-pyrazolyl or pyridinyl; or
(4)

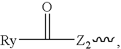

wherein [symbol] indicates the point of attachment, Z₂ is absent, and R_{y} is an optionally substituted group selected from (i) to (x):
   (i) optionally substituted (C₁-C₄)alkyl (for example (for example optionally substituted methyl, ethyl, propyl, isopropyl, butyl isobutyl or tert butyl);
   (ii) optionally substituted (C₃-C₆)cycloalkyl (for example optionally substituted cyclohexyl);
   (iii) optionally substituted (C₃-C₆)cycloalkylmethyl (for example optionally substituted cyclohexylmethyl);
   (iv) optionally substituted heterocycloalkyl (for example optionally substituted 1,1-dioxotetrahydrothiopyranyl or tetrahydropyranyl);
   (v) optionally substituted phenyl;
   (vi) optionally substituted heteroaryl (for example an optionally substituted 5 or 6 membered heteroaryl such as optionally substituted thienyl, 1H-pyrazolyl, 1H-imidazolyl, 1,3-thiazolyl, isoxazolyl, 1,2,5-thiadiazolyl, 1,2,3-thiadiazolyl, furanyl, pyrrolyl or pyridinyl; or an optionally substituted bicyclic heteroaryl group such as optionally substituted isoquinolinyl, quinolinyl, cinnolinyl, 1H-benzimidazolyl, benzofuranyl, 2,1,3-benzoxadiazolyl, 2,1-benzisoxazolyl, 1H-indazolyl or 1H-indolyl);
   (vii) optionally substituted benzyl;
   (viii) optionally substituted heteroarylmethyl (for example optionally substituted 1H-pyrazolylmethyl, 1,3-thiazolylmethyl or 1,2-benzisoxazolylmethyl(C₁-C₃)alkyl;
   (ix) optionally substituted heterocycloalkylmethyl (for example optionally substituted 1,1-dioxotetrahydrothienylmethyl); and
   (x) NR′R″, wherein R′ is H and R″ is optionally substituted phenyl or benzyl;
and wherein the optional substituents that may be present on R₁ are as hereinbefore defined, for example the optional substituents on any alkyl, cycloalkyl, cycloalkyl-alkylene, heterocycloalkyl or heterocycloalkyl(C₁-C₃)alkyl group in R₁ are independently selected from 1 or more (for example 1, 2 or 3) substituents selected from hydroxy, cyano, —CF₃, (C₁-C₃)alkoxy, or —NHC(O)R⁶; and the optional substituents that may be present on any phenyl, benzyl, heteroaryl or heteroarylmethyl group in R₁ are independently selected from 1 or more (for example 1, 2 or 3) substituents selected from (C₁-C₃)alkyl, (C₁-C₃)alkoxy, halo (such as fluoro, chloro or bromo), cyano, hydroxy, —CF₃, —NR₆R₇ (for example, —NH₂, —NH(C₁-C₃)alkyl or —N[(C₁-C₃)alkyl)₂), —NHC(O)R⁶, —SO₂R₆, hydroxy-(C₁-C₃)alkyl- and (C₁-C₃)alkoxy-(C₁-C₃)alkyl-, wherein R₆ and R₇ are independently hydrogen or (C₁-C₃)alkyl, or R₆ and R₇ together with the nitrogen to which they are attached form a azetidinyl, pyrrolidinyl or piperidinyl ring); or two adjacent substituents on a phenyl ring in R₁ form a methylenedioxy or ethylenedioxy group.

In one embodiment of the invention X is NR₁ and R₁ is an optionally substituted group selected from (C₁-C₆)alkyl, (C₃-C₆)cycloalkyl, heterocycloalkyl, (C₃-C₆)cycloalkyl(C₁-C₆)alkyl, heterocycloalkyl(C₁-C₆)alkyl, aryl, heteroaryl, aralkyl, or heteroaralkyl.

In a further embodiment of the invention X is NR₁ and R₁ is aralkyl which optionally bears 1, 2 or 3 substituents selected from (C₁-C₃)alkyl, (C₁-C₃)alkoxy, halo, cyano, —OH, —CF₃, —OCF₃, —NR⁶R⁷ (for example, —NH₂, —NH(C₁-C₆)alkyl or —N[(C₁-C₆)alkyl)]₂), —NHCOR⁶, —N[(C₁-C₆)alkyl]C(O)R⁶, —C(O)NR⁶R⁷, —C(O)(C₁-C₄)alkyl, —SO₂(C₁-C₄)alkyl and —SO₂NR⁶R⁷; wherein R⁶ and R⁷ are independently selected from hydrogen and (C₁-C₄)alkyl, or R₆ and R₇ together with the nitrogen to which they are attached form a 4- to 6-membered heterocycloalkyl group, for example an azetidinyl, pyrrolidinyl, piperidinyl, piperazinyl or morpholinyl ring;

or two adjacent substituents on an aryl group within an R₁ group form a (C₁-C₄)alkylenedioxy group such as methylenedioxy.

In a further embodiment of the invention X is NR₁ and R₁ is benzyl which is optionally substituted as hereinbefore defined, for example R₁ is benzyl optionally substituted by 1, 2 or 3 substituents selected from (C₁-C₃)alkyl, (C₁-C₃)alkoxy, halo (such as fluoro, chloro or bromo), cyano, hydroxy, —CF₃, —NHC(O)R⁶, —SO₂R₆, hydroxy-(C₁-C₃)alkyl- and (C₁-C₃)alkoxy-(C₁-C₃)alkyl-, wherein R₆ and R₇ are independently hydrogen or (C₁-C₃)alkyl; or two adjacent substituents on a phenyl ring in R₁ form a methylenedioxy or ethylenedioxy group.

In another embodiment X is NR₁ and R₁ is benzyl.

In a further embodiment of the invention R₁ is phenyl.

Other specific values for R₁ include:

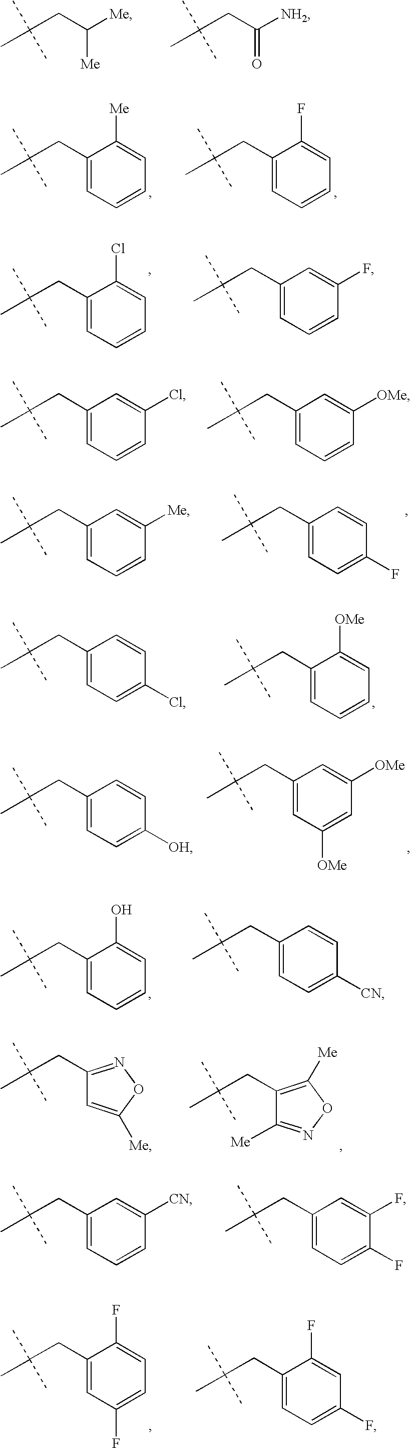

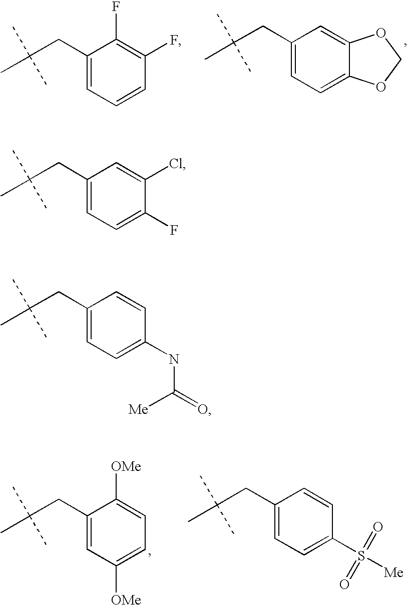

In still other compounds of the invention X is NR₁ and R₁ is

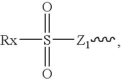

wherein Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl.

In another embodiment of the invention X is NR₁ and R₁ is

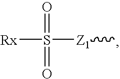

wherein [symbol] indicates the point of attachment, Z₁ is absent and Rₓ is an optionally substituted group selected from (i) to (vii):
(i) optionally substituted (C₁-C₄)alkyl;
(ii) optionally substituted (C₃-C₆)cycloalkyl (for example cyclopropyl, cyclobutyl, cyclopentyl or cyclohexyl);
(iii) optionally substituted (C₃-C₆)cycloalkyl(C₁-C₃)alkylene (for example optionally substituted cyclopropylmethyl, cyclobutylmethyl, cyclopentylmethyl or cyclohexylmethyl);
(iv) optionally substituted phenyl;
(v) optionally substituted heteroaryl (for example an optionally substituted 5 or 6 membered heteroaryl such as optionally substituted thienyl, 1H-pyrazolyl, 1H-imidazolyl, 1,3-thiazolyl, isoxazolyl, 1,2,5-thiadiazolyl, 1,2,3-thiadiazolyl, furanyl, pyrrolyl, pyridinyl or pyrazinyl; or an optionally substituted bicyclic heteroaryl group such as optionally substituted isoquinolinyl, quinolinyl, cinnolinyl, 1H-benzimidazolyl, benzofuranyl, 2,1,3-benzoxadiazolyl, 2,1-benzisoxazolyl, 1H-indazolyl or 1H-indolyl);
(vi) optionally substituted benzyl; and
(vii) optionally substituted heteroaryl(C₁-C₃)alkylene (for example optionally substituted 1H-pyrazolyl(C₁-C₃)alkyl, 1,3-thiazolyl(C₁-C₃)alkyl, isoxazolyl(C₁-C₃)alkyl, 1H-1,2,4-triazolyl(C₁-C₃)alkyl, pyridinyl(C₁-C₃)alkyl, benzisoxazolyl(C₁-C₃)alkyl or benzimidazolyl(C₁-C₃)alkyl);

wherein Rₓ is optionally substituted as hereinbefore defined. For example the optional substituents that may be present on Rₓ are independently selected from (C₁-C₃)alkyl, (C₁-C₃)alkoxy, halo, cyano, —OH, —CF₃, —OCF₃, —NR⁶R⁷ (for example, —NH₂, —NH(C₁-C₆)alkyl or —N[(C₁-C₆)alkyl)]₂), —NHCOR⁶, —N[(C₁-C₆)alkyl]C(O)R⁶, —C(O)NR⁶R⁷, —C(O)(C₁-C₄)alkyl, —SO₂(C₁-C₄)alkyl and —SO₂NR⁶R⁷; wherein R⁶ and R⁷ are independently selected from hydrogen and (C₁-C₄)alkyl, or R₆ and R₇ together with the nitrogen to which they are attached form a 4- to 6-membered heterocycloalkyl group, for example an azetidinyl, pyrrolidinyl, piperidinyl, piperazinyl or morpholinyl ring;

or two adjacent substituents on an aryl group within an Rₓ group form a (C₁-C₄)alkylenedioxy group such as methylenedioxy.

A specific value for

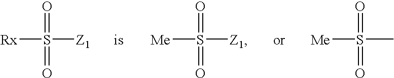

wherein Z₁ is absent.

Another specific value for

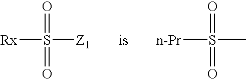

wherein Z₁ is absent.

Other specific values for

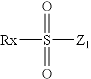

include the following groups:

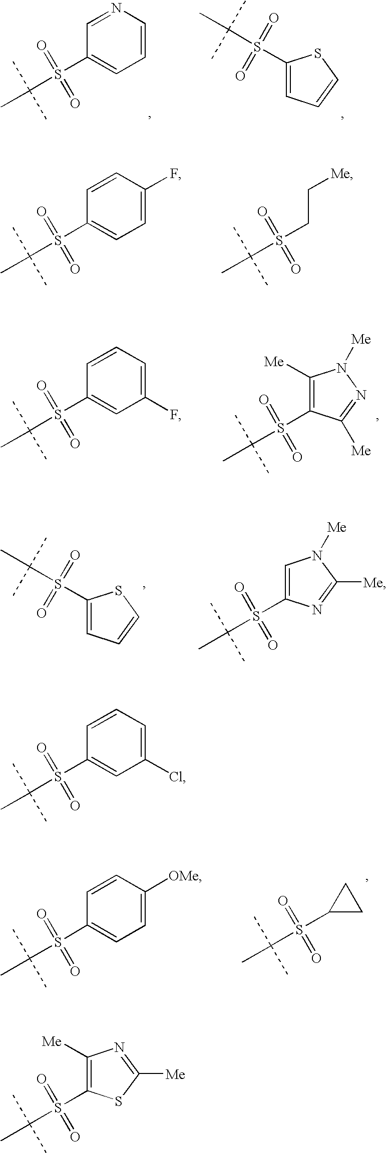

In another embodiment of the invention X is NR₁ and R₁ is

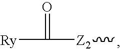

wherein [symbol] indicates the point of attachment, Z₂ is an optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent. R_{y} is an optionally substituted group selected from (C₁-C₆)alkyl, (C₁-C₆)alkoxy, (C₃-C₆)cycloalkyl, heterocycloalkyl, (C₃-C₆)cycloalkyl(C₁-C₆)alkylene, heterocycloalkyl(C₁-C₆)alkyl, aryl, heteroaryl, aralkyl, heteroaralkyl, or NR′R″, wherein R′ and R″ are each independently H or (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring.

In another embodiment of the invention X is NR₁ and R₁ is

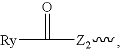

wherein [symbol] indicates the point of attachment Z₂ is absent and R_{y} is an optionally substituted group selected from (i) to (x):
(i) optionally substituted (C₁-C₄)alkyl;
(ii) optionally substituted (C₃-C₆)cycloalkyl (for example optionally substituted cyclopropyl, cyclobutyl, cyclopentyl or cyclohexyl);
(iii) optionally substituted (C₃-C₆)cycloalkyl(C₁-C₃)alkylene (for example optionally substituted cyclopropylmethyl, cyclobutylmethyl, cyclopentylmethyl or cyclohexylmethyl);
(iv) optionally substituted heterocycloalkyl (for example optionally substituted 1,1-dioxotetrahydrothiopyranyl, tetrahydropyranyl, 1,1-dioxotetrahydrothienyl or piperidinyl);
(v) optionally substituted phenyl;
(vi) optionally substituted heteroaryl (for example an optionally substituted 5 or 6 membered heteroaryl such as optionally substituted thienyl, 1H-pyrazolyl, 1H-imidazolyl, 1,3-thiazolyl, isoxazolyl, 1,2,5-thiadiazolyl, 1,2,3-thiadiazolyl, furanyl, pyrrolyl, pyridinyl or pyrazinyl; or an optionally substituted bicyclic heteroaryl group such as optionally substituted isoquinolinyl, quinolinyl, cinnolinyl, 1H-benzimidazolyl, benzofuranyl, 2,1,3-benzoxadiazolyl, 2,1-benzisoxazolyl, 1H-indazolyl or 1H-indolyl);
(vii) optionally substituted benzyl;
(viii) optionally substituted heteroaryl(C₁-C₃)alkyl (for example optionally substituted 1H-pyrazolyl(C₁-C₃)alkyl, 1,3-thiazolyl(C₁-C₃)alkyl, isoxazolyl(C₁-C₃)alkyl, 1H-1,2,4-triazolyl(C₁-C₃)alkyl, pyridinyl(C₁-C₃)alkyl, benzisoxazolyl(C₁-C₃)alkyl or benzimidazolyl(C₁-C₃)alkyl);
(ix) optionally substituted heterocycloalkyl(C₁-C₃)alkyl (for example optionally substituted 1,1-dioxotetrahydrothiopyranylmethyl, 1,1-dioxotetrahydrothienylmethyl or tetrahydropyranylmethyl); and
(x) NR′R″, wherein R′ and R″ are each independently H or optionally substituted (C₁-C₄)alkyl, phenyl or benzyl or R′ and R″ taken together with the nitrogen to which they are attached form an optionally substituted azetidinyl, pyrrolidinyl, piperidinyl or morpholinyl ring;

and wherein the optional substituents that may be present on R_{y} are as hereinbefore defined. For example the optional substituents that may be present on R_{y} are independently selected from (C₁-C₃)alkyl, (C₁-C₃)alkoxy, halo, cyano, —OH, —CF₃, —OCF₃, —NR⁶R⁷ (for example, —NH₂, —NH(C₁-C₆)alkyl or —N[(C₁-C₆)alkyl)]₂), —NHCOR⁶, —N[(C₁-C₆)alkyl]C(O)R⁶, —C(O)NR⁶R⁷, —C(O)(C₁-C₄)alkyl, —SO₂(C₁-C₄)alkyl and —SO₂NR⁶R⁷; wherein R⁶ and R⁷ are independently selected from hydrogen and (C₁-C₄)alkyl, or R₆ and R₇ together with the nitrogen to which they are attached form a 4- to 6-membered heterocycloalkyl group, for example an azetidinyl, pyrrolidinyl, piperidinyl, piperazinyl or morpholinyl ring;

or two adjacent substituents on an aryl group within an R_{y} group form a (C₁-C₄)alkylenedioxy group such as methylenedioxy.

For example, a specific value for

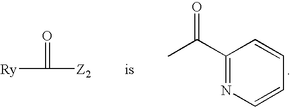

Other specific values for

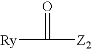

include, for example, the following groups:

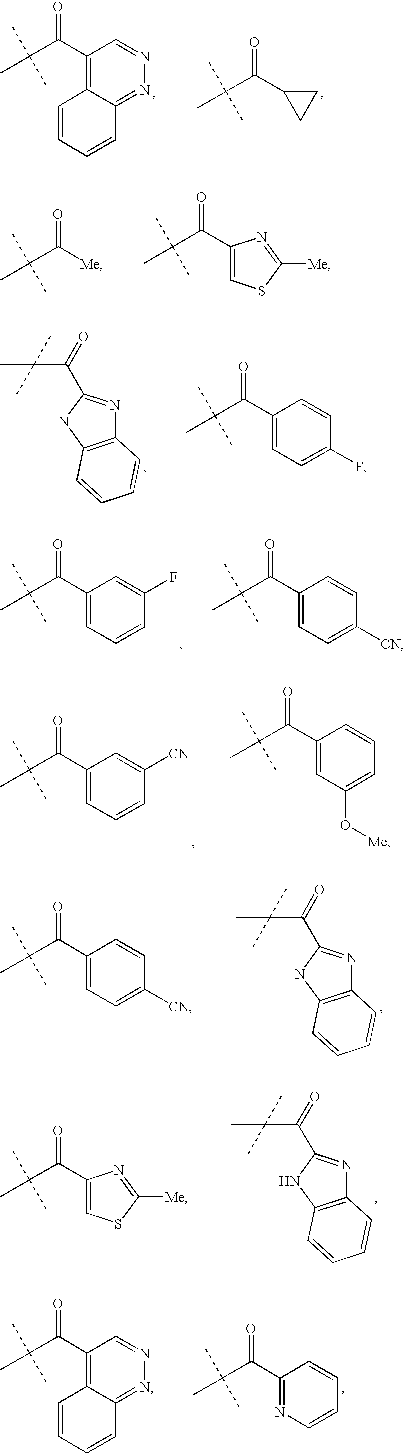

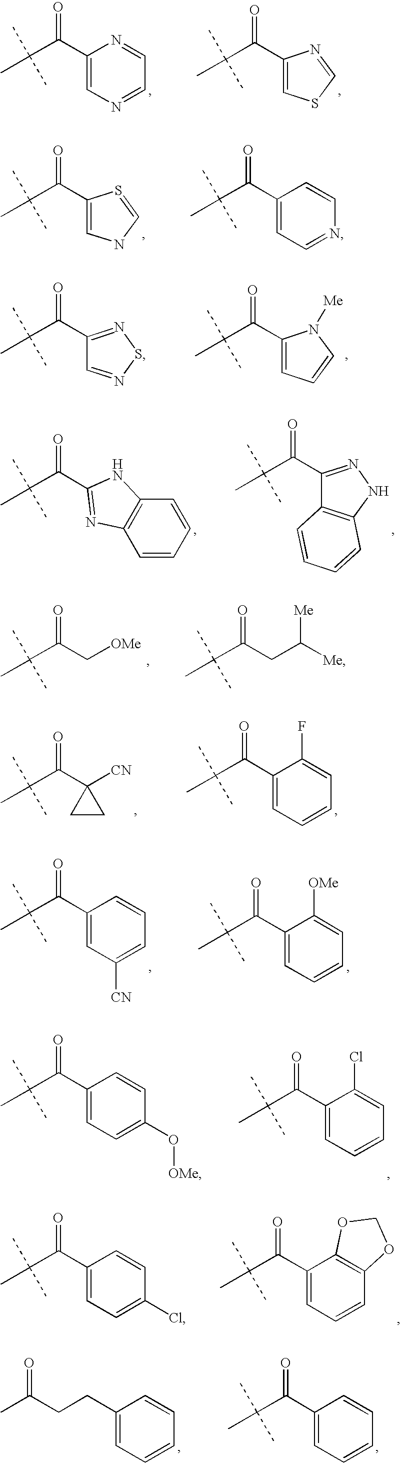

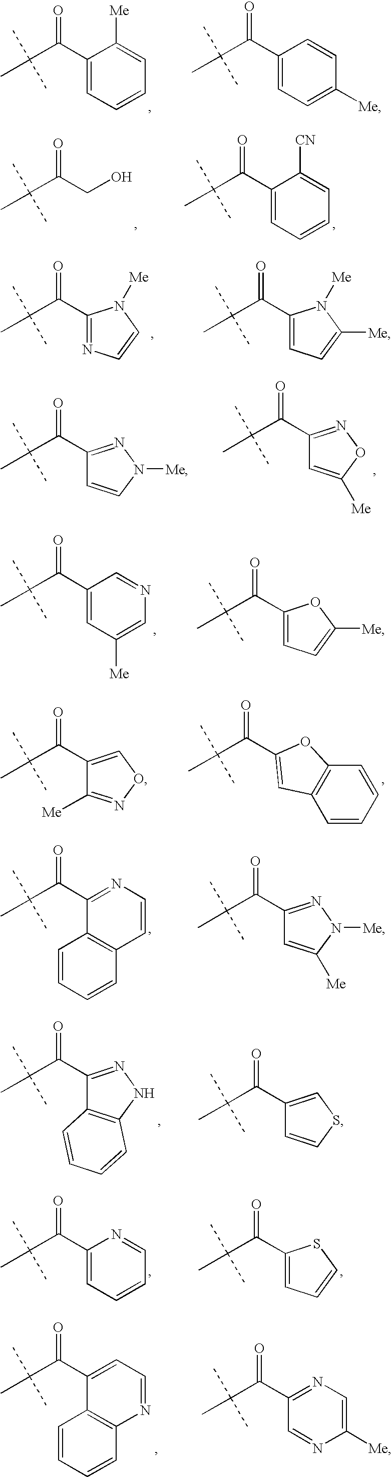

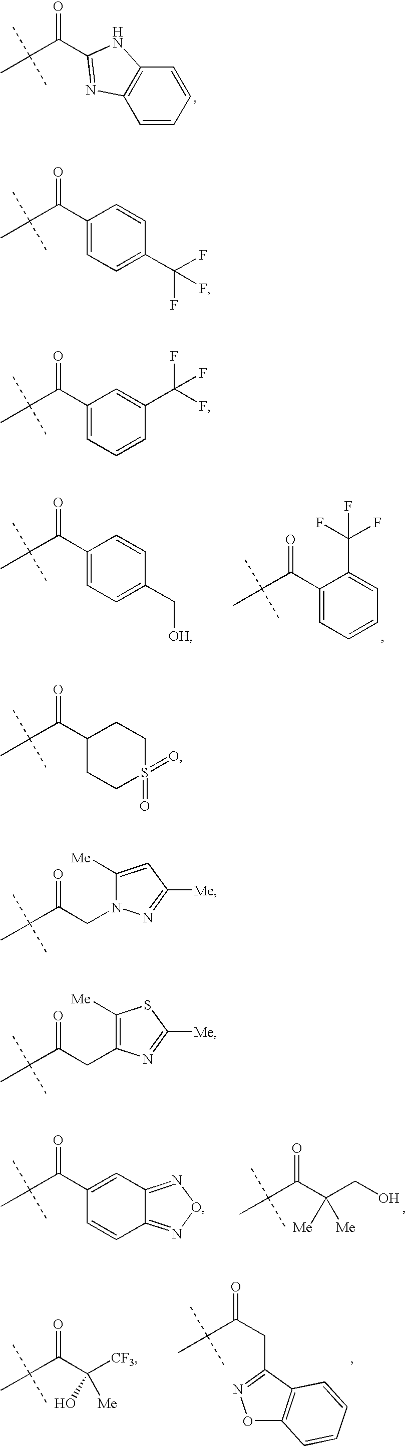

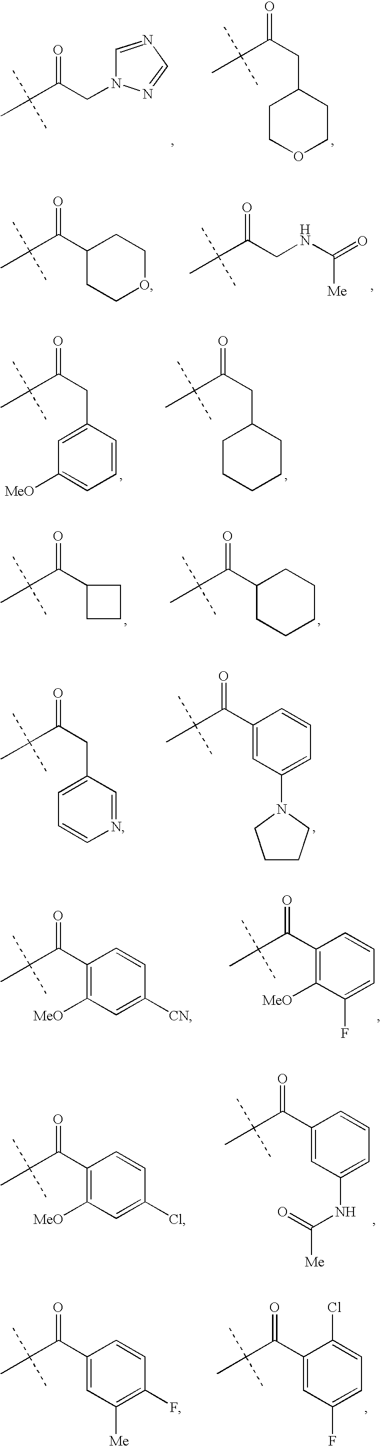

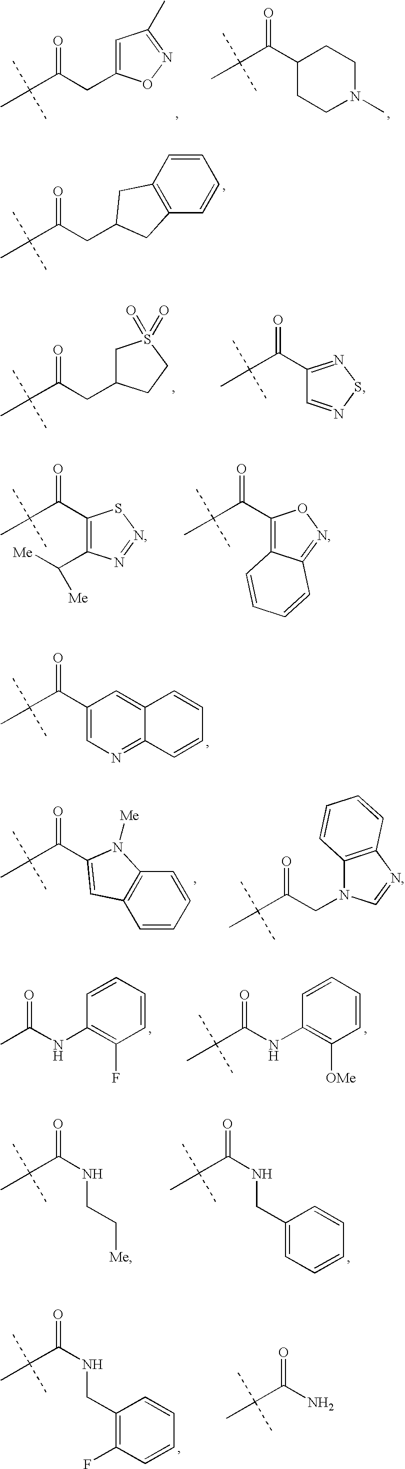

In other compounds of the invention, R₁ is R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring. For example, R₁ₐ is H or (C₁-C₃)alkoxy. For example, a specific value for R₁ is 2-hydroxyethyl. Another specific value for R₁ is 2-methoxyethyl.

In a compound of formula I, a specific value for

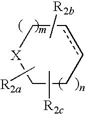

is:

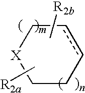

wherein m and n are each independently 0, 1, or 2 and X, R₂ₐ and R_{2b} have any of the values defined herein.

Another specific value for

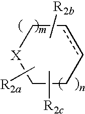

is:

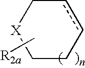

wherein n is 0, 1, or 2 and X, R₂ₐ and R_{2b} have any of the values defined herein.

Other specific values for

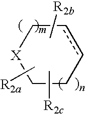

include:

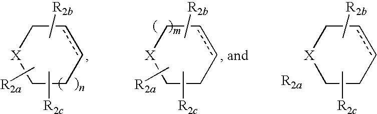

wherein m and n are each independently 0, 1, or 2 and X, R₂ₐ, R_{2b} and R_{2c} have any of the values defined herein.

For example,

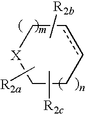

is:

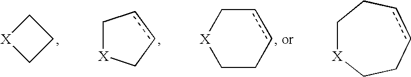

any of which may be substituted by 1, 2, or 3 substitutents R₂ₐ, R_{2b}, and R_{2c} as defined herein, for example 1, 2 or 3 substituents selected from H, halo, hydroxy, (C₁-C₃)alkyl, or (C₁-C₃)alkoxy, or if two of R₂ₐ, R_{2b}, and R_{2c} are attached to the same carbon, they may form oxo; and wherein X has any of the values defined herein.

In a specific embodiment of the invention

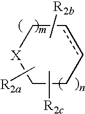

is:

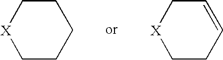

any of which may be substituted by 1, 2, or 3 substitutents R₂ₐ, R_{2b}, and R_{2c} as defined herein; and wherein X has any of the values defined herein.

In a particular embodiment of the invention R₂ₐ, R_{2b}, and R_{2c} are each independently selected from H, halo, (C₁-C₃)alkyl, or (C₁-C₃)alkoxy. More particularly R₂ₐ, R_{2b}, and R_{2c} are independently H or (C₁-C₃)alkyl. For example R₂ₐ, R_{2b}, and R_{2c} are all H.

In a compound of formula I, a specific value for

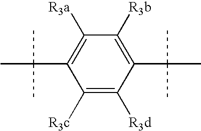

is:

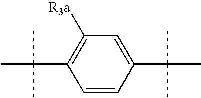

wherein R₃ₐ is as hereinbefore defined.

Other specific values for

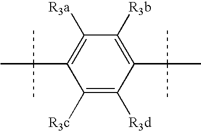

include:

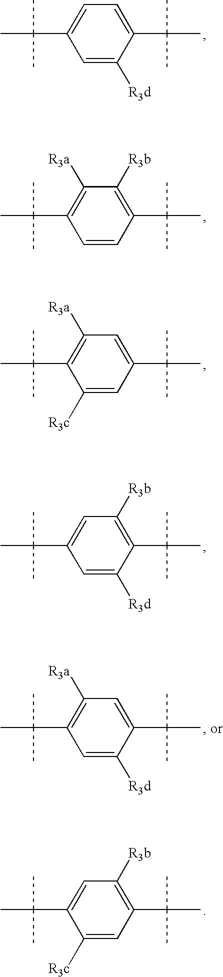

In these specific values, R₃ₐ, R_{3b}, R_{3c}, and R_{3d} are each independently H, halo, (C₁-C₃)alkyl, or (C₁-C₃)alkoxy. Particularly R₃ₐ, R_{3b}, R_{3c}, and R_{3d} are each independently H or (C₁-C₃)alkyl, for example R₃ₐ, R_{3b}, R_{3c}, and R_{3d} are all H.

In one embodiment of the invention R₄ is H or (C₁-C₄)alkyl. For example R₄ is methyl.

In a compound of formula I, a specific value for R₄ is H.

In another embodiment of the invention R₅ is a group of the formula:

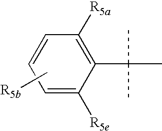

wherein R₅ₐ is chloro or (C₁-C₃)alkyl;

R₅ₑ is H chloro or (C₁-C₃)alkyl;

R_{5b} is H, halo (for example fluoro, chloro or bromo), cyano, (C₁-C₃)alkyl or (C₁-C₃)alkoxy; and

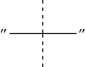

indicates the point of attachment. In this embodiment a particular value for R₅ₐ is chloro and R₅ₑ is selected from chloro and methyl. In this embodiment a particular value for R₅ₑ is chloro or (C₁-C₃)alkyl. In this embodiment a particular value for R_{5b} is H or (C₁-C₃)alkoxy, particularly R_{5b} is H or methoxy. More particularly R_{5b} is H. For example R₅ₐ is chloro, R_{5b} is H and R₅ₑ is chloro or methyl. In another example, R_{5b} is H and R₅ₐ and R₅ₑ are both chloro.

In a compound of formula I, a specific value for R₅ is

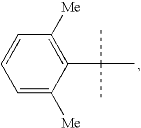

wherein

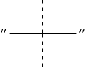

indicates the point of attachment. Other specific values for R₅ include

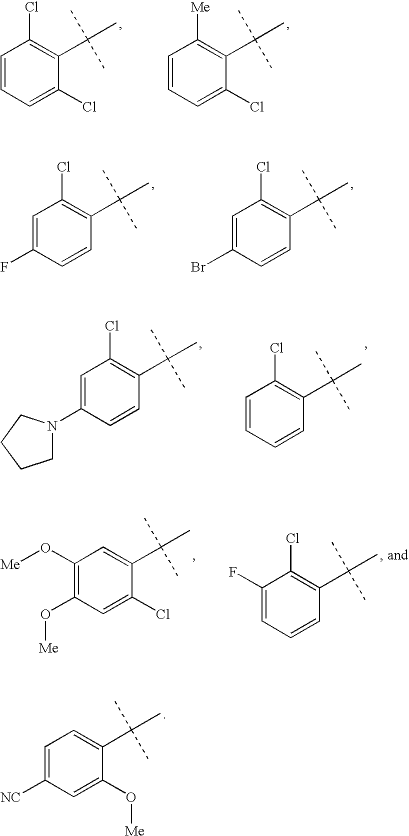

In a compound of formula I, a specific value for R₅ is

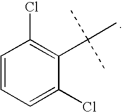

In one embodiment, a compound of formula I is a compound wherein X, R₄, and R₅ are as provided in the preceding paragraphs and

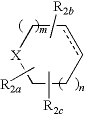

is:

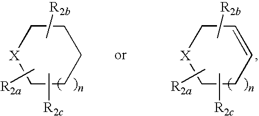

wherein X is O, N—R₁, S(O), or S(O)₂ (particularly X is O or NR₁, more particularly X is NR₁), n is 0, 1 or 2; and R₂ₐ, R_{2b}, R_{2c} and R₁ are as hereinbefore defined; and

the group of the formula:

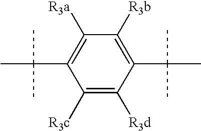

is:

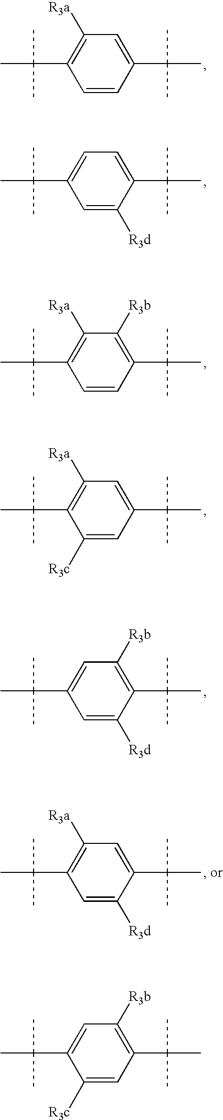

wherein R₃ₐ, R_{3b}, R_{3c}, and R_{3d} are each independently H, halo, (C₁-C₃)alkyl, or (C₁-C₃)alkoxy (particularly R₃ₐ, R_{3b}, R_{3c} and R_{3d} are independently H or methyl, more particularly H).

In another embodiment, a compound of formula I is a compound wherein X, R₄, and R₅ are as provided in the preceding paragraphs and R₂ₐ, R_{2b} and R_{2c}, are each independently H, halo, hydroxyl, (C₁-C₃)alkyl, or (C₁-C₃)alkoxy, or if two of R₂ₐ and R_{2b} are attached to the same carbon, they may form oxo. Particularly R₂ₐ, R_{2b} and R_{2c}, are each independently H, halo, (C₁-C₃)alkyl or (C₁-C₃)alkoxy. More particularly R₂ₐ, R_{2b} and R_{2c}, are each independently H, halo or (C₁-C₃)alkyl. Still more particularly R₂ₐ, R_{2b} and R_{2c}, are all H.

In another embodiment, a compound of formula I is a compound wherein X is O.

In another embodiment, a compound of formula I is a compound wherein X is N—R₁, wherein R₁ is an optionally substituted group selected from aralkyl or heteroaralkyl, or is

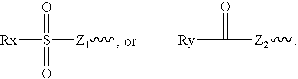

wherein Rₓ, R_{y}, Z₁ and Z₂ have any of the meanings defined herein. When Rₓ is methyl, R₅ is

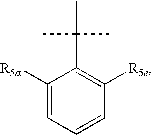

wherein R₅ₐ and R₅ₑ are each independently halo or (C₁-C₃)alkyl.

In another embodiment, a compound of formula I is a compound of formula IA, wherein R₂ₐ, R_{2b}, R₄, and R₅ are as defined above.

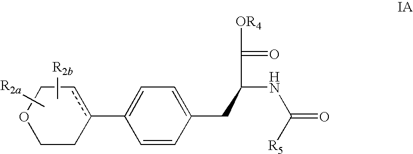

wherein R₂ₐ, R_{2b}, R₄ and R₅ are as defined above;

or a pharmaceutically acceptable salt, prodrug or hydrate thereof.

In this embodiment R₂ₐ and R_{2b} are suitably H, halo or (C₁-C₃)alkyl, more particularly R₂ₐ and R_{2b} are both H.

In another embodiment, a compound of formula I is a compound of formula IB, wherein R₂ₐ, R_{2b}, R₄, and R₅ are as defined above and R_{c} is an optionally substituted group selected from aralkyl, aryl or heteroaryl.

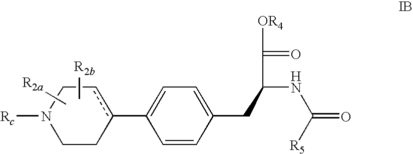

or a pharmaceutically acceptable salt, prodrug or hydrate thereof.

In the compound of the formula IB one particular value for R_{c} is aryl or heteroaryl.

In the compound of the formula IB another value for R_{c} is optionally substituted aralkyl, for example optionally substituted benzyl. For example R_{c} is optionally substituted aralkyl, particularly benzyl, which optionally bears 1 or more (for example 1, 2 or 3) substituents selected from (C₁-C₃)alkyl, (C₁-C₃)haloalkyl (such as CF₃), (C₁-C₃)alkoxy, (C₁-C₃)alkylthio, halo, nitro, cyano, hydroxy, —C(O)OR₆ (for example —C(O)OH and —C(O)O(C₁-C₆)alkyl), —NR₆R₇ (for example, —NH₂, —NH(C₁-C₃)alkyl or —N[(C₁-C₃)alkyl)₂), —C(O)NR⁶R⁷, —NHC(O)R⁶, —N[(C₁-C₃)alkyl]C(O)R₆, —SO₂R₆, hydroxy-(C₁-C₃)alkyl-, (C₁-C₃)alkoxy-(C₁-C₃)alkyl- and NR₆R₇—(C₁-C₃)alkyl-; wherein R₆ and R₇ are independently hydrogen or (C₁-C₃)alkyl, or R₆ and R₇ together with the nitrogen to which they are attached form a 4- to 6-membered ring (for example a monocyclic nitrogen containing heterocycloalkyl group, such as azetidinyl, pyrrolidinyl, piperidinyl, piperazinyl or morpholinyl); or two adjacent substituents on a phenyl ring in R_{c} form a methylenedioxy or ethylenedioxy group.

Particular examples of R_{c} in the compound of the formula IB include benzyl, 2-fluorobenzyl, 4-fluorobenzyl, 2-chlorobenzyl, 3-chlorobenzyl, 4-chlorobenzyl, 2-methylbenzyl, 3-methylbenzyl, 2-methoxybenzyl, 3-methoxybenzyl, 3-cyanobenzyl, 4-hydroxybenzyl, 4-methylsulfonylbenzyl, 3,5-dimethoxybenzyl, 2,5-dimethoxybenzyl, 3,4-difluorobenzyl, 2,5-difluorobenzyl, 3,4-methylenedioxybenzyl, 3-chloro-4-fluorobenzyl or 3-acetylaminobenzyl.

In another embodiment, a compound of formula I is a compound of formula IC, wherein R₂ₐ, R_{2b}, R₄, and R₅ are as defined above and Rₓ is an optionally substituted group selected from (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl.

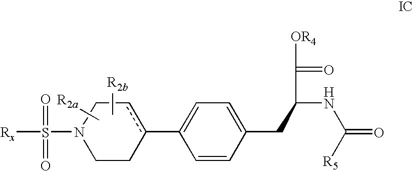

or a pharmaceutically acceptable salt, prodrug or hydrate thereof.

In this embodiment, in the compound of formula IC a particular value for Rₓ is an optionally substituted group selected from (i) to (vii):
(i) optionally substituted (C₁-C₄)alkyl;
(ii) optionally substituted (C₃-C₆)cycloalkyl (for example cyclopropyl, cyclobutyl, cyclopentyl or cyclohexyl);
(iii) optionally substituted (C₃-C₆)cycloalkyl(C₁-C₃)alkylene (for example optionally substituted cyclopropylmethyl, cyclobutylmethyl, cyclopentylmethyl or cyclohexylmethyl);
(iv) optionally substituted phenyl;
(v) optionally substituted heteroaryl (for example an optionally substituted 5 or 6 membered heteroaryl such as optionally substituted thienyl, 1H-pyrazolyl, 1H-imidazolyl, 1,3-thiazolyl, isoxazolyl, 1,2,5-thiadiazolyl, 1,2,3-thiadiazolyl, furanyl, pyrrolyl, pyridinyl or pyrazinyl; or an optionally substituted bicyclic heteroaryl group such as optionally substituted isoquinolinyl, quinolinyl, cinnolinyl, 1H-benzimidazolyl, benzofuranyl, 2,1,3-benzoxadiazolyl, 2,1-benzisoxazolyl, 1H-indazolyl or 1H-indolyl);
(vi) optionally substituted benzyl; and
(vii) optionally substituted heteroaryl(C₁-C₃)alkylene (for example optionally substituted 1H-pyrazolyl(C₁-C₃)alkyl, 1,3-thiazolyl(C₁-C₃)alkyl, isoxazolyl(C₁-C₃)alkyl, 1H-1,2,4-triazolyl(C₁-C₃)alkyl, pyridinyl(C₁-C₃)alkyl, benzisoxazolyl(C₁-C₃)alkyl or benzimidazolyl(C₁-C₃)alkyl).

and wherein the optional substituents on any alkyl, cycloalkyl, cycloalkyl-alkylene, heterocycloalkyl or heterocycloalkyl(C₁-C₃)alkyl group in Rₓ are independently selected from 1 or more (for example 1, 2 or 3) substituents selected from hydroxy, cyano, —CF₃, (C₁-C₃)alkoxy, —NR₆R₇ (for example, —NH₂, —NH(C₁-C₃)alkyl or —N[(C₁-C₃)alkyl)₂), —C(O)NR⁶R⁷, —NHC(O)R⁶ or —N[(C₁-C₃)alkyl]C(O)R₆; and wherein the optional substituents which may be present on any phenyl, benzyl, heteroaryl or heteroaryl(C₁-C₃)alkyl group in Rₓ are independently selected from 1 or more (for example 1, 2 or 3) substituents selected from (C₁-C₃)alkyl, (C₁-C₃)haloalkyl (such as CF₃), (C₁-C₃)alkoxy, (C₁-C₃)alkylthio, halo, nitro, cyano, hydroxy, —C(O)OR₆ (for example —C(O)OH and —C(O)O(C₁-C₆)alkyl), —NR₆R₇ (for example, —NH₂, —NH(C₁-C₆)alkyl or —N[(C₁-C₆)alkyl)₂), —C(O)NR⁶R⁷, —NHC(O)R⁶, —N[(C₁-C₆)alkyl]C(O)R₆, —SO₂R₆, hydroxy-(C₁-C₃)alkyl-, (C₁-C₃)alkoxy-(C₁-C₃)alkyl- and NR₆R₇—(C₁-C₃)alkyl-; wherein R₆ and R₇ are independently hydrogen or (C₁-C₃)alkyl, or R₆ and R₇ together with the nitrogen to which they are attached form a 4- to 6-membered ring (for example a monocyclic nitrogen containing heterocycloalkyl group, such as azetidinyl, pyrrolidinyl, piperidinyl, piperazinyl or morpholinyl); or two adjacent substituents on a phenyl ring in Rₓ form a methylenedioxy or ethylenedioxy group.

Particular examples of Rx in the compound of the formula IC include propyl, butyl, 2-thienyl, 1,3,5-trimethyl-1H-pyrazol-4-yl and 3-pyridinyl,

In another embodiment, a compound of formula I is a compound of formula ID, wherein R₂ₐ, R_{2b}, R₄, and R₅ are as defined above and R_{y} is an optionally substituted group selected from (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or NR′R″, wherein R′ and R″ are each independently H or (C₁-C₆)alkyl, aryl, heteroaryl, aralkyl, or heteroaralkyl, (C₁-C₆)alkoxy, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl or taken together with the nitrogen to which they are attached form an optionally substituted 3, 4, 5, 6, or 7-membered ring.

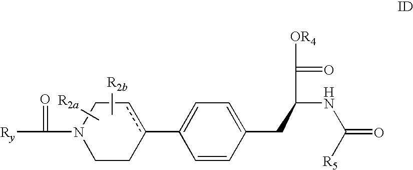

or a pharmaceutically acceptable salt, prodrug or hydrate thereof.

In this embodiment, in the compound of formula ID a particular value for R_{y} is an optionally substituted group selected from (i) to (x):
(i) optionally substituted (C₁-C₄)alkyl;
(ii) optionally substituted (C₃-C₆)cycloalkyl (for example optionally substituted cyclopropyl, cyclobutyl, cyclopentyl or cyclohexyl);
(iii) optionally substituted (C₃-C₆)cycloalkyl(C₁-C₃)alkylene (for example optionally substituted cyclopropylmethyl, cyclobutylmethyl, cyclopentylmethyl or cyclohexylmethyl);
(iv) optionally substituted heterocycloalkyl (for example optionally substituted 1,1-dioxotetrahydrothiopyranyl, tetrahydropyranyl, 1,1-dioxotetrahydrothienyl or piperidinyl);
(v) optionally substituted phenyl;
(vi) optionally substituted heteroaryl (for example an optionally substituted 5 or 6 membered heteroaryl such as optionally substituted thienyl, 1H-pyrazolyl, 1H-imidazolyl, 1,3-thiazolyl, isoxazolyl, 1,2,5-thiadiazolyl, 1,2,3-thiadiazolyl, furanyl, pyrrolyl, pyridinyl or pyrazinyl; or an optionally substituted bicyclic heteroaryl group such as optionally substituted isoquinolinyl, quinolinyl, cinnolinyl, 1H-benzimidazolyl, benzofuranyl, 2,1,3-benzoxadiazolyl, 2,1-benzisoxazolyl, 1H-indazolyl or 1H-indolyl);
(vii) optionally substituted benzyl;
(viii) optionally substituted heteroaryl(C₁-C₃)alkyl (for example optionally substituted 1H-pyrazolyl(C₁-C₃)alkyl, 1,3-thiazolyl(C₁-C₃)alkyl, isoxazolyl(C₁-C₃)alkyl, 1H-1,2,4-triazolyl(C₁-C₃)alkyl, pyridinyl(C₁-C₃)alkyl, benzisoxazolyl(C₁-C₃)alkyl or benzimidazolyl(C₁-C₃)alkyl);
(ix) optionally substituted heterocycloalkyl(C₁-C₃)alkyl (for example optionally substituted 1,1-dioxotetrahydrothiopyranylmethyl, 1,1-dioxotetrahydrothienylmethyl or tetrahydropyranylmethyl); and
(x) NR′R″, wherein R′ and R″ are each independently H or optionally substituted (C₁-C₄)alkyl, phenyl or benzyl or R′ and R″ taken together with the nitrogen to which they are attached form an optionally substituted azetidinyl, pyrrolidinyl, piperidinyl or morpholinyl ring;

and wherein the optional substituents that may be present on R₁ are as hereinbefore defined, for example the optional substituents on any alkyl, cycloalkyl, cycloalkyl-alkylene, heterocycloalkyl or heterocycloalkyl(C₁-C₃)alkyl group in R₁ are independently selected from 1 or more (for example 1, 2 or 3) substituents selected from hydroxy, cyano, —CF₃, (C₁-C₃)alkoxy, -Nr₆R₇ (for example, —NH₂, —NH(C₁-C₃)alkyl or —N[(C₁-C₃)alkyl)₂), —C(O)NR⁶R⁷, —NHC(O)R⁶ or —N[(C₁-C₃)alkyl]C(O)R₆; and wherein the optional substituents which may be present on any phenyl, benzyl, heteroaryl or heteroaryl(C₁-C₃)alkyl group in R₁ are independently selected from 1 or more (for example 1, 2 or 3) substituents selected from (C₁-C₃)alkyl, (C₁-C₃)haloalkyl (such as CF₃), (C₁-C₃)alkoxy, (C₁-C₃)alkylthio, halo, nitro, cyano, hydroxy, —C(O)OR₆ (for example —C(O)OH and —C(O)O(C₁-C₃)alkyl), —NR₆R₇ (for example, —NH₂, —NH(C₁-C₆)alkyl or —N[(C₁-C₆)alkyl)₂), —C(O)NR⁶R⁷, —NHC(O)R⁶, —N[(C₁-C₃)alkyl]C(O)R₆, —SO₂R₆, hydroxy-(C₁-C₃)alkyl-, (C₁-C₃)alkoxy-(C₁-C₃)alkyl- and NR₆R₇—(C₁-C₃)alkyl-; wherein R₆ and R₇ are independently hydrogen or (C₁-C₃)alkyl, or R₆ and R₇ together with the nitrogen to which they are attached form a 4- to 6-membered ring (for example a monocyclic nitrogen containing heterocycloalkyl group, such as azetidinyl, pyrrolidinyl, piperidinyl, piperazinyl or morpholinyl); or two adjacent substituents on a phenyl ring in R₁ form a methylenedioxy or ethylenedioxy group.

Particular examples of R_{y} in the compound of the formula ID include and of (i) to (vii):

(i) methyl, ethyl, propyl, isopropyl, butyl, isobutyl or tert-butyl each of which optionally bears 1 or 2 substituents selected from hydroxy, methoxy, —CF₃, or acetylamino;

(ii) cyclohexyl or cyclohexylmethyl;

(iii) a heterocycloalkyl group selected from 1,1dioxotetrahydrothiopyran-4yl and tetrahydropyran-4-yl;

(iv) an optionally substituted phenyl group selected from phenyl, 3-fluorophenyl, 2-chlorophenyl, 4-chlorophenyl, 3-cyanophenyl, 2-methylphenyl, 3-methoxyphenyl, 3-(trifluoromethyl)phenyl, 4-(trifluoromethyl)phenyl, 4-(hydroxymethyl)phenyl, 3-pyrrolidinylphenyl, 4-cyano-2-methoxyphenyl and 3-fluoro-2-methoxyphenyl;

(v) an optionally substituted heteroaryl group selected from 1-methylpyrrol-2-yl, 1,2-dimethylpyrrol-5-yl, 1-methyl-1H-pyrazol-3-yl, 1,5-dimethyl-1H-pyrazol-3-yl, 1-methyl-1H-imidazol-2-yl, 5-methylisoxazol-3-yl, 3-methylisoxazol-5-yl, 2-thienyl, 3-thienyl, 1,3-thiazol-4-yl, 5-methyl-1,3-thiazol-4-yl, 5-methylfuran-2-yl, 1,2,5-thiadiazol-3-yl, 4-isopropyl-1,2,3-thiadiazol-5-yl, 5-methylpyridin-2-yl, 1H-indazol-3-yl, 1-methyl-1H-indol-2-yl, 2,1,3-benzoxadiazol-5-yl, 2,1-benzisoxazol-3-yl, benzimidazol-2-yl, benzofuran-2-yl, isoquinolin-1-yl, isoquinolin-4-yl, quinolin-3-yl, quinolin-4-yl and cinnolin-4-yl;

(vi) an optionally substituted benzyl group selected from benzyl, 2-fluorobenzyl, 3-fluorobenzyl, 4-chlorobenzyl, 2-methylbenzyl, 2-methoxybenzyl, 3-methoxybenzyl, 2-methoxybenzyl, 2-cyanobenzyl and 3-cyanobenzyl;

(vii) optionally substituted heteroarylmethyl group selected from 3,5-dimethylpyrazol-1-ylmethyl, 2,5-dimethyl-1,3-thiazol-4-ylmethyl and 1,2-benzisoxazol-3-ylmethyl; and

(viii) 1,1-dioxotetrahydrothien-3-ylmethyl.

In further embodiments of the invention, in the compounds of the formulae IA, IB, IC and ID:

R₂ₐ and R_{2b} are independently H or methyl (particularly H);

R₄ is H or (C₁-C₆)alkyl (particularly H);

“-----” is absent or is a bond; and

R₅ is a group of the formula:

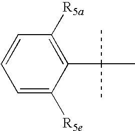

wherein R₅ₐ and R₅ₑ independently are chloro or (C₁-C₃)alkyl (particularly R₅ₐ and R₅ₑ are both chloro); and

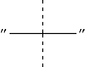

indicates the point of attachment.

In another embodiment, a compound of the invention is a compound of formula II

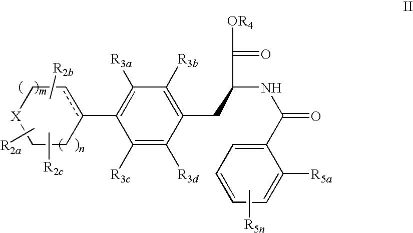

or a pharmaceutical acceptable salt, prodrug, or hydrate thereof, wherein:
X is O, N—R₁, or S(O)ₓ, wherein x is 0, 1, or 2;
m and n are each independently 0, 1, or 2;
“----” is a bond or is absent;
R₁ is (a) H, or an optionally substituted group selected from (C₁-C₆)alkyl, (C₃-C₆)cycloalkyl, heterocycloalkyl, (C₃-C₆)cycloalkyl(C₁-C₆)alkyl, heterocycloalkyl(C₁-C₆)alkyl, aryl, heteroaryl, aralkyl, or heteroaralkyl; or R₁ is
   (b)

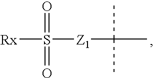

wherein

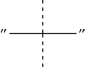

indicates the point of attachment and Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl; or R₁ is
(c)

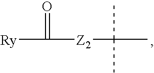

wherein

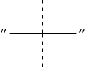

indicates the point of attachment and Z₂ is optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent and R_{y} is an optionally substituted group selected from (C₁-C₆)alkyl, (C₁-C₆)alkoxy, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or NR′R″, wherein R′ and R″ are each independently H or (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring; or R₁ is
(d) R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring;
R₂ₐ, R_{2b}, and R_{2c} are each independently H, halo, hydroxy, (C₁-C₃)alkyl, or (C₁-C₃)alkoxy, or if two of R₂ₐ, R_{2b}, and R_{2c} are attached to the same carbon, they may form oxo;
R₃ₐ, R_{3b}, R_{3c}, and R_{3d} are each independently H, halo, (C₁-C₃)alkyl, or (C₁-C₃)alkoxy;
R₄ is H, (C₁-C₆)alkyl, aryl, heteroaryl, aralkyl, heteroaralkyl; and
R₅ₐ is halo or (C₁-C₆)alkyl and R₅ₙ is one or two groups selected from halo, (C₁-C₆)alkyl, and (C₁-C₆)alkoxy, provided that when X is N—S(O)₂Me, R₅ is

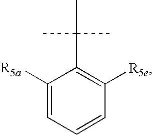

wherein R₅ₐ and R₅ₑ are each independently halo or (C₁-C₃)alkyl.

In another embodiment, a compound of the invention is a compound of formula III

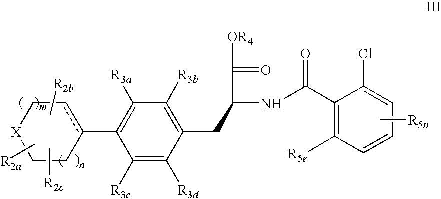

or a pharmaceutical acceptable salt, prodrug, or hydrate thereof, wherein:
X is O, N—R₁, or S(O)ₓ, wherein x is 0, 1, or 2;
m and n are each independently 0, 1, or 2;
“ [symbol] ” is a bond or is absent;
R₁ is (a) H, or an optionally substituted group selected from (C₁-C₆)alkyl, (C₃-C₆)cycloalkyl, heterocycloalkyl, (C₃-C₆)cycloalkyl(C₁-C₆)alkyl, heterocycloalkyl(C₁-C₆)alkyl, aryl, heteroaryl, aralkyl, or heteroaralkyl; or R₁ is
   (b)

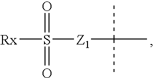

wherein

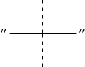

indicates the point of attachment and Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl; or R₁ is
(c)

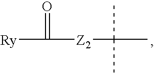

wherein

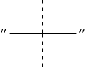

indicates the point of attachment and Z₂ is optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent and R_{y} is an optionally substituted group selected from (C₁-C₆)alkyl, (C₁-C₆)alkoxy, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or NR′R″, wherein R′ and R″ are each independently H or (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring; or R₁ is
(d) R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring;
R₂ₐ, R_{2b}, and R_{2c} are each independently H, halo, hydroxy, (C₁-C₃)alkyl, or (C₁-C₃)alkoxy, or if two of R₂ₐ, R_{2b}, and R_{2c} are attached to the same carbon, they may form oxo;
R₃ₐ, R_{3b}, R_{3c}, and R_{3d} are each independently H, halo, (C₁-C₃)alkyl, or (C₁-C₃)alkoxy;
R₄ is H, (C₁-C₆)alkyl, aryl, heteroaryl, aralkyl, heteroaralkyl; and
R₅ₑ is H, halo, or (C₁-C₆)alkyl and R₅ₙ is one or two groups selected from halo, (C₁-C₆)alkyl, and (C₁-C₆)alkoxy, provided that when X is N—S(O)₂Me, R₅ is

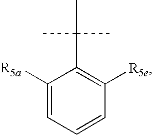

wherein R₅ₐ and R₅ₑ are each independently halo or (C₁-C₃)alkyl.

In another embodiment, a compound of the invention is a compound of formula IV

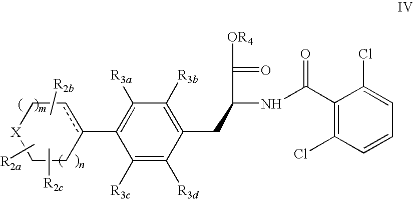

or a pharmaceutical acceptable salt, prodrug, or hydrate thereof, wherein:
X is O, N—R₁, or S(O)ₓ, wherein x is 0, 1, or 2;
m and n are each independently 0, 1, or 2;
“ [symbol] ” is a bond or is absent;
R₁ is (a) H, or an optionally substituted group selected from (C₁-C₆)alkyl, (C₃-C₆)cycloalkyl, heterocycloalkyl, (C₃-C₆)cycloalkyl(C₁-C₆)alkyl, heterocycloalkyl(C₁-C₆)alkyl, aryl, heteroaryl, aralkyl, or heteroaralkyl;
   (b)

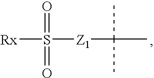

wherein

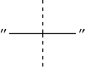

indicates the point of attachment and Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl;
(c)

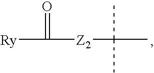

wherein

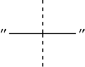

indicates the point of attachment and Z₂ is optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent and R_{y} is an optionally substituted group selected from (C₁-C₆)alkyl, (C₁-C₆)alkoxy, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or NR′R″, wherein R′ and R″ are each independently H or (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring; or R₁ is
(d) R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring;
R₂ₐ, R_{2b}, and R_{2c} are each independently H, halo, hydroxy, (C₁-C₃)alkyl, or (C₁-C₃)alkoxy, or if two of R₂ₐ, R_{2b}, and R_{2c} are attached to the same carbon, they may form oxo;
R₃ₐ, R_{3b}, R_{3c}, and R_{3d} are each independently H, halo, (C₁-C₃)alkyl, or (C₁-C₃)alkoxy; and
R₄ is H, (C₁-C₆)alkyl, aryl, heteroaryl, aralkyl, heteroaralkyl.

Particular compounds of the formulae II, III and IV are those wherein R₂ₐ, R_{2b}, R_{2c} R₃ₐ, R_{3b}, R_{3c}, and R_{3d} are each independently H, halo, (C₁-C₃)alkyl, or (C₁-C₃)alkoxy and R₄ is H. More particularly those compounds of the formulae II, III and IV wherein R₂ₐ, R_{2b}, R_{2c} R₃ₐ, R_{3b}, R_{3c}, R_{3d} and R₄ are all H.

### Preparation of Invention Compounds

Compounds of the invention can, for example, be prepared as provided in Schemes 1-4. In the Schemes, “P” as used, for example, in the structure

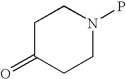

designates a suitable protecting group as found in Green, which is referenced supra. “R₄ₐ” represents an alkyl group such as methyl, ethyl, or the like, or another carboxy protecting group. R₈ is as hereinafter defined in relation to Process (a). The Schemes depict the synthesis of invention compounds incorporating a piperidine ring, but may be readily adapted to homologous invention compounds such as those containing a pyrrolidine or azepine ring, and so on, by using the appropriate cyclic amine starting material.

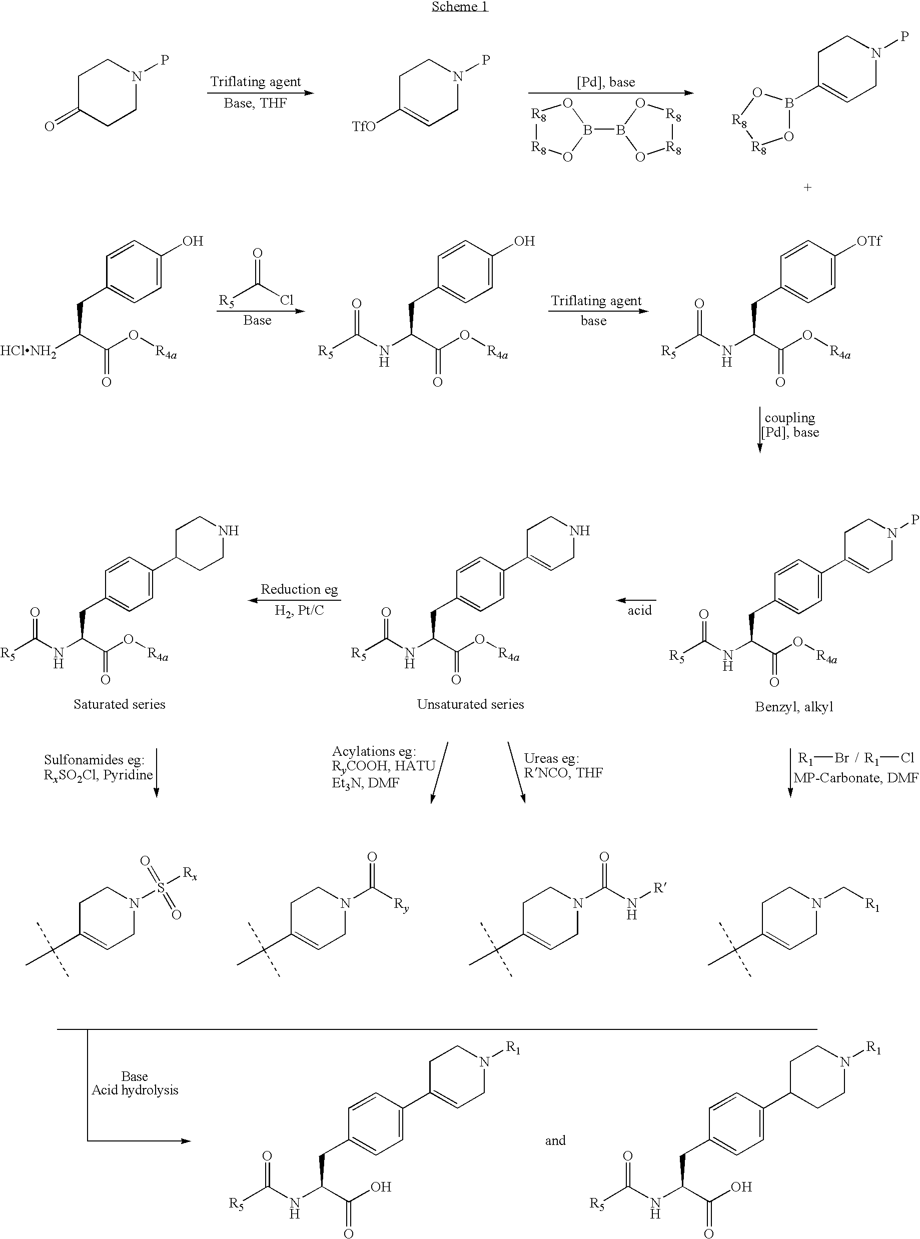

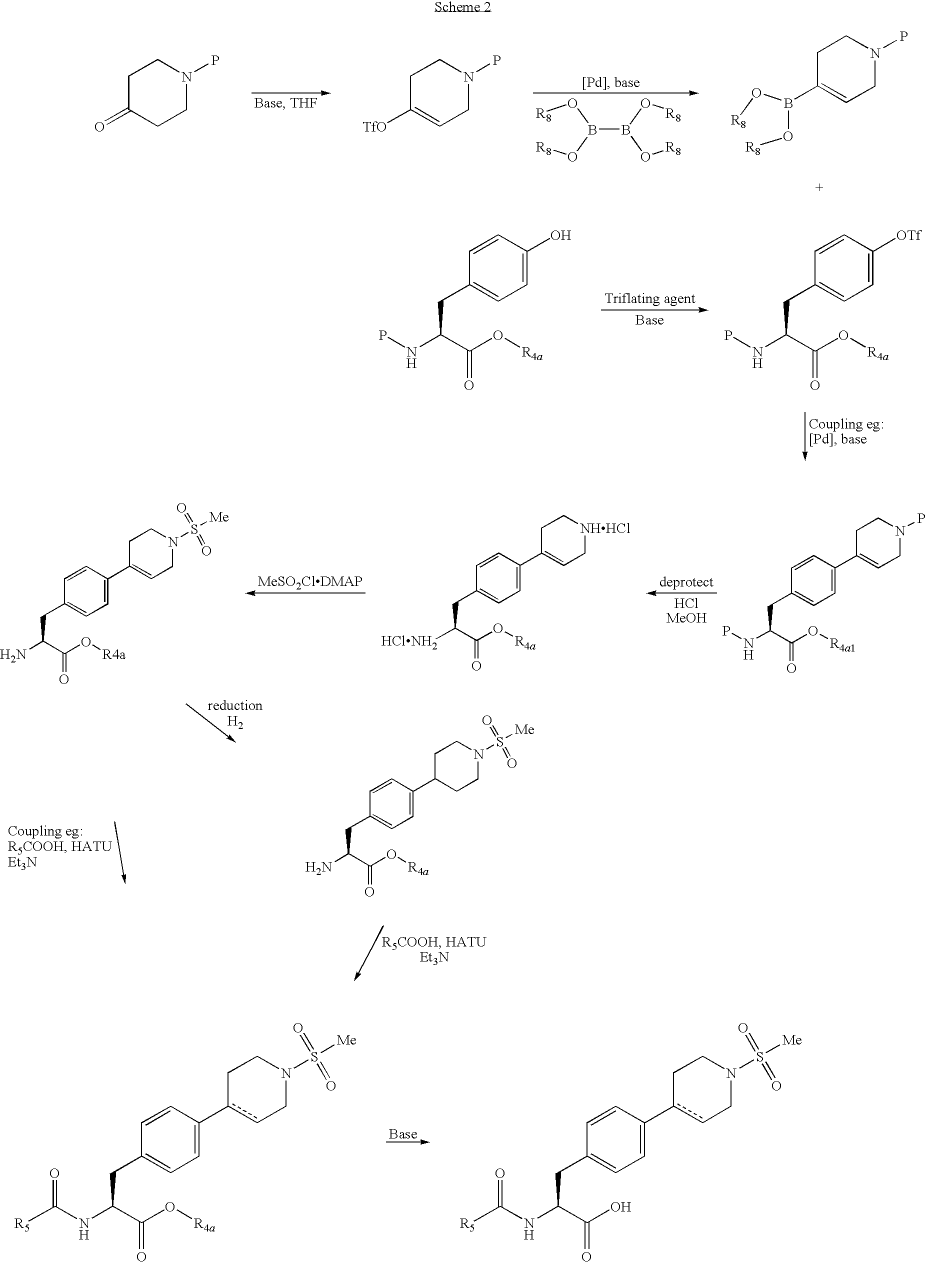

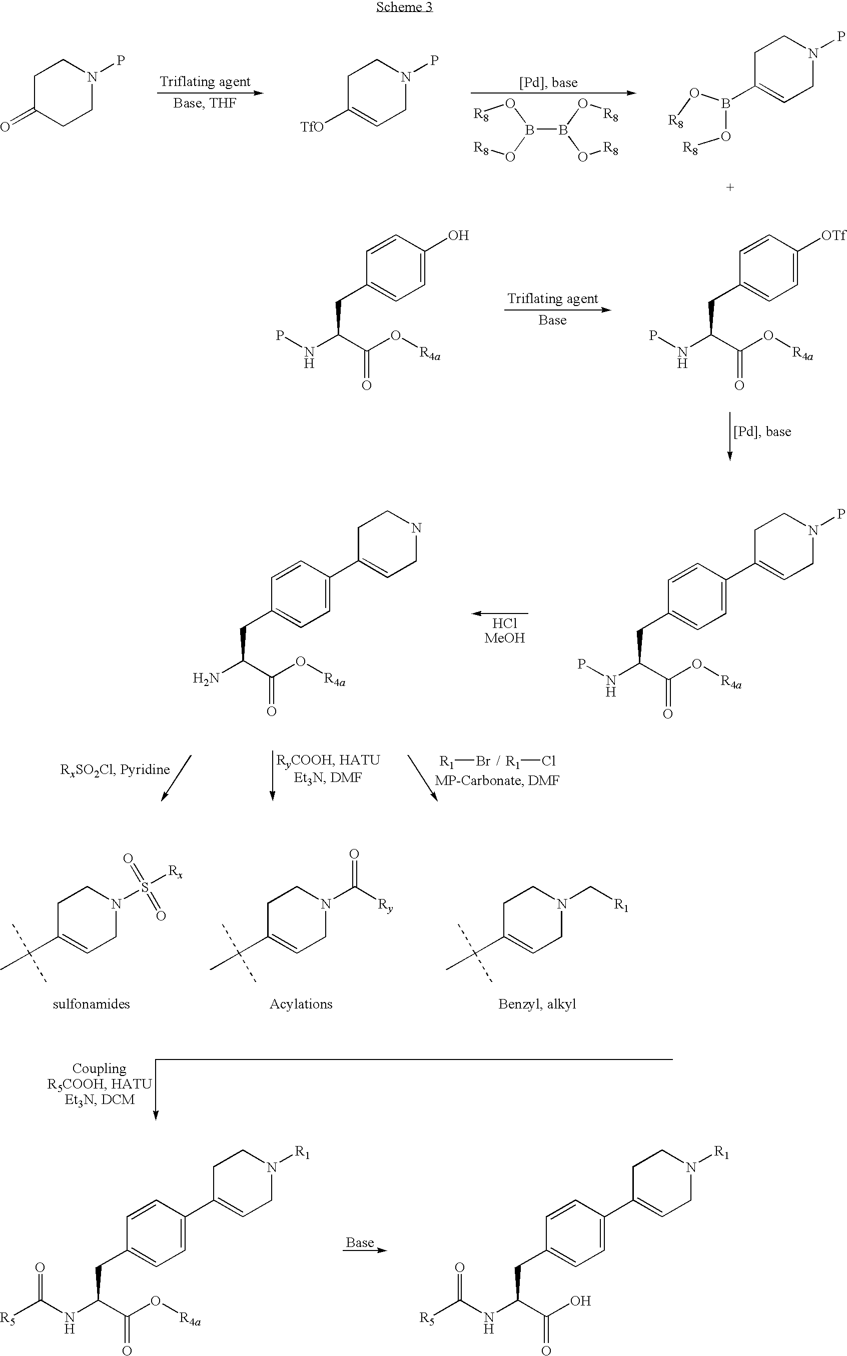

Scheme 4 depicts a possible synthesis of invention compounds containing azetidine rings.

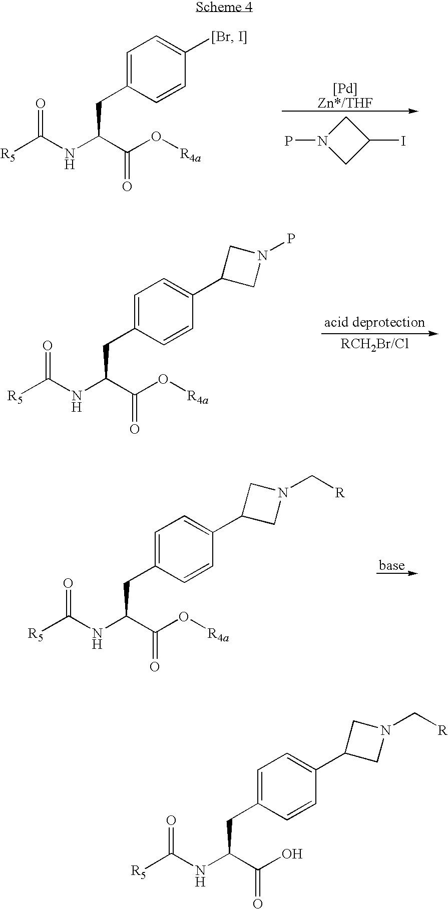

Scheme 4 illustrates the preparation of an azetidine compound substituted by an optionally substituted alkyl group. However, as will be realised, compounds with other “R₁” groups may be prepared using analogous methods to those described herein, and illustrated in Schemes 1 to 3 above.

The compounds of the present invention can be prepared in a number of ways using methods analogous to well known methods of organic synthesis. More specifically, the novel compounds of this invention may be prepared using the reactions and techniques described herein. In the description of the synthetic methods described below, it is to be understood that all proposed reaction conditions, including choice of solvent, reaction atmosphere, reaction temperature, duration of the experiment and workup procedures, are chosen to be the conditions standard for that reaction. It is understood by one skilled in the art of organic synthesis that the functionality present on various portions of the molecule must be compatible with the reagents and reactions proposed. Such restrictions to the substituents, which are not compatible with the reaction conditions, will be apparent to one skilled in the art and alternate methods must then be used.

It will be appreciated that during certain of the following processes certain substituents may require protection to prevent their undesired reaction. The skilled chemist will appreciate when such protection is required and how such protecting groups may be put in place and later removed.

For examples of protecting groups see one of the many general texts on the subject, for example, ‘Protective Groups in Organic Synthesis’ by Theodora Green (publisher: John Wiley & Sons). Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or with hydrazine.

A suitable protecting group for a hydroxy group is, for example, an acyl group, for example an alkanoyl group such as acetyl, an aroyl group, for example benzoyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide or for example a t-butyl group which may be removed, for example, by treatment with an acid, for example an organic acid such as trifluoroacetic acid or for example a benzyl group which may be removed, for example, by hydrogenation over a catalyst such as palladium-on-carbon.

Resins may also be used as a protecting group.

The protecting groups may be removed at any convenient stage in the synthesis using conventional techniques well known in the chemical art.

Compounds of the formula I or pharmaceutically-acceptable salts, prodrugs or hydrates thereof, may be prepared by any process known to be applicable to the preparation of chemically-related compounds. Such processes, when used to prepare a compound of the formula I, or a pharmaceutically-acceptable salt, prodrug or hydrate thereof, are provided as a further feature of the invention and are illustrated by the following representative examples. Necessary starting materials may be obtained by standard procedures of organic chemistry (see, for example, Advanced Organic Chemistry (Wiley-Interscience), Jerry March). The preparation of such starting materials is described within the accompanying non-limiting Examples. Alternatively, necessary starting materials are obtainable by analogous procedures to those illustrated which are within the ordinary skill of an organic chemist.

The present invention also provides that compounds of the formula I, or pharmaceutically acceptable salts or prodrugs thereof, can be prepared by a process (a) to (i) as follows (wherein the variables are as defined above unless otherwise stated):

Process (a) for the preparation of those compounds of formula I “----” is a bond, the coupling in the presence of a suitable catalyst, of a compound of the formula VI or an ester thereof:

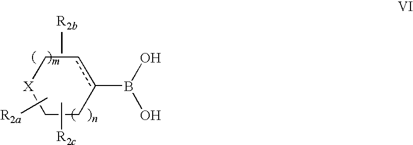

wherein X, R₂ₐ, R_{2b}, R_{2c}, m and n are as hereinbefore defined, except any functional group is protected if necessary,

with a compound of the formula VII:

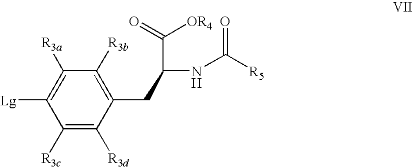

wherein R₃ₐ, R_{3b}, R_{3c}, R_{3d}, R₄ and R₅ are as hereinbefore defined, except any functional group is protected if necessary,

and Lg is a leaving group; or

Process (b) for the preparation of those compounds of formula I wherein X is NR₁ and R₁ is a group of the formula RₓS(O)₂—, the reaction, conveniently in the presence of a suitable base, of a compound of the formula I of the formula Ia:

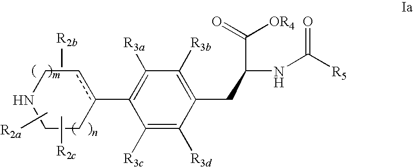

wherein R₂ₐ, R_{2b}, R_{2c}, R₃ₐ, R_{3b}, R_{3c}, R_{3d}, R₄, R₅, X, m and n are as hereinbefore defined, except any functional group is protected if necessary,

with a compound of the formula VIII:

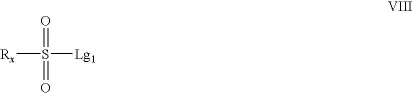

wherein Rₓ is as hereinbefore defined, except any functional group is protected if necessary,
and Lg₁ is a leaving group; or
Process (c) for the preparation of those compounds of formula I wherein X is NR₁ and R₁ is a group of the formula R_{y}C(O)—, the coupling, conveniently in the presence of a suitable base of a compound of the formula I of the formula Ia as hereinbefore defined in relation to Process (b) with a compound of the formula IX or a reactive derivative thereof:

R_{y}COOH IX

or
Process (d) for the preparation of those compounds of formula I wherein “-----” in the compounds of formula I is absent, the reduction of a compound of the formula I wherein “----” is a bond; or
Process (e) the coupling of a compound of the formula X:

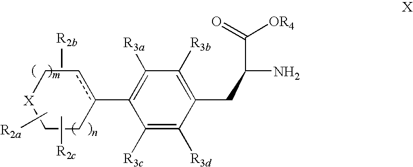

wherein R₂ₐ, R_{2b}, R_{2c}, R₃ₐ, R_{3b}, R_{3c}, R_{3d}, R₄, X, m and n are as hereinbefore defined, except any functional group is protected if necessary,

with a compound of the formula XI or a reactive derivative thereof:

R₅COOH XI

wherein R₅ is as hereinbefore defined, except any functional group is protected if necessary; or

Process (f) for the preparation of those compounds of formula I wherein X is NR₁ and R₁ is optionally substituted (C₁-C₆)alkyl, (C₃-C₆)cycloalkyl, heterocycloalkyl, (C₃-C₆)cycloalkyl(C₁-C₆)alkyl, heterocycloalkyl(C₁-C₆)alkyl, aralkyl or heteroaralkyl, the reaction, conveniently in the presence of a suitable base, of a compound of the formula I of the formula Ia as hereinbefore defined in relation to Process (b), with a compound of the formula XII:

R₁-Lg₂ XII

wherein R₁ is optionally substituted (C₁-C₆)alkyl, (C₃-C₆)cycloalkyl, heterocycloalkyl, (C₃-C₆)cycloalkyl(C₁-C₆)alkyl, heterocycloalkyl(C₁-C₆)alkyl, aralkyl or heteroaralkyl and

Lg₂ is a suitable leaving group; or

Process (g) for the preparation of those compounds of formula I wherein X is NR₁ and R₁ is a group of the formula R′HNC(O)—, the reaction of a compound of the formula I of the formula Ia as hereinbefore defined in relation to Process (b) with an isocyanate of the formula XIII:

R′N═C(O) XIII

wherein R′ is as hereinbefore defined, except any functional group is protected if necessary; or
Process (h) for the preparation of those compounds of formula I wherein X is NR₁ and R₁ is aryl or heteroaryl, the coupling in the presence of a suitable catalyst, of a compound of the formula I of the formula Ia as hereinbefore defined in relation to Process (b) with an aryl or heteroaryl boronic acid, or an ester thereof; or
Process (i) for the preparation of those compounds of formula I wherein “----” is a bond, the coupling in the presence of a suitable catalyst of a compound of the formula XIV:

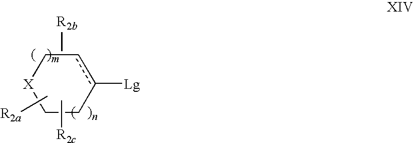

wherein X, R₂ₐ, R_{2b}, R_{2c}, m and n are as hereinbefore defined, except any functional group is protected if necessary, and Lg is a leaving group,

with a compound of the formula XV or an ester thereof:

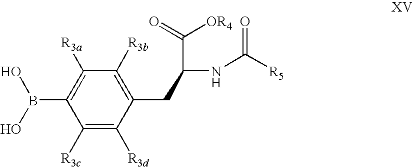

wherein R₃ₐ, R_{3b}, R_{3c}, R_{3d}, R₄ and R₅ are as hereinbefore defined, except any functional group is protected if necessary,

and Lg is a leaving group;

and thereafter, if necessary (in any order):

(i) converting a compound of the formula I into another compound of the formula I;
(ii) removing any protecting groups; and
(iii) forming a pharmaceutically acceptable salt of the compound of formula I.

Specific conditions for the above reactions are as follows.

### Reaction Conditions for Process (a)

Lg is a suitable leaving group such as halo (for example bromo) or an alkanesulfonyloxy (for example trifluoromethanesulfonyloxy).

The coupling is generally known in the art as a Suzuki Coupling (See A. Suzuki, Handbook of Organopalladium Chemistry for Organic Synthesis, (2002), 1, 249-262. Publisher John Wiley).

The reaction is suitably performed in the presence of a transition metal catalyst. A number of transition metal catalysts are known in the art to be generally useful in Suzuki couplings, for example a palladium catalyst such as 1,1′-Bis(diphenylphosphino)ferrocene-palladium(II)dichloride dichloromethane complex.

Suitably the reaction is conveniently performed in the presence of a suitable base, for example a carbonate such as a carbonate for example potassium carbonate or cesium carbonate.

The reaction is suitably carried out in the presence of a suitable inert solvent, for example a dipolar aprotic solvent such as N,N-dimethylformamide, N,N-dimethylacetamide, N-methylpyrrolidin-2-one or dimethylsulfoxide. The reaction is conveniently effected at an elevated temperature, such as a temperature in the range of, for example, 50 to 120° C.

Suitable esters of the compound of the formula VI are esters of boronic acid in the compound of formula VI. Suitable boronic acid esters include compounds of the formula VIa:

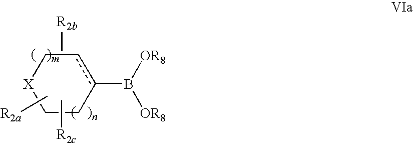

wherein X, R₂ₐ, R_{2b}, R_{2c}, m and n are as hereinbefore defined, except any functional group is protected if necessary and

each R₈ independently is (C₁-C₆)alkyl or the two OR₈ groups together with the boron atom to which they are attached form a ring. A particular ester derivative of the compound of formula VI is the compound of the formula VIb:

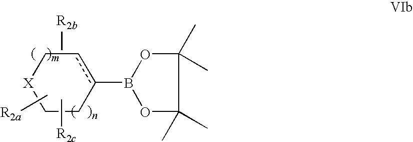

wherein X, R₂ₐ, R_{2b}, R_{2c}, m and n are as hereinbefore defined, except any functional group is protected if necessary.

Compounds of the formula VI are commercially available or they are known in the literature or they can be prepared by standard processes known in the art, such as those illustrated in Reaction Scheme 1. For example by reacting a compound of the formula XIV as hereinbefore defined in relation to Process (i), wherein Lg is for example a triflate group (or other suitable leaving group) with boronic acid or a boronic acid derivative such as bis(pinacolato)diboron. The reaction is suitably performed in the presence of a suitable transition metal catalyst, such as palladium, for example 1,1′-Bis(diphenylphosphino)ferrocene-palladium(II)dichloride dichloromethane complex and, 1,1′-bis(diphenylphosphino)ferrocene. The reaction is suitably carried out in the presence of a base.

Compounds of the formula VII may be prepared using methods well known to those skilled in organic chemistry. Representative methods are illustrated in the Examples described herein.

### Reaction Conditions for Process (b)

Lg₁ is for example halo such as chloro.

The reaction is advantageously carried out in the presence of base. A suitable base is, for example, an organic amine base such as, for example, pyridine, 2,6-lutidine, collidine, 4-dimethylaminopyridine, triethylamine, diisopropylethylamine, N-methylmorpholine or diazabicyclo[5.4.0]undec-7-ene or an alkali metal or alkaline earth metal carbonate or hydroxide, for example sodium carbonate, potassium carbonate, cesium carbonate, calcium carbonate, sodium hydroxide or potassium hydroxide or MP-carbonate. Alternatively such a base is, for example, an alkali metal hydride, for example sodium hydride, an alkali metal or alkaline earth metal amide, for example sodium amide or sodium bis(trimethylsilyl)amide or a sufficiently basic alkali metal halide, for example cesium fluoride or sodium iodide

The reaction is suitable carried out in an inert solvent such as pyridine.

The reaction is suitable performed at ambient temperature.

Compounds of the formula VIII are commercially available or they are known in the literature or they can be prepared by standard processes known in the art.

Particular compounds of the formula Ia for use in Process (b) include methyl N-(2,6-dichlorobenzoyl)-4-(1,2,3,6-tetrahydropyridin-4-yl)-L-phenylalaninate and methyl N-(2,6-dichlorobenzoyl)-4-piperidin-4-yl-L-phenylalaninate, or a salt thereof.

Reaction Conditions for Process (c)

The coupling reaction may be carried out using standard methods for the coupling of acids and amines. The coupling reaction is conveniently carried out in the presence of a suitable coupling reagent. Standard peptide coupling reagents known in the art can be employed as suitable coupling reagents for example O-(benzotriazol-1-yl)-N,N,N′,N′-tetramethyluronium tetrafluoroborate (TBTU) or O-(7-azabenzotriazol-1-yl)-N,N,N′,N′-tetramethyluronium hexafluoro-phosphate (HATU) or for example carbonyldiimidazole, dicyclohexylcarbodiimide and N-ethyl-N′-(3-dimethylaminopropyl)carbodiimide, optionally in the presence of a catalyst such as dimethylaminopyridine, 4-pyrrolidinopyridine or 2-hydroxy-pyridine-N-oxide, optionally in the presence of a base for example triethylamine, diisopropylethylamine, N-methylmorpholine, pyridine or 2,6-di-alkyl-pyridines such as 2,6-lutidine or 2,6-di-tert-butylpyridine. The reaction is conveniently performed in the present of a suitable inert solvent. Suitable solvents include N,N-dimethylacetamide, dichloromethane, benzene, tetrahydrofuran and N,N-dimethylformamide. The coupling reaction is conveniently performed at a temperature in the range of −40 to 40° C.

A “reactive derivative” of the acid of the formula IX is a carboxylic acid derivative that will react with the amine of the formula Ia to give the corresponding amide. A suitable reactive derivative of a carboxylic acid of the formula IX is, for example, an acyl halide, for example an acyl chloride formed by the reaction of the acid and an inorganic acid chloride, for example thionyl chloride; a mixed anhydride, for example an anhydride formed by the reaction of the acid and a chloroformate such as isobutyl chloroformate; an active ester, for example an ester formed by the reaction of the acid and a phenol such as pentafluorophenol, an ester such as pentafluorophenyl trifluoroacetate or an alcohol such as methanol, ethanol, isopropanol, butanol or N-hydroxybenzotriazole; or an acyl azide, for example an azide formed by the reaction of the acid and azide such as diphenylphosphoryl azide; an acyl cyanide, for example a cyanide formed by the reaction of an acid and a cyanide such as diethylphosphoryl cyanide. The reaction of such reactive derivatives of carboxylic acid with amines is well known in the art, for example they may be reacted in the presence of a base, such as those described above and in a suitable solvent, such as those described above. The reaction may conveniently be performed at a temperature as described above.

Compounds of the formula IX are commercially available or they are known in the literature or they can be prepared by standard processes known in the art.

### Reaction Conditions for Process (d)

The reduction may be effected by for example hydrogenation over a suitable catalyst, for example a platinum or palladium on carbon catalyst.

### Reaction Conditions for Process (e)

The coupling may be carried out under analogous conditions to those described above in relation to Process (c) for the coupling of acids and amines. Suitable reactive derivatives of the compound of the formula XI are carboxylic acid derivatives such as those described in relation to reactive derivatives of the compound of formula IX described hereinbefore.

Compounds of the formula X may be prepared using methods well known to those skilled in organic chemistry. For example as illustrated in Reaction Scheme 2 herein.

Compounds of the formula XI are commercially available or they are known in the literature or they can be prepared by standard processes known in the art.

### Reaction Conditions for Process (f)

Lg₂ is a suitable leaving group for example halo such as chloro or bromo.

The reaction is suitably carried out in the presence of a base, for example one of the bases described in relation to Process (b).

The reaction is suitably carried out in an inert solvent such as acetonitrile.

The reaction is suitably performed at ambient temperature.

### Reaction Conditions for Process (g)

The reaction is suitably carried out in the presence of an inert solvent, for example an ether such as tetrahydrofuran. The reaction is suitably performed at ambient temperature.

### Reaction Conditions for Process (h)

Suitable an aryl or heteroaryl boronic acids for use in this reaction are compounds of the formula R₁B(OH)₂, wherein R₁ is optionally substituted aryl or heteroaryl as defined herein. Esters of boronic acid may also be used, for example compounds of the formula R₁B(OR₉)₂, wherein each R₉ independently is (C₁-C₆)alkyl or the two OR₉ groups together with the boron atom to which they are attached form a ring such as 4,4,5,5-tetramethyl-1,3,2-dioxaborolan-2-yl.

The coupling reaction is suitably performed in the presence of a transition metal catalyst, such as a copper catalyst, for example copper acetate.

The reaction is suitably performed in the presence of a base, for example 2,6-lutidine.

The reaction is conveniently performed in the present of a suitable inert solvent, for example a chlorinated solvent such as dichloromethane. The reaction may be carried out at ambient temperature.

### Reaction Conditions for Process (i)

Lg is a suitable leaving group such as halo (for example bromo) or an alkanesulfonyloxy (for example trifluoromethanesulfonyloxy).

The coupling reaction may be performed using analogous conditions to those described in relation to Process (a) above.

Suitable esters of the compound of the formula XV are esters of boronic acid in the compound of formula XV, for example analogous ester groups of the formula ORS described in relation to the compounds of formula VIa in Process (a) wherein the OH group of the boronic acid is OR₈.

Compounds of the formula XIV are commercially available or they are known in the literature or they can be prepared by standard processes known in the art.

Compounds of the formula XV may be prepared using methods well known to those skilled in organic chemistry. For example a compound of formula XV may be prepared by reacting a compound of the formula VII with boronic acid, or a derivative thereof, using analogous methods to those described for the preparation of compounds of the formula VI in Process (a).

Compounds of the formula I may also be obtained by modifying a substituent in or introducing a substituent into another compound of formula I or a pharmaceutically acceptable salt or prodrug thereof. Suitable chemical transformations are well known to those in the art of organic chemistry. For example, when R⁴ is (1-6C)alkyl in a compound of formula I, the alkyl group may be replaced by hydrogen by hydrolysis of the compound of formula I to give another compound of formula I in which R⁴ is hydrogen. Suitably the hydrolysis is carried out in the presence of a suitable base such as lithium hydroxide. Further representative transformations include the removal of an alkoxycarbonyl group such as tert-butoxycarbonyl, from a compound of the formula I wherein X is NR₁ and R₁ is alkoxycarbonyl. The alkoxycarbonyl group may be removed by treating the compound of formula i with a suitable acid, for example hydrochloric acid.

It will be appreciated that certain of the various ring substituents in the compounds of the present invention may be introduced by standard aromatic substitution reactions or generated by conventional functional group modifications either prior to or immediately following the processes mentioned above and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halo group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulfinyl or alkylsulfonyl.

When a pharmaceutically acceptable salt of a compound of the formula I is required, for example an acid or base addition salt, it may be obtained by, for example, reaction of the compound of formula I with a suitable acid or base using a conventional procedure. Methods for the preparation of pharmaceutically acceptable salts are well known in the art. For example, the salts may be formed by reacting the free base or free acid form of the product with one or more equivalents of the appropriate acid or base in a solvent or medium in which the salt is insoluble or in a solvent such as water, which is removed in vacuo or by freeze drying or by exchanging the anions of an existing salt for another anion on a suitable ion-exchange resin.

To facilitate isolation of a compound of the formula I during its preparation, the compound may be prepared in the form of a salt that is not a pharmaceutically acceptable salt. The resulting salt can then be modified by conventional techniques to give a pharmaceutically acceptable salt of the compound. Such salt modification techniques are well known and include, for example ion exchange techniques or re-precipitation of the compound from solution in the presence of a pharmaceutically acceptable counter ion as described above, for example by re-precipitation in the presence of a suitable pharmaceutically acceptable acid to give the required pharmaceutically acceptable acid addition salt of a compound of the formula I.

Stereoisomers of compounds of formula I may be separated using conventional techniques, e.g. chromatography or fractional crystallisation. The enantiomers may be isolated by separation of a racemate for example by fractional crystallisation, resolution or HPLC. The diastereoisomers may be isolated by separation by virtue of the different physical properties of the diastereoisomers, for example, by fractional crystallisation, HPLC or flash chromatography. Alternatively particular stereoisomers may be made by chiral synthesis from chiral starting materials under conditions that will not cause racemisation or epimerisation or by derivatisation, with a chiral reagent. When a specific stereoisomer is isolated it is suitably isolated substantially free from other stereoisomers, for example containing less than 20%, particularly less than 10% and more particularly less than 5% by weight of other stereoisomers.

In the synthesis section above the expression “inert solvent” refers to a solvent which does not react with the starting materials, reagents, intermediates or products in a manner which adversely affects the yield of the desired product.

Persons skilled in the art will appreciate that, in order to obtain compounds of the invention in an alternative and in some occasions, more convenient manner, the individual process steps mentioned hereinbefore may be performed in different order and/or the individual reactions may be performed at different stage in the overall route (i.e. chemical transformations may be performed upon different intermediates to those associated hereinbefore with a particular reaction).

Certain intermediates used in the processes described above form a further feature of the present invention. Accordingly there is provided a compound selected from a compound the formula VII, X and XV, or a salt thereof as hereinbefore defined or a salt thereof.

A particular compound of the formula VII is a compound of the formula VIIa:

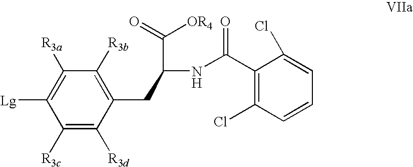

wherein R₃ₐ, R_{3b}, R_{3c}, R_{3d} and R₄ are as hereinbefore defined, except any functional group is protected if necessary; and

Lg is a suitable leaving group, for example halo (such as bromo), alkanesulfonyloxy (such as trifluoromethanesulfonyloxy) or arylsulfonyloxy (such as phenylsulfonyloxy);

or a salt thereof.

Particularly in the compounds of formula VII and VIIa R₄ is H or (C₁-C₃)alkyl. More particularly, in the compounds of formula VII and VIIa R₃ₐ, R_{3b}, R_{3c}, R_{3d} are H and R₄ is H or (C₁-C₃)alkyl for example R₄ is methyl).

A particular compound of the formula VIIa is methyl N-(2,6-dichlorobenzoyl)-O-[(trifluoromethyl)sulfonyl]-L-tyrosinate, or a salt thereof.

A particular compound of the formula X is a compound of the formula Xa:

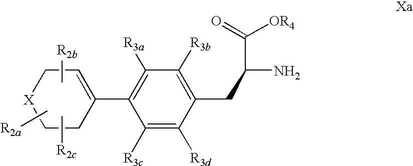

wherein R₂ₐ, R_{2b}, R_{2c}, R₃ₐ, R_{3b}, R_{3c}, R_{3d}, R₄ and X are as hereinbefore defined, except any functional group is protected if necessary,

or a salt thereof.

Particular compounds of the formula Xa are those in which X is NR₁ or O, wherein R₁ is as hereinbefore defined and R₄ is H or (C₁-C₃)alkyl, or a salt thereof. For example X is NR₁ or O, wherein R₁ is as hereinbefore defined, R₂ₐ, R_{2b}, R_{2c}, R₃ₐ, R_{3b}, R_{3c} and R_{3d} are H and R₄ is H or (C₁-C₃)alkyl.

Examples of compounds of the formula X include a compound selected from;
methyl 4-(1,2,3,6-tetrahydropyridin-4-yl)-L-phenylalaninate;
methyl 4-[1-(methylsulfonyl)piperidin-4-yl]-L-phenylalaninate;
methyl 4-[1-(methylsulfonyl)-1,2,3,6-tetrahydropyridin-4-yl]-L-phenylalaninate;
methyl 4-[1-(pyridin-2-ylcarbonyl)-1,2,3,6-tetrahydropyridin-4-yl]-L-phenylalaninate;
methyl 4-[1-(cinnolin-4-ylcarbonyl)-1,2,3,6-tetrahydropyridin-4-yl]-L-phenylalaninate;
methyl 4-[1-(cyclopropylcarbonyl)-1,2,3,6-tetrahydropyridin-4-yl]-L-phenylalaninate;
methyl 4-(1-acetyl-1,2,3,6-tetrahydropyridin-4-yl)-L-phenylalaninate;
methyl 4-{1-[(2-methyl-1,3-thiazol-4-yl)carbonyl]-1,2,3,6-tetrahydropyridin-4-yl}-L-phenylalaninate;
methyl 4-[1-(1H-benzimidazol-2-ylcarbonyl)-1,2,3,6-tetrahydropyridin-4-yl]-L-phenylalaninate;
methyl 4-[1-(4-fluorobenzoyl)-1,2,3,6-tetrahydropyridin-4-yl]-L-phenylalaninate;
methyl 4-[1-(3-fluorobenzoyl)-1,2,3,6-tetrahydropyridin-4-yl]-L-phenylalaninate;
methyl 4-[1-(4-cyanobenzoyl)-1,2,3,6-tetrahydropyridin-4-yl]-L-phenylalaninate;
methyl 3-[1-(3-cyanobenzoyl)-1,2,3,6-tetrahydropyridin-4-yl]-L-phenylalaninate;
methyl 4-[1-(3-methoxybenzoyl)-1,2,3,6-tetrahydropyridin-4-yl]-L-phenylalaninate;
methyl 4-[1-(4-fluorobenzyl)-1,2,3,6-tetrahydropyridin-4-yl]-L-phenylalaninate;
methyl 4-[1-(pyridin-3-ylsulfonyl)-1,2,3,6-tetrahydropyridin-4-yl]-L-phenylalaninate;
methyl 4-(tetrahydro-2H-pyran-4-yl)-L-phenylalaninate; and
methyl 4-(3,6-dihydro-2H-pyran-4-yl)-L-phenylalaninate;

or a salt thereof.

### Pharmaceutical Formulations

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of infection is an amount sufficient to symptomatically relieve in a warm-blooded animal, particularly a human the symptoms of the disease, to slow the progression of the disease or to reduce in patients with symptoms of the disease the risk of getting worse.

For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well known principles of medicine.

In using a compound of the formula I for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration is however preferred, particularly in tablet form. Typically, unit dosage forms will contain about 0.5 mg to 0.5 g of a compound of this invention.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, is flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. Examples of such acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Base salts include ammonium salts, alkali metal salts such as sodium, lithium and potassium salts, alkaline earth metal salts such as aluminum, calcium and magnesium salts, salts with organic bases such as dicyclohexylamine salts, N-methyl-D-glucamine, and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates like dimethyl, diethyl, dibutyl; diamyl sulfates; long chain halides such as decyl, lauryl, myristyl and stearyl halides; aralkyl halides like benzyl bromide and others. Non-toxic physiologically-acceptable salts are preferred, although other salts are also useful, such as in isolating or purifying the product.

In order to use a compound of the formula (I) or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

### Combinations

The anti-cancer treatment defined herein may be applied as a sole therapy or may involve, in addition to the compound of the invention, conventional surgery or radiotherapy or chemotherapy. Such chemotherapy may include one or more of the following categories of anti-tumour agents:

(i) antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and metalloproteinase inhibitors like marimastat, inhibitors of urokinase plasminogen activator receptor function or antibodies to Heparanase);
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stem et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

### Biological Activity

The following assays can be used to measure the effects of the compounds of the present invention as a5b1 integrin inhibitors.

### (a) In Vitro Binding Assay

The assay determined the ability of compounds to inhibit binding of α5β1 integrin to a cognate ligand, a fragment of human fibronectin. The assay used Origen technology (IGEN International) to measure the compound activity. Briefly, α5β1 integrin was coated onto epoxy-paramagnetic beads (Dynal Biotech UK, Bromborough, Wirral, CH62 3QL, UK, Catalogue No 143.02) and biotinylated-fibronectin ligand was coupled to streptavidin labelled BV-Tag-NHS-Ester (BioVeris Corporation, Witney, Oxfordshire, OX28 4GE, UK, Catalogue No JSF396). The ruthenium-labelled BV-Tag emits a electrochemiluminescence signal upon stimulation which is detected by the Origen reader. Thus, interaction of integrin and ligand causes association of bead and tag, and the resulting electrochemiluminescence signal reflects the level of integrin interaction with fibronectin.

12 μg of human α5β1 purified from placenta (Chemicon, Chandlers Ford, Hampshire, SO53 4NF, UK, Catalogue No CC1055-K) was coated onto surface-activated 3 mg of epoxy-paramagnetic beads in PBS and 1M ammonium sulfate following manufacturers instructions at 4° C. for 24 hours. Coated beads were then washed into Assay Buffer (25 mM Hepes, 150 mM NaCl, 1 μM MgCl, 1 mM MnCl, 0.1% Tween, pH7.4) to give a final concentration of 20 μg of α5β1/ml. Immediately prior to the assay, the beads were further diluted ×40 fold in Assay Buffer to give a concentration of 0.5 μg α5β1/ml.

A DNA fragment encoding the domains 9-10 (amino-acids 1325-1509) of human fibronectin (Swiss-Prot Accession No. P02751) was isolated from cDNA libraries using standard molecular biology and PCR cloning techniques. The cDNA fragment was sub-cloned into a pT73.3 expression vector containing a GST-epitope tag (developed at AstraZeneca; Bagnall et al., Protein Expression and Purification, 2003, 27: 1-11). Following expression in E. coli, the expressed protein, termed Fn9-10, was purified using the GST-tag using standard purification techniques. The recombinant Fn9-10 was subsequently biotinylated using a EZ-link Sulfo-NHS-LC-Biotinylation kit (Perbio Science UK Ltd., Cramlington, Northumberland, NE23 1WA, UK, Catalogue No. 21335) and made to give a final concentration of approximately 1 mg/ml. BV-Tag-NHS-Ester was labelled with streptavidin by incubation at room temperature following manufacturers instructions and buffer-exchanged into PBS to give a concentration of 0.5 mg/ml. Immediately prior to the assay, biotinylated-Fn9-10 and Streptavidin-labelled BV-Tag were diluted in Assay Buffer to give a final concentrations of 0.6 ug/ml and 1.5 ug/ml respectively. The Fn9-10 and BV-Tag solutions were then mixed together in equal volumes and incubated on ice for at least 30 minutes prior to the assay.

Test compounds were prepared as 10 mM stock solutions in DMSO (Sigma-Aldrich Company Ltd, Gillingham, Dorset SP8 4XT Catalogue No. 154938) and serially diluted with 4% DMSO to give a range of test concentrations at ×4 required final concentration. Aliquots (20 μl) of each compound dilution were placed into each well of a 384-well round bottomed polypropylene plate (Matrix Technologies, Wilmslow, Cheshire, SK9 3LP, Catalogue No. 4340 384). Each plate also contained control wells: maximum signal was created using wells containing 20-μl of 4% DMSO, and minimum signal corresponding to no binding was created using wells containing 20 μl of 80 mM EDTA (Sigma Catalogue No. E7889).

For the assay, 20 μl of a5b1-bead suspension and 40 μl of the Fn9-10/BV-Tag preincubated solution were added to each well containing 20 μl of compound or control solution. Assay plates were then incubated at room temperature for a minimum of 6 hours before being analysed on the Origen plate reader. The minimum value was subtracted from all values, and the signal was plotted against compound concentration to generate IC₅₀ data.

In this assay, compounds of the invention typically exhibit IC₅₀ values in the range of 0.01 to 300 μM, for example 0.01 to 100 μM.

### (b) In Vitro Cell Adhesion Assay

The assay determined the ability of compounds to inhibit the α5β1 integrin mediated adhesion of K562 cells to the ligand, a fragment of human fibronectin. The human K562 erythroleukaemia cell line (LGC Promochem, Teddington, Middlesex, UK, Catalogue No. CCL-243) was routinely maintained in RPMI 1640 medium (Sigma-Aldrich Company Ltd, Gillingham, Dorset SP8 4XT, Catalogue No. R0883) containing 10% heat-inactivated foetal calf serum (PAA lab GmbH, Pasching, Austria Catalogue No. PAA-A15-043) and 1% glutamax-1 (Invitrogen Ltd. Paisley, UK Catalogue No. 35050-038) at 37° C. with 5% CO₂ at densities between 1×10⁵ and 1×10⁶ cells/ml.

A DNA fragment encoding the domains 9-10 (amino-acids 1325-1509) of human fibronectin (Swiss-Prot Accession No. P02751) was isolated from cDNA libraries using standard molecular biology and PCR cloning techniques. The cDNA fragment was sub-cloned into a pT7#3.3 expression vector containing a GST-epitope tag (developed at AstraZeneca; Bagnall et al., Protein Expression and Purification, 2003, 27: 1-11), and the fragment termed Fn9-10. Following expression in E. coli, the expressed protein was purified using the GST-tag using standard purification techniques.

For adhesion assay, a 96-well flat bottomed plate (Greiner Bio one ltd., Gloucester GL10 3SX Catalogue No. 655101) was coated overnight at 4° C. with 100 μl of 20 μg/ml Fn9-10 ligand in Dulbecco's PBS (Gibco #14190-94). The plate was then washed twice with 200 μl of PBS and blocked with 100 μl of 3% BSA (SigmaA7888) in PBS for 1 hour at 37° C. The plates were then washed again 3 times with 200 μl of PBS and left empty.

Test compounds were prepared as 10 mM stock solutions in DMSO (Sigma-Aldrich Company Ltd, Gillingham, Dorset SP8 4XT Catalogue No. 154938) and serially diluted with HBSS (Hanks Buffered Salt solution (Gibco Catalogue No. 14170-088)/2% DMSO to give a range of test concentrations at twice required final concentration. Aliquots (50 μl) of each compound dilution were placed into each well of the Fn9-10 coated plates. Each plate also contained control wells: maximum adhesion signal was created using wells containing 50 μl HBSS/2% DMSO, and minimum signal corresponding to no adhesion was created using wells containing 50 μl HBSS/2% DMSO/20 mM EDTA (Sigma Catalogue No. E7889).

The K562 cells were cultured to ˜1×10⁶ cells/ml, and each culture suspension pooled. Cells were centrifuged at 1200 rpm for 2 mins, and the pellets washed with HBSS followed by HBSS/50 mM HEPES (Sigma Catalogue No. H0887). Cell pellets were pooled and resuspended in HBSS/0.4 mM manganese chloride/50 mM HEPES (MnCl; Sigma Catalogue No. M1787) to give a final concentration of 4×10⁶ cells/ml.

The assay was initiated by the addition of 50 μl of cell suspension into each coated well (200,000 cells/well), thus resulting in final desired compound concentration and a final MnCl concentration of 0.2 mM. The plates were incubated for 45 minutes at 37° C. 5% CO₂. After this time, the solution was flicked off as waste, and the remaining cell layer carefully washed twice with 200 μl of PBS, and then fixed with 200 μl of 100% ethanol for 30 minutes.

After fixation, the ethanol was flicked off to waste and 100 μl of 0.1% Crystal violet stain was added to each well, and incubated at ambient temperature for 15 minutes. Excess stain was removed by rinsing ˜3 times under cold slow running water. The plates were blotted over tissue then solubilised by adding 50 μl of 1% Triton X100 (Sigma Catalogue No. T9284) and shaking at 500 rpm for 30 mins on plate shaker. Finally, 100 μl of deionised water was added to each well and the absorbance was determined at 590 nM on a spectrophotometer. The minimum value was subtracted from all values, and the absorbance signal was plotted against compound concentration to generate IC₅₀ data.

In this assay, compounds of the invention typically exhibit IC₅₀ values in the range of 0.01 to 50 μM, for example 0.254 μM to 30 μM.

Although the pharmacological properties of the compounds of the formula I vary with structural change as expected, in general activity possessed by the compounds of the formula I, may be demonstrated in one or more of the above tests (a) and (b).

By way of example, activity data for the following compounds was observed.

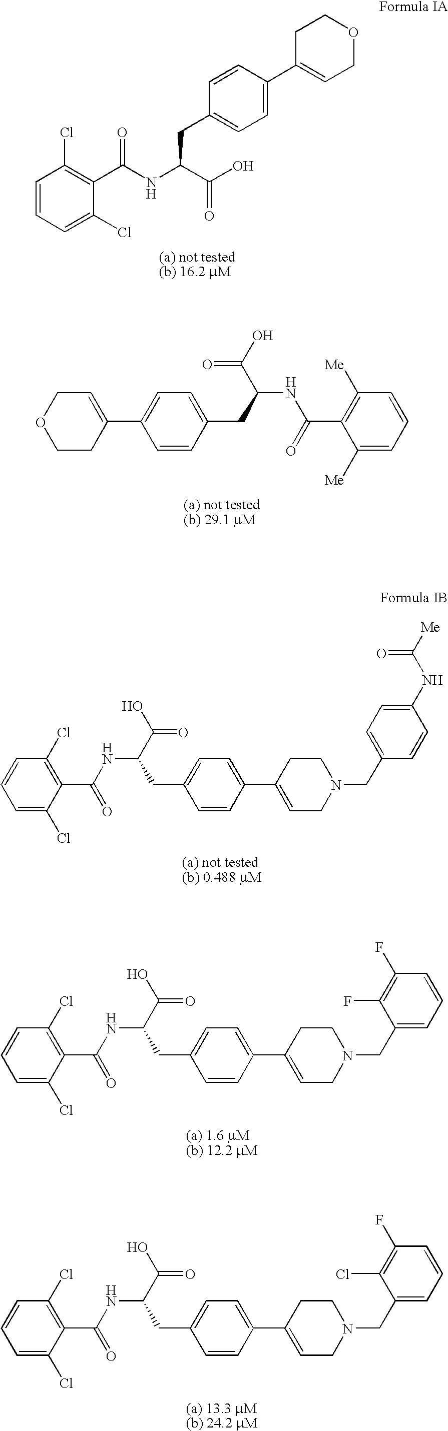

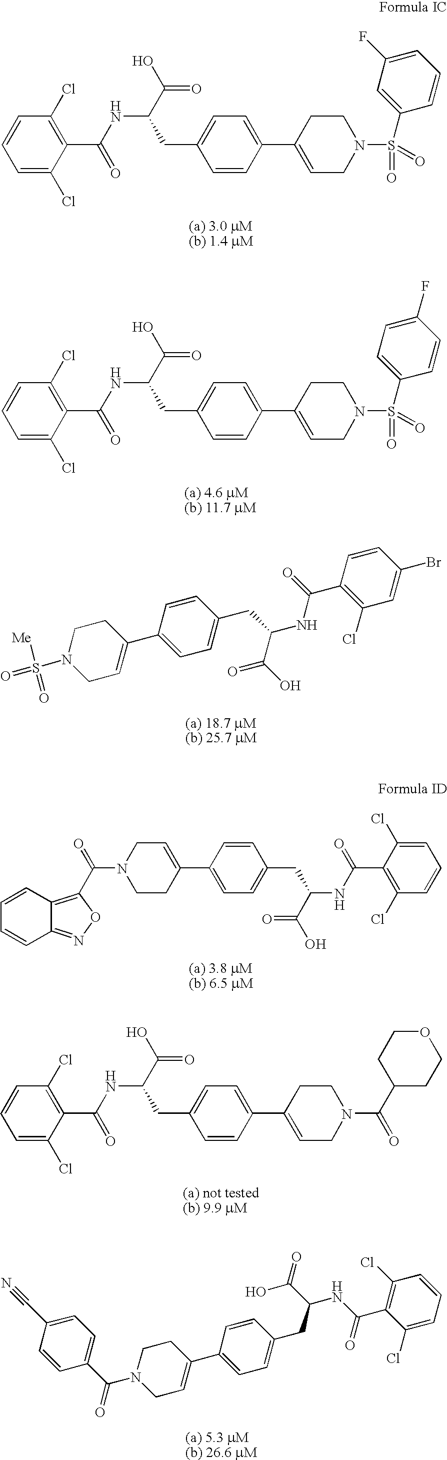

The following compounds did not achieve an IC₅₀ of less than 30 μM in the in vitro cell adhesion assay and as such are not preferred compounds according to the invention:
N-(2-chloro-6-methylbenzoyl)-4-[1-(4-fluorobenzyl)-1,2,3,6-tetrahydropyridin-4-yl]-L-phenylalanine;
N-(2,6-dichlorobenzoyl)-4-[1-(2-fluorobenzyl)piperidin-4-yl]-L-phenylalanine; and
N-(2,6-dichlorobenzoyl)-4-[1-(3-methoxybenzyl)piperidin-4-yl]-L-phenylalanine.

The compounds of the present invention are expected to possess, amongst others, anti-angiogenic properties such as anti-cancer properties that are believed to arise from their a5b1 inhibitory properties. Whilst not wising to be bound by theory, the compounds according to the invention are thought to produce an a5b1 inhibitory effect by acting as antagonists to the binding of a5b1 to fibronectin. The compounds according to the present invention may be useful for the effective treatment of, for example a5b1 driven tumours.

Accordingly, the compounds of the present invention are expected to be useful in the treatment of diseases or medical conditions mediated alone or in part by a5b1 integrin, i.e. the compounds may be used to produce an a5b1 inhibitory effect in a warm-blooded animal in need of such treatment. Thus the compounds of the present invention provide a method for the treatment of malignant cells characterised by inhibition of a5b1. Particularly the compounds of the invention may be used to produce anti-angiogenic and/or an anti-proliferative and/or anti-invasive effect mediated alone or in part by the inhibition of a5b1. Particularly, the compounds of the present invention are expected to be useful in the prevention or treatment of those tumours that are sensitive to inhibition of a5b1 that are involved in for example angiogenesis, proliferation the signal transduction steps which drive proliferation, invasion and particularly angiogenesis of these tumour cells. Accordingly the compounds of the present invention may be useful in the treatment of hyperproliferative disorders, including psoriasis, benign prostatic hyperplasia (BPH), atherosclerosis and restenosis and/or cancer by providing an anti-proliferative effect, particularly in the treatment of a5b1 sensitive cancers. Such benign or malignant tumours may affect any tissue and include non-solid tumours such as leukaemia, multiple myeloma or lymphoma and, particularly, solid tumours, for example bile duct, bone, bladder, brain/CNS, breast, colorectal, endometrial, gastric, head and neck, hepatic, lung, neuronal, oesophageal, ovarian, pancreatic, prostate, renal, skin, testicular, thyroid, uterine and vulval cancers. The compounds of the invention are expected to be useful in the treatment or prophylaxis of pathogenic angiogenesis, for example in the treatment of cancers as hereinbefore described and other diseases in which inappropriate or pathogenic angiogenesis occurs, for example age-related macular degeneration (AMD), particularly wet AMD. The compounds of the invention may also be useful in the treatment or prophylaxis of other conditions in which a5b1 may be implicated, for example thrombosis, coronary heart diseases including cardiac infarction, arteriosclerosis or atherosclerosis, tumours, osteoporosis, inflammations including irritable bowel syndrome, autoimmune diseases such as multiple sclerosis, or infections. For example, the compounds according to the invention may be useful in the treatment or prophylaxis of the following conditions:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthropathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositis and polymyositis; polymyalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthralgias, tendonititides, and myopathies;
3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritis, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions; and
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal and bacterial.

In another aspect of the present invention there is provided a compound of formula I or a pharmaceutically acceptable salt, prodrug or hydrate thereof, as defined hereinbefore for use as a medicament.

In another embodiment the present invention provides a compound of formula I or a pharmaceutically acceptable salt, prodrug or hydrate thereof for use in the treatment or prophylaxis of a cancer, for example a cancer involving a solid tumour.

In another embodiment the present invention provides a compound of formula I or a pharmaceutically acceptable salt, prodrug or hydrate thereof for use in the treatment or prophylaxis of neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system and other tumor types such as melanoma, multiple myeloma, fibrosarcoma and osteosarcoma and malignant brain tumors.

In still another embodiment the present invention provides a compound of formula I or a pharmaceutically acceptable salt, prodrug or hydrate thereof for use in the treatment or prophylaxis of pathologically angiogenic diseases, thrombosis, coronary heart diseases including cardiac infarction, arteriosclerosis, atherosclerosis, tumours, osteoporosis, inflammations including irritable bowel syndrome, autoimmune diseases such as multiple sclerosis, or infections.

In another embodiment the present invention provides a compound of formula I or a pharmaceutically acceptable salt, prodrug or hydrate thereof for use in the inhibition of a5b1 activity.

In another embodiment the present invention provides a compound of formula I or a pharmaceutically acceptable salt, prodrug or hydrate thereof for use as an antiangiogenic agent in the treatment of a solid tumour.

In another embodiment the present invention provides the use of a compound of formula I or a pharmaceutically acceptable salt, prodrug or hydrate thereof in the preparation of a medicament for the treatment or prophylaxis of a cancer, for example a cancer involving a solid tumour.

In another embodiment the present invention provides the use of a compound of formula I or a pharmaceutically acceptable salt, prodrug or hydrate thereof in the preparation of a medicament for the treatment or prophylaxis of neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system and other tumor types such as melanoma, multiple myeloma, fibrosarcoma and osteosarcoma and malignant brain tumors.

In still another embodiment the present invention provides the use of a compound of formula I or a pharmaceutically acceptable salt, prodrug or hydrate thereof in the preparation of a medicament for the treatment or prophylaxis of pathologically angiogenic diseases, thrombosis, coronary heart diseases including cardiac infarction, arteriosclerosis, atherosclerosis, tumours, osteoporosis, inflammations including irritable bowel syndrome, autoimmune diseases such as multiple sclerosis, or infections.

In another embodiment the present invention provides the use of a compound of formula I or a pharmaceutically acceptable salt, prodrug or hydrate thereof in the preparation of a medicament for use in the inhibition of a5b1 activity.

In another embodiment the present invention provides the use of a compound of formula I or a pharmaceutically acceptable salt, prodrug or hydrate thereof in the manufacture of a medicament for use as an antiangiogenic agent in the treatment of a solid tumour.

In a further aspect of the invention there is provided a pharmaceutical composition which comprises a compound of the formula I or a pharmaceutically acceptable salt, prodrug or hydrate thereof, as defined herein before in association with a pharmaceutically-acceptable diluent or carrier for use in the production of an a5b1 inhibitory effect in a warm-blooded animal such as man.

In a further aspect of the invention there is provided a pharmaceutical composition which comprises a compound of the formula I or a pharmaceutically acceptable salt, prodrug or hydrate thereof, as defined herein before in association with a pharmaceutically acceptable diluent or carrier for use in the production of an anti-cancer effect in a warm-blooded animal such as man.

In a further aspect of the invention there is provided a pharmaceutical composition which comprises a compound of the formula I or a pharmaceutically acceptable salt, prodrug or hydrate thereof, as defined herein before in association with a pharmaceutically acceptable diluent or carrier for use as an antiangiogenic agent in the treatment of a solid tumour.

In a further aspect of the invention there is provided a pharmaceutical composition which comprises a compound of the formula I or a pharmaceutically acceptable salt, prodrug or hydrate thereof, as defined herein before in association with a pharmaceutically-acceptable diluent or carrier for use in the treatment or prophylaxis of pathologically angiogenic diseases, thrombosis, coronary heart diseases including cardiac infarction, arteriosclerosis, atherosclerosis, tumours, osteoporosis, inflammations including irritable bowel syndrome, autoimmune diseases such as multiple sclerosis, or infections.

In another embodiment the present invention provides a method of inhibiting pathogenic angiogenesis in a human or animal comprising administering to said human or animal a therapeutically effective amount of a compound of formula I or a pharmaceutically acceptable salt, prodrug or hydrate thereof.

In a further embodiment the present invention provides a method of inhibiting a5b1 comprising administering to an animal or human in need of said inhibiting a therapeutically effective amount of a compound of formula I or a pharmaceutically acceptable salt, prodrug or hydrate thereof.

In a further embodiment the present invention provides a method of prophylaxis or treatment of a disease mediated in part or alone by a5b1 comprising administering to an animal or human in need of said inhibiting a therapeutically effective amount of a compound of formula I or a pharmaceutically acceptable salt, prodrug or hydrate thereof.

In another embodiment the present invention provides a method of treatment of a human or animal suffering from cancer comprising administering to said human or animal a therapeutically effective amount of a compound of formula I or a pharmaceutically acceptable salt, prodrug or hydrate thereof.

In further embodiment the present invention provides a method of prophylaxis treatment of cancer comprising administering to a human or animal in need of such treatment a therapeutically effective amount of a compound of formula I or a pharmaceutically acceptable salt, prodrug or hydrate thereof.

In another embodiment the present invention provides a method of treatment of a human or animal suffering from a neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukaemias and lymphomas including CLL and CML, tumours of the central and peripheral nervous system and other tumour types such as melanoma, multiple myeloma, fibrosarcoma and osteosarcoma and malignant brain tumours, comprising administering to said human or animal a therapeutically effective amount of a compound of formula I or a pharmaceutically acceptable salt, prodrug or hydrate thereof.

In another embodiment the present invention provides a method of treatment of a human or animal suffering from a pathologically angiogenic disease, thrombosis, coronary heart disease including cardiac infarction, arteriosclerosis, atherosclerosis, tumours, osteoporosis, inflammations including irritable bowel syndrome, autoimmune disease such as multiple sclerosis, or infection, comprising administering to said human or animal a therapeutically effective amount of a compound of formula I or a pharmaceutically acceptable salt, prodrug or hydrate thereof.

### EXAMPLES

The invention will now be illustrated in the following Examples in which, generally:

(i) operations were carried out at ambient temperature, i.e. in the range 17 to 25° C. and under an atmosphere of an inert gas such as nitrogen or argon unless otherwise stated;

(ii) in general, the course of reactions was followed by thin layer chromatography (TLC) and/or analytical high pressure liquid chromatography (HPLC); the reaction times that are given are not necessarily the minimum attainable;

(iii) when necessary, organic solutions were dried over anhydrous magnesium sulfate, work-up procedures were carried out using traditional layer separating techniques or an ALLEXIS (MTM) automated liquid handler, evaporations were carried out either by rotary evaporation in vacuo or in a Genevac HT-4/EZ-2.

(iv) yields, where present, are not necessarily the maximum attainable, and when necessary, reactions were repeated if a larger amount of the reaction product was required;

(v) in general, the structures of the end-products of the Formula I were confirmed by nuclear magnetic resonance (NMR) and/or mass spectral techniques; electrospray mass spectral data were obtained using a Waters ZMD or Waters ZQ LC/mass spectrometer acquiring both positive and negative ion data, generally, only ions relating to the parent structure are reported; proton NMR chemical shift values were measured on the delta scale using either a Bruker Spectrospin DPX300 spectrometer operating at a field strength of 300 MHz, a Bruker Dpx400 operating at 400 MHz or a Bruker Advance operating at 500 MHz. The following abbreviations have been used: s, singlet; d, doublet; t, triplet; q, quartet; m, multiplet; br, broad;

(vi) unless stated otherwise compounds containing an asymmetric carbon and/or sulfur atom were not resolved;

(vii) intermediates were not necessarily fully purified but their structures and purity were assessed by TLC, analytical HPLC, infra-red (IR) and/or NMR analysis;

(viii) unless otherwise stated, column chromatography (by the flash procedure) and medium pressure liquid chromatography (MPLC) were performed on Merck Kieselgel silica (Art. 9385);

(ix) preparative HPLC was performed on C18 reversed-phase silica, for example on a Waters ‘Xterra’ preparative reversed-phase column (5 microns silica, 19 mm diameter, 100 mm length) using decreasingly polar mixtures as eluent, for example decreasingly polar mixtures of water (containing 1% acetic acid or 1% aqueous ammonium hydroxide (d=0.88)) and acetonitrile;

(x) the following analytical HPLC methods were used; in general, reversed-phase silica was used with a flow rate of about 1 ml per minute and detection was by Electrospray Mass Spectrometry and by UV absorbance at a wavelength of 254 nm; for each method Solvent A was water and Solvent B was acetonitrile; the following columns and solvent mixtures were used:—
Preparative HPLC was performed on C18 reversed-phase silica, on a Phenomenex “Gemini” preparative reversed-phase column (5 microns silica, 110A, 21.1 mm diameter, 100 mm length) using decreasingly polar mixtures as eluent, for example decreasingly polar mixtures of water (containing 0.1% formic acid or 0.1% ammonia) as solvent A and acetonitrile as solvent B; either of the following preparative HPLC methods were used:

Method A: a solvent gradient over 9.5 minutes, at 25 mls per minute, from a 85:15 mixture of solvents A and B respectively to a 5:95 mixture of solvents A and B.

Method B: a solvent gradient over 9.5 minutes, at 25 mls per minute, from a 60:40 mixture of solvents A and B respectively to a 5:95 mixture of solvents A and B.

(xi) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;

(xii) the following abbreviations have been used:—
DCM Dichloromethane
DMA N-dimethylacetamide
DMF N,N-dimethylformamide
DMSO dimethylsulfoxide
HATU O-(7-Azabenzotriazol-1-yl)-N,N,N′,N′-Tetramethyluronium
Hexafluoro-Phosphate
MP-Carbonate triethylaminecarbonate
THF tetrahydrofuran

### tert-Butyl 4-{[(trifluoromethyl)sulfonyl]oxy}-3,6-dihydropyridine-1(2H)-carboxylate

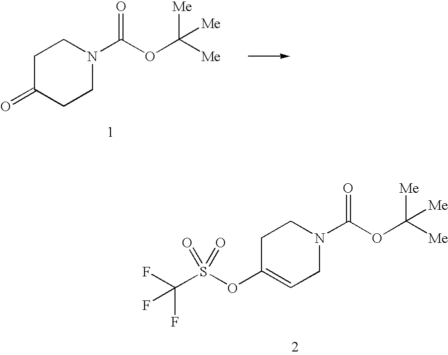

Di-iso-propylamine (22 ml) was dissolved in dry THF (125 ml) and cooled to −78° C. n-Butyllithium (62.5 ml, 2.5M) was added dropwise. The solution was stirred for 15 minutes, then tert-butyl 4-oxopiperidine-1-carboxylate (28.32 g) in THF (100 ml) was added dropwise. The reaction mixture was stirred for 1 hour at −78° C., then N-phenyltrifluoromethanesulfonimide (53.8 g) in THF (150 ml) was added dropwise. The reaction mixture was stirred at −78° C. for 2 hours and allowed to warm up to room temperature and stirred overnight. The reaction mixture was then concentrated in vacuo and the residue dissolved in ether (1000 ml). This was washed with water (500 ml), 2M sodium hydroxide solution (3×500 ml), water (500 ml) and brine (500 ml) then dried over magnesium sulfate and concentrated to give the title compound as a pale brown oil (45.38 g, 96%) which was used without further purification; ¹H NMR (CDCl₃) δ1.48 (9H, s), 2.44 (2H, m), 3.63 (2H, t), 4.00 (2H, q), 5.70 (1H, br m).

### tert-Butyl 4-(4,4,5,5-tetramethyl-1,3,2-dioxaborolan-2-yl)-3,6-dihydropyridine-1(2H)-carboxylate

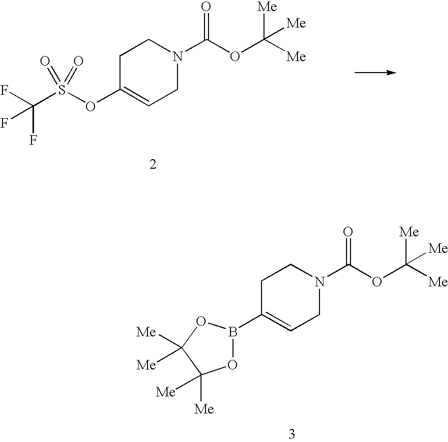

1,1′-Bis(diphenylphosphino)ferrocene-palladium(II)dichloride dichloromethane complex (3.3 g) was added to a degassed solution of tert-butyl 4-{[(trifluoromethyl)sulfonyl]oxy}-3,6-dihydropyridine-1(2H)-carboxylate (45.38 g), 1,1′-bis(diphenylphosphino)ferrocene (2.2 g), potassium acetate (40.1 g) and bis(pinacolato) diboron (38 g). The reaction mixture was then heated at 80° C. under argon for 3.5 hours. The reaction mixture was concentrated in vacuo and the residue dissolved in ethyl acetate (750 ml). This was washed with water, dried over magnesium sulfate and then filtered through a pad of Celite. Concentration of this solution gave a brown solid that was triturated with acetonitrile, then filtered and washed with cold acetonitrile to give the title compound as a white solid (11.85 g, 28%). The filtrate was concentrated in vacuo to give a brown oil that was purified by chromatography using iso-hexane-10% ethyl acetate as eluent to give further product (8.6 g, 20%); ¹H NMR (CDCl₃) δ 1.26 (12H, s), 1.46 (9H, s), 2.22 (2H, m), 3.44 (2H, t), 3.95 (2H, q), 6.46 (1H, br m).

### Methyl N-(tert-butoxycarbonyl)-O-[(trifluoromethyl)sulfonyl]-L-tyrosinate

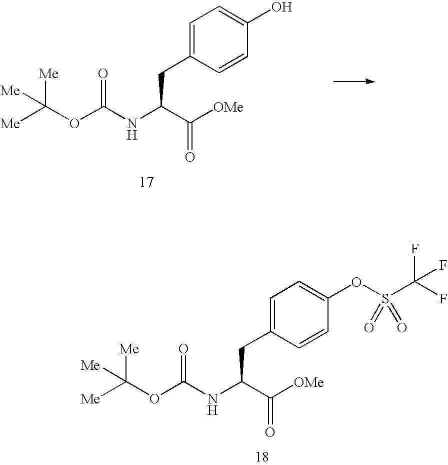

N-(tert-Butoxycarbonyl)tyrosine methyl ester (75 g) was dissolved in dry DCM (1000 ml) under nitrogen and 2,6-lutidine (44.4 ml) was added in a single portion. The solution was then cooled to 0° C. and trifluoromethanesulfonic anhydride (51.3 ml) was added slowly. The reaction mixture was stirred overnight and the solution washed successively with water, 1N citric acid solution and brine, then dried and concentrated to give a red oil. The oil was dissolved in iso-hexane (400 ml) at reflux, filtered, then concentrated in vacuo to give the title compound as an orange oil that slowly crystallised upon standing, (107.93 g, 99%); ¹H NMR (CDCl₃) δ 1.41 (9H, s), 3.03 (1H, dd), 3.17 (1H, dd), 3.71 (3H, s), 4.60 (1H, br m), 5.02 (1H, br m), 7.22 (4H, m); Mass Spectrum M−H⁺=426.39.

### Methyl N-(tert-butoxycarbonyl)-4-[1-(tert-butoxycarbonyl)-1,2,3,6-tetrahydropyridin-4-yl]-L-phenylalaninate

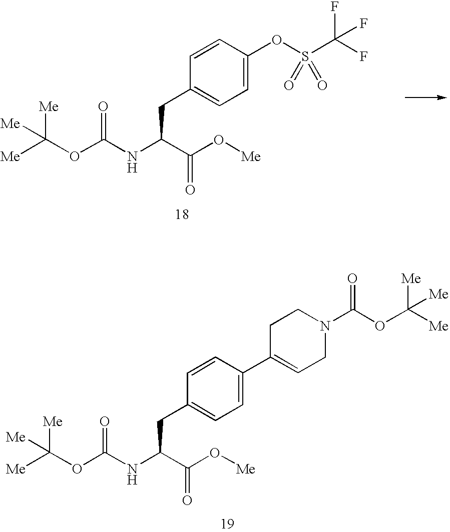

1,1′-Bis(diphenylphosphino)ferrocene-palladium(II)dichloride dichloromethane complex (380 mg) was added to a degassed solution of N-(tert-butoxycarbonyl)-O-[(trifluoromethyl)sulfonyl]-L-tyrosinate (5.00 g), tert-butyl 4-(4,4,5,5-tetramethyl-1,3,2-dioxaborolan-2-yl)-3,6-dihydropyridine-1(2H)-carboxylate (4.70 g) and potassium carbonate (4.86 g) in DMF (50 ml) under argon. The reaction mixture was heated at 85° C. for one hour then cooled to room temperature and concentrated in vacuo. The residue was partitioned between water and ethyl acetate. The organic layer was washed twice with water and dried over magnesium sulfate. Concentration gave a dark brown oil that was purified by chromatography using 5-20% ethyl acetate in iso-hexane as eluent to afford the title compound as an oil (3.54 g, 66%); ¹H NMR (CDCl₃) δ 1.30 (9H, s), 1.40 (9H, s), 2.40 (2H, m), 2.90-3.10 (2H, m), 3.60 (2H, m), 3.70 (3H, s), 3.90-4.10 (2H, m), 4.50 (1H, m), 4.90 (1H, m), 5.90 (1H, br s), 7.00 (2H, d), 7.25 (2H, d); Mass Spectrum M+Na⁺=483.53.

### Methyl 4-(1,2,3,6-tetrahydropyridin-4-yl)-L-phenylalaninate dihydrochloride

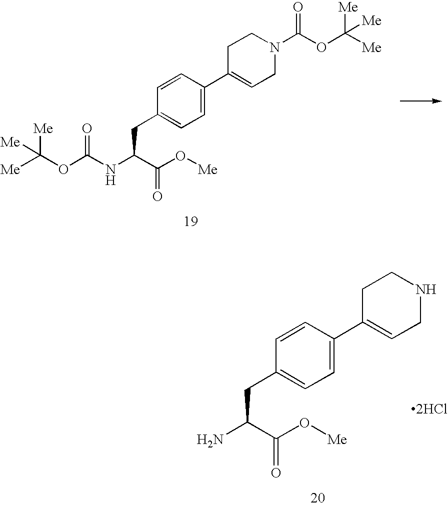

Concentrated HCl (43 ml) was added to a solution of methyl N-(tert-butoxycarbonyl)-4-[1-(tert-butoxycarbonyl)-1,2,3,6-tetrahydropyridin-4-yl]-L-phenylalaninate (115 g) in methanol (1000 ml) and the resulting reaction mixture was heated at reflux for 3 hours. The reaction was cooled, concentrated in vacuo and the residue triturated with iso-propanol to give the title compound as a pale green solid (50.8 g, 61%); ¹H NMR (CDCl₃) δ 1.30 (2H, m), 2.70 (2H, m), 3.30 (2H, m), 3.60-3.80 (5H, m), 4.30 (1H, m), 6.20 (1H, m), 7.20 (2H, m), 7.40 (2H, m), 8.70 (2H, br s), 9.50 (1H, br s); Mass Spectrum MH⁺=261.56.

### Methyl 4-[1-(methylsulfonyl)-1,2,3,6-tetrahydropyridin-4-yl]-L-phenylalaninate

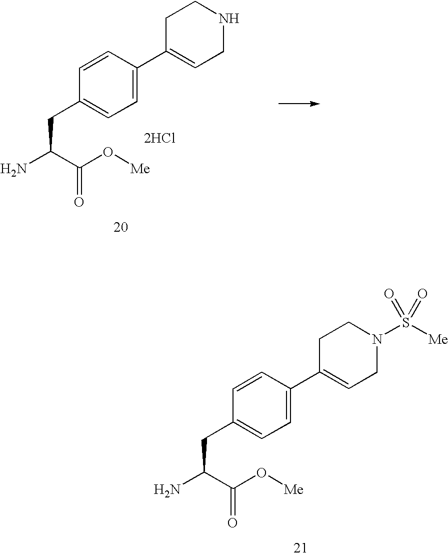

4-Dimethylaminopyridine (28 g) was added to a suspension of methyl 4-(1,2,3,6-tetrahydropyridin-4-yl)-L-phenylalaninate dihydrochloride (25 g) in DCM (1000 ml) and the reaction stirred for 1 hour, then cooled to −70° C. Methanesulfonyl chloride (4.1 ml) in DCM (10 ml) was added dropwise and the resulting reaction mixture was stirred at −70° C. for 2 hours, then −45° C. for 1 hour. Further methanesulfonyl chloride (1.36 ml) was added and the reaction mixture was stirred for 2 hours. Piperidine (5 ml) was added and the reaction mixture was allowed to warm to room temperature and stirred overnight. The reaction mixture was then washed with water and brine, dried and concentrated to give a brown solid that was triturated with ether and filtered to give the title compound as a beige solid (11.05 g, 43%); ¹H NMR (DMSO-d6) 2.60 (2H, m), 2.70-2.90 (2H, m), 2.95 (3H, s), 3.40 (2H, t), 3.60 (4H, m), 3.90 (2H, m), 6.20 (1H, m), 7.20 (2H, d), 7.40 (2H, d); Mass Spectrum MH⁺=339.47

### Example 1

### Preparation of Compounds of the Formula

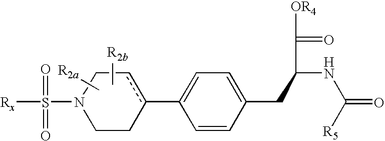

### Example 1.1

### N-(2,6-dimethylbenzoyl)-4-[1-(methylsulfonyl)-1,2,3,6-tetrahydropyridin-4-yl]-L-phenylalanine

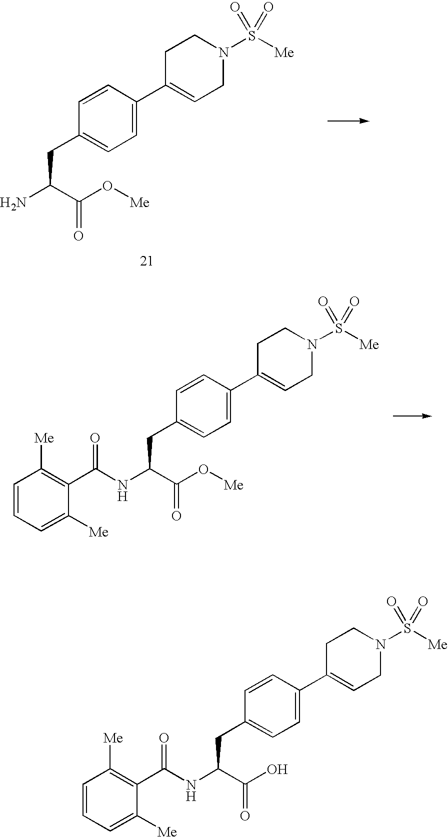

HATU (190 mg) and diisopropylethylamine (87 μl) was added to 2,6-dimethylbenzoic acid (75 mg) in anhydrous DMF (2 ml). The reaction mixture was stirred for 30 minutes at 25° C. before addition of methyl 4-[1-(methylsulfonyl)-1,2,3,6-tetrahydropyridin-4-yl]-L-phenylalaninate (85 mg) in a single portion. The reaction mixture was then stirred at room temperature for 72 hours, then quenched with water which resulted in formation of a pale brown precipitate. This was collected by filtration and dissolved in methanol (2 ml) and acetonitrile (1 ml). Lithium hydroxide dihydrate (31 mg) was dissolved in water (500 μl) and added and the resulting solution stirred at room temperature for 1 hour. The reaction was then concentrated, dissolved in water and acidified to a pH of approximately 1 by dropwise addition of concentrated HCl. The resulting precipitate was collected by filtration and dried to afford the title compound as a pale brown powder (48 mg, 42%);

¹H NMR (DMSO-d6, 300 MHz) δ 1.96 (6H, s), 2.59 (2H, m), 2.87 (1H, m), 2.94 (3H, s), 3.16-3.22 (1H, m), 3.39 (2H, t), 3.86 (2H, d), 4.68-4.76 (1H, m), 6.16 (1H, d), 6.95 (2H, d), 7.11 (1H, t), 7.29 (2H, d), 7.40 (2H, d), 8.57 (1H, d); Mass Spectrum M−H⁺=455.58.

The procedure described above was repeated by coupling methyl 4-[1-(methylsulfonyl)-1,2,3,6-tetrahydropyridin-4-yl]-L-phenylalaninate with the appropriate carboxylic acid to obtain the compounds listed in Table 1.

TABLE 1

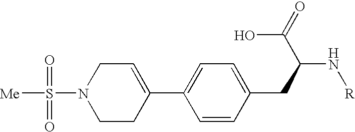

| Example | | | Mass | NMR Data |
|---|---|---|---|---|
| Number | R | Name | Ion | (400 MHz, DMSO-d₆) |
| 1.2 | 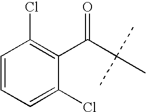 | N-(2,6-Dichlorobenzoyl)-4- [1-(methylsulfonyl)-1,2,3,6- tetrahydropyridin-4-yl]-L- phenylalanine | 495.29 (M − H⁺) | 2.55 (2H, m), 2.79 (1H, m), 2.94 (3H, s), 3.15-3.22 (1H, d), 3.40 (2H, m), 3.88 (2H, d), 4.68-4.76 (1H, m), 6.18 (1H, m), 7.30 (1H, m), 7.37-7.45 (5H, m), 7.55-7.60 (1H, m), 9.05 (1H, d) |
| 1.3 | 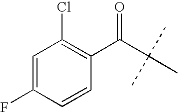 | N-(2-Chloro-4- fluorobenzoyl)-4-[1- (methylsulfonyl)-1,2,3,6- tetrahydropyridin-4-yl]-L- phenylalanine | 481.53 (MH⁺) | — |
| 1.4 | 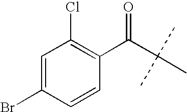 | N-(4-Bromo-2- chlorobenzoyl)-4-[1- (methylsulfonyl)-1,2,3,6- tetrahydropyridin-4-yl]-L- phenylalanine | 543.92 (MH⁺) | — |
| 1.5 | 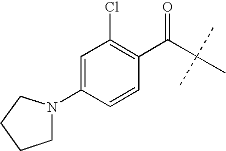 | N-(2-Chloro-4-pyrrolidin-1- ylbenzoyl)-4-[1- (methylsulfonyl)-1,2,3,6- tetrahydropyridin-4-yl]-L- phenylalanine | 532.57 (MH⁺) | 1.94-1.99 (7H, m), 2.54 (1H, d), 2.59-2.60 (1H, m), 2.94 (3H, d), 3.25 (3H, t), 3.38 (2H, t), 3.86 (2H, t), 6.18 (1H, s), 6.48- 6.50 (1H, m), 6.49-6.51 (2H, m), 6.53-6.55 (1H, m), 7.25 (1H, s), 7.28 (2H, d), 7.39 (2H, d), 7.77 (1H, d) |

### Methyl 4-[1-(methylsulfonyl)piperidin-4-yl]-L-phenylalaninate

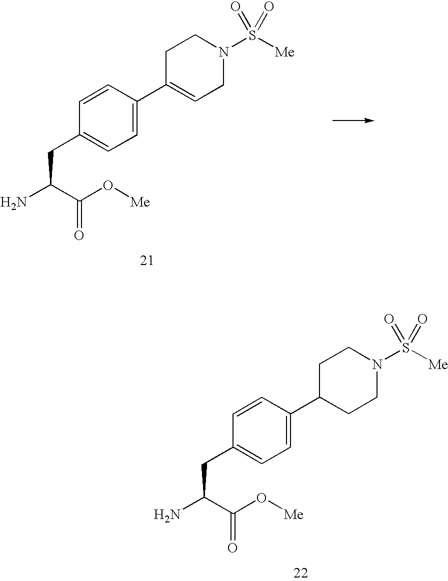

Methyl 4-[1-(methylsulfonyl)-1,2,3,6-tetrahydropyridin-4-yl]-L-phenylalaninate was dissolved in methanol (5 ml) and acetic acid (5 ml). To this was added 10% Pd/C (40 mg) and the reaction mixture was stirred under hydrogen at atmospheric pressure for 18 hours. The solution was filtered through a pad of Celite and the filtrate was concentrated in vacuo to afford an oil. This was dissolved in methanol (10 ml) and MP-Carbonate resin was added until the solution was pH 8. The mixture was filtered and the filtrate was concentrated in vacuo to afford an oil. This was triturated with diethyl ether and a few drops of acetonitrile to afford the title compound as a pale brown solid (150 mg, 75%); ¹H NMR (DMSO-d6) δ 1.60-1.75 (2H, m), 1.80 (2H, d), 2.60-2.80 (2H, m), 2.70-2.90 (7H, m), 3.50 (3H, m), 3.70 (2H, d), 7.20 (4H, s); Mass Spectrum MH⁺=341.54

### Example 2.1

### N-(2,6-dichlorobenzoyl)-4-[1-(methylsulfonyl)piperidin-4-yl]-L-phenylalanine

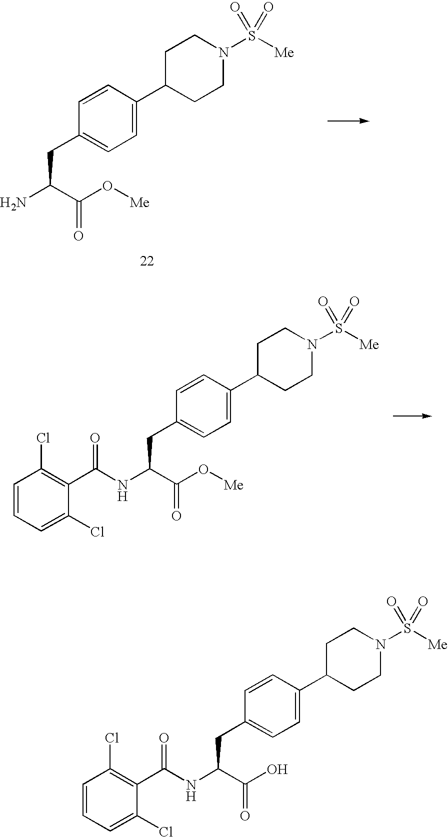

2,6-Dichlorobenzoic acid (169 mg) was dissolved in DMF (2 ml) and HATU (338 mg) and triethylamine (124 μl) were added as single portions. After stirring for 0.5 hour, methyl 4-[1-(methylsulfonyl)piperidin-4-yl]-L-phenylalaninate (200 mg) was added as a single portion and the resulting reaction mixture was left to stir overnight. After this time, the reaction mixture was quenched into water (50 ml) and the resulting suspension was stirred vigorously for 0.5 hour. The solid was then collected by filtration and dissolved in methanol (10 ml) and lithium hydroxide monohydrate (74 mg) was added as a single portion. A little water was then added and the reaction mixture was left to stir overnight. The reaction was concentrated, dissolved in water and the solution filtered. This was then acidified to pH1. The resulting precipitate was collected by filtration and dried to afford the title compound as a white solid (154 mg, 52%); ¹H NMR (DMSO-d6) δ 1.65 (m, 2H), 1.84 (br d, 2H), 2.60 (m, 1H), 2.83 (m, 2H), 2.90 (s, 3H), 3.14 (dd, 2H), 3.68 (br d, 2H), 4.69 (ddd, 1H), 7.17 (d, 2H), 7.24 (d, 2H), 7.40 (m, 3H), 9.01 (d, 1H), 12.72 (br s, 1H); Mass Spectrum M−H⁺=497.30.

The procedure described above was repeated by coupling methyl 4-[1-(methylsulfonyl)piperidin-4-yl]-L-phenylalaninate with the appropriate carboxylic acid to obtain the compounds listed in Table 2.

TABLE 2

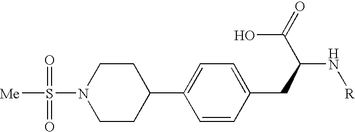

| Example | | | | NMR Data |
|---|---|---|---|---|
| Number | R | Name | Mass Ion | (400 MHz, DMSO-d₆) |
| 2.2 | 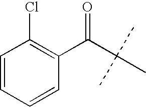 | N-(2-chlorobenzoyl)-4-[1- (methylsulfonyl)piperidin- 4-yl]-L-phenylalanine | 465.5 (M + H⁺) | 1.55-1.70 (2H, m), 1.80-1.88 (2H, d), 2.50-2.60 (2H, m), 2.70 (3H, s), 2.75-2.85 (2H, m), 2.90-3.00 (1H, dd), 3.10-3.20 (1H, dd), 3.60-3.70 (2H, d), 4.30- 4.40 (1H, m), 7.10-7.20 (4H, m), 7.20-7.25 (1H, dd), 7.30-7.45 (3H, m), 7.90 (1H, s) |

### Methyl 4-(1,2,3,6-tetrahydropyridin-4-yl)-L-phenylalaninate

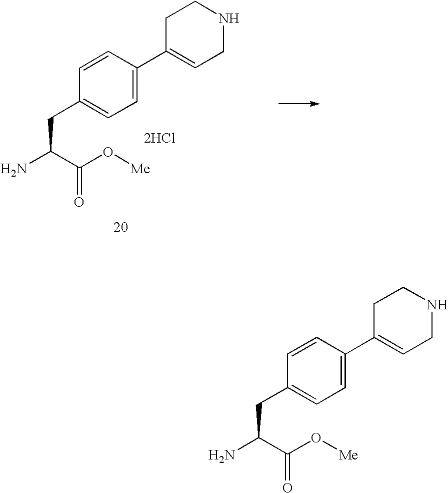

MP-Carbonate (36 g, 2.74 mmol/g) was added to a solution of methyl 4-(1,2,3,6-tetrahydropyridin-4-yl)-L-phenylalaninate dihydrochloride (12 g) in methanol (600 ml) and stirred at room temperature for 1 hour. The MP carbonate was removed by filtration and the filtrate concentrated under reduced pressure to give a brown glass-like solid (9.6 g, 100%); ¹H NMR: (DMSO-d6) δ 2.30 (2H, m), 2.65-2.90 (2H, m), 3.30 (2H, m), 3.45 (2H, m), 3.50 (1H, m), 3.60 (s, 3H), 6.20 (1H, m), 7.20 (2H, m), 7.40 (2H, m); Mass Spectrum MH⁺=261.56.

### Methyl 4-[1-(pyridin-2-ylcarbonyl)-1,2,3,6-tetrahydropyridin-4-yl]-L-phenylalaninate

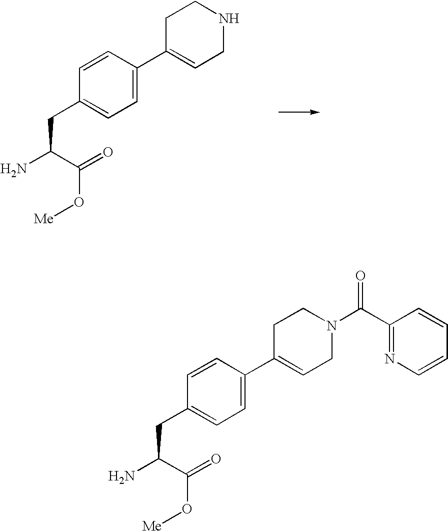

Methyl 4-(1,2,3,6-tetrahydropyridin-4-yl)-L-phenylalaninate (1.84 g), diisopropylethylamine (3.70 ml) and picolinic acid (695 mg) were dissolved in DCM (60 ml). The resulting mixture was cooled to less than 0° C. for 1 hour. HATU (2.69 g) was added in one portion and the mixture was stirred for 1.5 hours, then allowed to warm to room temperature. The reaction was washed twice with a saturated sodium bicarbonate solution and brine before being dried and concentrated in vacuo to give an oil. The oil was purified by chromatography using a DCM to ethyl acetate to 10% methanol/ethyl acetate as eluent to give the title compound as a yellow oil (1.12 g, 43%); ¹H NMR (DMSO-d6) δ 2.60 (m, 2H), 2.70-2.95 (m, 2H), 3.50-3.70 (m, 5H), 3.90 (m, 1H), 4.1-4.35 (m, 1H), 6.0-6.25 (m, 1H), 7.10 (d, 2H), 7.40 (m, 2H), 7.50 (m, 1H), 7.60 (d, 1H), 8.00 (m, 1H), 8.60 (m, 1H); Mass Spectrum MH⁺=366.52.

The procedure described above was repeated using the appropriate carboxylic acid. Thus were obtained the intermediates described Table 3 below:

TABLE 3

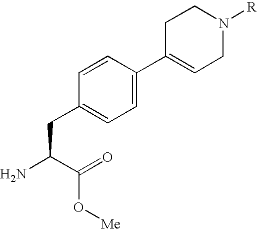

| | | Mass | NMR Data |
|---|---|---|---|
| R | Name | Ion | (400 MHz, DMSO-d₆) |
| 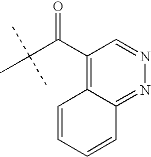 | Methyl 4-[1-(cinnolin- 4-ylcarbonyl)-1,2,3,6- tetrahydropyridin-4- yl]-L-phenylalaninate | 417.56 (M + H⁺) | 2.40 (1H, m), 2.70 (1H, m), 2.80- 2.95 (2H, m), 3.30 (2H, m), 3.60 (3H, s), 3.70 (1H, t), 3.80 (1H, m), 4.50 (1H, m), 5.9-6.3 (1H, m), 7.10 (2H, d), 7.30 (2H, d) 7.80-8.10 (3H, m), 8.60 (1H, m), 9.40 (1H, d) |
| 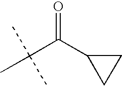 | Methyl 4-[1-(cyclopro- pylcarbonyl)-1,2,3,6- tetrahydropyridin-4- yl]-L-phenylalaninate | 329.53 (M + H⁺) | 0.70-0.79 (4H, m), 1.20 (1H, m), 1.90-2.10 (1H, m), 2.56 (2H, m), 2.74-2.83 (1H, m), 2.86-2.92 (1H, m), 3.59 (3H, s), 3.66 (1H, m), 3.84-3.87 (1H, m), 4.10 (1H, m), 4.37 (1H, m), 6.17 (1H, s), 7.17 (2H, d), 7.37 (2H, d) |
| 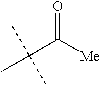 | Methyl 4-(1-acetyl- 1,2,3,6- tetrahydropyridin-4- yl)-L-phenylalaninate | 303.51 (M + H⁺) | 1.99-2.09 (4H, m), 2.15 (1H, s), 2.28 (0H, d), 2.73 (0H, s), 2.74- 2.81 (1H, m), 2.84-2.91 (1H, m), 3.56-3.67 (5H, m), 4.07-4.14 (2H, m), 6.14 (1H, s), 7.16 (2H, d), 7.33-7.36 (2H, m) |
| 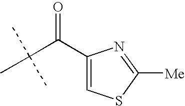 | Methyl 4-{1-[(2- methyl-1,3-thiazol-4- yl)carbonyl]-1,2,3,6- tetrahydropyridin-4- yl}-L-phenylalaninate | 386.46 (M + H⁺) | 2.50 (2H, m), 2.60 (3H, S), 2.74- 2.95 (2H, m), 3.5 (3H, s), 3.56- 3.67 (1H, m), 3.85 (2H, t), 4.20- 4.3 (2H, m), 6.14 (1H, m), 7.16 (2H, d), 7.30 (2H, d), 8.0 (1H, s) |
| 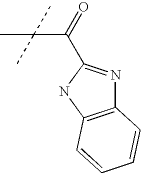 | Methyl 4-[1-(1H- benzimidazol-2- ylcarbonyl)-1,2,3,6- tetrahydropyridin-4- yl]-L-phenylalaninate | 405.49 (M + H⁺) | 2.50 (2H, m), 2.74-2.95 (2H, m), 3.0-3.2 (2H, m), 3.50 (m, 2H), 3.6 (3H, s), 3.67 (1H, m), 6.14 (1H, bs), 7.16 (2H, d), 7.30 (2H, m), 7.40 (2H, m), 7.70 (2H, m), 7.8 (1H, m) |

### Methyl-4-[1-(4-fluorobenzoyl)-1,2,3,6-tetrahydropyridin-4-yl]-L-phenylalaninate

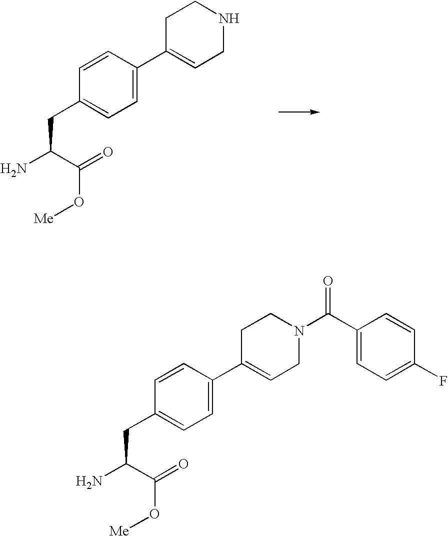

Methyl 4-(1,2,3,6-tetrahydropyridin-4-yl)-L-phenylalaninate (950 mg) was dissolved in DCM (50 ml) and diisopropylethylamine (1.26 ml) was added and the mixture cooled to −50° C. 4-Fluorobenzoyl chloride (315 μl) was added dropwise and the reaction mixture was stirred at −50° C. for 1.5 hours. The reaction mixture was quenched with piperazine (314 mg) then allowed to warm to room temperature, and washed with brine (2×25 ml), dried and concentrated in vacuo to give an oil. The oil was purified by chromatography using ethyl acetate to DCM-7% methanol as eluent to give the title compound as a pale brown glass (680 mg, 47%); ¹H NMR (DMSO-d6, 300 MHz) δ 1.75 (2H, s), 2.54 (2H, s), 2.73-2.80 (1H, m), 2.83-2.90 (1H, m), 3.30 (2H, m), 3.58 (3H, s), 4.04 (1H, q), 4.21 (2H, br s), 6.18 (1H, m), 7.16 (2H, d), 7.26-7.37 (4H, m), 7.50-7.57 (2H, m); Mass Spectrum MH⁺=383.48.

The procedure described above was repeated using the appropriate benzoyl chloride to obtain the intermediates listed in Table 3a.

TABLE 3a

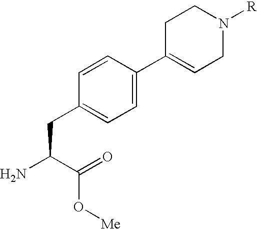

| | | | NMR Data |
|---|---|---|---|
| R | Name | Mass Ion | (400 MHz, DMSO-d₆) |
| 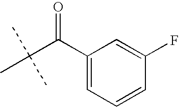 | Methyl 4-[1-(3- fluorobenzoyl)-1,2,3,6- tetrahydropyridin-4- yl]-L- phenylalaninate | 383.44 (M + H⁺) | (CDCl₃) 1.52 (2H, m), 2.58 (2H, m), 2.82-2.90 (1H, m), 3.05- 3.12 (1H, m), 3.46 (2H, m), 3.69- 3.78 (3H, m), 3.97-4.08 (1H, m), 4.37 (2H, br s), 6.13 (1H, m), 7.10-7.15 (1H, m), 7.16- 7.18 (3H, m), 7.21-7.25 (1H, m), 7.32 (2H, d), 7.41 (1H, t) |
| 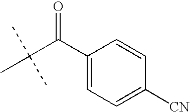 | Methyl 4-[1-(4- cyanobenzoyl)-1,2,3,6- tetrahydropyridin-4- yl]-L- phenylalaninate | 390.49 (M + H⁺) | (At 373K) 1.69 (2H, s), 2.54 (2H, m), 2.74-2.81 (1H, m), 2.88-2.90 (1H, m), 2.94-2.98 (2H, m), 3.60-3.61 (5H, m), 4.16 (1H, m), 6.11 (1H, s), 7.17 (2H, d), 7.34 (2H, d), 7.60-7.63 (2H, m), 7.88 (2H, d) |
| 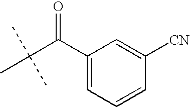 | Methyl 3-[1-(3- cyanobenzoyl)-1,2,3,6- tetrahydropyridin-4- yl]-L- phenylalaninate | 390.46 (M + H⁺) | (At 373K) 2.54 (2H, m), 2.74- 2.81 (1H, m), 2.88-2.90 (1H, m), 2.94-2.98 (2H, m), 3.60 (3H, s), 3.62-3.70 (3H, m), 4.20 (2H, m), 6.11 (1H, s), 7.17 (2H, d), 7.34 (2H, d), 7.60-7.63 (1H, m), 7.75 (1H, m), 7.80-7.9 (2H, m) |
| 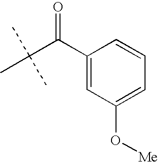 | Methyl 4-[1-(3- methoxybenzoyl)-1,2,3,6- tetrahydropyridin-4- yl]-L- phenylalaninate | 395.47 (M + H⁺) | 1.95 (2H, m), 2.70-2.90 (2H, m), 3.25 (4H, m), 3.60 (3H, s), 3.80 (3H, s), 4.25 (1H, m), 6.0-6.20 (1H, m), 6.90-7.10 (3H, m), 7.20 (2H, d), 7.30-7.40 (3H, m) |

### Methyl 4-[1-(4-fluorobenzyl)-1,2,3,6-tetrahydropyridin-4-yl]-L-phenylalaninate

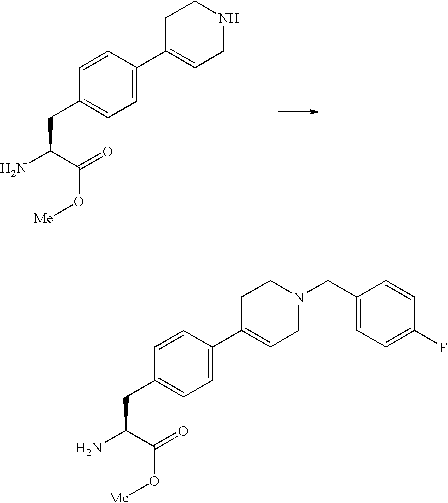

Methyl 4-(1,2,3,6-tetrahydropyridin-4-yl)-L-phenylalaninate (1.77 g) was dissolved in DMF (50 ml) and MP-Carbonate (5.47 g, 2.74 mmol/g) was added. The reaction mixture was cooled in an ice bath for 1 hour then 4-fluorobenzyl bromide (0.5 ml) was added dropwise. The mixture was stirred for 1.5 hours. Piperazine (587 mg) was added and the reaction mixture was allowed to warm to room temperature. The MP-Carbonate was filtered off, washing with DMF and the filtrate concentrated under reduced pressure to give an oil. The oil was purified by chromatography using ethyl acetate to DCM-7% methanol as eluent to give the title compound as a pale brown glass (940 mg, 64%); ¹H NMR-(DMSO-d6) δ 1.70 (2H, m), 2.30 (2H, m), 2.60 (m, 2H), 2.70-2.90 (2H, m), 3.45-3.60 (6H, m), 6.20 (1H, m), 7.0-7.15 (4H, m), 7.20-7.40 (4H, m); Mass Spectrum MH⁺=MH+ 369.57.

### Methyl 4-[1-(pyridin-3-ylsulfonyl)-1,2,3,6-tetrahydropyridin-4-yl]-L-phenylalaninate

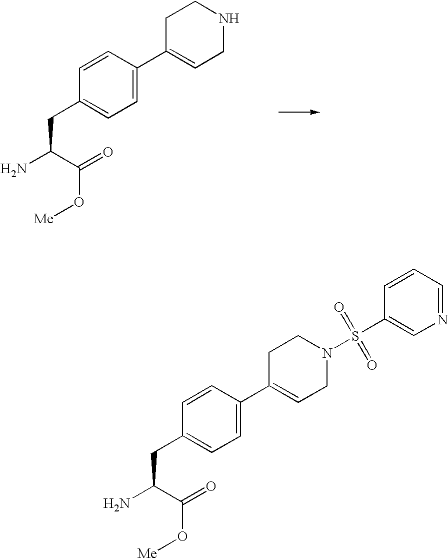

Methyl 4-(1,2,3,6-tetrahydropyridin-4-yl)-L-phenylalaninate (1.63 g) was suspended in DCM (50 ml), diisopropylethylamine (3.27 ml) was added and the mixture cooled to −50° C. 3-Pyridylsulfonylchloride hydrochloride (1.07 g) was added portionwise and stirred at −50° C. for 1 hour. Piperazine (930 mg) was added and the reaction mixture was allowed to warm to room temperature. The reaction mixture was washed with brine (2×25 ml), dried and concentrated in vacuo to give an oil. The oil was purified by chromatography using ethyl acetate to DCM-7% methanol as eluent to give the title compound as a beige solid (930 mg, 46%); ¹H NMR (DMSO-d6, 300 MHz) δ 1.75 (2H, bs), 2.70-2.80 (1H, m), 2.83-2.90 (1H, m), 3.30 (2H, m), 3.55 (1H, m), 3.58 (3H, s), 3.80 (2H, m), 6.18 (1H, bs), 7.16 (2H, d), 7.25 (2H, d), 7.70 (1H, m), 8.30 (m, 1H), 8.95 (1H, m), 9.0 (1H, d); Mass Spectrum MH⁺=402.45.

### Example 4.1

### N-(2-Chloro-6-methylbenzoyl)-4-[1-(pyridin-3-ylsulfonyl)-1,2,3,6-tetrahydropyridin-4-yl]-L-phenylalanine

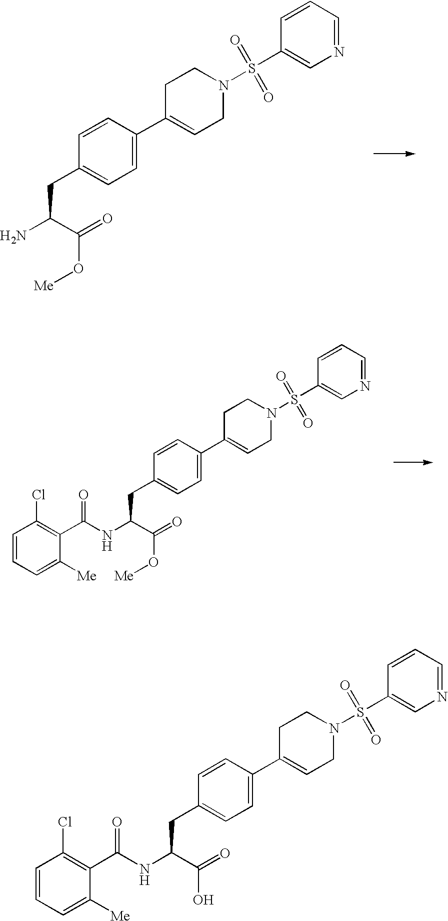

Methyl 4-[1-(pyridin-3-ylsulfonyl)-1,2,3,6-tetrahydropyridin-4-yl]-L-phenylalaninate (80 mg) and triethylamine (33 μl) in DMF (1 ml) was added to 2-chloro-6-methylbenzoic acid (40 mg) followed by a solution of HATU (91 mg) in DMF (1 ml). The mixture was shaken overnight at room temperature then concentrated. The residue was dissolved in ethyl acetate and washed with water (2×5 ml). The organic layer was concentrated and the residue was dissolved in 2:1 acetonitrile-methanol (3 ml) and a solution of lithium hydroxide monohydrate (31 mg) in water (200 μl) was added. The mixture was shaken at room temperature for 12 hours and the solvent removed in vacuo. The crude product was purified by reverse phase preparative HPLC to afford the title compound (79 mg, 72%); ¹H NMR (DMSO-d6) δ 2.03 (3H, s), 2.86-2.92 (1H, m), 3.13-3.18 (1H, m), 3.28-3.31 (1H, m), 3.34-3.37 (1H, m), 3.79-3.82 (2H, m), 4.64-4.71 (1H, m), 6.05-6.08 (1H, m), 7.13-7.16 (1H, m), 7.22-7.28 (6H, m), 7.67-7.71 (1H, m), 8.24-8.27 (1H, m), 8.81 (1H, d), 8.86-8.89 (1H, m), 9.00 (1H, d); Mass Spectrum MH⁺ 540.56.

The procedure described above was repeated using the appropriate methyl 4-[1-(substituted)-1,2,3,6-tetrahydropyridin-4-yl]-L-phenylalaninate intermediate and appropriate carboxylic acid to obtain the compounds listed in Table 4.

TABLE 4

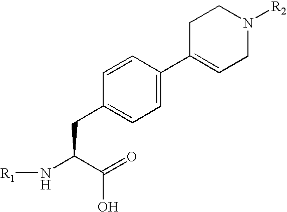

| Example | | | | Mass | |
|---|---|---|---|---|---|
| Number | R₁ | R₂ | Name | Ion | NMR^{a} Data |
| 4.2 | 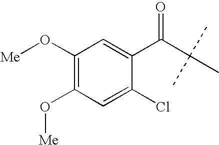 | 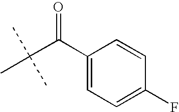 | N-(2-chloro- 4,5- dimethoxy- benzoyl)-4-[1-(4- fluorobenzoyl)- 1,2,3,6- tetrahydropy- ridin-4-yl]-L- phenylalanine | 567.38 (M + H⁺) | 2.98-3.01 (1H, m), 3.20 (1H, m), 3.55 (2H, m), 3.74 (3H, s), 3.80 (3H, s), 4.10 (1H, m), 4.25 (1H, m), 4.65 (1H, m), 6.15 (1H, m), 6.75 (1H, s), 7.00 (1H, s), 7.30-7.31 (4H, m), 7.39 (2H, d), 7.52-7.56 (1H, m), 8.50 (1H, d) |
| 4.3 | 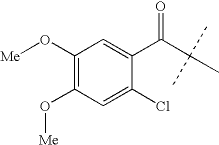 | 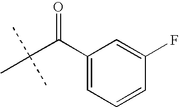 | N-(2-chloro- 4,5- dimethoxy- benzoyl)-4-[1-(3- fluorobenzoyl)- 1,2,3,6- tetrahydropy- ridin-4-yl]-L- phenylalanine | 567.59 (M + H⁺) | 3.01 (1H, m), 3.17 (1H, m), 3.25 (2H, obscured by water), 3.50 (2H, m), 3.74 (3H, s), 3.80 (3H, s), 4.05 (1H, m), 4.25 (1H, m), 4.60 (1H, m), 6.05-6.25 (1H, m), 6.75 (1H, d), 7.01 (1H, d), 7.29-7.33 (5H, m), 7.39 (2H, d), 7.53 (1H, m), 8.51 (1H, d) |
| 4.4 | 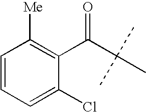 | 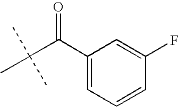 | N-(2-chloro-6- methylbenzoyl)- 4-[1-(3- fluorobenzoyl)- 1,2,3,6- tetrahydropy- ridin-4-yl]-L- phenylalanine | 521.62 (M + H⁺) | 2.06 (3H, d), 2.55 (1H, s), 2.91-2.95 (1H, m), 3.15 (1H, dd), 3.50 (2H, m), 3.85 (1H, m), 4.10 (1H, m), 4.30 (1H, m), 4.70 (1H, m), 6.20 (1H, d), 7.15 (1H, d), 7.23-7.38 (9H, m), 7.54 (1H, d), 8.84 (1H, d) |
| 4.5 | 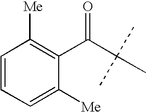 | 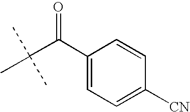 | 4-[1-(4- cyanobenzoyl)- 1,2,3,6- tetrahydropy- ridin-4-yl]-N- (2,6- dimethylbenzoyl)- L- phenylalanine | 508.57 (M + H⁺) | |
| 4.6 | 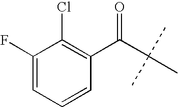 | 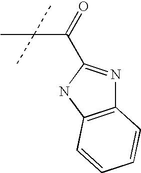 | 4-[1-(1H- benzimidazol- 2-ylcarbonyl)- 1,2,3,6- tetrahydropy- ridin-4-yl]-N- (2-chloro-3- fluorobenzoyl)- L- phenylalanine | 547.56 (M + H⁺) | 2.59-2.65 (1H, m), 2.66-2.73 (1H, m), 2.93- 3.00 (1H, m), 3.16-3.22 (1H, m), 3.94-3.99 (1H, m), 4.36- 4.40 (1H, m), 4.60-4.67 (2H, m), 5.10-5.14 (1H, m), 6.27 (1H, s), 7.07- 7.12 (1H, m), 7.26-7.36 (4H, m), 7.40-7.49 (4H, m), 7.53- 7.58 (1H, m), 7.75-7.81 (1H, m), 8.87 (1H, d) |
| 4.7 | 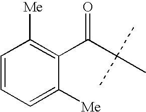 | 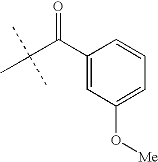 | N-(2,6- dimethylbenzoyl)- 4-[1-(3- methoxybenzoyl)- 1,2,3,6- tetrahydropy- ridin-4-yl]-L- phenylalanine | 513.53 (M + H⁺) | 1.97 (6H, s), 2.85 (1H, dd), 3.18 (1H, dd), 3.55 (2H, m), 3.80 (5H, m), 4.10 (1H, m), 4.25 (1H, m), 4.70 (1H, m), 6.15 (1H, d), 6.95-7.06 (4H, 7.29 (2H, d), 7.39 (3H, m), 8.59 (1H, d) |
| 4.8 | 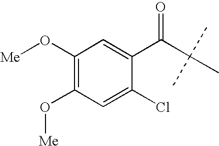 | 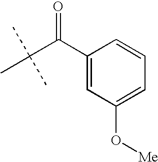 | N-(2-chloro- 4,5- dimethoxyben- zoyl)-4-[1-(3- methoxybenzoyl)- 1,2,3,6- tetrahydropy- ridin-4-yl]-L- phenylalanine | 579.47 (M + H⁺) | 3.01 (1H, d), 3.17 (1H, dd), 3.50 (2H, m), 3.74 (3H, s), 3.80 (6H, s), 3.80-3.84 (2H, m), 4.10 (1H, m), 4.25 (1H, m), 4.60 (1H, m), 6.20 (1H, m), 6.75 (1H, s), 6.97-7.06 (4H, m), 7.30 (2H, d), 7.40 (3H, t), 8.52 (1H, d) |
| 4.9 | 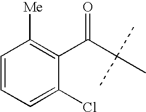 | 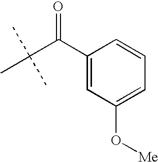 | N-(2-chloro-6- methylbenzoyl)- 4-[1-(3- methoxybenzoyl)- 1,2,3,6- tetrahydropy- ridin-4-yl]-L- phenylalanine | 533.49 (M + H⁺) | 2.06 (3H, s), 2.55 (2H, s), 2.90 (1H, dd), 3.18 (1H, dd), 3.55 (1H, m), 3.80 (5H, m), 4.10 (1H, m), 4.25 (1H, m), 4.70 (1H, m), 6.20 (1H, d), 6.98-7.06 (3H, m), 7.16 (1H, d), 7.24-7.29 (4H, m), 7.37-7.41 (3H, m), 8.80 (1H, d) |
| 4.10 | 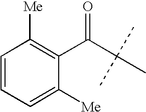 | 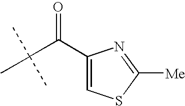 | N-(2,6- dimethylbenzoyl)- 4-{1-[(2- methyl-1,3- thiazol-4- yl)carbonyl]- 1,2,3,6- tetrahydropy- ridin-4-yl}-L- phenylalanine | 504.61 (MH⁺) | 1.97 (6H, s), 2.67-2.72 (2H, m), 2.73 (3H, s), 2.84-2.91 (1H, m), 3.17-3.22 (1H, m), 3.85 (2H, t), 4.25- 4.40 (1H, m), 4.69-4.76 (1H, m), 6.12 (0.5H, s), 6.21 (0.5H, s), 6.96 (2H, d), 7.09-7.13 (1H, m), 7.29 (2H, d), 7.39 (2H, d), 8.59 (1H, d) |
| 4.11 | 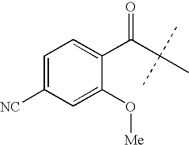 | 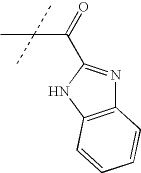 | 4-[1-(1H- benzimidazol- 2-ylcarbonyl)- 1,2,3,6- tetrahydropy- ridin-4-yl]-N- (4-cyano-2- methoxybenzoyl)- L- phenylalanine | 550.64 (MH⁺) | 2.61-2.71 (1H, m), 3.16-3.21 (1H, m), 3.85- 3.90 (1H, m), 3.88 (3H, s), 3.94-3.97 (1H, m), 4.36-4.39 (1H, m), 4.61- 4.71 (2H, m), 5.10-5.13 (1H, m), 6.26-6.29 (1H, m), 7.25- 7.33 (4H, m), 7.43-7.51 (5H, m), 7.62-7.69 (3H, m), 7.77- 7.79 (1H, m), 8.45 (1H, d) |
| 4.12 | 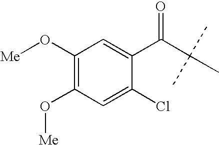 | 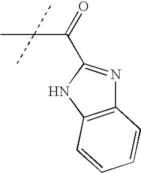 | 4-[1-(1H- benzimidazol- 2-ylcarbonyl)- 1,2,3,6- tetrahydropy- ridin-4-yl]-N- (2-chloro-4,5- dimethoxybenzoyl)- L- phenylalanine | 589.63 (MH⁺) | |
| 4.13 | 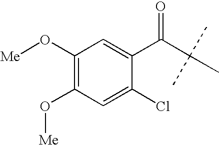 | 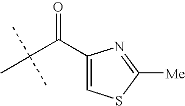 | N-(2-chloro- 4,5- dimethoxybenzoyl)- 4-{1- [(2-methyl- 1,3-thiazol-4- yl)carbonyl]- 1,2,3,6- tetrahydropy- ridin-4-yl}-L- phenylalanine | 570.59 (MH⁺) | 2.72 (3H, s), 2.95-3.02 (1H, m), 3.16-3.21 (1H, m), 3.74 (3H, s), 3.80 (3H, s), 3.83- 3.86 (2H, m), 4.26-4.38 (2H, m), 4.58-4.64 (1H, m), 6.13 (0.5H, s), 6.22 (0.5H, s), 6.75 (1H, s), 7.01 (1H, s), 7.30 (2H, d), 7.40 (2H, d), 7.96 (1H, s), 8.52 (1H, d) |
| 4.14 | 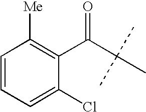 | 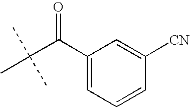 | N-(2-chloro-6- methylbenzoyl)- 4-[1-(3- cyanobenzoyl)- 1,2,3,6- tetrahydropy- ridin-4-yl]-L- phenylalanine | 526.61 (MH)⁻ | 2.06 (3H, s), 2.60 (1H, m), 2.89-2.95 (1H, m), 3.13-3.20 (1H, m), 3.5 (1H, m), 4.03- 4.10 (1H, m), 4.25-4.32 (1H, m), 4.64-4.73 (1H, m), 6.03 (0.4H, s), 6.22 (0.6H, s), 7.14- 7.17 (1H, m), 7.23-7.32 (4H, m), 7.36-7.39 (2H, m), 7.67- 7.72 (1H, m), 7.80-7.83 (1H, m), 7.95-7.98 (2H, m), 8.83 (1H, d) |
| 4.15 | 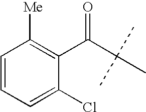 | 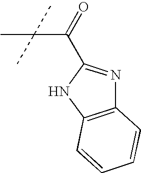 | 4-[1-(1H- benzimidazol- 2-ylcarbonyl)- 1,2,3,6- tetrahydropy- ridin-4-yl]-N- (2-chloro-6- methylbenzoyl)- L- phenylalanine | 543.63 (MH⁺) | |
| 4.16 | 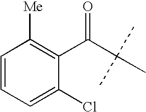 | 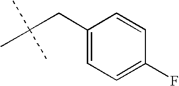 | N-(2-chloro-6- methylbenzoyl)- 4-[1-(4- fluorobenzyl)- 1,2,3,6- tetrahydropyridin- 4-yl]-L- phenylalanine | 507.61 (MH⁺) | (300 MHz) 2.06 (3H, s), 2.76 (2H, d), 2.95 (1H, m), 3.21 (1H, m), 3.68- 3.77 (4H, m), 4.41 (2H, m), 4.70 (1H, m), 6.13 (1H, s), 7.15 (1H, s), 7.22-7.27 (1H, m), 7.31-7.41 (4H, m), 7.63- 7.67 (1H, m), 8.83 (1H, d) |
| 4.17 | 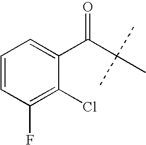 | 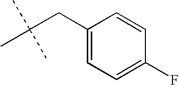 | N-(2-chloro-3- fluorobenzoyl)- 4-[1-(4- fluorobenzyl)- 1,2,3,6- tetrahydropyridin- 4-yl]-L- phenylalanine | 511.58 (MH⁺) | |
| 4.18 | 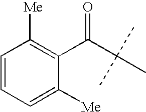 | 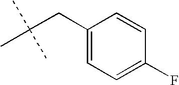 | N-(2,6- dimethylbenzoyl)- 4-[1-(4- fluorobenzyl)- 1,2,3,6- tetrahydropyridin- 4-yl]-L- phenylalanine | 487.64 (M + H⁺) | |
| 4.19 | 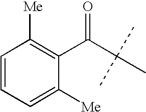 | 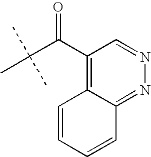 | 4-[1- (cinnolin-4- ylcarbonyl)- 1,2,3,6- tetrahydropyridin- 4-yl]-N- (2,6- dimethylbenzoyl)- L- phenylalanine | 535.65 (M + H⁺) | |
| 4.20 | 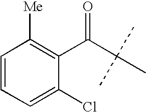 | 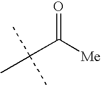 | 4-(1-acetyl- 1,2,3,6- tetrahydropyridin- 4-yl)-N- (2-chloro-6- methylbenzoyl)- L- phenylalanine | 441.57 (M + H⁺) | 2.06 (6H, m), 2.44 (2H, m), 2.90-2.94 (1H, m), 3.15 (1H, dd), 3.62-3.67 (2H, m), 4.14 (2H, d), 4.70 (1H, m), 6.13 (1H, d), 7.14 (2H, d), 7.23- 7.28 (4H, m), 7.30-7.38 (3H, m), 8.82-8.84 (1H, m) |
| 4.21 | 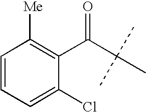 | 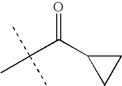 | N-(2-chloro-6- methylbenzoyl)- 4-[1- (cyclopropyl- carbonyl)- 1,2,3,6- tetrahydropyridin- 4-yl]-L- phenylalanine | 467.58 (M + H⁺) | 0.72-0.77 (4H, m), 2.05-2.06 (3H, m), 2.92 (1H, m), 3.16 (1H, m), 3.69 (2H, br s), 3.85 (1H, br s), 4.11 (1H, br s), 4.40 (1H, br s), 4.60- 4.80 (1H, m), 6.16 (1H, m), 7.14 (1H, m), 7.22-7.31 (5H, m), 7.34-7.38 (2H, m), 8.83- 9.00 (1H, d) |
| 4.22 | 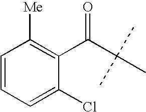 | 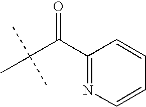 | N-(2-chloro-6- methylbenzoyl)- 4-[1- (pyridin-2- ylcarbonyl)- 1,2,3,6- tetrahydropyridin- 4-yl]-L- phenylalanine | 504.60 (M + H⁺) | 2.06 (3H, s), 2.54 (2H, d), 2.85-2.95 (1H, m), 3.15 (1H, m), 3.61 (1H, t), 3.90 (1H, s), 4.14 (1H, d), 4.31 (1H, d), 4.70 (1H, m), 6.23 (1H, s), 7.15 (1H, d), 7.24 (2H, m), 7.29-7.31 (2H, m), 7.37 (2H, d), 7.51 (1H, m), 7.63 (1H, d), 7.96 (1H, m), 8.63 (1H, m), 8.83 (1H, d) |
| 4.23 | 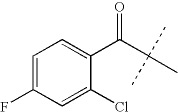 | 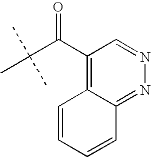 | N-(2-chloro-4- fluorobeuzoyl)- 4-[1- (cinnolin-4- ylcarbonyl)- 1,2,3,6- tetrahydropyridin- 4-yl]-L- phenylalanine | 559.62 (M + H⁺) | 2.70 (1H, m), 2.96-2.99 (1H, m), 3.15 (1H, dd), 3.38 (2H, t), 3.75 (1H, m), 4.00 (1H, m), 4.45 (1H, m), 4.65 (1H, m), 5.95 (0.33H, s), 6.30 (0.67H, s), 7.27-7.30 (4H, m), 7.37-7.40 (2H, m), 7.45- 7.48 (1H, dd), 7.90-8.10 (3H, m), 8.60 (1H, m), 8.77 (1H, d), 9.47 (1H, s) |
| 4.24 | 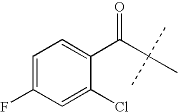 | 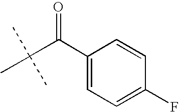 | N-(2-chloro-4- fluorobenzoyl)- 4-[1-(4- fluorobenzoyl)- 1,2,3,6- tetrahydropyridin- 4-yl]-L- phenylalanine | 525.61 (M + H⁺) | 2.95 (1H, m), 3.18 (2H, m), 3.55 (2H, m), 3.85 (1H, m), 4.10 (1H, m), 4.20 (1H, m), 4.60 (1H, m), 6.20 (1H, br d), 7.28-7.56 (10H, m), 7.90 (1H, m), 8.75 (1H, d) |
| 4.25 | 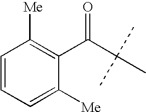 | 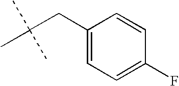 | N-(2,6- dimethylphenyl)- 4-[1-(4- fluorobenzyl)- 1,2,3,6- tetrahydropyridin- 4-yl]-L- phenylalanine | 487.64 (M + H⁺) | (300 MHz) 2.30 (2H, m), 2.60 (6H, s), 2.77- 2.80 (2H, m), 2.90-2.88 (1H, m), 3.02-3.06 (1H, m), 3.51 (2H, m), 3.70 (1H, m), 4.50 (1H, m), 4.70- 4.80 (1H, m) 6.15 (1H, s), 6.95 (2H, d), 7.15-7.19 (2H, m), 7.30-7.50 (4H, m), 7.60- 7.70 (3H, m), 8.60 (1H, d) |

^{a}(400 MHz, DMSO-d₆)

### Example 5.1

### N-(2,6-dichlorobenzoyl)-4-[1-(pyrazin-2-ylcarbonyl)piperidin-4-yl]-L-phenylalanine

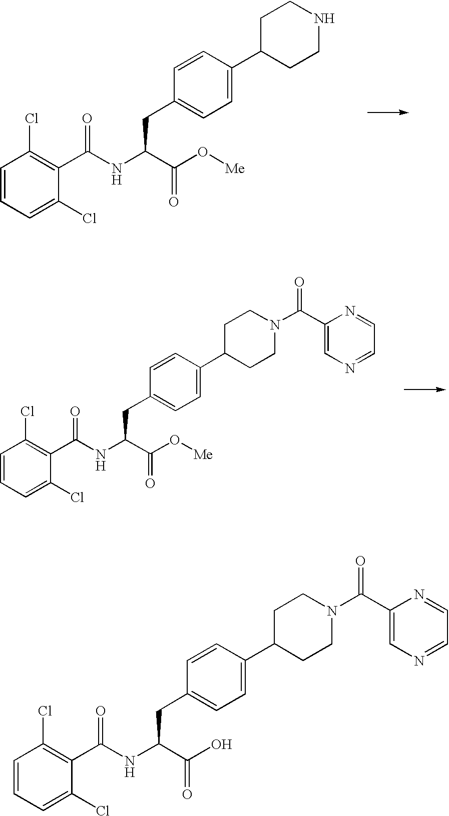

A solution of methyl N-(2,6-dichlorobenzoyl)-4-piperidin-4-yl-L-phenylalaninate (80 mg) and diisopropylethylamine (35 μl) in DMF (1 ml) was added to pyrazine-2-carboxylic acid (27 mg) followed by a solution of HATU (77 mg) in DMF (1 ml). The reaction mixture was shaken overnight at room temperature then concentrated in vacuo. The residue was dissolved in ethyl acetate and washed with water (2×5 ml). The organic layer was concentrated and the residue was dissolved in 1:1 acetonitrile-methanol (2 ml) and a solution of lithium hydroxide monohydrate (31 mg) in water (200 μl) was added. The mixture was shaken at room temperature for 12 hours and the solvent removed. The crude product was purified by reverse phase preparative HPLC to afford the title compound (33 mg, 34%); ¹H NMR (DMSO-d6) δ 1.70-1.90 (4H, m), 2.80-3.03 (4H, m), 3.11-3.12 (1H, m), 3.77 (1H, d), 4.60-4.80 (2H, m), 7.17-7.25 (4H, m), 7.40-7.46 (3H, m), 8.70 (1H, s), 8.75 (1H, d), 8.88 (1H, d), 9.05 (1H, d); Mass Spectrum MH⁺ 527.42.

The methyl N-(2,6-dichlorobenzoyl)-4-piperidin-4-yl-L-phenylalaninate used as the starting material was prepared as follows:

### Methyl N-(2,6-dichlorobenzoyl)-L-tyrosinate

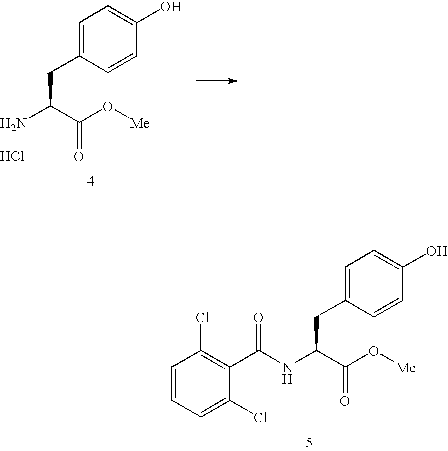

Triethylamine (132 ml) was added to a suspension of L-tyrosine methyl ester hydrochloride (100 g) in DCM (1800 ml) at −10° C. The mixture was stirred at −10° C. for 30 minutes. 2,6-Dichlorobenzoyl chloride (61.8 ml) in DCM (200 ml) was added at −10° C. and the reaction was left to stir for 16 hours at room temperature. The solution was washed with water and brine. A solid product precipitated out and was filtered off. The organic layer was dried and concentrated in vacuo to give a white solid. The two solid products were combined and triturated with diethyl ether and iso-hexane, filtered then dried to give the title compound as a white solid (132.52 g, 83%); ¹H NMR (DMSO-d6) δ 2.85 (1H, dd), 3.0 (1H, dd), 3.65 (3H, s), 4.65 (1H, m), 6.65 (2H, d), 7.05 (2H, d), 7.45 (3H, m), 9.10 (1H, d), 9.15 (1H, s); Mass Spectrum M−H⁺=365.99.

### Methyl N-(2,6-dichlorobenzoyl)-O-[(trifluoromethyl)sulfonyl]-L-tyrosinate

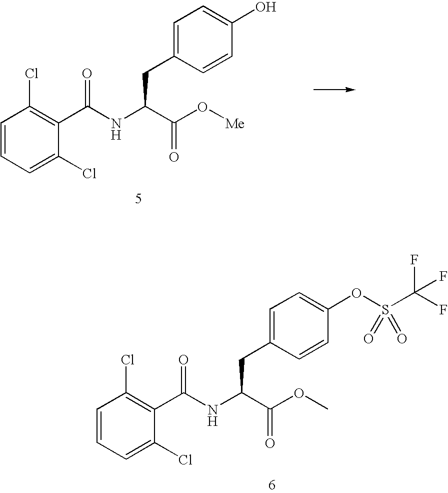

2,6-Lutidine (28.4 ml) was added to a suspension of methyl N-(2,6-dichlorobenzoyl)-L-tyrosinate (60 g) in dry DCM (160 ml) under nitrogen. The solution was cooled to 0° C. and trifluoromethanesulfonic anhydride (32.9 ml) was added slowly. The reaction mixture was stirred for 1.5 hours then concentrated in vacuo. The residue was dissolved in ethyl acetate, washed with water, 1N hydrochloric acid solution and brine, then dried and concentrated to give an orange oil. The oil was dissolved in diethyl ether (400 ml) then iso-hexane added to give a solid which was filtered off and dried to give the title compound (62.5 g, 77%); ¹H NMR (DMSO-d6) δ 2.90 (1H, dd), 3.30 (1H, dd), 3.70 (3H, s), 4.90 (1H, m), 7.30-7.50 (7H, m), 9.20 (1H, d); Mass Spectrum M−H⁺=500.31.

### Methyl-4-[1-(tert-butoxycarbonyl)-1,2,3,6-tetrahydropyridin-4-yl]-N-(2,6-dichlorobenzoyl)-L-phenylalaninate

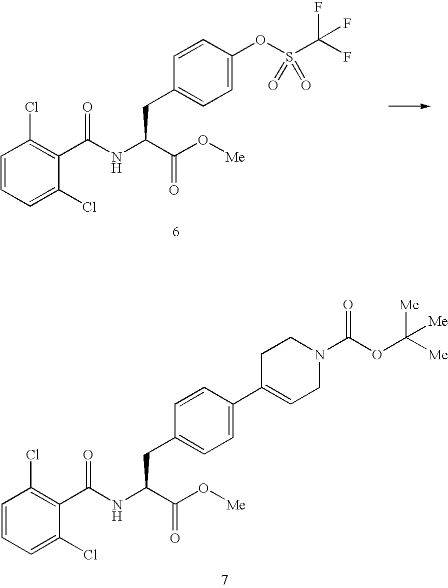

1,1′-Bis(diphenylphosphino)ferrocene-palladium(II)dichloride dichloromethane complex (1.3 g) was added to a degassed solution of methyl N-(2,6-dichlorobenzoyl)-O-[(trifluoromethyl)sulfonyl]-L-tyrosinate (20 g), tert-butyl 4-(4,4,5,5-tetramethyl-1,3,2-dioxaborolan-2-yl)-3,6-dihydropyridine-1(2H)-carboxylate (12.36 g) and potassium carbonate (16.6 g) in DMF (200 ml) under argon. The reaction was heated at 85° C. for three hours then cooled to room temperature and concentrated in vacuo. The residue was partitioned between water and 10% ethyl acetate in diethyl ether. The organic layer was washed twice with water and dried. Concentration gave a dark brown oil which was purified by chromatography using 0-70% ethyl acetate in iso-hexane as eluent to afford the title compound as white solid (14.15 g, 66%); ¹H NMR (DMSO-d6) δ 1.40 (9H, S), 2.20 (2H, m), 2.90 (1H, dd), 3.30 (1H, dd), 3.50 (2H, t), 3.70 (3H, s), 4.0 (m, 2H), 4.80 (1H, m), 6.10 (1H, bs), 7.30 (2H, d), 7.40 (2H, d), 7.50 (3H, m), 9.20 (1H, d); Mass Spectrum M+Na⁺=555.54.

### Methyl N-(2,6-dichlorobenzoyl)-4-(1,2,3,6-tetrahydropyridin-4-yl)-L-phenylalaninate

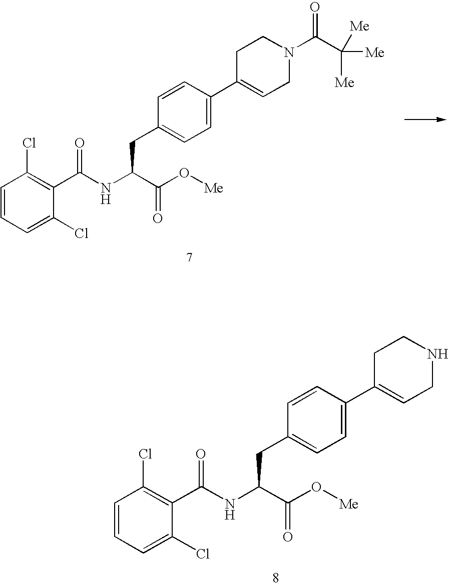

Concentrated HCl (5.61 ml) was added to a solution methyl-4-[1-(tert-butoxycarbonyl)-1,2,3,6-tetrahydropyridin-4-yl]-N-(2,6-dichlorobenzoyl)-L-phenylalaninate (16.5 g) in methanol (500 ml) and the reaction heated at reflux for 3.5 hours. The reaction was cooled, concentrated in vacuo and partitioned between aqueous sodium bicarbonate solution and 10% methanol in ethyl acetate. The organic layer was dried, filtered and concentrated in vacuo to give a yellow solid (12.32 g, 92%); ¹H NMR (DMSO-d6) δ 2.30 (2H, m), 2.80-3.0 (3H, m), 3.10 (1H, m), 3.40 (2H, m), 3.60 (3H, s), 4.80 (1H, m), 6.10 (1H, bs), 7.20 (2H, d), 7.35 (2H, d), 7.40 (3H, m), 9.20 (1H, d); Mass Spectrum M+H⁺=433.5

### Methyl N-(2,6-dichlorobenzoyl)-4-piperidin-4-yl-L-phenylalaninate

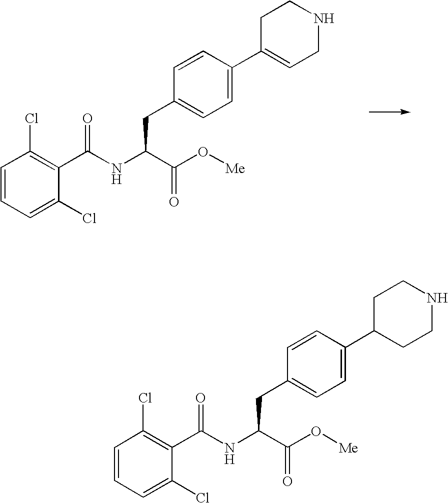

Methyl N-(2,6-dichlorobenzoyl)-4-(1,2,3,6-tetrahydropyridin-4-yl)-L-phenylalaninate (3 g) was dissolved in ethanol (75 ml) and acetic acid (7.5 ml). 10% Platinum on carbon (approximately 50% moisture (750 mg)) was added, and then the solution was degassed, purged with nitrogen, and hydrogenated at atmospheric pressure for 5 hours. The reaction was filtered through celite and the solvent was removed in vacuo. Methanol (5 ml) and ethyl acetate (100 ml) were added, followed by aqueous saturated sodium carbonate solution (100 ml) and the resulting mixture was stirred for 1 hour. The organic layer was separated, washed with water (2×50 ml), dried, and the solvent was removed in vacuo to give the title compound as a yellow solid (2.70 g, 90%); ¹H NMR (DMSO-d6) δ 1.40-1.80 (4H, m), 2.60 (2H, m), 2.90 (1H, m), 3.00-3.20 (3H, m), 3.70 (3H, s), 4.80 (1H, m), 7.10 (2H, d), 7.20 (2H, d), 7.40 (3H, m), 9.20 (1H, d); Mass Spectrum MH⁺ 435.39.

The procedure described above for Example 5.1 was repeated using the appropriate carboxylic acid and methyl N-(2,6-dichlorobenzoyl)-4-piperidin-4-yl-L-phenylalaninate to obtain the compounds listed in Table 5.

TABLE 5

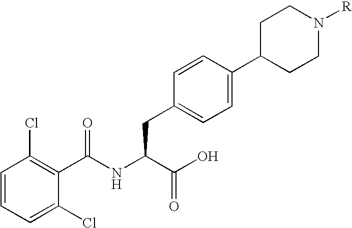

| Example | | | Mass | NMR Data |
|---|---|---|---|---|
| Number | R | Name | Ion | (400 MHz, DMSO-d₆) |
| 5.2 | 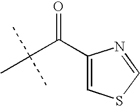 | N-(2,6- dichlorobenzoyl)- 4-[1-(1,3- thiazol-4- ylcarbonyl) piperidin-4-yl]-L- phenylalanine | 532.49 (M + H⁺) | 1.55 (2H, m), 1.80 (2H, m), 2.82 (1H, m), 2.86- 2.92 (2H, m), 3.11-3.15 (2H, m), 4.25 (1H, m), 4.70 (2H, m), 7.16-7.19 (2H, m), 7.22-7.25 (2H, m), 7.38-7.45 (3H, m), 8.16 (1H, d), 9.02 (1H, d), 9.18 (1H, d) |
| 5.3 | 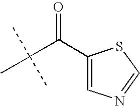 | N-(2,6- dichlorobenzoyl)- 4-[1-(1,3- thiazol-5- ylcarbonyl) piperidin-4-yl]-L- phenylalanine | 532.49 (M + H⁺) | 1.70 (2H, s), 1.70-1.90 (2H, m), 2.80-3.00 (4H, m), 3.19 (2H, m), 4.75 (2H, m), 7.15-7.26 (4H, m), 7.38-7.45 (3H, m), 8.22 (1H, m), 9.02 (1H, d), 9.24 (1H, s) |
| 5.4 | 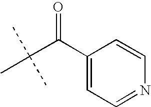 | N-(2,6- dichlorobenzoyl)- 4-(1- isonicotinoyl piperidin-4-yl)-L- phenylalanine | 526.56 (M + H⁺) | 1.66 (2H, d), 1.85 (2H, m), 2.86-2.92 (3H, m), 3.11- 3.16 (1H, m), 3.40 (2H, obscured by water peak), 4.65 (2H, m), 7.19-7.25 (4H, m), 7.37-7.44 (3H, m), 7.51-7.53 (2H, m), 8.71-8.73 (2H, d), 9.00 (1H, d) |
| 5.5 | 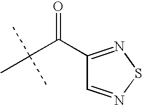 | N-(2,6- dichlorobenzoyl)- 4-[1-(1,2,5- thiadiazol-3- ylcarbonyl) piperidin-4-yl]-L- phenylalanine | 533.52 (M + H⁺) | 1.64-1.69 (2H, m), 1.76 (1H, m), 1.88 (1H, m), 2.80 3.00 (4H, m), 3.11- 3.16 (1H, m), 4.04 (1H, d), 4.67-4.71 (2H, m), 7.18 (2H, d), 7.24 (2H, d), 7.37-7.45 (3H, m), 9.02 (1H, d), 9.12 (1H, s) |
| 5.6 | 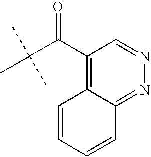 | 4-[1-(cinnolin- 4- ylcarbonyl) piperidin-4-yl]-N- (2,6- dichlorobenzoyl)- L- phenylalanine | 577.61 (M + H⁺) | 1.40 (1H, m), 1.60 (1H, d), 1.80 (1H, m), 1.90 (1H, d), 2.80-2.95 (3H, m), 3.00 (1H, d), 3.16 (1H, dd), 3.28 (1H, under water), 4.70 (1H, m), 4.80 (1H, d), 7.24 (4H, m), 7.39-7.43 (3H, m), 7.95-8.06 (3H, m), 8.57 (1H, d), 9.02 (1H, d), 9.40 (1H, m) |
| 5.7 | 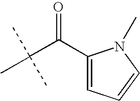 | N-(2,6- dichlorobenzoyl)- 4-{1-[(1- methyl-1H- pyrrol-2- yl)carbonyl] piperidin-4-yl}-L- phenylalanine | 528.59 (M + H⁺) | (500 MHz) 1.54-1.61 (2H, m), 1.80 (2H, m), 2.50 (1H, under DMSO), 2.87-2.92 (2H, m), 3.00 (1H, s), 3.11-3.14 (1H, m), 3.68-3.70 (3H, s), 4.40 (2H, m), 4.66-4.71 (1H, m), 6.04 (1H, t), 6.33 (1H, d), 6.88 (1H, s), 7.18 (2H, d), 7.23 (2H, d), 7.40- 7.44 (1H, m), 7.42-7.43 (2H, m), 9.00 (1H, d) |
| 5.8 | 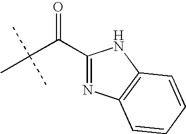 | 4-[1-(1H- benzimidazol-2- ylcarbonyl) piperidin-4-yl]-N- (2,6- dichlorobenzoyl)- L- phenylalanine | 565.61 (M⁺) | (500 MHz) 1.60-1.80 (2H, m), 1.92 (2H, m), 2.74-2.92 (3H, m), 3.11- 3.15 (1H, m), 4.67-4.71 (2H, m), 5.75 (1H, m), 7.19-7.42 (8H, m), 7.55 (1H, d), 7.75 (1H, d), 9.00 (1H, d) |
| 5.9 | 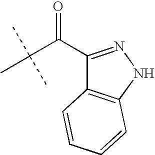 | N-(2,6- dichlorobenzoyl)- 4-[1-(1H- indazol-3- ylcarbonyl) piperidin-4-yl]-L- phenylalanine | 565.60 (M⁺) | (500 MHz) 1.65-1.68 (2H, s), 1.81-1.88 (2H, m), 2.50 2.55 (2H, m), 2.74-2.91 (2H, m), 3.11- 3.15 (1H, m), 4.67-4.71 (1H, m), 4.79 4.84 (2H, m), 7.20 (5H, m), 7.35- 7.45 (4H, m), 7.61 (1H, d), 7.98 (1H, d), 9.01 (1H, d), 13.45 (1H, s) |
| 5.10 | 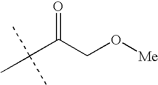 | N-(2,6- dichlorobenzoyl)- 4-[1- (methoxyacetyl) piperidin-4-yl]- L-phenylalanine | 493.38 (M + H⁺) | (500 MHz) 1.40 (1H, m), 1.60 (1H, m), 1.75 (2H, m), 2.55 (3H, s), 2.64 (1H, m), 2.76 (1H, m), 2.88- 2.93 (1H, m), 3.10-3.14 (2H, m), 3.90 (1H, m), 4.10 (2H, m), 4.45 (1H, m), 4.63 (1H, m), 7.13 (2H, d), 7.22 (2H, d), 7.42- 7.43 (3H, m), 8.86 (1H, s) |
| 5.11 | 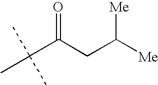 | N-(2,6- dichlorobenzoyl)- 4-[1-(3- methylbutanoyl) piperidin-4-yl]- L-phenylalanine | 505.46 (M + H⁺) | (500 MHz) 0.93 (6H, d), 1.40 (1H, m), 1.51 (1H, d), 1.80 (2H, m), 1.99-2.04 (1H, m), 2.23 (2H, m), 2.60 (1H, m), 2.75 (1H, m), 2.87-2.92 (1H, m), 3.10-3.14 (2H, m), 4.00 (1H, m), 4.55 (1H, m), 4.65 (1H, m), 7.13 (2H, d), 7.22 (2H, d), 7.38-7.43 (3H, m), 8.91 (1H, br s) |
| 5.12 | 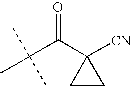 | 4-{1-[(1- cyanocyclopropyl) carbonyl]piperidin- 4-yl}-N- (2,6- dichlorobenzoyl)- L- phenylalanine | 514.37 (M⁺) | (500 MHz) 1.53 (2H, s), 1.60 (4H, d), 1.82 (2H, s), 2.79-2.91 (3H, d), 3.11- 3.14 (1H, m), 3.67 (1H, m), 4.40 (2H, m), 4.67- 4.71 (1H, m), 7.17 (2H, d), 7.24 (2H, d), 7.40-7.43 (3H, m), 9.01 (1H, d) |
| 5.13 | 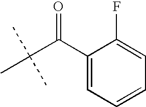 | N-(2,6- dichlorobenzoyl)- 4-[1-(2- fluorobenzoyl) piperidin-4-yl]- L-phenylalanine | 543.51 (MH⁺) | (500 MHz) 1.41-1.54 (2H, m), 1.59-1.67 (1H, m), 1.75-1.81 (1H, m), 2.69-2.75 (1H, m), 2.76- 2.84 (2H, m), 3.02-3.07 (1H, m), 3.37-3.43 (1H, m), 4.56-4.64 (2H, m), 7.09 (2H, d), 7.16 (2H, d), 7.21-7.26 (2H, m), 7.27- 7.36 (4H, m), 7.40-7.46 (1H, m), 8.93 (1H, d) |
| 5.14 | 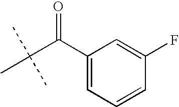 | N-(2,6- dichlorobenzoyl)- 4-[1-(3- fluorobenzoyl) piperidin-4-yl]- L-phenylalanine | 543.52 (MH⁺) | (500 MHz) 1.50-1.59 (3H, m), 1.70-1.79 (1H, m), 2.67-2.75 (1H, m), 2.79-2.84 (1H, m), 3.02- 3.08 (1H, m), 3.50-3.58 (1H, m), 4.49-4.57 (1H, m), 4.59-4.64 (1H, m), 7.11-7.16 (2H, m), 7.19- 7.23 (2H, m), 7.28-7.36 (6H, m), 7.41-7.46 (1H, m), 8.94 (1H, d) |
| 5.15 | 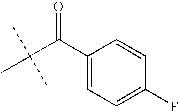 | N-(2,6- dichlorobenzoyl)- 4-[1-(4- fluorobenzoyl) piperidin-4-yl]- L-phenylalanine | 543.52 (MH⁺) | (500 MHz) 1.49-1.58 (2H, m), 1.59-1.77 (2H, m), 2.66-2.74 (1H, m), 2.78-2.84 (1H, m), 3.03- 3.07 (1H, m), 3.56-3.62 (1H, m), 4.48-4.57 (1H, m), 4.57-4.64 (1H, m), 7.10-7.12 (2H, m), 7.15- 7.16 (2H, m), 7.19-7.22 (2H, m), 7.31-7.36 (3H, m), 7.42-7.45 (2H, m), 8.94 (1H, d) |
| 5.16 | 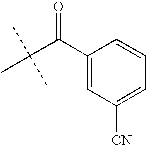 | 4-[1-(3- cyanobenzoyl) piperidin-4-yl]- N-(2,6- dichlorobenzoyl)- L- phenylalanine | 550.52 (MH⁺) | (500 MHz) 1.52-1.62 (3H, m), 1.71-1.80 (1H, m), 2.67-2.75 (1H, m), 2.79-2.85 (1H, m), 3.02- 3.07 (1H, m), 3.45-3.52 (1H, m), 4.51-4.58 (1H, m), 4.58-4.65 (1H, m), 7.11-7.18 (4H, m), 7.28- 7.36 (3H, m), 7.58-7.62 (1H, m), 7.71 (1H, d), 7.84- 7.88 (2H, m), 8.94 (1H, d) |
| 5.17 | 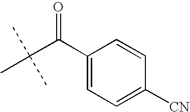 | 4-[1-(4- cyanobenzoyl) piperidin-4-yl]- N-(2,6- dichlorobenzoyl)- L- phenylalanine | 550.52 (MH⁺) | (500 MHz) 1.50-1.61 (3H, m), 1.73-1.80 (1H, m), 2.68-2.75 (1H, m), 2.78-2.84 (1H, m), 3.02- 3.07 (1H, m), 3.07-3.14 (1H, m), 3.41-3.48 (1H, m), 4.52-4.57 (1H, m), 4.58-4.64 (1H, m), 7.11- 7.16 (4H, m), 7.29-7.36 (3H, m), 7.57 (2H, d), 7.86 (2H, d), 8.93 (1H, d) |
| 5.18 | 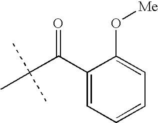 | N-(2,6- dichlorobenzoyl)- 4-[1-(2- methoxybenzoyl) piperidin-4- yl]-L- phenylalanine | 555.53 (MH⁺) | (500 MHz) 1.32-1.63 (3H, m), 1.73-1.78 (1H, m), 2.69-2.75 (1H, m), 2.78-2.83 (1H, m), 2.94- 3.08 (2H, m), 3.30-3.33 (1H, m), 3.70-3.76 (3H, m), 4.56-4.65 (2H, m), 6.91-6.95 (1H, m), 7.00- 7.02 (1H, m), 7.08-7.09 (2H, m), 7.14-7.21 (3H, m), 7.28-7.37 (4H, m), 8.93 (1H, d) |
| 5.19 | 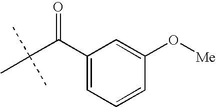 | N-(2,6- dichlorobenzoyl)- 4-[1-(3- methoxybenzoyl) piperidin-4- yl]-L- phenylalanine | 555.54 (MH⁺) | (500 MHz) 1.48-1.56 (2H, m), 1.57-1.79 (2H, m), 2.67-2.72 (1H, m), 2.79-2.84 (1H, m), 3.03- 3.06 (1H, m), 3.72 (3H, s), 4.51-4.57 (1H, m), 4.58- 4.64 (1H, m), 6.89-6.94 (3H, m), 7.11-7.16 (4H, m), 7.28-7.36 (4H, m), 8.93 (1H,d) |
| 5.20 | 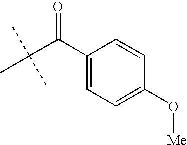 | N-(2,6- dichlorobenzoyl)- 4-[1-(4- methoxybenzoyl) piperidin-4- yl]-L- phenylalanine | 555.55 (MH⁺) | (500 MHz) 1.47-1.56 (2H, m), 1.64-1.72 (2H, m), 2.67-2.73 (1H, m), 2.78-2.84 (1H, m), 3.02- 3.07 (1H, m), 3.73 (3H, s), 4.59-4.64 (1H, m), 6.92 (2H, d), 7.10-7.16 (4H, m), 7.31-7.36 (5H, m), 8.93 (1H, d) |
| 5.21 | 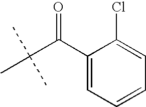 | 4-[1-(2- chlorobenzoyl) piperidin-4-yl]- N-(2,6- dichlorobenzoyl)- L- phenylalanine | 561.48 (MH⁺) | (500 MHz) 1.40-1.65 (3H, m), 1.74-1.83 (1H, m), 2.67-2.75 (1H, m), 2.76-2.84 (2H, m), 3.02- 3.14 (1H, m), 4.56-4.65 (2H, m), 7.05-7.11 (2H, m), 7.15 (2H, d), 7.26- 7.40 (6H, m), 7.41-7.49 (1H, m), 8.93 (1H, d) |
| 5.22 | 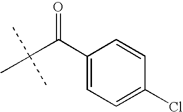 | 4-[1-(4- chlorobenzoyl) piperidin-4-yl]- N-(2,6- dichlorobenzoyl)- L- phenylalanine | 561.45 (MH⁺) | (500 MHz) 1.49-1.57 (2H, m), 1.58-1.79 (2H, m), 2.68-2.73 (1H, m), 2.79-2.84 (1H, m), 3.02- 3.07 (1H, m), 3.51-3.60 (1H, m), 4.50-4.56 (1H, m), 4.59-4.64 (1H, m), 7.11 (2H, d), 7.15 (2H, d), 7.28-7.36 (3H, m), 7.40 (2H, d), 7.44 (2H, d), 8.93 (1H, d) |
| 5.23 | 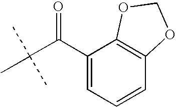 | 4-[1-(1,3- benzodioxol-4- ylcarbonyl) piperidin-4-yl]-N- (2,6- dichlorobenzoyl)- L- phenylalanine | 569.5 (MH⁺) | 1.44-1.53 (2H, m), 1.64- 1.67 (1H, m), 1.76-1.79 (1H, m), 2.68-2.76 (1H, m), 2.78-2.84 (1H, m), 3.02-3.13 (1H, m), 3.56- 3.59 (1H, m), 4.53-4.57 (1H, m), 4.58-4.63 (1H, m), 6.00 (2H, s), 6.79- 6.85 (2H, m), 6.92 (1H, d), 7.08 (2H, d), 7.15 (2H, d), 7.28-7.36 (3H, m), 8.93 (1H, d) |

### Example 6.1

### 4-[1-(Butylsulfonyl)piperidin-4-yl]-N-(2,6-dichlorobenzoyl)-L-phenylalanine

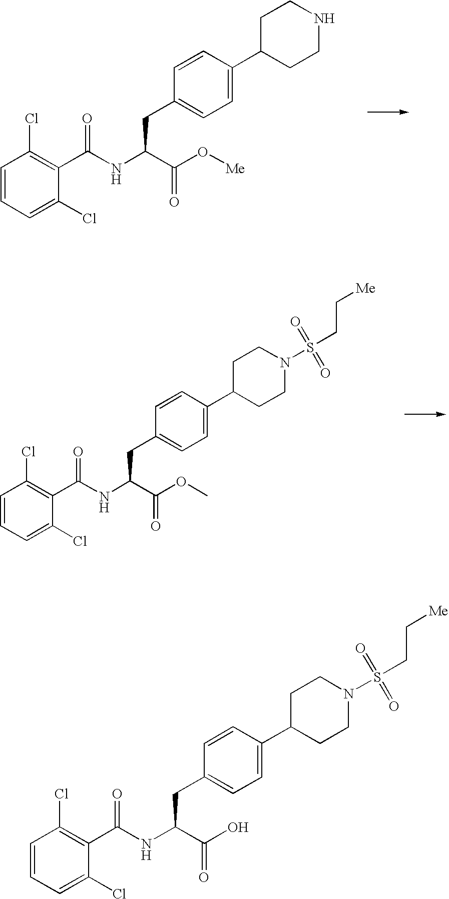

A solution of methyl N-(2,6-dichlorobenzoyl)-4-piperidin-4-yl-L-phenylalaninate (80 mg) in pyridine (3 ml) was added to butane-1-sulfonyl chloride (30 mg). The mixture was shaken overnight at room temperature, then concentrated and the residue was dissolved in 2:1 acetonitrile-methanol (3 ml). A solution of lithium hydroxide monohydrate (24 mg) in water (200 μl) was added and the mixture was shaken at room temperature for 12 hours. The solvent was removed under reduced pressure and the crude product was purified by reverse phase preparative HPLC to afford the title compound (21 mg, 21%); ¹H NMR (DMSO-d6, 500 MHz) δ 0.84 (3H, t), 1.31-1.38 (2H, m), 1.50-1.63 (4H, m), 1.75 (2H, d), 2.51-2.58 (2H, m), 2.82 (3H, t), 2.96-2.99 (2H, m), 3.02-3.08 (1H, m), 3.63 (2H, d), 4.57-4.63 (1H, m), 7.08 (2H, d), 7.16 (2H, d), 7.29-7.36 (3H, m), 8.91 (1H, d); Mass Spectrum MH⁺ 541.54.

The procedure described above was repeated using methyl N-(2,6-dichlorobenzoyl)-4-piperidin-4-yl-L-phenylalaninate and the appropriate sulfonyl chloride to obtain the compounds listed in Table 6.

TABLE 6

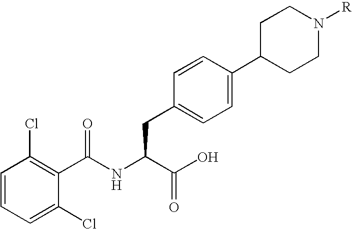

| Example | | | Mass | NMR Data |
|---|---|---|---|---|
| Number | R | Name | Ion | (500 MHz, DMSO-d₆) |
| 6.2 | 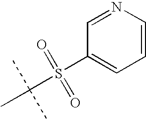 | N-(2,6- dichlorobenzoyl)- 4-[1-(pyridin- 3- ylsulfonyl)piper idin-4-yl]-L- phenylalanine | 561.54 (MH⁺) | 1.53-1.60 (2H, m), 1.69- 1.72 (2H, m), 2.24-2.39 (2H, m), 2.78-2.83 (1H, m), 3.00- 3.05 (1H, m), 3.71 (2H, d), 4.55-4.62 (1H, m), 7.00- 7.02 (2H, m), 7.12 (2H, d), 7.29-7.35 (3H, m), 7.58- 7.72 (4H, m), 8.90 (2H, d), (4H, m), 8.90 (2H, d) |
| 6.3 | 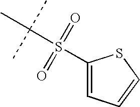 | N-(2,6- dichlorobenzoyl)- 4-[1-(2- thienylsulfonyl) piperidin-4-yl]- L-phenylalanine | 567.49 (M + H⁺) | 1.56-1.64 (2H, m), 1.75 (2H, d), 2.33-2.40 (2H, m), 2.78- 2.83 (1H, m), 3.01-3.05 (1H, m), 3.69 (2H, d), 4.56-4.62 (1H, m), 7.03-7.04 (2H, m), 7.13 (2H, d), 7.22-7.25 (1H, m), 7.28-7.37 (3H, m), 7.59 (1H, d), 7.99 (1H, d), 8.91 (1H, d) |

### Example 7.1

### 4-(1-Benzylpiperidin-4-yl)-N-(2,6-dichlorobenzoyl)-L-phenylalanine

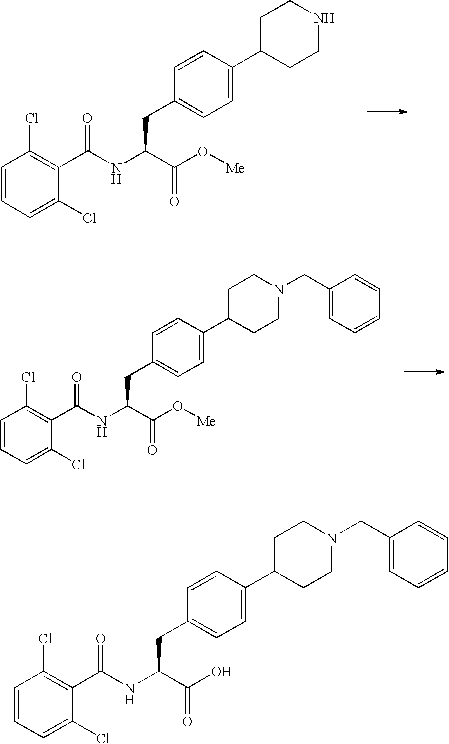

A solution of the methyl N-(2,6-dichlorobenzoyl)-4-piperidin-4-yl-L-phenylalaninate (80 mg) in DMF (1 ml) was added to benzyl bromide (34 mg) then MP-Carbonate (150 mg, 2.74 mmol/g) was added. The mixture was shaken overnight at room temperature then concentrated and the residue was dissolved in 2:1 acetonitrile-methanol (3 ml). A solution of lithium hydroxide monohydrate (24 mg) in water (200 μl) was added and the mixture was shaken at room temperature for 12 hours. The solvent was removed under reduced pressure and the crude product was purified by reverse phase preparative HPLC to afford the title compound (42 mg, 45%); ¹H NMR (DMSO-d6, 500 MHz) δ 1.52-1.65 (4H, m), 1.95-2.03 (2H, m), 2.79-2.87 (3H, m), 3.01-3.05 (1H, m), 3.44 (2H, s), 4.55-4.61 (1H, m), 7.06 (2H, d), 7.13 (2H, d), 7.16-7.20 (1H, m), 7.22-7.35 (7H, m), 8.87 (1H, d); Mass Spectrum MH⁺ 511.41.

The procedure described above was repeated using methyl N-(2,6-dichlorobenzoyl)-4-piperidin-4-yl-L-phenylalaninate and the appropriate benzyl bromide to obtain the compounds listed in Table 7.

TABLE 7

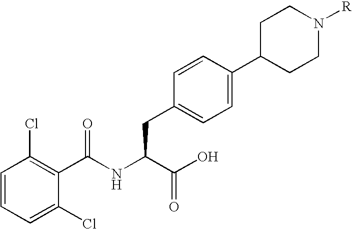

| Example | | | Mass | NMR Data |
|---|---|---|---|---|
| Number | R | Name | Ion | (400 MHz, DMSO-d₆) |
| 7.2 | 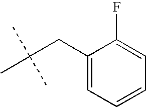 | N-(2,6- dichlorobenzoyl)- 4-[1-(2- fluorobenzyl) piperidin-4-yl]-L- phenylalanine | 529.41 (M⁺) | (500 MHz) 1.63 (2H, d), 1.71 (2H, d), 2.10 (2H, d), 2.55 (2H, m), 2.90-2.94 (2H, d), 3.09-3.13 (1H, m), 3.57 (2H, s), 4.67 (1H, d), 7.14 (2H, d), 7.20 (4H, t), 7.33 (1H, t), 7.37 (1H, d), 7.42-7.46 (3H, m), 8.98 (1H, d) |
| 7.3 | 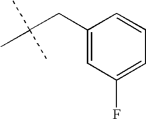 | N-(2,6- dichlorobenzoyl)- 4-[1-(3- fluorobenzyl) piperidin-4-yl]-L- phenylalanine | 529.27 (MH⁺) | 1.53-1.67 (4H, m), 1.96- 2.05 (2H, m), 2.78-2.87 (3H, m), 3.00-3.07 (1H, m), 3.46 (2H, s), 4.55-4.63 (1H, m), 6.97-7.03 (1H, m), 7.07- 7.14 (6H, m), 7.26-7.36 (4H, m), 8.91 (1H, d) |
| 7.4 | 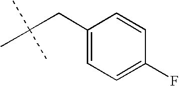 | N-(2,6- dichlorobenzoyl)- 4-[1-(3- fluorobenzyl) piperidin-4-yl]-L- phenylalanine | 529.45 (MH⁺) | 1.51-1.65 (4H, m), 1.96- 2.02 (2H, m), 2.78-2.84 (3H, m), 3.01-3.05 (1H, m), 3.42 (2H, s), 4.56-4.62 (1H, m), 7.05-7.09 (4H, m), 7.13 (2H, d), 7.27-7.35 (5H, m), 8.89 (1H, d) |
| 7.5 | 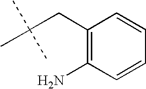 | 4-[1-(2- aminobenzyl) piperidin-4-yl]-N- (2,6- dichlorobenzoyl)- L- phenylalanine | 536.49 (M⁺) | (500 MHz) 1.64 (2H, d), 1.72 (2H, d), 2.17 (1H, d), 2.55 (2H, m), 2.92 (3H, d), 3.10 (1H, d), 3.69 (2H, s), 4.68 (1H, s), 7.15 (2H, d), 7.21 (2H, d), 7.38 (1H, d), 7.42-7.43 (2H, m), 7.48 (1H, t), 7.63 (1H, d), 7.70 (1H, t), 7.82 (1H, d), 8.98 (1H, d) |
| 7.6 | 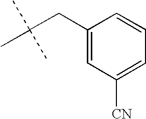 | 4-[1-(3- cyanobenzyl) piperidin-4-yl]-N- (2,6- dichlorobenzoyl)- L- phenylalanine | 536.50 (MH⁺) | 1.54-1.66 (4H, m), 2.02 (2H, t), 2.79-2.83 (3H, m), 3.01- 3.06 (1H, m), 3.50 (2H, s), 4.57-4.63 (1H, m), 7.07 (2H, d), 7.13 (2H, d), 7.28-7.35 (3H, m), 7.46-7.51 (1H, m), 7.61-7.67 (2H, m), 7.69 (1H, s), 8.91 (1H, d) |
| 7.7 | 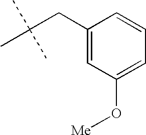 | N-(2,6- dichlorobenzoyl)- 4-[1-(3- methoxybenzyl) piperidin-4-yl]- L-phenylalanine | 541.54 (M⁺) | (500 MHz) 1.65 (2H, d), 1.72 (2H, d), 2.08 (2H, d), 2.55 (2H, m), 2.89-2.95 (2H, m), 3.10-3.14 (1H, m), 3.51 (2H, s), 3.76 (3H, d), 4.67 (1H, d), 6.83 (1H, d), 6.91 (2H, d), 7.15 (2H, d), 7.21-7.22 (2H, m), 7.26 (1H, d), 7.42 (3H, d), 8.98 (1H, d) |
| 7.8 | 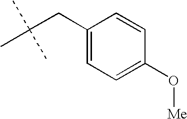 | N-(2,6- dichlorobenzoyl)- 4-[1-(4- methoxybenzyl) piperidin-4-yl]- L-phenylalanine | 541.55 (M⁺) | (500 MHz) 1.62 (1H, s), 1.65 (1H, d), 1.71 (2H, d), 2.08 (2H, m), 2.55 (2H, m), 2.89- 2.95 (2H, m), 3.10-3.13 (1H, m), 3.49 (2H, s), 3.75 (3H, d), 4.67 (1H, d), 6.89 (2H, t), 7.14 (2H, d), 7.20 (1H, s), 7.22 (1H, s), 7.25 (2H, d), 7.40-7.43 (3H, m), 8.96 (1H, d) |
| 7.9 | 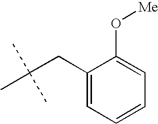 | N-(2,6- dichlorobenzoyl)- 4-[1-(2- methoxybenzyl) piperidin-4-yl]- L-phenylalanine | 541.56 (M⁺) | (500 MHz) 1.66 (2H, d), 1.70 (2H, s), 1.72 (1H, s), 2.13 (2H, s), 2.96 (3H, d), 3.03 (1H, m), 3.53 (2H, s), 3.79 (3H, d), 4.60 (1H, m), 6.95 (2H, d), 6.99 (2H, d), 7.15 (3H, d), 7.20- 7.38 (4H, m), 8.90 (1H, m) |
| 7.10 | 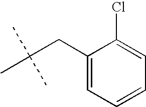 | 4-[1-(2- chlorobenzyl) piperidin-4-yl]-N- (2,6- dichlorobenzoyl)- L- phenylalanine | 547.49 (MH⁺) | (500 MHz) 1.66 (2H, d), 1.72 (2H, d), 2.16 (2H, t), 2.55 (2H, m), 2.95 (2H, d), 3.10-3.11 (1H, m), 3.61 (2H, s), 4.68 (1H, d), 7.16 (2H, d), 7.22 (2H, d), 7.30 (1H, t), 7.35- 7.37 (1H, m), 7.40 (1H, s), 7.43 (3H, t), 7.54 (1H, d), 8.99 (1H, d) |
| 7.11 | 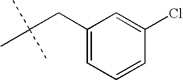 | 4-[1-(3- chlorobenzyl) piperidin-4-yl]-N- (2,6- dichlorobenzoyl)- L- phenylalanine | 547.49 (MH⁺) | (500 MHz) 1.65 (2H, d), 1.71 (2H, d), 2.06 (2H, m), 2.55 (2H, m), 2.90 (3H, d), 3.53 (2H, s), 4.67 (1H, s), 7.15 (2H, d), 7.21 (2H, d), 7.32 (2H, t), 7.37 (1H, d), 7.38 (1H, m), 7.39 (1H, m), 7.42 (1H, m), 7.43 (1H, m), 8.99 (1H, d) |
| 7.12 | 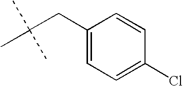 | 4-[1-(4- chlorobenzyl) piperidin-4-yl]-N- (2,6- dichlorobenzoyl)- L- phenylalanine | 547.51 (MH⁺) | 1.51-1.65 (4H, m), 1.98 (2H, t), 2.78-2.83 (3H, m), 3.01- 3.06 (1H, m), 3.42 (2H, s), 4.55-4.62 (1H, m), 7.06 (2H, d), 7.13 (2H, d), 7.27-7.35 (7H, m), 8.88 (1H, d) |

### Example 8.1

### N-(2,6-dichlorobenzoyl)-4-[1-(3-phenylpropanoyl)-1,2,3,6-tetrahydropyridin-4-yl]-L-phenylalanine

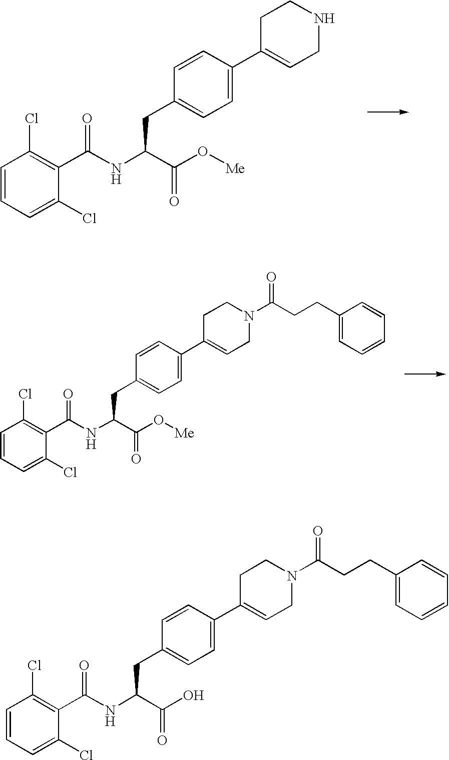

A solution of the methyl N-(2,6-dichlorobenzoyl)-4-(1,2,3,6-tetrahydropyridin-4-yl)-L-phenylalaninate (80 mg) and triethylamine (31 mg) in DMF (1 ml) was added to 3-phenylpropionic acid (29 mg), followed by a solution of HATU (84 mg) in DMF (1 ml). The solution was shaken overnight at room temperature. The reaction mixture was concentrated in vacuo. The residue was dissolved in ethyl acetate and washed with water (2×5 ml). The organic layer was concentrated and the residue was dissolved in 2:1 acetonitrile-methanol (3 ml) and a solution of lithium hydroxide monohydrate (24 mg) in water (200 μl) was added. The mixture was shaken at room temperature for 16 hours and the solvent removed in vacuo. The crude product was purified by reverse phase preparative is HPLC to afford the title compound (41, 40%); ¹H NMR (DMSO-d6, 500 MHz) δ 1.70-1.90 (4H, m), 2.80-3.03 (4H, m), 3.11-3.12 (1H, m), 3.77 (1H, d), 4.60-4.80 (2H, m), 7.17-7.25 (4H, m), 7.40-7.46 (3H, m), 8.70 (1H, s), 8.75 (1H, d), 8.88 (1H, d), 9.05 (1H, d); Mass Spectrum MH⁺ 551.60.

The procedure described above was repeated using methyl N-(2,6-dichlorobenzoyl)-4-(1,2,3,6-tetrahydropyridin-4-yl)-L-phenylalaninate and the appropriate carboxylic acid to obtain the compounds listed in Table 8.

TABLE 8

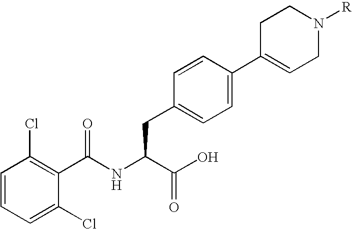

| Example | | | Mass | NMR Data |
|---|---|---|---|---|
| Number | R | Name | Ion | (400 MHz, DMSO-d₆) |
| 8.2 | 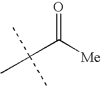 | 4-(1-acetyl-1,2,3,6- tetrahydropyridin-4- yl)-N-(2,6- dichlorobenzoyl)-L- phenylalanine | 461.37 (MH⁺) | (300 MHz) 2.10 (3H, d), 2.45 (2H, m), 2.95- 3.05 (1H, m), 3.15- 3.25 (1H, m), 3.70 (2H, m), 4.70 (1H, m), 6.15 (1H, m), 7.25-7.50 (7H, m), 9.10 (1H, d) |
| 8.3 | 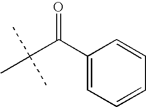 | N-(2,6- dichlorobenzoyl)-4- [1-(benzoyl)-1,2,3,6- tetrahydropyridin-4- yl]-L-phenylalanine | 523.86 (MH⁺) | |
| 8.4 | 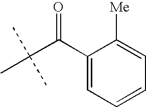 | N-(2,6- dichlorobenzoyl)-4- [1-(2- methylbenzoyl)- 1,2,3,6- tetrahydropyridin-4- yl]-L-phenylalanine | 537.89 (MH⁺) | |
| 8.5 | 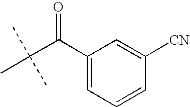 | N-(2,6- dichlorobenzoyl)-4- [1-(3- cyanobenzoyl)- 1,2,3,6- tetrahydropyridin-4- yl]-L-phenylalanine | 548.89 (MH⁺) | |
| 8.6 | 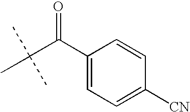 | N-(2,6- dichlorobenzoyl)-4- [1-(4- cyanobenzoyl)- 1,2,3,6- tetrahydropyridin-4- yl]-L-phenylalanine | 548.94 (MH⁺) | |
| 8.7 | 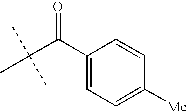 | N-(2,6- dichlorobenzoyl)-4- [1-(4- methylbenzoyl)- 1,2,3,6- tetrahydropyridin-4- yl]-L-phenylalanine | 537.99 (MH⁺) | |
| 8.8 | 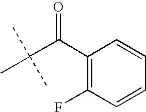 | N-(2,6- dichlorobenzoyl)-4- [1-(2- fluorobenzoyl)- 1,2,3,6- tetrahydropyridin-4- yl]-L-phenylalanine | 541.99 (MH⁺) | |
| 8.9 | 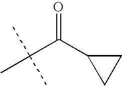 | 4-[1- (cyclopropylcarbonyl)- 1,2,3,6- tetrahydropyridin-4- yl]-N-(2,6- dichlorobenzoyl)-L- phenylalanine | 487.97 (MH⁺) | |
| 8.10 | 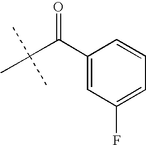 | N-(2,6- dichlorobenzoyl)-4- [1-(3- fluorobenzoyl)- 1,2,3,6- tetrahydropyridin-4- yl]-L-phenylalanine | 542.01 (MH⁺) | |
| 8.11 | 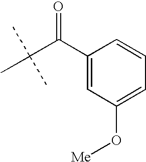 | N-(2,6- dichlorobenzoyl)-4- [1-(3- methoxybenzoyl)- 1,2,3,6- tetrahydropyridin-4- yl]-L-phenylalanine | 554.06 (MH⁺) | |
| 8.12 | 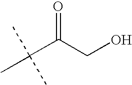 | N-(2,6- dichlorobenzoyl)-4- [1-(hydroxyacetyl)- 1,2,3,6- tetrahydropyridin-4- yl]-L-phenylalanine | 477.94 (M + H⁺) | |
| 8.13 | 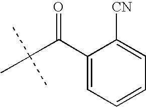 | N-(2,6- dichlorobenzoyl)-4- [1-(2- cyanobenzoyl)- 1,2,3,6- tetrahydropyridin-4- yl]-L-phenylalanine | 548.03 (MH⁺) | |
| 8.14 | 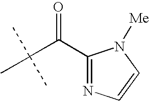 | N-(2,6- dichlorobenzoyl)-4- {1-[(1-methyl-1H- imidazol-2- yl)carbonyl]-1,2,3,6- tetrahydropyridin-4- yl}-L-phenylalanine | 527.53 (MH⁺) | 2.57-2.62 (2H, m), 2.89-2.98 (1H, m), 3.12-3.18 (1H, m), 3.81 (3H, s), 3.85-3.87 (1H, m), 4.03-4.08 (1H, m), 4.26-4.29 (1H, m), 4.56-4.61 (1H, m), 4.66-4.74 (1H, m), 6.14 (0.5H, s), 6.22 (0.5H, s), 7.02- 7.10 (2H, m), 7.27- 7.30 (2H, m), 7.33- 7.45 (5H, m), 9.06 (1H, d) |
| 8.15 | 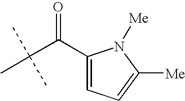 | N-(2,6- dichlorobenzoyl)-4- {1-[(1,5-dimethyl- 1H-pyrrol-2- yl)carbonyl]-1,2,3,6- tetrahydropyridin-4- yl}-L-phenylalanine | 540.55 (MH⁺) | 2.22 (3H, s), 2.53-2.58 (2H, m), 2.90-2.97 (1H, m), 3.11-3.17 (1H, m), 3.57 (3H, s), 3.83 (2H, t), 4.26-4.30 (2H, m), 4.64-4.70 (1H, m), 5.87 (1H, d), 6.16 (1H, s), 6.33 (1H, d), 7.28 (2H, d), 7.35- 7.46 (7H, m), 8.97 (1H, d) |
| 8.16 | 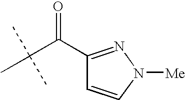 | N-(2,6- dichlorobenzoyl)-4- {1-[(1-methyl-1H- pyrazol-3- yl)carbonyl]-1,2,3,6- tetrahydropyridin-4- yl}-L-phenylalanine | 527.52 (MH⁺) | 2.54-2.58 (1H, m), 2.90-2.99 (1H, m), 3.12-3.17 (1H, m), 3.81-3.87 (1H, m), 3.92 (3H, s), 4.07-4.12 (1H, m), 4.24-4.27 (1H, m), 4.58-4.62 (1H, m), 4.63-4.71 (1H, m), 6.13-6.21 7.28 (2H, d), 7.35-7.46 (5H, m), 7.78 (1H, s), 8.98 (1H, d) |
| 8.17 | 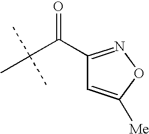 | N-(2,6- dichlorobenzoyl)-4- {1-[(5- methylisoxazol-3- yl)carbonyl]-1,2,3,6- tetrahydropyridin-4- yl}-L-phenylalanine | 528.51 (MH⁺) | 2.47-2.49 (2H, m), 2.56 (3H, s), 2.91-3.00 (1H, m), 3.12-3.18 (1H, m), 3.76 (1H, t), 3.88 (1H, t), 4.26-4.32 (1H, m), 4.60-4.67 (1H, m), 6.11 (0.5H, s), 6.21 (0.5H, s), 6.51 (1H, d), 7.29 (2H, d), 7.34-7.49 (5H, m), 8.86-8.92 (1H, m) |
| 8.18 | 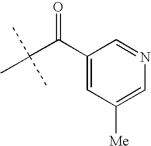 | N-(2,6- dichlorobenzoyl)-4- {1-[(5- methylpyridin-3- yl)carbonyl]-1,2,3,6- tetrahydropyridin-4- yl}-L-phenylalanine | 538.56 (MH⁺) | 2.38 (3H, s), 2.54-2.59 (2H, m), 2.91-3.00 (1H, m), 3.12-3.17 (1H, m), 3.51-3.58 (1H, m), 3.85-3.89 (1H, m), 4.07-4.12 (1H, m), 4.26-4.31 (1H, m), 4.64-4.70 (1H, m), 6.06 (0.5H, s), 6.24 (0.5H, s), 7.29 (2H, d), 7.34-7.46 (5H, m), 7.73 (1H, s), 8.47 (1H, s), 8.52 (1H, s), 8.98 (1H, d) |
| 8.19 | 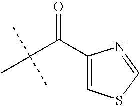 | N-(2,6- dichlorobenzoyl)-4- [1(1,3-thiazol-4- ylcarbonyl)-1,2,3,6- tetrahydropyridin-4- yl]-L-phenylalanine | 530.47 (MH⁺) | 2.56-2.59 (2H, m), 2.92-3.00 (1H, m), 3.11-3.18 (1H, m), 3.83-3.89 (2H, m), 4.31 (1H, s), 4.39 (1H, s), 4.63-4.70 (1H, m), 6.11 (0.5H, s), 6.24 (0.5H, s), 7.29 (2H, d), 7.35-7.46 (5H, m), 8.22 (1H, d), 8.96 (1H, d), 9.23 (1H, s) |
| 8.20 | 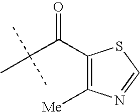 | N-(2,6- dichlorobenzoyl)-4- {1-[(4-methyl-1,3- thiazol-5- yl)carbonyl]-1,2,3,6- tetrahydropyridin-4- yl}-L-phenylalanine | 544.49 (MH⁺) | |
| 8.21 | 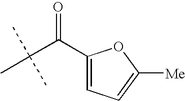 | N-(2,6- dichlorobenzoyl)-4- [1-(5-methyl-2- furoyl)-1,2,3,6- tetrahydropyridin-4- yl]-L-phenylalanine | 527.50 (MH⁺) | 2.37 (3H, s), 2.55-2.60 (2H, m), 2.90-2.98 (1H, m), 3.11-3.18 (1H, m), 3.84-3.91 (2H, m), 4.30-4.37 (2H, m), 4.64-4.73 6.28 (1H, d), 6.95 (1H, d), 7.28 (2H, d), 7.35- 7.46 (5H, m), 9.00 (1H, d) |
| 8.22 | 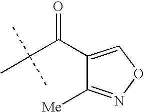 | N-(2,6- dichlorobenzoyl)-4- {1-[(3- methylisoxazol-4- yl)carbonyl]-1,2,3,6- tetrahydropyridin-4- yl}-L-phenylalanine | 528.77 (MH⁺) | 2.31 (3H, s), 2.54-2.62 (2H, m), 2.91-2.98 (1H, m), 3.12-3.17 (1H, m), 3.69-3.84 (2H, m), 4.22-4.26 (2H, m), 4.63-4.70 (1H, m), 6.20 (1H, s), 7.29 (2H, d), 7.35-7.46 (5H, m), 8.98 (1H, d), 9.22 (1H, s) |
| 8.23 | 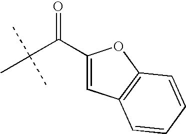 | 4-[1-(1-benzofuran- 2-ylcarbonyl)- 1,2,3,6- tetrahydropyridin-4- yl]-N-(2,6- dichlorobenzoyl)-L- phenylalanine | 564.04 (MH⁺) | |
| 8.24 | 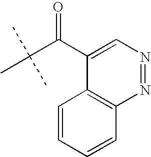 | 4-[1-(cinnolin-4- ylcarbonyl)-1,2,3,6- tetrahydropyridin-4- yl]-N-(2,6- dichlorobenzoyl)-L- phenylalanine | 576.04 (MH⁺) | 2.91-3.01 (1H, m), 3.11-3.19 (1H, m), 3.38 (2H, t), 3.82-3.89 (1H, m), 4.46-4.52 (1H, m), 4.63-4.78 (2H, m), 5.94 (0.3H, s), 6.29 (0.7H, s), 7.28- 7.31 (2H, m), 7.35- 7.38 (2H, m), 7.38- 7.46 (3H, m), 7.88- 8.09 (3H, m), 8.54- 8.63 (1H, m), 9.04 (1H, d), 9.37-9.47 (1H, m) |
| 8.25 | 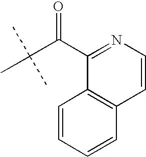 | N-(2,6- dichlorobenzoyl)-4- [1-(isoquinolin-1- ylcarbonyl)-1,2,3,6- tetrahydropyridin-4- yl]-L-phenylalanine | 575.03 (MH⁺) | |
| 8.26 | 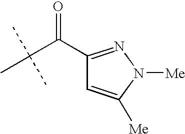 | N-(2,6- dichlorobenzoyl)-4- {1-[(1,5-dimethyl- 1H-pyrazol-3- yl)carbonyl]-1,2,3,6- tetrahydropyridin-4- yl}-L-phenylalanine | 542.01 (MH⁺) | |
| 8.27 | 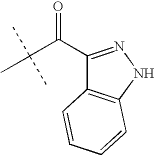 | N-(2,6- dichlorobenzoyl)-4- [1-(1H-indazol-3- ylcarbonyl)-1,2,3,6- tetrahydropyridin-4- yl]-L-phenylalanine | 564.03 (MH⁺) | 2.55-2.61 (2H, m), 2.83-2.90 (1H, m), 3.03-3.10 (1H, m), 3.85-3.91 (1H, m), 4.10-4.15 (1H, m), 4.28-4.32 (1H, m), 4.56-4.65 (2H, m), 6.08 (0.5H, s), 6.18 (0.5H, s), 7.14-7.22 (4H, m), 7.28-7.38 (7H, m), 7.55 (1H, d), 7.93 (1H, d), 8.94 (1H, d) |
| 8.28 | 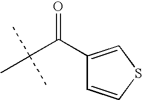 | N-(2,6- dichlorobenzoyl)-4- [1-(3- thienylcarbonyl)- 1,2,3,6- tetrahydropyridin-4- yl]-L-phenylalanine | 529.96 (MH⁺) | |
| 8.29 | 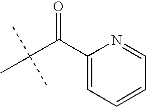 | N-(2,6- dichlorobenzoyl)-4- ylcarbonyl)-1,2,3,6- tetrahydropyridin-4- yl]-L-phenylalanine | 524.99 (MH⁺) | |
| 8.30 | 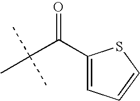 | N-(2,6- dichlorobenzoyl)-4- [1-(2- thienylcarbonyl)- 1,2,3,6- tetrahydropyridin-4- yl]-L-phenylalanine | 529.96 (MH⁺) | |
| 8.31 | 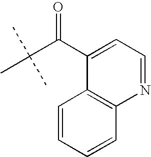 | N-(2,6- dichlorobenzoyl)-4- [1-(quinolin-4- ylcarbonyl)-1,2,3,6- tetrahydropyridin-4- yl]-L-phenylalanine | 565.95 (MH⁺) | |
| 8.32 | 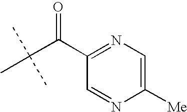 | N-(2,6- dichlorobenzoyl)-4- {1-[(5- methylpyrazin-2- yl)carbonyl]-1,2,3,6- tetrahydropyridin-4- yl}-L-phenylalanine | 540.03 (MH⁺) | 2.55-2.61 (2H, m), 2.7 (3H, s), 3.03-3.10 (1H, m), 3.20 (1H, m), 3.65 (0.7H, t), 3.90 (0.3H, t), 4.30-4.15 (2H, m), 4.70 (1H, m), 6.08 (0.5H, s), 6.18 (0.5H, s), 7.25 (2H, m), 7.28- 7.40 (6H, m), 8.55 (1H, d), 8.7 (1H, d), 9.10 (1H, d) |
| 8.33 | 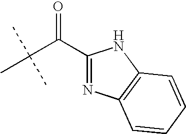 | 4-[1-(1H- benzimidazol-2- ylcarbonyl)-1,2,3,6- tetrahydropyridin-4- yl]-N-(2,6- dichlorobenzoyl)-L- phenylalanine | 564.04 (MH⁺) | |
| 8.34 | 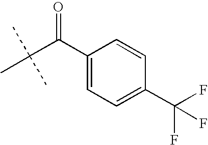 | N-(2,6- dichlorobenzoyl)-4- {1-[4- (trifluoromethyl) benzoyl]-1,2,3,6- tetrahydropyridin-4- yl}-L-phenylalanine | 591.50 (MH⁺) | |
| 8.35 | 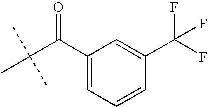 | N-(2,6- dichlorobenzoyl)-4- {1-[3- (trifluoromethyl) benzoyl]-1,2,3,6- tetrahydropyridin-4- yl}-L-phenylalanine | 591.50 (MH⁺) | |
| 8.36 | 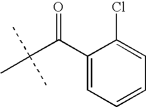 | N-(2,6- dichlorobenzoyl)-4- {1-[2- chlorobenzoyl]- 1,2,3,6- tetrahydropyridin-4- yl}-L-phenylalanine | 559.44 (MH⁺) | |
| 8.37 | 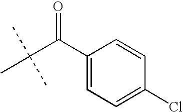 | N-(2,6- dichlorobenzoyl)-4- {1-[3- chlorobenzoyl]- 1,2,3,6- tetrahydropyridin-4- yl}-L-phenylalanine | 559.45 (MH⁺) | |
| 8.38 | 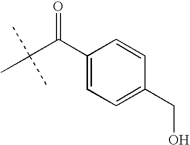 | N-(2,6- dichlorobenzoyl)-4- {1-[4- (hydroxymethyl) benzoyl]-1,2,3,6- tetrahydropyridin-4- yl}-L-phenylalanine | 553.48 (MH⁺) | |
| 8.39 | 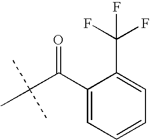 | N-(2,6- dichlorobenzoyl)-4- {1-[2- (trifluoromethyl) benzoyl]-1,2,3,6- tetrahydropyridin-4- yl}-L-phenylalanine | 591.50 (MH⁺) | |
| 8.40 | 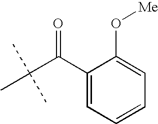 | N-(2,6- dichlorobenzoyl)-4- {1-[2- methoxybenzoyl]- 1,2,3,6- tetrahydropyridin-4- yl}-L-phenylalanine | 553.50 (MH⁺) | |
| 8.41 | 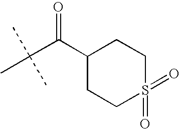 | N-(2,6- dichlorobenzoyl)-4- {1-[(1,1- dioxidotetrahydro- 2H-thiopyran-4- yl)carbonyl]-1,2,3,6- tetrahydropyridin-4- yl}-L-phenylalanine | 579.57 (MH⁺) | |
| 8.42 | 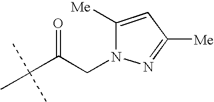 | N-(2,6- dichlorobenzoyl)-4- {1-[(3,5-dimethyl- 1H-pyrazol-1- yl)acetyl]-1,2,3,6- tetrahydropyridin-4- yl}-L-phenylalanine | 555.48 (M⁺) | |
| 8.43 | 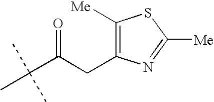 | N-(2,6- dichlorobenzoyl)-4- {1-[(2,5-dimethyl- 1,3-thiazol-4- yl)acetyl]-1,2,3,6- tetrahydropyridin-4- yl}-L-phenylalanine | 572.48 (M⁺) | |
| 8.44 | 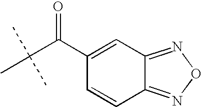 | 4-[1-(2,1,3- benzoxadiazol-5- ylcarbonyl)-1,2,3,6- tetrahydropyridin-4- yl]-N-(2,6- dichlorobenzoyl)-L- phenylalanine | 565.41 (M⁺) | |
| 8.45 | 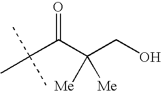 | N-(2,6- dichlorobenzoyl)-4- [1-(3-hydroxy-2,2- dimethylpropanoyl)- 1,2,3,6- tetrahydropyridin-4- yl]-L-phenylalanine | 519.55 (M + H⁺) | |
| 8.46 | 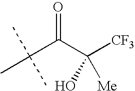 | N-(2,6- dichlorobenzoyl)-4- {1-[(2R)-3,3,3- trifluoro-2-hydroxy- 2-methylpropanoyl]- 1,2,3,6- tetrahydropyridin-4- yl}-L-phenylalanine | 560.13 (M + H⁺) | |
| 8.47 | 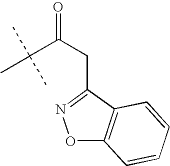 | 4-[1-(1,2- benzisoxazol-3- ylacetyl)-1,2,3,6- tetrahydropyridin-4- yl]-N-(2,6- dichlorobenzoyl)-L- phenylalanine | 578.56 (M⁺) | |
| 8.48 | 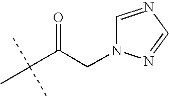 | N-(2,6- dichlorobenzoyl)-4- [1-(1H-1,2,4-triazol- 1-ylacetyl)-1,2,3,6- tetrahydropyridin-4- yl]-L-phenylalanine | 528.54 (M⁺) | |
| 8.49 | 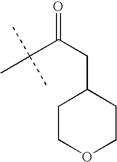 | N-(2,6- dichlorobenzoyl)-4- [1-(tetrahydro-2H- pyran-4-ylacetyl)- 1,2,3,6- tetrahydropyridin-4- yl]-L-phenylalanine | 545.62 (M⁺) | |
| 8.50 | 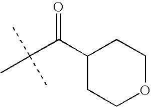 | N-(2,6- dichlorobenzoyl)-4- [1-(tetrahydro-2H- pyran-4-ylcarbonyl)- 1,2,3,6- tetrahydropyridin-4- yl]-L-phenylalanine | 531.58 (M⁺) | |
| 8.51 | 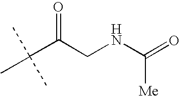 | 4-[1-(N- acetylglycyl)- 1,2,3,6- tetrahydropyridin-4- yl]-N-(2,6- dichlorobenzoyl)-L- phenylalanine | 518.56 (M⁺) | 1.89 (3H, s), 2.96 (1H, m), 2.99-3.01 (1H, m), 3.17 (1H, m), 3.23 (3H, t), 3.63 (1H, s), 3.64- 3.67 (1H, m), 3.96- 4.10 (2H, m), 4.50- 4.60 (1H, m), 6.13 (1H, m), 7.27-7.29 (4H, m), 7.32-7.45 (3H, m), 8.00 (0.5H, m), 8.85 (0.5H, d) |
| 8.52 | 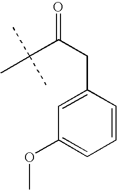 | N-(2,6- dichlorobenzoyl)-4- {1-[(3- methoxyphenyl) acetyl]1,2,3,6- tetrahydropyridin-4- yl}-L-phenylalanine | 567.59 (M⁺) | 2.40 (2H, m), 2.94 (1H, m), 3.12 (1H, d), 3.68- 3.78 (7H, m), 4.13 (1H, m), 4.19 (1H, m), 4.68 (1H, s), 6.14 (1H, d), 6.81-6.85 (3H, m), 7.22-7.45 (8H, m), 9.05 (1H, d) |
| 8.53 | 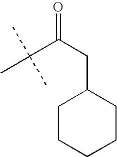 | 4-[1- (cyclohexylacetyl)- 1,2,3,6- tetrahydropyridin-4- yl]-N-(2,6- dichlorobenzoyl)-L- phenylalanine | 543.63 (M⁺) | 0.93-0.97 (2H, m), 1.10-1.30 (3H, m), 1.55-1.80 (6H, m), 2.27 (2H, dd), 2.42 (2H, m), 2.91-2.97 (1H, m), 3.12-3.16 (1H, m), 3.66-3.69 (2H, m), 4.11 (1H, s), 4.16 (1H, s), 4.69 (1H, m), 6.14 (1H, d), 7.28 (2H, d), 7.35 (2H, d), 7.40-7.45 (3H, m), 9.01 (1H, d) |
| 8.54 | 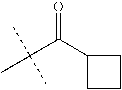 | 4-[1- (cyclobutylcarbonyl)- 1,2,3,6- tetrahydropyridin-4- yl]-N-(2,6- dichlorobenzoyl)-L- phenylalanine | 543.64 (MH⁺ + CH₃CN) | 1.75 (1H, m), 1.90 (1H, m), 2.10-2.25 (4H, m), 2.40 (2H, m), 2.95 (2H, m), 3.13 (1H, dd), 3.53 (1H, t), 3.67 (1H, t), 4.00-4.09 (2H, d), 4.70 (1H, m), 6.13 (1H, d), 7.28 (2H, d), 7.33- 7.36 (2H, m), 7.37- 7.45 (3H, m), 9.04 (1H, d) |
| 8.55 | 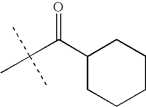 | 4-[1- (cyclohexylcarbonyl)- 1,2,3,6- tetrahydropyridin-4- yl]-N-(2,6- dichlorobenzoyl)-L- phenylalanine | 529.61 (M⁺) | 1.10-1.40 (6H, m), 1.65-1.75 (5H, m), 2.44 (2H, m), 2.95 (1H, m), 3.12 (1H, dd), 3.68 (2H, d), 4.09-4.19 (2H, d), 4.70 (1H, m), 6.15 (1H, s), 7.28 (2H, d), 7.36 (2H, d), 7.37- 7.45 (3H, m), 9.04 (1H, d) |
| 8.56 | 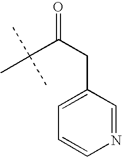 | N-(2,6- dichlorobenzoyl)-4- [1-(pyridin-3- ylacetyl)-1,2,3,6- tetrahydropyridin-4- yl]-L-phenylalanine | 538.56 (M⁺) | 3.00 (3H, s), 3.18 (1H, s), 3.67 (1H, m), 3.77 (1H, m), 3.86 (1H, s), 3.91 (1H, s), 4.10 (1H, s), 4.25 (1H, s), 4.75 (1H, m), 6.16 (1H, s), 7.28-7.30 (2H, m), 7.34-7.45 (3H, m), 7.42-7.46 (3H, m), 7.75 (1H, m), 8.50- 8.51 (2H, m), 9.05 (1H, d) |
| 8.57 | 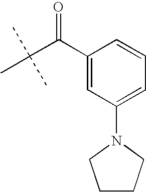 | N-(2,6- dichlorobenzoyl)-4- [1-(3-pyrrolidin-1- ylbenzoyl)-1,2,3,6- tetrahydropyridin-4- yl]-L-phenylalanine | 592.39 (M⁺) | (500 MHz) 1.97 (4H, s), 2.54 (2H, m), 2.95 (1H, m), 3.15 (1H, m), 3.30 (3H, m), 3.50 (2H, m), 3.80 (1H, m), 4.10 (1H, m), 4.25 (1H, m), 4.70 (1H, m), 6.05- 6.25 (1H, br d), 6.51 (1H, s), 6.61-6.62 (1H, d), 7.20-7.30 (3H, m), 7.44-7.50 (5H, m), 9.04 (1H, d) |
| 8.58 | 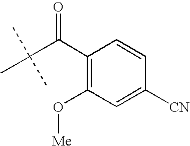 | 4-[1-(4-cyano-2- methoxybenzoyl)- 1,2,3,6- tetrahydropyridin-4- yl]-N-(2,6- dichlorobenzoyl)-L- phenylalanine | 578.41 (M⁺) | (500 MHz) 2.54 (2H, m), 2.95-3.00 (1H, m), 3.12 (1H, m), 3.77 (1H, s), 3.85 (1H, s), 3.89 (4H, d), 4.28 (1H, s), 4.69 (1H, d), 6.00- 6.20 (1H, d), 7.28 (2H, d), 7.36 (2H, t), 7.44 (4H, t), 7.63 (2H, d), 9.04 (1H, d) |
| 8.59 | 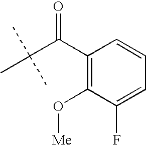 | N-(2,6- dichlorobenzoyl)-4- [1-(3-fluoro-2- methoxybenzoyl)- 1,2,3,6- tetrahydropyridin-4- yl]-L-phenylalanine | 571.52 (M⁺) | (500 MHz) 2.54 (2H, m), 2.95 (1H, m), 3.00- 3.16 (3H, m), 3.40 (1H, d), 3.87 (4H, s), 4.70 (1H, d), 6.12-6.22 (1H, d), 7.07-7.48 (9H, m), 7.68 (1H, m), 9.08 (1H, d) |
| 8.60 | 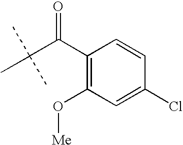 | 4-[1-(4-chloro-2- methoxybenzoyl)- 1,2,3,6- tetrahydropyridin-4- yl]-N-(2,6- dichlorobenzoyl)-L- phenylalanine | 589.44 (MH⁺) | (500 MHz) 2.54 (2H, m), 2.95 (1H, d), 3.00- 3.15 (3H, m), 3.72 (1H, s), 3.81 (1H, s), 3.85 (3H, m), 4.69 (1H, d), 6.19-6.25 (1H, d), 7.07-7.09 (1H, m), 7.21-7.44 (8H, s), 7.65 (1H, m), 9.04 (1H, d) |
| 8.61 | 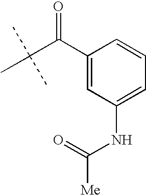 | 4-{1-[3- (acetylamino)benzoyl]- 1,2,3,6- tetrahydropyridin-4- yl}-N-(2,6- dichlorobenzoyl)-L- phenylalanine | 580.54 (M⁺) | (500 MHz) 2.06 (5H, d), 2.54 (2H, m), 2.95 (1H, m), 3.00-3.20 (3H, m), 4.70 (1H, s), 6.30-6.50 (1H, d), 7.07-7.48 (9H, m), 7.63 (1H, d), 7.74-7.76 (1H, m), 9.04 (1H, m), 10.05 (1H, m) |
| 8.62 | 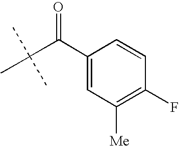 | N-(2,6- dichlorobenzoyl)-4- [1-(4-fluoro-3- methylbenzoyl) 1,2,3,6- tetrahydropyridin-4- yl]-L-phenylalanine | 555.53 (M⁺) | (500 MHz) 2.28 (3H, d), 2.54 (2H, m), 2.95 (1H, m), 3.00-3.20 (2H, m), 3.20 (2H obscured by water peak), 3.60-3.70 (1H, m), 4.75 (1H, m), 7.23- 7.48 (8H, s), 7.65- 7.90 (2H, m), 9.05- 9.08 (1H, m) |
| 8.63 | 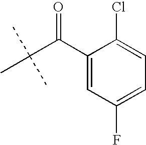 | 4-[1-(2-chloro-5- fluorobenzoyl)- 1,2,3,6- tetrahydropyridin-4- yl]-N-(2,6- dichlorobenzoyl)-L- phenylalanine | 577.22 (MH⁺) | (500 MHz) 2.54 (2H, m), 3.00-3.03 (4H, m), 3.67 (2H, d), 4.70 (1H, m), 6.32 (1H, d), 7.26- 7.42 (8H, m), 7.69 (2H, d), 9.20 (1H, s) |
| 8.64 | 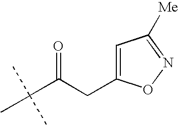 | N-(2,6- dichlorobenzoyl)-4- {1-[(3- methylisoxazol-5- yl)acetyl]-1,2,3,6- tetrahydropyridin-4- yl}-L-phenylalanine | 542.40 (MH⁺) | (500 MHz) 2.21 (3H, s), 2.92-2.97 (1H, m), 3.11-3.17 (1H, m), 3.64-3.67 (1H, m), 3.69-3.73 (2H, m), 4.00 (1H, s), 4.05 (1H, s), 4.11-4.14 (1H, m), 4.22-4.24 (1H, m), 4.67-4.72 (1H, m), 6.14 (1H, s), 6.21 (1H, d), 7.28-7.29 (3H, m), 7.34-7.45 (5H, m), 9.03 (1H, d) |
| 8.65 | 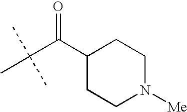 | N-(2,6- dichlorobenzoyl)-4- [1-(1- methylpiperidin-4- ylcarbonyl)-1,2,3,6- tetrahydropyridin-4- yl]-L-phenylalanine | 544.62 (MH⁺) | |
| 8.66 | 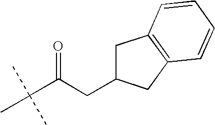 | N-(2,6- dichlorobenzoyl)-4- [1-(2,3-dihydro-1H- inden-2-ylacetyl)- 1,2,3,6- tetrahydropyridin-4- yl]-L-phenylalanine | 577.63 (MH⁺) | (500 MHz) 2.58-2.66 (2H, m), 2.74-2.82 (1H, m), 2.91-2.97 (1H, m), 3.03-3.16 (2H, m), 3.28-3.32 (2H, m), 3.64-3.73 (3H, m), 4.11-4.16 (2H, m), 4.65-4.72 6.16 (0.5H, s), 7.09- 7.12 (2H, m), 7.18- 7.21 (2H, m), 7.27- 7.29 (2H, m), 7.34- 7.44 (5H, m), 9.03 (1H, d) |
| 8.67 | 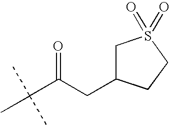 | N-(2,6- dichlorobenzoyl)-4- {1-[(1,1- dioxidotetrahydro-3- thienyl)acetyl] 1,2,3,6- tetrahydropyridin-4- yl}-L-phenylalanine | 579.53 | |
| 8.68 | 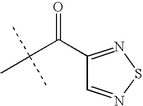 | N-(2,6- dichlorobenzoyl)-4- [1-(1,2,5-thiadiazol- 3-ylcarbonyl)- 1,2,3,6- tetrahydropyridin-4- yl]-L-phenylalanine | 531.36 (MH⁺) | (500 MHz) 2.52 (2H, m), 2.80 (1H, m), 3.10 (1H, m), 3.70-3.93 (2H, m), 4.35 (2H, m), 4.69 (1H, m), 6.10 (0.4H, s), 6.23 (0.6H, s) (1H), 7.29 (2H, m), 7.38-7.41 (5H, m), 9.02 (1H, s), 9.14 (1H, d) |
| 8.69 | 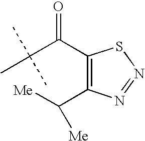 | N-(2,6- dichlorobenzoyl)-4- {1-[(4-isopropyl- 1,2,3-thiadiazol-5- yl)carbonyl]-1,2,3,6- tetrahydropyridin-4- yl}-L-phenylalanine | 573.50 (MH⁺) | (500 MHz) 1.25-1.36 (6H, m), 2.47-2.58 (2H, m), 2.82-2.89 (1H, m), 3.03-3.09 (1H, m), 3.33-3.39 (2H, m), 3.79-3.85 (1H, m), 4.25 (1H, m), 4.59-4.64 (1H, m), 6.13 (0.6H, s), 6.29 (0.4H, s), 7.21 (2H, m), 7.27-7.36 (5H, m), 8.97 (1H, d) |
| 8.70 | 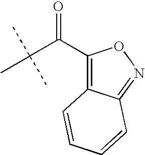 | 4-[1-(2,1- benzisoxazol-3- ylcarbonyl)-1,2,3,6- tetrahydropyridin-4- yl]-N-(2,6- dichlorobenzoyl)-L- phenylalanine | 564.53 (MH⁺) | (500 MHz) 2.60-2.70 (2H, m), 2.90-2.97 (1H, m), 3.11-3.18 (1H, m), 3.95 (2H, t), 4.36-4.53 (2H, m), 4.66-4.74 (1H, m), 6.12-6.28 (1H, m), 7.24-7.31 (3H, m), 7.36-7.45 (5H, m), 7.48-7.53 (1H, m), 7.77 (1H, d), 7.85 (1H, d), 9.06 (1H, d) |
| 8.71 | 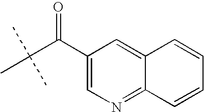 | N-(2,6- dichlorobenzoyl)-4- [1-(quinolin-3- ylcarbonyl)-1,2,3,6- tetrahydropyridin-4- yl]-L-phenylalanine | 574.57 (MH⁺) | (500 MHz) 2.50-2.58 (2H, m), 2.82-2.90 (1H, m), 3.04-3.09 (1H, m), 3.52-3.91 (2H, m), 4.11-4.30 (2H, m), 4.59-4.65 (1H, m), 5.97 (0.4H, s), 6.18 (0.6H, s), 7.21- 7.22 (2H, m), 7.30- 7.36 (5H, m), 7.61- 7.65 (1H, m), 7.78- 7.81 (1H, m), 8.01- 8.05 (2H, m), 8.47 (1H, s), 8.90 (1H, s), 8.98 (1H, d) |
| 8.72 | 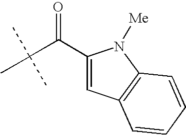 | N-(2,6- dichlorobenzoyl)-4- {1-[(1-methyl-1H- indol-2-yl)carbonyl]- 1,2,3,6- tetrahydropyridin-4- yl}-L-phenylalanine | 576.57 (MH⁺) | (500 MHz) 2.51-2.55 (2H, m), 2.84-2.89 (1H, m), 3.04-3.09 (1H, m), 3.71 (3H, s), 3.82 (2H, s), 4.26 (2H, s), 4.60-4.64 (1H, m), 6.34 (1H, s), 6.69 (1H, s), 7.04 (1H, t), 7.17- 7.22 (3H, m), 7.30- 7.37 (5H, m), 7.45 (1H, d), 7.56 (1H, d), 8.98 (1H, d) |
| 8.73 | 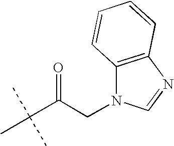 | 4-[1-(1H- benzimidazol-1- ylacetyl)-1,2,3,6- tetrahydropyridin-4- yl]-N-(2,6- dichlorobenzoyl)-L- phenylalanine | 577.57 (MH⁺) | |

### Example 9.1

### N-(2,6-dichlorobenzoyl)-4-[1-(phenylsulfonyl)-1,2,3,6-tetrahydropyridin-4-yl]-L-phenylalanine

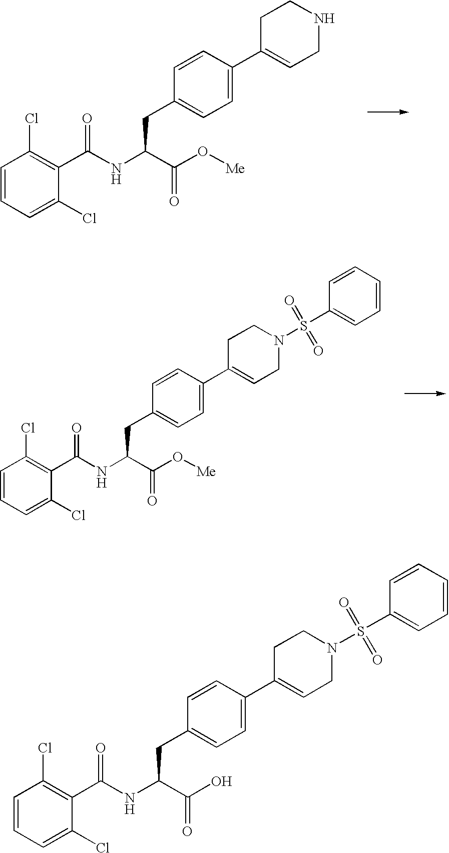

A solution of methyl N-(2,6-dichlorobenzoyl)-4-(1,2,3,6-tetrahydropyridin-4-yl)-L-phenylalaninate (80 mg) in pyridine (3 ml) was added to phenylsulfonyl chloride (35 mg). The mixture was shaken overnight at room temperature and then was concentrated and the residue was dissolved in 2:1 acetonitrile-methanol (3 ml). A solution of lithium hydroxide monohydrate (24 mg) in water (200 μl) was added and the mixture was shaken at room temperature for 12 hours. The solvent was removed under reduced pressure and the crude product was purified by reverse phase preparative HPLC to afford the title compound (19 mg, 17%); ¹H NMR (DMSO-d6, 300 MHz) δ 2.40 (2H, m), 2.93-3.00 (1H, m), 3.08-3.14 (1H, m), 3.24 (2H, t), 3.66-3.72 (2H, m), 4.38-4.45 (1H, m), 6.10 (1H, s), 7.20 (4H, m), 7.33-7.43 (3H, m), 7.62-7.75 (4H, m), 7.80-7.83 (2H, m), 8.27 (1H, d), 8.34 (1H, s); Mass Spectrum MH⁺ 559.35.

The procedure described above was repeated using methyl N-(2,6-dichlorobenzoyl)-4-(1,2,3,6-tetrahydropyridin-4-yl)-L-phenylalaninate and the appropriate sulfonyl chloride to obtain the compounds listed in Table 9.

TABLE 9

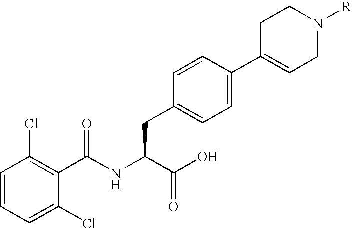

| Example | | | Mass | NMR Data |
|---|---|---|---|---|
| Number | R | Name | Ion | (400 MHz, DMSO-d₆) |
| 9.2 | 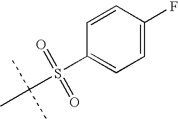 | N-(2,6- dichlorobenzoyl)- 4-{1-[(4- fluorophenyl)sulfonyl]- 1,2,3,6- tetrahydropyridin- 4-yl}-L- phenylalanine | 577.93 (MH⁺) | (300 MHz) 2.89-2.95 (1H, m), 3.11-3.15 (1H, m), 3.25-3.30 (2H, m), 3.70-3.74 (2H, m), 4.65-4.71 (1H, m), 6.06 (1H, s), 7.25-7.30 (4H, m), 7.38-7.45 (3H, m), 7.47-7.52 (2H, m), 7.89-7.93 (2H, m), 9.03 (1H, d) |
| 9.3 | 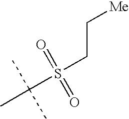 | N-(2,6- dichlorobenzoyl)- 4-[1- (propylsulfonyl)- 1,2,3,6- tetrahydropyridin- 4-yl]-L- phenylalanine | 525.96 (MH⁺) | 0.99 (3H, t), 1.65-1.78 (2H, m), 2.96-3.17 (4H, m), 3.43 (2H, t), 3.87-3.91 (2H, m), 4.39-4.46 (1H, m), 7.24-7.32 (4H, m), 7.36-7.44 (3H, m), 8.28-8.33 (1H, m) |
| 9.4 | 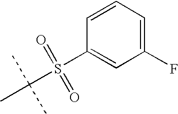 | N-(2,6- dichlorobenzoyl)- 4-{1-[(3- fluorophenyl)sulfonyl]- 1,2,3,6- tetrahydropyridin- 4-yl}-L- phenylalanine | 578.01 (MH⁺) | 2.52-2.58 (2H, m), 2.89-2.95 (1H, m), 3.10-3.15 (1H, m), 3.74-3.76 (2H, m), 4.65-4.71 (1H, m), 6.06 (1H, s), 7.25-7.30 (4H, m), 7.37-7.46 (3H, m), 7.58-7.63 (1H, m), 7.65-7.75 (3H, m), 9.03 (1H, d) |
| 9.5 | 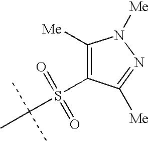 | N-(2,6- dichlorobenzoyl)- 4-{1-[(1,3,5- trimethyl-1H- pyrazol-4- yl)sulfonyl]- 1,2,3,6- tetrahydropyridin- 4-yl}-L- phenylalanine | 591.51 (MH⁺) | 2.31 (3H, s), 2.45 (3H, s), 2.53-2.56 (2H, m), 2.90-2.96 (1H, m), 3.11-3.16 (1H, m), 3.23 (2H, t), 3.64-3.66 (2H, m), 3.73 (3H, s), 4.64-4.72 (1H, m), 6.09-6.11 (1H, m), 7.27 (2H, d), 7.31 (2H, d), 7.37-7.45 (3H, m), 9.01 (1H, d) |
| 9.6 | 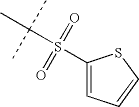 | N-(2,6- dichlorobenzoyl)- 4-[1-(2- thienylsulfonyl)- 1,2,3,6- tetrahydropyridin- 4-yl}-L- phenylalanine | 565.40 (MH⁺) | |
| 9.7 | 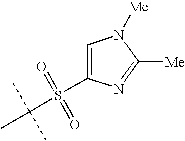 | N-(2,6- dichlorobenzoyl)- 4-{1-[(1,2- dimethyl-1H- imidazol-4- yl)sulfonyl]- 1,2,3,6- tetrahydropyridin- 4-yl}-L- phenylalanine | 577.51 (MH⁺) | 2.31 (3H, s), 2.90-2.96 (2H, m), 3.11-3.16 (2H, m), 3.26 (2H, t), 3.63 (3H, s), 3.74-3.77 (2H, m), 4.65-4.71 (1H, m), 6.07-6.09 (1H, m), 7.27 (2H, d), 7.31 (2H, d), 7.36-7.46 (3H, m), 7.78 (1H, s), 9.01 (1H, d) |
| 9.8 | 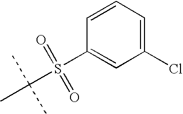 | N-(2,6- dichlorobenzoyl)- 4-{1-[(3- chlorophenyl)sulfonyl]- 1,2,3,6- tetrahydropyridin- 4-yl}-L- phenylalanine | 593.65 (MH⁻) | 2.89-2.97 (1H, m), 3.10-3.16 (1H, m), 3.31-3.36 (2H, m), 3.73-3.78 (2H, m), 4.65-4.71 (1H, m), 6.06 (1H, bs), 7.23- 7.30 (4H, m), 7.37- 7.45 (3H, m), 7.67- 7.71 (1H, m), 7.79- 7.85 (3H, m), 9.01 (1H, d) |
| 9.9 | 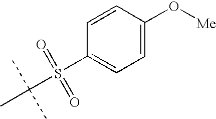 | N-(2,6- dichlorobenzoyl)- 4-{1-[(4- methoxyphenyl)sulfonyl]- 1,2,3,6- tetrahydropyridin- 4-yl}-L- phenylalanine | 589.52 (MH⁻) | 2.90-2.97 (1H, m), 3.10-3.15 (1H, m), 3.20 (2H, t), 3.63-3.67 (2H, m), 3.87 (3H, s), 4.64-4.71 (1H, m), 6.07 (1H, s), 7.14-7.19 (2H, m), 7.24-7.30 (4H, m), 7.36-7.45 (3H, m), 7.73-7.78 (2H, m), 9.01 (1H, d) |
| 9.10 | 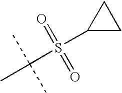 | 4-[1- cyclopropylsulfonyl)- 1,2,3,6- tetrahydropyridin- 4-yl]-N-(2,6- dichlorobenzoyl)- L-phenylalanine | 521.66 (MH⁻) | 0.97-1.01 (4H, m), 2.57-2.61 (2H, m), 2.64-2.70 (1H, m), 2.91-2.97 (1H, m), 3.12-3.17 (1H, m), 3.46 (2H, t), 3.93-3.96 (2H, m), 4.67-4.74 (1H, m), 6.16-6.18 (1H, m), 7.29 (2H, d), 7.36-7.45 (5H, m), 9.06 (1H, d) |
| 9.11 | 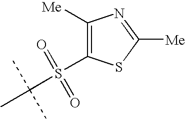 | N-(2,6- dichlorobenzoyl)- 4-{1-[(2,4- dimethyl-1,3- thiazol-5- yl)sulfonyl]- 1,2,3,6- tetrahydropyridin- 4-yl}-L- phenylalanine | 592.70 (MH⁻) | 2.59 (3H, s), 2.68 (3H, s), 2.91-2.97 (1H, m), 3.12-3.16 (1H, m), 3.36 (2H, t), 3.80-3.83 (2H, m), 4.63-4.69 (1H, m), 6.10-6.13 (1H, m), 7.27 (2H, d), 7.31 (2H, d), 7.37-7.45 (3H, m), 8.97 (1H, d) |

### Example 10.1

### N-(2,6-dichlorobenzoyl)-4-(1-{[(2-fluorophenyl)amino]carbonyl}-1,2,3,6-tetrahydropyridin-4-yl)-L-phenylalanine

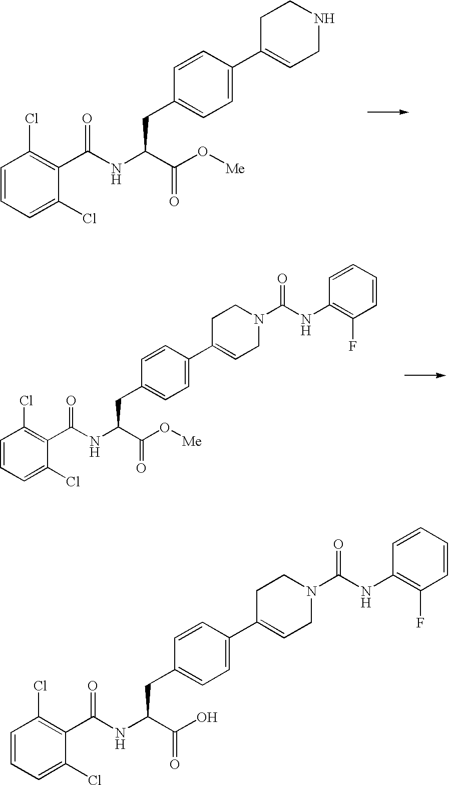

A solution of methyl N-(2,6-dichlorobenzoyl)-4-(1,2,3,6-tetrahydropyridin-4-yl)-L-phenylalaninate (80 mg) in THF (3 ml) was added to 2-fluorophenylisocyanate (28 mg). The mixture was shaken overnight at room temperature. A solution of lithium hydroxide monohydrate (24 mg) in water (200 μl) was added and the mixture was shaken at room temperature for 12 hours. The solvent was removed under reduced pressure and the crude product was purified by reverse phase preparative HPLC to afford the title compound (61 mg, 59%); ¹H NMR (DMSO-d6) δ 2.96-3.02 (1H, m), 3.12-3.18 (1H, m), 3.26-3.36 (4H, m), 3.68 (2H, t), 4.51-4.59 (2H, m), 6.19 (1H, s), 7.10-7.14 (2H, m), 7.17-7.23 (1H, m), 7.28 (2H, d), 7.36 (2H, d), 7.39-7.51 (4H, m), 8.31 (0.5H, s), 8.7 (0.5H, s); Mass Spectrum MH⁺ 556.90.

The procedure described above was repeated using methyl N-(2,6-dichlorobenzoyl)-4-(1,2,3,6-tetrahydropyridin-4-yl)-L-phenylalaninate and the appropriate isocyanate to obtain the compounds listed in Table 10.

TABLE 10

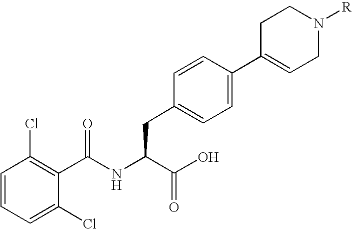

| Example | | | Mass | NMR Data |
|---|---|---|---|---|
| Number | R | Name | Ion | (400 MHz, DMSO-d₆) |
| 10.2 | 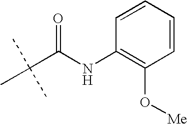 | N-(2,6- dichlorobenzoyl)-4- (1-{[(2- methoxyphenyl)amino] carbonyl}- 1,2,3,6- tetrahydropyridin-4- yl)-L-phenylalanine | 556.90 (MH⁺) | 2.95-3.01 (1H, m), 3.13-3.18 (1H, m), 3.24-3.36 (2H, m), 3.66 (2H, t), 3.84 (3H, s), 4.12-4.16 (2H, m), 4.55-4.62 (1H, m), 6.17 (1H, s), 6.87-6.90 (1H, m), 7.02 (2H, d), 7.28 (2H, d), 7.36 (2H, d), 7.38-7.46 (4H, m), 8.70 (1H, bs) |
| 10.3 | 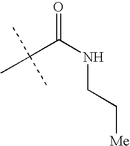 | N-(2,6- dichlorobenzoyl)-4- {1- [(propylamino)carbonyl]- 1,2,3,6- tetrahydropyridin-4- yl}-L-phenylalanine | 505.04 (MH⁺) | 0.76 (3H, t), 1.35 (2H, q), 2.32-2.37 (2H, m), 2.84-2.96 (3H, m), 3.02-3.08 (1H, m), 3.43 (2H, t), 3.86-3.90 (2H, m), 4.51-4.59 (1H, m), 6.04 (1H, s), 6.38 (1H, t), 7.19 (2H, d), 7.26 (2H, d), 7.31- 7.37 (3H, m), 8.80 (1H, s) |
| 10.4 | 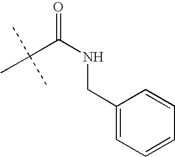 | 4-{1- [(benzylamino)carbonyl]- 1,2,3,6- tetrahydropyridin-4- yl}-N-(2,6- dichlorobenzoyl)-L- phenylalanine | 553.11 (MH⁺) | 2.44-2.48 (2H, m), 2.90-2.97 (1H, m), 3.12-3.17 (1H, m), 3.58 (2H, t), 4.02-4.04 (2H, m), 4.28-4.30 (2H, m), 4.66-4.72 (1H, m), 6.15 (1H, s), 7.10 (1H, t), 7.20-7.24 (1H, m), 7.27-7.32 (6H, m), 7.35-7.45 (5H, m), 9.03 (1H, d) |
| 10.5 | 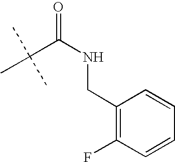 | N-(2,6- dichlorobenzoyl)-4- (1-{[(2- fluorobenzyl)amino] carbonyl}-1,2,3,6- tetrahydropyridin-4- yl)-L-phenylalanine | 571.11 (MH⁺) | 2.44-2.48 (2H, m), 2.90-2.97 (1H, m), 3.12-3.17 (1H, m), 3.58 (2H, t), 4.02-4.04 (2H, m), 4.28-4.30 (2H, m), 4.66-4.72 (1H, m), 6.15 (1H, s), 7.10 (1H, t), 7.20-7.24 (2H, m), 7.27-7.32 (3H, m), 7.35-7.45 (6H, m), 9.03 (1H, d) |
| 10.6 | 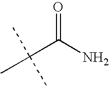 | 4-[1- (aminocarbonyl)- 1,2,3,6- tetrahydropyridin-4- yl]-N-(2,6- dichlorobenzoyl)-L- phenylalanine | 462.44 (MH⁻) | (300 MHz, DMSO + AcOD) 3.03- 3.08 (2H, m), 3.53- 3.61 (1H, m), 3.74- 3.80 (1H, m), 4.14 (2H, t), 4.57-4.62 (2H, m), 5.27-5.34 (1H, m), 6.62 (0.3H, s), 6.75 (0.7H, s), 7.90 (2H, d), 7.97-8.08 (5H, m) |

### Example 11.1

### N-(2,6-dichlorobenzoyl)-4-[1-(2-hydroxyethyl)-1,2,3,6-tetrahydropyridin-4-yl]-L-phenylalanine

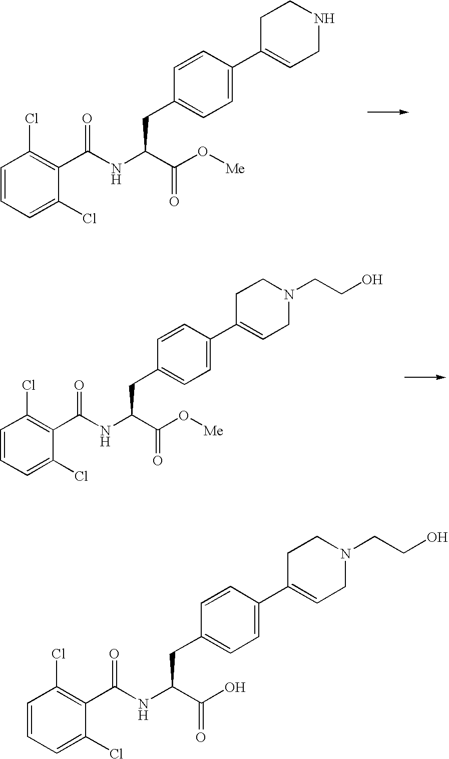

A solution of methyl N-(2,6-dichlorobenzoyl)-4-(1,2,3,6-tetrahydropyridin-4-yl)-L-phenylalaninate (80 mg) in DMF (1 ml) was added to 2-chloroethanol (21 mg), then MP-Carbonate (150 mg, 2.74 mmol/g) was added. The mixture was shaken overnight at room temperature then filtered and concentrated. The residue was dissolved in 2:1 acetonitrile-methanol (3 ml). A solution of lithium hydroxide monohydrate (24 mg) in water (200 μl) was added and the mixture was shaken at room temperature for 16 hours. The solvent removed under reduced pressure and the crude product was purified by reverse phase preparative HPLC to afford the title compound (25 mg, 29%); ¹H NMR (DMSO-d6+AcOD) (300 MHz) δ 2.35-2.50 (2H, m), 2.64-2.73 (2H, m), 2.90-2.97 (1H, m), 3.09-3.18 (2H, m), 3.30-3.34 (1H, m), 3.70-3.79 (2H, m), 4.66-4.71 (1H, m), 6.15 (1H, s), 7.28-7.32 (2H, m), 7.36-7.43 (5H, m), 8.14 (1H, s); Mass Spectrum MH⁺ 463.95.

The procedure described above was repeated using methyl N-(2,6-dichlorobenzoyl)-4-(1,2,3,6-tetrahydropyridin-4-yl)-L-phenylalaninate and the appropriate alkyl chloride to obtain the compounds listed in Table 11.

TABLE 11

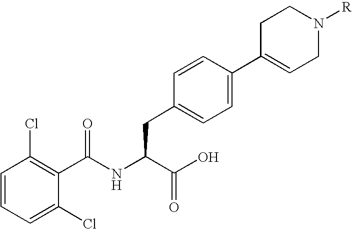

| Example | | | Mass | NMR Data |
|---|---|---|---|---|
| Number | R | Name | Ion | (400 MHz, DMSO-d₆) |
| 11.2 | 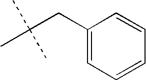 | 4-(1-benzyl-1,2,3,6- tetrahydropyridin-4- yl)-N-(2,6- dichlorobenzoyl)-L- phenylalanine | 509.62 (MH⁺) | (500 MHz) 2.47-2.47 (2H, m), 2.55-2.57 (2H, m), 2.84-2.88 (1H, m), 2.97-2.98 (2H, m), 3.02-3.06 (1H, m), 3.51 (2H, s), 4.45-4.54 (1H, m), 6.02 (1H, s), 7.15-7.19 (3H, m), 7.20-7.27 (6H, m), 7.30-7.41 (3H, m), 9.09 (1H, d) |
| 11.3 | 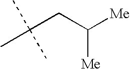 | N-(2,6- dichlorobenzoyl)-4- (1-isobutyl-1,2,3,6- tetrahydropyridin-4- yl)-L-phenylalanine | 475.99 (MH⁺) | |
| 11.4 | 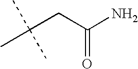 | 4-[1-(2-amino-2- oxoethyl)-1,2,3,6- tetrahydropyridin-4- yl]-N-(2,6- dichlorobenzoyl)-L- phenylalanine | 476.96 (MH⁺) | |
| 11.5 | 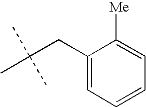 | N-(2,6- dichlorobenzoyl)-4- [1-(2- methylbenzyl)- 1,2,3,6- tetrahydropyridin-4- yl]-L-phenylalanine | 523.62 (MH⁺) | |
| 11.6 | 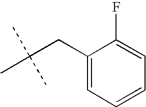 | N-(2,6- dichlorobenzoyl)-4- [1-(2-fluorobenzyl)- 1,2,3,6- tetrahydropyridin-4- yl]-L-phenylalanine | 527.63 (MH⁺) | |
| 11.7 | 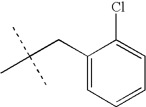 | N-(2,6- dichlorobenzoyl)-4- [1-(2- chlorobenzyl)- 1,2,3,6- tetrahydropyridin-4- yl]-L-phenylalanine | 543.58 (MH⁺) | |
| 11.8 | 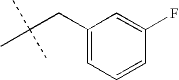 | N-(2,6- dichlorobenzoyl)-4- [1-(3-fluorobenzyl)- 1,2,3,6- tetrahydropyridin-4- yl]-L-phenylalanine | 527.61 (MH⁺) | (500 MHz) 2.76-2.79 (2H, m), 2.91-2.97 (1H, m), 3.11-3.16 (1H, m), 3.21-3.24 (2H, m), 3.79 (2H, s), 4.66-4.72 (1H, m), 6.15 (1H, s), 6.75-6.81 (2H, m), 7.11-7.17 (2H, m), 7.27 (2H, d), 7.37 (2H, d), 7.39-7.46 (3H, m), 9.04 (1H, d) |
| 11.9 | 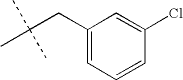 | N-(2,6- dichlorobenzoyl)-4- [1-(3- chlorobenzyl)- 1,2,3,6- tetrahydropyridin-4- yl]-L-phenylalanine | 543.59 (MH⁺) | 2.46 (2H, s), 2.67 (2H, t), 2.94-2.97 (1H, m), 3.11-3.15 (3H, m), 3.60 (2H, s), 4.65 (1H, m), 6.11 (1H, s), 6.94 (1H, t), 7.00 (1H, d), 7.25 (3H, d), 7.33 (2H, d), 7.35-7.37 (1H, m), 7.40 (1H, d), 7.43-7.44 (2H, m), 8.98 (1H, m) |
| 11.10 | 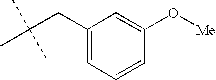 | N-(2,6- dichlorobenzoyl)-4- [1-(3- methoxybenzyl)- 1,2,3,6- tetrahydropyridin-4- yl]-L-phenylalanine | 539.62 (MH⁺) | |
| 11.11 | 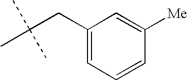 | N-(2,6- dichlorobenzoyl)-4- [1-(3- methylbenzyl)- 1,2,3,6- tetrahydropyridin-4- yl]-L-phenylalanine | 523.62 (MH⁺) | 2.32 (3H, s), 2.38 (2H, s), 2.46 (2H, s), 2.65 (1H, t), 2.90-3.00 (1H, m), 3.06 (1H, d), 3.11- 3.15 (1H, m), 3.56 (2H, s), 4.66 (1H, d), 6.10 (1H, d), 7.08 (1H, d), 7.13 (1H, s), 7.16 (1H, d), 7.23 (3H, t), 7.33 (2H, d), 7.37-7.45 (3H, m), 8.96 (1H, d) |
| 11.12 | 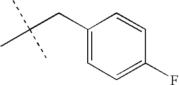 | N-(2,6- dichlorobenzoyl)-4- [1-(4-fluorobenzyl)- 1,2,3,6- tetrahydropyridin-4- yl]-L-phenylalanine | 527.62 (MH⁺) | (300 MHz) 2.40 (2H, m), 2.60 (2H, t), 3.05 (2H, m), 3.10 (2H, t), 3.59 (2H, s), 4.23 (1H, s), 6.10 (1H, d), 7.25 (2H, m), 7.20 (4H, m), 7.37-7.44 (5H, m), 7.70 (1H, d) |
| 11.13 | 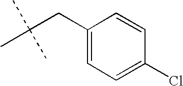 | N-(2,6- dichlorobenzoyl)-4- [1-(4- chlorobenzyl)- 1,2,3,6- tetrahydropyridin-4- yl]-L-phenylalanine | 543.57 (MH⁺) | 2.46 (2H, s), 2.55 (2H, m), 2.90-2.96 (1H, m), 3.06-3.07 (2H, m), 3.11-3.15 (1H, m), 3.59 (2H, s), 4.65 (1H, s), 6.10 (1H, d), 7.25 (2H, d), 7.33 (2H, d), 7.35-7.41 (1H, m), 7.37-7.44 (6H, m), 8.95 (1H, d) |
| 11.14 | 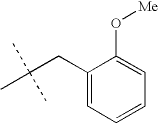 | N-(2,6- dichlorobenzoyl)-4- [1-(2- methoxybenzyl)- 1,2,3,6- tetrahydropyridin-4- yl]-L-phenylalanine | 539.62 (MH⁺) | (500 MHz) 2.45-2.49 (2H, m), 2.66 (2H, t), 2.91-2.97 (1H, m), 3.07-3.15 (3H, m), 3.67 (3H, s), 4.63-4.68 (1H, m), 6.11 (1H, bs), 7.26 (2H, d), 7.33 (2H, d), 7.39-7.45 (4H, m), 7.55-7.59 (1H, m), 7.71-7.76 (2H, m), 8.95 (1H, d) |
| 11.15 | 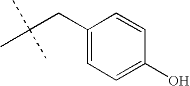 | N-(2,6- dichlorobenzoyl)-4- [1-(4- hydroxybenzyl)- 1,2,3,6- tetrahydropyridin-4- yl]-L-phenylalanine | 525.44 (MH⁺) | |
| 11.16 | 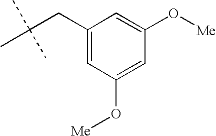 | N-(2,6- dichlorobenzoyl)-4- [1-(3,5- dimethoxybenzyl)- 1,2,3,6- tetrahydropyridin-4- yl]-L-phenylalanine | 569.66 (MH⁺) | 2.55 (2H, s), 2.64 (3H, t), 2.95-2.97 (2H, m), 3.09-3.15 (1H, m), 3.53 (2H, s), 3.74 (6H, d), 4.64 (1H, d), 6.11 (1H, s), 6.40 (1H, t), 6.49-6.52 (2H, m), 6.74 (1H, d), 7.25 (2H, d), 7.33 (2H, d), 7.38- 7.45 (3H, m), 8.90 (1H, m) |
| 11.17 | 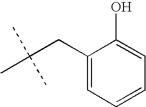 | N-(2,6- dichlorobenzoyl)-4- [1-(2- hydroxybenzyl)- 1,2,3,6- tetrahydropyridin-4- yl]-L-phenylalanine | 525.62 (MH⁺) | 2.55 (2H, m), 2.72 (2H, s), 2.94 (1H, d), 3.11- 3.16 (3H, m), 3.63 (2H, s), 4.70 (1H, m), 6.11 (1H, s), 7.25 (1H, s), 7.28 (3H, d), 7.34 (2H, d), 7.37-7.41 (1H, m), 7.43-7.43 (1H, m), 7.43 (1H, s), 7.56 (2H, d), 9.04 (1H, d), 9.92 (1H, s) |
| 11.18 | 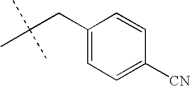 | N-(2,6- dichlorobenzoyl)-4- [1-(4-cyanobenzyl)- 1,2,3,6- tetrahydropyridin-4- yl]-L-phenylalanine | 534.61 (MH⁺) | |
| 11.19 | 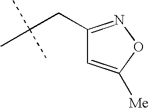 | N-(2,6- dichlorobenzoyl)-4- {1-[(5- methylisoxanol-3- yl)methyl]-1,2,3,6- tetrahydropyridin-4- yl}-L-phenylalanine | 514.59 (MH⁺) | 2.44-2.49 (2H, m), 2.56 (3H, s), 2.68 (2H, t), 2.90-2.96 (1H, m), 3.09-3.12 (2H, m), 3.14-3.16 (1H, m), 3.62 (2H, s), 4.64-4.70 (1H, m), 6.10 (1H, bs), 7.25 (2H, d), 7.33 (2H, d), 7.37-7.45 (3H, m), 8.17 (1H, s), 8.98 (1H, d) |
| 11.20 | 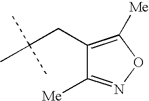 | N-(2,6- dichlorobenzoyl)-4- {1-[(3,5- dimethylisoxazol-4- yl)methyl]-1,2,3,6- tetrahydropyridin-4- yl}-L-phenylalanine | 528.61 (MH⁺) | |
| 11.21 | 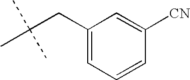 | N-(2,6- dichlorobenzoyl)-4- [1-(3-cyanobenzyl)- 1,2,3,6- tetrahydropyridin-4- yl]-L-phenylalanine | 534.63 (MH⁺) | (500 MHz) 2.45-2.48 (2H, m), 2.66 (2H, t), 2.91-2.96 (1H, m), 3.07-3.10 (2H, m), 3.12-3.16 (1H, m), 3.62 (2H, s), 4.62-4.68 (1H, m), 6.09-6.12 (1H, m), 7.08-7.12 (1H, m), 7.15-7.22 (2H, m), 7.26 (2H, d), 7.33 (2H, d), 7.36-7.47 (4H, m), 8.94 (1H, d) |
| 11.22 | 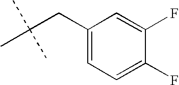 | N-(2,6- dichlorobenzoyl)-4- [1-(3,4- difluorobenzyl)- 1,2,3,6- tetrahydropyridin-4- yl]-L-phenylalanine | 545.58 (MH⁺) | |
| 11.23 | 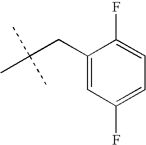 | N-(2,6- dichlorobenzoyl)-4- [1-(2,5- difluorobenzyl)- 1,2,3,6- tetrahydropyridin-4- yl]-L-phenylalanine | 545.58 (MH⁺) | (300 MHz, DMSO + AcOD) 2.44- 2.48 (2H, m), 2.68 (2H, t), 2.90-2.97 (1H, m), 3.09-3.13 (3H, m), 3.64 (2H, s), 4.58-4.62 (1H, m), 6.10 (1H, s), 7.12-7.20 (1H, m), 7.21-7.33 (6H, m), 7.35-7.43 (3H, m) |
| 11.24 | 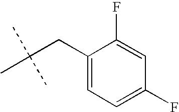 | N-(2,6- dichlorobenzoyl)-4- [1-(2,4- difluorobenzyl)- 1,2,3,6- tetrahydropyridin-4- yl]-L-phenylalanine | 545.58 (MH⁺) | (500 MHz) 2.46-2.49 (2H, m), 2.66 (2H, t), 2.90-2.96 (1H, m), 3.06-3.09 (2H, m), 3.11-3.15 (1H, m), 3.62 (2H, s), 4.65-4.70 (1H, m), 6.12 (1H, s), 7.24-7.27 (2H, m), 7.32-7.39 (4H, m), 7.41-7.45 (4H, m), 9.00 (1H, d) |
| 11.25 | 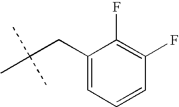 | N-(2,6- dichlorobenzoyl)-4- [1-(2,3- difluorobenzyl)- 1,2,3,6- tetrahydropyridin-4- yl]-L-phenylalanine | 545.57 (MH⁺) | (500 MHz) 2.46-2.49 (2H, m), 2.67 (2H, t), 2.90-2.96 (1H, m), 3.06-3.09 (3H, m), 3.70 (2H, s), 4.65-4.70 (1H, m), 6.12 (1H, s), 7.24-7.27 (3H, m), 7.32-7.39 (2H, d), 7.41- 7.45 (5H, m), 9.00 (1H, d) |
| 11.26 | 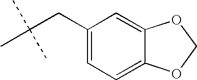 | 4-[1-(1,3- benzodioxol-5- ylmethyl)-1,2,3,6- tetrahydropyridin-4- yl]-N-(2,6- dichlorobenzoyl)-L- phenylalanine | 553.58 (MH⁺) | 2.46 (2H, d), 2.54 (2H, d), 2.68 (1H, s), 2.69 (2H, t), 2.94-2.96 (1H, m), 3.11 (1H, s), 3.11- 3.15 (2H, m), 3.70 (2H, s), 6.10 (1H, s), 7.20- 7.22 (1H, m), 7.24- 7.29 (3H, m), 7.33 (3H, d), 7.40 (1H, d), 7.43- 7.44 (2H, m), 9.00 (1H, d) |
| 11.27 | 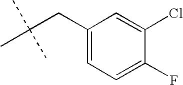 | N-(2,6- dichlorobenzoyl)-4- [1-(3-chloro-4- fluorobenzyl)- 1,2,3,6- tetrahydropyridin-4- yl]-L-phenylalanine | 563.52 (MH⁺) | |
| 11.28 | 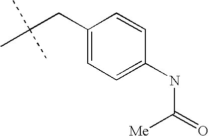 | 4-{1-[4- (acetylamino)benzyl]- 1,2,3,6 tetrahydropyridin-4- yl}-N-(2,6- dichlorobenzoyl)-L- phenylalanine | 566.48 (MH⁺) | |
| 11.29 | 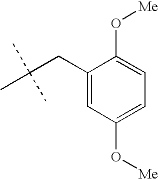 | N-(2,6- dichlorobenzoyl)-4- [1-(2,5- dimethoxybenzyl)- 1,2,3,6- tetrahydropyridin-4- yl]-L-phenylalanine | 569.49 (MH⁺) | 2.47 (2H, m), 2.67 (2H, t), 2.95 (1H, d), 3.11 (1H, m), 3.13 (2H, d), 3.58 (2H, s), 3.72-3.75 (3H, m), 3.71-3.77 (3H, m), 4.68 (1H, d), 6.12 (1H, s), 6.79-6.82 (1H, m), 6.94 (2H, m), 7.26 (2H, d), 7.34 (2H, d), 7.38-7.44 (3H, m), 9.00 (1H, d) |
| 11.30 | 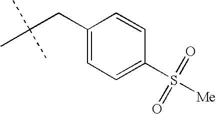 | N-(2,6- dichlorobenzoyl)-4- {1-[4- (methylsulfonyl)benzyl]- 1,2,3,6- tetrahydropyridin-4- yl}-L-phenylalanine | 587.57 (MH⁺) | 2.54 (2H, m), 2.69 (2H, t), 2.93-2.96 (1H, m), 3.11-3.12 (3H, m), 3.15 (3H, s), 3.74 (2H, s), 4.70 (1H, m), 6.12 (1H, m), 7.26 (2H, d), 7.35 (2H, d), 7.40-7.45 (3H, m), 7.65 (2H, d), 7.90-7.93 (2H, m), 9.05 (1H, d) |

### Example 12.1

### N-(2,6-dichlorobenzoyl)-4-(1-phenyl-1,2,3,6-tetrahydropyridin-4-yl)-L-phenylalanine

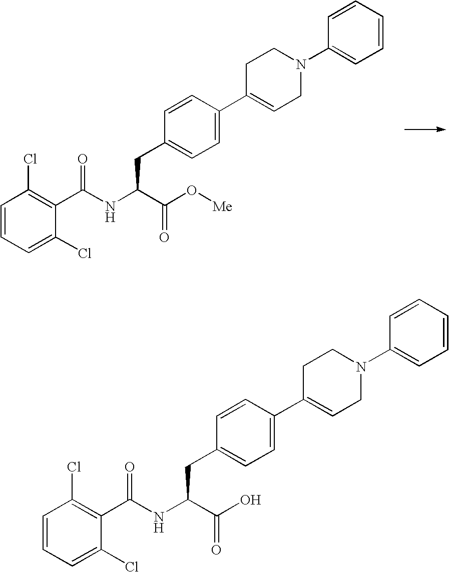

Methyl N-(2,6-dichlorobenzoyl)-4-(1-phenyl-1,2,3,6-tetrahydropyridin-4-yl)-L-phenylalaninate (34 mg), lithium hydroxide (8 mg) and water (0.05 ml) were dissolved in acetonitrile-methanol 2:1 (3 ml) and stirred at 40° C. for 1 hour. The solvent was removed and the residue acidified with 2N HCl to give a solid which was filtered off and washed with water and dried to give the title compound as a white solid (16 mg, 48%); ¹H NMR (DMSO-d6, 500 MHz) δ 2.60-2.55 (2H, m), 3.15-3.11 (1H, m), 3.50 (2H, t), 3.75 (2H, m), 4.60-4.70 (1H, m), 6.30 (1H, s), 6.70-6.77 (1H, m), 7.0 (2H, d), 7.20-7.40 (4H, m), 7.40-7.46 (5H, m), 9.0 (1H, bs); Mass Spectrum MH⁺ 495.98.

The methyl N-(2,6-dichlorobenzoyl)-4-(1-phenyl-1,2,3,6-tetrahydropyridin-4-yl)-L-phenylalaninate used as the starting material was prepared as follows:

### Methyl N-(2,6-dichlorobenzoyl)-4-(1-phenyl-1,2,3,6-tetrahydropyridin-4-yl)-L-phenylalaninate

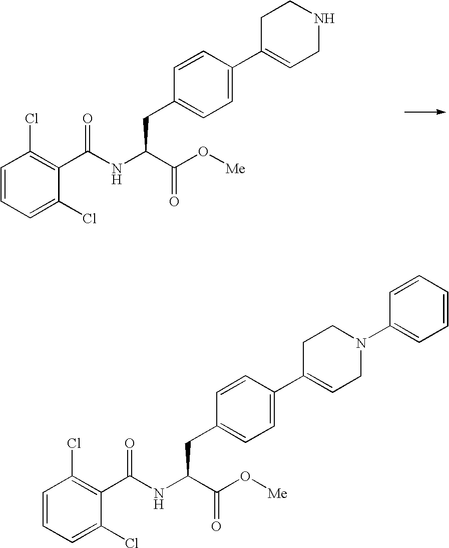

A solution of methyl N-(2,6-dichlorobenzoyl)-4-(1,2,3,6-tetrahydropyridin-4-yl)-L-phenylalaninate (100 mg), phenylboronic acid (42 mg), 2,6-lutidine (46 mg) and copper acetate (41 mg) in DCM (3 ml) were stirred vigorously overnight at room temperature. The reaction mixture was purified by chromatography using 1:1 iso-hexane-ethyl acetate as eluent to afford the title compound (21 mg, 18%); ¹H NMR (DMSO-d6, 500 MHz) δ 2.60-2.55 (2H, m), 3.15-3.11 (1H, m), 3.45 (2H, t), 3.70 (3H, s), 3.75 (2H, m), 4.60-4.80 (1H, m), 6.30 (1H, s), 6.70-6.77 (1H, m), 7.20 (2H, d), 7.20-7.40 (4H, m), 7.40-7.46 (5H, m), 9.20 (1H, d); Mass Spectrum MH⁺ 509.47.

### Example 13.1

### N-(2,6-Dichlorobenzoyl)-4-(3,6-dihydro-2H-pyran-4-yl)-L-phenylalanine

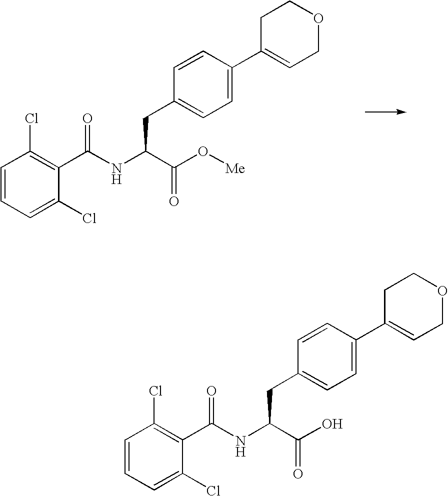

Lithium hydroxide (44 mg) in water (500 μl) was added to methyl 2,6-(dichlorobenzoyl)-4-(3,6-dihydro-2H-pyran-4-yl)-L-phenylalaninate (150 mg) suspended in methanol (3 ml). Acetonitrile (500 μl) was added and the resulting solution stirred at room temperature for 18 hours. The solvent was removed in vacuo and water (4 ml) added. 2N HCl was added until the solution was pH 2. A precipitate was collected, dried under vacuum and washed with diethyl ether to afford the title compound as a white solid, (50 mg, 34%); ¹H NMR (DMSO-d6) δ 2.30 (2H, m), 2.90-3.00 (1H, dd), 3.10-3.20 (1H, dd), 3.80 (2H, t), 4.20 (2H, m), 4.50 (1H, m), 6.20 (1H, m), 7.20-7.30 (4H, m), 7.30-7.50 (3H, m), 8.60 (1H, d); Mass Spectrum MH⁺=420.38

The methyl 2,6-(dichlorobenzoyl)-4-(3,6-dihydro-2H-pyran-4-yl)-L-phenylalaninate used as the starting material was prepared as follows:

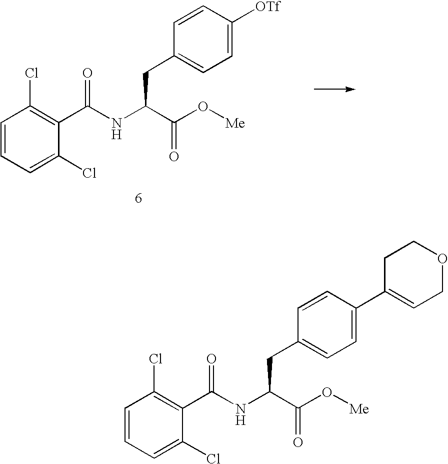

Methyl N-(2,6-dichlorobenzoyl)-O-[(trifluoromethyl)sulfonyl]-L-tyrosinate (300 mg), potassium carbonate (250 mg) and 4-(4,4,5,5-tetramethyl-1,3,2-dioxaborolan-2-yl)-3,6-dihydro-2H-pyran (Synthesis 2000, vol 6, p 778) (126 mg) were dissolved in anhydrous DMF (3 ml) and argon bubbled through the solution at 25° C. for 1 hour. 1,1′-Bis(diphenylphosphino)ferrocene-palladium(II)dichloride dichloromethane complex (20 mg) was then added and the reaction heated at 85° C. for 2 hours. DMF was removed in vacuo and water was added to the resulting oil. This was extracted with ethyl acetate and the organic layer washed with brine then dried and the solvent removed in vacuo to give a brown oil. The residue was purified by chromatography using 0-20% ethyl acetate in DCM as eluent to give the title compound as a white solid (150 mg, 58%); Mass Spectrum M+H=434.43.

### Example 14.1

### N-(2,6-Dichlorobenzoyl)-4-(tetrahydro-2H-pyran-4-yl)-L-phenylalanine

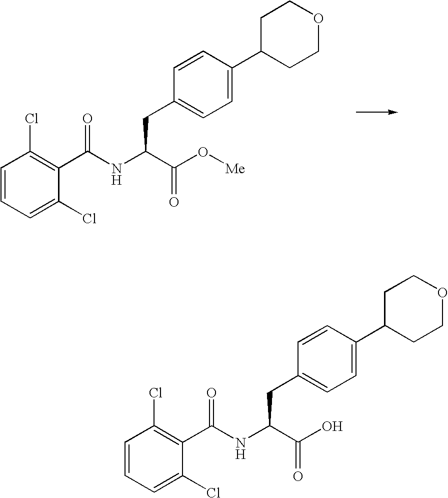

A solution of lithium hydroxide (46 mg) in water (200 μl) was added to methyl N-(2,6-dichlorobenzoyl)-4-(tetrahydro-2H-pyran-4-yl)-L-phenylalaninate (160 mg) suspended in methanol (5 ml). Acetonitrile (500 μl) was added and the resulting solution stirred at room temperature for 2 hours. The solvent was removed in vacuo and water (3 ml) added. 2N HCl (733 μl) was added until the solution was pH 2. A precipitate was collected and dried under vacuum to afford the title compound as a white solid, (130 mg, 84%); ¹H NMR (DMSO-d₆) δ 1.70 (4H, m), 2.70 (1H, m), 2.90-3.30 (2H, m), 3.90 (2H, m), 4.40 (1H, m), 7.10 (2H, d), 7.20 (2H, d), 7.40 (3H, m), 8.30 (1H, m); Mass Spectrum M⁺H=422.39.

The methyl N-(2,6-dichlorobenzoyl)-4-(tetrahydro-2H-pyran-4-yl)-L-phenylalaninate used as the starting material was prepared as follows:

### Methyl N-(tert-butoxycarbonyl)-4-(3,6-dihydro-2H-pyran-4-yl)-L-phenylalaninate

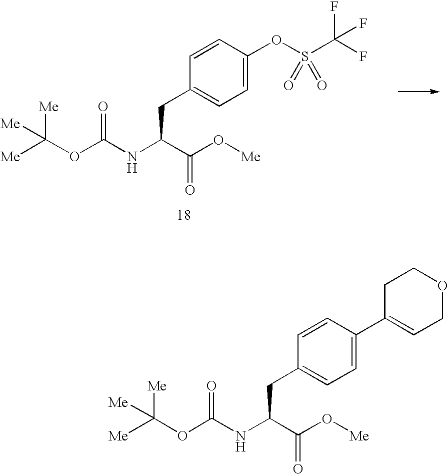

Methyl N-(tert-butoxycarbonyl)-O-[(trifluoromethyl)sulfonyl]-L-tyrosinate (500 mg), potassium carbonate (490 mg) and 4-(4,4,5,5-tetramethyl-1,3,2-dioxaborolan-2-yl)-3,6-dihydro-2H-pyran (320 mg) were dissolved in anhydrous DMF (5 ml) and argon bubbled through the solution at 25° C. for 1 hour. 1,1′-Bis(diphenylphosphino)ferrocene-palladium(II)dichloride dichloromethane complex (4 mg) was then added and the reaction heated at 85° C. for 2 hours. DMF was removed in vacuo and water was added to the resulting oil. This was extracted with ethyl acetate, and the organic layer washed with brine, dried, and the solvent removed in vacuo to give a brown oil that was purified by chromatography using 0-20% ethyl acetate in DCM as eluent to give the title compound as a white solid (330 mg, 79%); Mass Spectrum MH⁺-tert-butoxycarbonyl=262.54.

### Methyl N-(tert-butoxycarbonyl)-4-(tetrahydro-2H-pyran-4-yl)-L-phenylalaninate

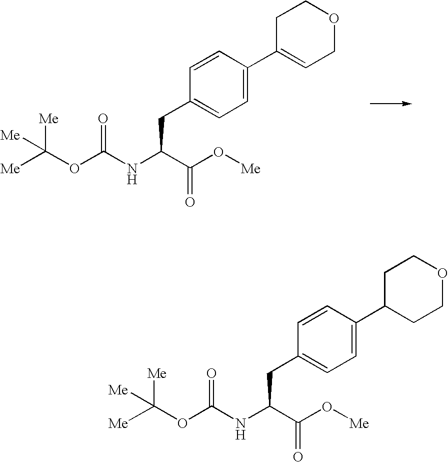

Methyl N-(tert-butoxycarbonyl)-4-(3,6-dihydro-2H-pyran-4-yl)-L-phenylalaninate (330 mg) was dissolved in ethanol (25 ml) and 10% Pd/C (55 mg) added. The resulting solution was stirred under hydrogen at atmospheric pressure for 18 hours. The reaction is mixture was filtered through Celite and solvent removed in vacuo to give the title compound as a colourless oil (300 mg, 90%); Mass Spectrum MH⁺-tert-butoxycarbonyl=264.51.

### Methyl 4-(tetrahydro-2H-pyran-4-yl)-L-phenylalaninate

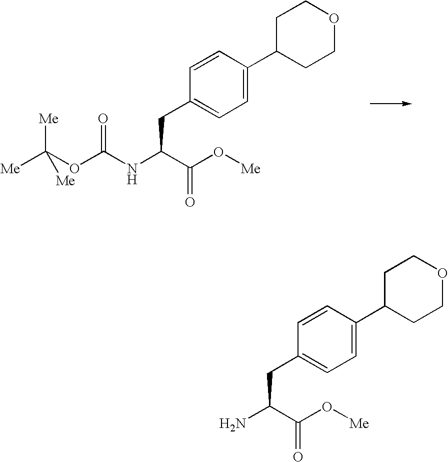

Methyl N-(tert-butoxycarbonyl)-4-(tetrahydro-2H-pyran-4-yl)-L-phenylalaninate (330 mg) was dissolved in methanol (15 ml) and concentrated HCl (51 μl) added. The reaction was heated at reflux for 3 hours. The solvent was removed in vacuo to give the title compound as an oil that was used without further purification (243 mg, 89%); Mass Spectrum MH⁺=264.51.

Methyl N-(2,6-dichlorobenzoyl)-4-(tetrahydro-2H-pyran-4-yl)-L-phenylalaninate

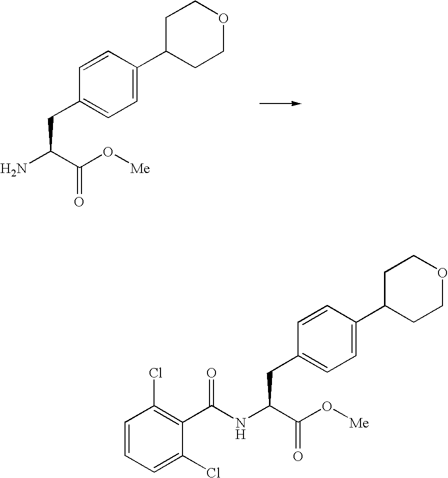

Methyl 4-(tetrahydro-2H-pyran-4-yl)-L-phenylalaninate (243 mg) was dissolved in DCM (10 ml) and triethylamine (0.34 ml) was added. 2,6-Dichlorobenzoyl chloride (0.14 ml) was added dropwise to give a yellow solution that was stirred at room temperature for 2 hours. Water (10 ml) was added, the layers separated and the organic layer dried. The solvent was removed in vacuo to give an oil that was purified by chromatography using 0-20% ethyl acetate in DCM as eluent to give the title compound as a white solid (160 mg, 45%); Mass Spectrum M⁺H=436.34.

### Example 15.1

### 4-(3,6-dihydro-2H-pyran-4-yl)-N-(2,6-dimethylbenzoyl)-L-phenylalanine

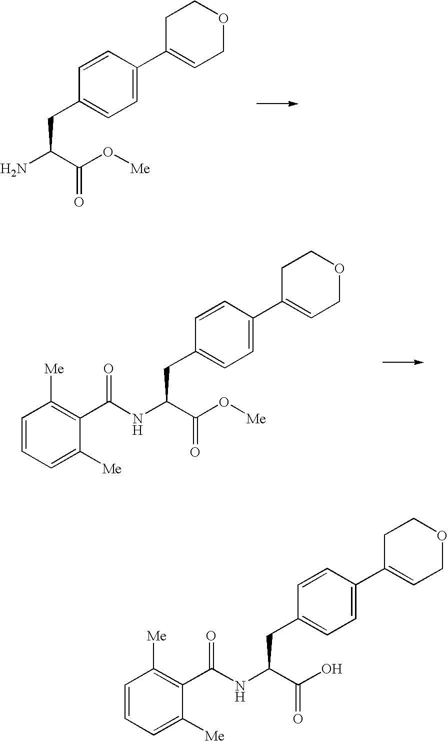

A solution of methyl 4-(3,6-dihydro-2H-pyran-4-yl)-L-phenylalaninate (80 mg) and triethylamine (33 μl) in DMF (1 ml) was added to 2,6-dimethylbenzoic acid (36 mg) followed by a solution of HATU (91 mg) in DMF (1 ml). The mixture was shaken overnight at room temperature then concentrated in vacuo. The residue was dissolved in ethyl acetate and washed with water (2×5 ml). The organic layer was concentrated and the residue was dissolved in 2:1 acetonitrile-methanol (3 ml) and a solution of lithium hydroxide monohydrate (31 mg) in water (200 μl) was added. The mixture was shaken at room temperature for 12 hours and the solvent removed. The crude product was purified by reverse phase preparative HPLC to afford the title compound (16 mg, 21%); ¹H NMR (DMSO-d6) δ 1.90-1.94 (2H, m), 1.99 (6H, s), 2.82-2.92 (1H, m), 3.14-3.22 (1H, m), 3.84 (2H, t), 4.19-4.25 (2H, m), 4.69-4.77 (1H, m), 6.19-6.23 (1H, m), 7.08-7.14 (3H, m), 7.29 (2H, d), 7.37 (2H, d), 8.58 (1H, d); Mass Spectrum MH⁺ 380.63.

The methyl 4-(3,6-dihydro-2H-pyran-4-yl)-L-phenylalaninate used as the starting material was prepared as follows:

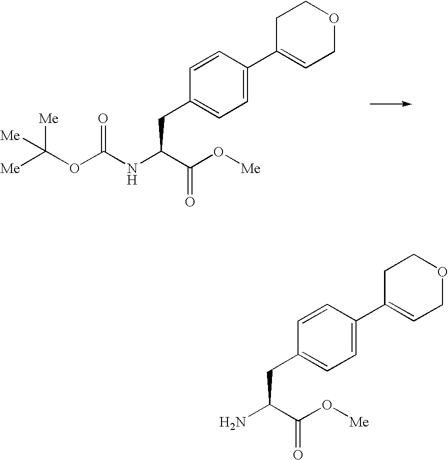

Methyl N-(tert-butoxycarbonyl)-4-(3,6-dihydro-2H-pyran-4-yl)-L-phenylalaninate was dissolved in methanol (30 ml) and concentrated HCl 0.28 ml was added. The mixture was heated at 65° C. for 12 hours. The reaction mixture was concentrated in vacuo. The residue was partitioned between 10% methanol in ethyl acetate and aqueous sodium carbonate, dried, filtered and evaporated to afford a yellow oil (1.23 g, 100%); ¹H NMR (DMSO-d6) δ1.80 (2H, br s), 2.40 (2H, m), 2.70-3.00 (2H, m), 3.60 (4H, m), 3.80 (2H, t), 4.20 (2H, m), 6.20 (1H, m), 7.15 (2H, m), 7.35 (2H, m); Mass Spectrum MH⁺ 262.54.

1-31. (canceled)

32. A compound of Formula I: 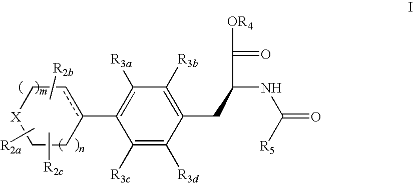
or a pharmaceutical acceptable salt, prodrug, or hydrate thereof, wherein:
X is O, N—R₁, or S(O)ₓ, wherein x is 0, 1, or 2;
m and n are each independently 0, 1, or 2;
“ [symbol] ” is a bond or is absent;
R₁ is (a) H, or an optionally substituted group selected from (C₁-C₆)alkyl, (C₃-C₆)cycloalkyl, heterocycloalkyl, (C₃-C₆)cycloalkyl(C₁-C₆)alkyl, heterocycloalkyl(C₁-C₆)alkyl, aryl, heteroaryl, aralkyl, or heteroaralkyl; or R₁ is
(b) 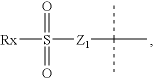
wherein 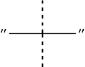
indicates the point of attachment and Z₁ is optionally substituted (C₁-C₆)alkylene, (C₁-C₆)alkenylene, (C₁-C₆)alkynylene, or is absent and Rₓ is an optionally substituted group selected from (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl; or R₁ is
(c) 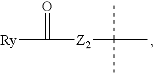
wherein 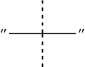
indicates the point of attachment and Z₂ is optionally substituted (C₁-C₆)alkylene, (C₁-C₆)alkenylene, (C₁-C₆)alkynylene, NR(C₁-C₆)alkylene, wherein R is H or (C₁-C₆)alkyl or is absent and R_{y} is an optionally substituted group selected from (C₁-C₆)alkyl, (C₁-C₆)alkoxy, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or NR′R″, wherein R′ and R″ are each independently H or (C₁-C₆)alkyl, (C₃-C₆)cycloalkyl, heterocycloalkyl, (C₃-C₆)cycloalkyl (C₁-C₆)alkylene, heterocycloalkyl(C₁-C₆)alkylene, aryl, heteroaryl, aralkyl, or heteroaralkyl, or taken together with the nitrogen to which they are attached, R′ and R″ form an optionally substituted 3, 4, 5, 6, or 7-membered ring; or R₁ is
(d) R₁ₐO—(C₁-C₆)alkylene, wherein R₁ₐ is H, (C₁-C₆)alkyl, (C₃-C₆)cycloalkyl, aryl, heteroaryl, (C₁-C₆)alkyl-C(═O)—, R_{1b}R_{1c}N—C(═O)—, wherein R_{1b} and R_{1c} are each independently H, (C₁-C₆)alkyl, (C₃-C₆)cycloalkyl, heterocycloalkyl, (C₃-C₆)cycloalkyl (C₁-C₆)alkylene, heterocycloalkyl(C₁-C₆)alkylene, aryl, heteroaryl, aralkyl, heteroaralkyl, or taken together with the nitrogen to which they are attached, R_{1b} and R_{1c} form an optionally substituted 3, 4, 5, 6, or 7-membered ring;
R₂ₐ, R_{2b}, and R_{2c} are each independently H, halo, hydroxy, (C₁-C₃)alkyl, or (C₁-C₃)alkoxy, or if two of R₂ₐ, R_{2b}, and R_{2c} are attached to the same carbon, they may form oxo;
R₃ₐ, R_{3b}, R_{3c}, and R_{3d} are each independently H, halo, (C₁-C₃)alkyl, or (C₁-C₃)alkoxy;
R₄ is H, (C₁-C₆)alkyl, aryl, heteroaryl, aralkyl, heteroaralkyl; and
R₅ is aryl which is ortho-substituted with at least one group selected from (C₁-C₃)alkyl, (C₁-C₃)alkoxy or halogen, and which is optionally additionally substituted with 1 or 2 groups selected from (C₁-C₃)alkyl, (C₁-C₃)alkoxy, halogen, cyano or heterocycloalkyl, provided that when X is N—S(O)₂Me, R₅ is 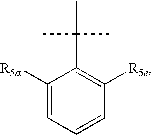
wherein R₅ₐ and R₅ₑ are each independently halo or (C₁-C₃)alkyl.

33. A compound according to claim 32 wherein R₅ is aryl which is ortho-substituted with at least one group selected from (C₁-C₃)alkyl or halogen, and which is optionally additionally substituted with 1 or 2 groups selected from (C₁-C₃)alkyl, (C₁-C₃)alkoxy or halogen, provided that when X is N—S(O)₂Me, R₅ is 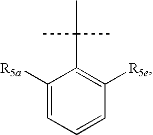
wherein R₅ₐ and R₅ₑ are each independently halo or (C₁-C₃)alkyl.

34. A compound according to claim 32 wherein “-----” is a bond.

35. A compound according to claim 32 wherein the group of the formula: 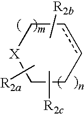
in the compound of formula I is: 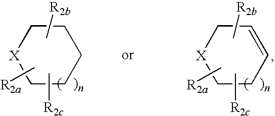
wherein X, R₂ₐ, R_{2b} and R_{2c} are as defined in claim 32; and
n is 0, 1 or 2;
and the group of the formula: 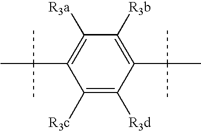
is: 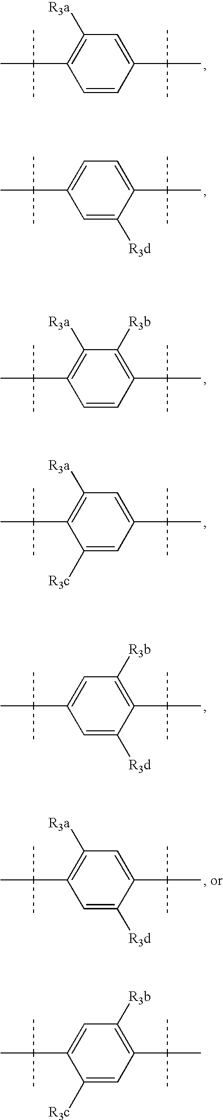
wherein R₃ₐ, R_{3b}, R_{3c}, and R_{3d} are as defined in claim 1.

36. A compound according to claim 32 wherein R₂ₐ, R_{2b} and R_{2c}, are each independently H, halo, (C₁-C₃)alkyl or (C₁-C₃)alkoxy.

37. A compound according to claim 32 wherein X is O.

38. A compound according to claim 32 wherein X is N—R₁, and R₁ is an optionally substituted group selected from aralkyl or heteroaralkyl, or is 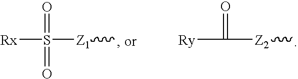
wherein Rₓ, R_{y}, Z₁ and Z₂ are as defined in claim 32.

39. A compound according to claim 32 wherein X is N—R₁, and R₁ is aralkyl which optionally bears 1, 2 or 3 substituents selected from (C₁-C₃)alkyl, (C₁-C₃)alkoxy, halo, cyano, —OH, —CF₃, —OCF₃, —NR⁶R⁷, —NHCOR⁶, —N[(C₁-C₆)alkyl]C(O)R⁶, —C(O)NR⁶R⁷, —C(O)(C₁-C₄)alkyl, —SO₂(C₁-C₄)alkyl and —SO₂NR⁶R⁷; wherein R⁶ and R⁷ are independently selected from hydrogen and (C₁-C₄)alkyl, or R₆ and R₇ together with the nitrogen to which they are attached form a 4- to 6-membered heterocycloalkyl group;
or two adjacent substituents on an aryl group within an R₁ group form a (C₁-C₄)alkylenedioxy group.

40. A compound according to claim 32 wherein X is N—R₁, and R₁ is 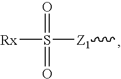
wherein [symbol] indicates the point of attachment and Z₁ and Rₓ are as defined in claim 32.

41. A compound according to claim 40 wherein Z₁ is absent and Rₓ is an optionally substituted group selected from (i) to (vii):
(i) optionally substituted (C₁-C₄)alkyl;
(ii) optionally substituted (C₃-C₆)cycloalkyl;
(iii) optionally substituted (C₃-C₆)cycloalkyl(C₁-C₃)alkylene;
(iv) optionally substituted phenyl;
(v) optionally substituted heteroaryl;
(vi) optionally substituted benzyl; and
(vii) optionally substituted heteroaryl(C₁-C₃)alkylene;
wherein the optional substituents that may be present on Rₓ are independently selected from (C₁-C₃)alkyl, (C₁-C₃)alkoxy, halo, cyano, —OH, —CF₃, —OCF₃, —NR⁶R⁷, —NHCOR⁶, —N[(C₁-C₆)alkyl]C(O)R⁶, —C(O)NR⁶R⁷, —C(O)(C₁-C₄)alkyl, —SO₂(C₁-C₄)alkyl and —SO₂NR⁶R⁷; wherein R⁶ and R⁷ are independently selected from hydrogen and (C₁-C₄)alkyl, or R₆ and R₇ together with the nitrogen to which they are attached form a 4- to 6-membered heterocycloalkyl group;
or two adjacent substituents on an aryl group within an Rₓ group form a (C₁-C₄)alkylenedioxy group.

42. A compound according to claim 32 wherein X is N—R₁, and R₁ is 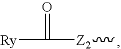
wherein [symbol] indicates the point of attachment, and Z₂ and R_{y} are as defined in claim 32.

43. A compound according to claim 42 wherein Z₂ is absent and R_{y} is an optionally substituted group selected from (i) to (x):
(i) optionally substituted (C₁-C₄)alkyl;
(ii) optionally substituted (C₃-C₆)cycloalkyl;
(iii) optionally substituted (C₃-C₆)cycloalkyl(C₁-C₃)alkylene;
(iv) optionally substituted heterocycloalkyl;
(v) optionally substituted phenyl;
(vi) optionally substituted heteroaryl;
(vii) optionally substituted benzyl;
(viii) optionally substituted heteroaryl(C₁-C₃)alkyl;
(ix) optionally substituted heterocycloalkyl(C₁-C₃)alkyl; and
(x) NR′R″, wherein R′ and R″ are each independently H or optionally substituted (C₁-C₄)alkyl, phenyl or benzyl or R′ and R″ taken together with the nitrogen to which they are attached form an optionally substituted azetidinyl, pyrrolidinyl, piperidinyl or morpholinyl ring;
and wherein the optional substituents that may be present on R_{y} are independently selected from (C₁-C₃)alkyl, (C₁-C₃)alkoxy, halo, cyano, —OH, —CF₃, —OCF₃, —NR⁶R⁷, —NHCOR⁶, —N[(C₁-C₆)alkyl]C(O)R⁶, —C(O)NR⁶R⁷, —C(O)(C₁-C₄)alkyl, —SO₂(C₁-C₄)alkyl and —SO₂NR⁶R⁷; wherein R⁶ and R⁷ are independently selected from hydrogen and (C₁-C₄)alkyl, or R₆ and R₇ together with the nitrogen to which they are attached form a 4- to 6-membered heterocycloalkyl group;
or two adjacent substituents on an aryl group within an R_{y} group form a (C₁-C₄)alkylenedioxy group.

44. A compound according to claim 32 wherein R₃ₐ, R_{3b}, R_{3c}, and R_{3d} are all H.

45. A compound according to claim 32 which is a compound of formula IA:

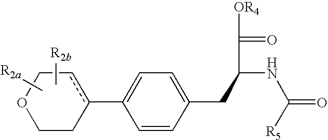

46. A compound according to claim 32, which is a compound of formula IB: 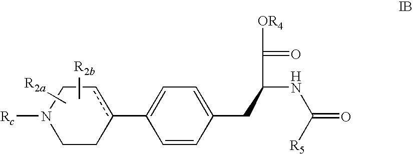
wherein R_{c} is an optionally substituted group selected from alkyl, aryl or heteroaryl.

47. A compound according to claim 32, which is a compound of formula IC: 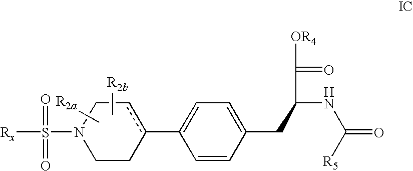
wherein Rₓ is an optionally substituted group selected from (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, or heteroaralkyl.

48. A compound according to claim 32, which is a compound of formula ID 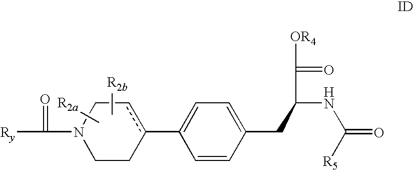
wherein R_{y} is an optionally substituted group selected from (C₁-C₆)alkyl, (C₃-C₆)cycloalkyl, heterocycloalkyl, (C₃-C₆)cycloalkyl(C₁-C₆)alkylene, heterocycloalkyl(C₁-C₆)alkylene, aryl, heteroaryl, aralkyl, heteroaralkyl, or NR′R″, wherein R′ and R″ are each independently H or (C₁-C₆)alkyl, aryl, heteroaryl, aralkyl, or heteroaralkyl, (C₁-C₆)alkoxy, (C₃-C₆)cycloalkyl, heterocycloalkyl, (C₃-C₆)cycloalkyl (C₁-C₆)alkylene, heterocycloalkyl(C₁-C₆)alkylene, aryl, heteroaryl, aralkyl, or heteroaralkyl or taken together with the nitrogen to which they are attached form an optionally substituted 3, 4, 5, 6, or 7-membered ring.

49. A compound according to claim 32 wherein R₅ is a group of the formula: 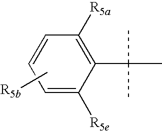
wherein R₅ₐ is chloro or (C₁-C₃)alkyl;
R₅ₑ is H chloro or (C₁-C₃)alkyl;
R_{5b} is H, halo, cyano, (C₁-C₃)alkyl or (C₁-C₃)alkoxy; and 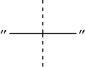
indicates the point of attachment.

50. A compound according to claim 49 wherein R₅ₐ is chloro, R_{5b} is H and R₅ₑ is chloro or methyl.

51. A compound according to claim 32 wherein R₄ is H.

52. A compound according to claim 32 other than:
N-(2-chloro-4-fluorobenzoyl)-4-{1-[(2R)-2-hydroxypropanoyl]-1,2,3,6-tetrahydropyridin-4-yl}-L-phenylalanine;
4-(1-acetyl-1,2,3,6-tetrahydropyridin-4-yl)-N-(4-chloro-2,5-difluorobenzoyl)-L-phenylalanine;
N-(2-chloro-3-fluorobenzoyl)-4-[1-(propylsulfonyl)-1,2,3,6-tetrahydropyridin-4-yl]-L-phenylalanine;
4-[1-(propylsulfonyl)-1,2,3,6-tetrahydropyridin-4-yl]-N-(2,3,5-trifluorobenzoyl)-L-phenylalanine;
4-(1-acetyl-1,2,3,6-tetrahydropyridin-4-yl)-N-(2-chloro-3-fluorobenzoyl)-L-phenylalanine;
N-(2-chloro-3-fluorobenzoyl)-4-[1-(pyridin-2-ylcarbonyl)-1,2,3,6-tetrahydropyridin-4-yl]-L-phenylalanine;
N-(2-chloro-6-methyl benzoyl)-4-[1-(4-fluorobenzyl)-1,2,3,6-tetrahydropyridin-4-yl]-L-phenylalanine;
N-(2,6-dichlorobenzoyl)-4-[1-(2-fluorobenzyl)piperidin-4-yl]-L-phenylalanine; and
N-(2,6-dichlorobenzoyl)-4-[1-(3-methoxybenzyl)piperidin-4-yl]-L-phenylalanine.

53. A compound selected from: 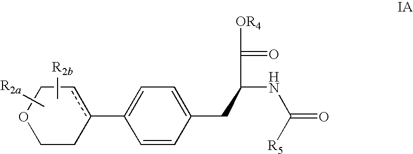
N-(2,6-dichlorobenzoyl)-4-(3,6-dihydro-2H-pyran-4-yl)-L-phenylalanine;
N-(2,6-dichlorobenzoyl)-4-(tetrahydro-2H-pyran-4-yl)-L-phenylalanine;
4-(3,6-dihydro-2H-pyran-4-yl)-N-(2,6-dimethylbenzoyl)-L-phenylalanine; 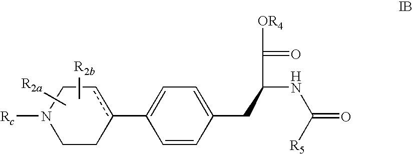
4-(1-benzylpiperidin-4-yl)-N-(2,6-dichlorobenzoyl)-L-phenylalanine;
N-(2,6-dichlorobenzoyl)-4-[1-(3-fluorobenzyl)piperidin-4-yl]-L-phenylalanine;
N-(2,6-dichlorobenzoyl)-4-[1-(4-fluorobenzyl)piperidin-4-yl]-L-phenylalanine;
4-[1-(2-aminobenzyl)piperidin-4-yl]-N-(2,6-dichlorobenzoyl)-L-phenylalanine;
4-[1-(3-cyanobenzyl)piperidin-4-yl]-N-(2,6-dichlorobenzoyl)-L-phenylalanine;
N-(2,6-dichlorobenzoyl)-4-[1-(4-methoxybenzyl)piperidin-4-yl]-L-phenylalanine;
N-(2,6-dichlorobenzoyl)-4-[1-(2-methoxybenzyl)piperidin-4-yl]-L-phenylalanine;
4-[1-(2-chlorobenzyl)piperidin-4-yl]-N-(2,6-dichlorobenzoyl)-L-phenylalanine;
4-[1-(3-chlorobenzyl)piperidin-4-yl]-N-(2,6-dichlorobenzoyl)-L-phenylalanine;
4-[1-(4-chlorobenzyl)piperidin-4-yl]-N-(2,6-dichlorobenzoyl)-L-phenylalanine;
N-(2,6-dichlorobenzoyl)-4-[1-(2-hydroxyethyl)-1,2,3,6-tetrahydropyridin-4-yl]-L-phenylalanine;
4-(1-benzyl-1,2,3,6-tetrahydropyridin-4-yl)-N-(2,6-dichlorobenzoyl)-L-phenylalanine;
N-(2,6-dichlorobenzoyl)-4-(1-isobutyl-1,2,3,6-tetrahydropyridin-4-yl)-L-phenylalanine;
4-[1-(2-amino-2-oxoethyl)-1,2,3,6-tetrahydropyridin-4-yl]-N-(2,6-dichlorobenzoyl)-L-phenylalanine;
N-(2,6-dichlorobenzoyl)-4-[1-(2-methylbenzyl)-1,2,3,6-tetrahydropyridin-4-yl]-L-phenylalanine;
N-(2,6-dichlorobenzoyl)-4-[1-(2-fluorobenzyl)-1,2,3,6-tetrahydropyridin-4-yl]-L-phenylalanine;
N-(2,6-dichlorobenzoyl)-4-[1-(2-chlorobenzyl)-1,2,3,6-tetrahydropyridin-4-yl]-L-phenylalanine;
N-(2,6-dichlorobenzoyl)-4-[1-(3-fluorobenzyl)-1,2,3,6-tetrahydropyridin-4-yl]-L-phenylalanine;
N-(2,6-dichlorobenzoyl)-4-[1-(3-chlorobenzyl)-1,2,3,6-tetrahydropyridin-4-yl]-L-phenylalanine;
N-(2,6-dichlorobenzoyl)-4-[1-(3-methoxybenzyl)-1,2,3,6-tetrahydropyridin-4-yl]-L-phenylalanine;
N-(2,6-dichlorobenzoyl)-4-[1-(3-methylbenzyl)-1,2,3,6-tetrahydropyridin-4-yl]-L-phenylalanine;
N-(2,6-dichlorobenzoyl)-4-[1-(4-fluorobenzyl)-1,2,3,6-tetrahydropyridin-4-yl]-L-phenylalanine;
N-(2,6-dichlorobenzoyl)-4-[1-(4-chlorobenzyl)-1,2,3,6-tetrahydropyridin-4-yl]-L-phenylalanine;
N-(2,6-dichlorobenzoyl)-4-[1-(2-methoxybenzyl)-1,2,3,6-tetrahydropyridin-4-yl]-L-phenylalanine;
N-(2,6-dichlorobenzoyl)-4-[1-(4-hydroxybenzyl)-1,2,3,6-tetrahydropyridin-4-yl]-L-phenylalanine;
N-(2,6-dichlorobenzoyl)-4-[1-(3,5-dimethoxybenzyl)-1,2,3,6-tetrahydropyridin-4-yl]-L-phenylalanine;
N-(2,6-dichlorobenzoyl)-4-[1-(2-hydroxybenzyl)-1,2,3,6-tetrahydropyridin-4-yl]-L-phenylalanine;
N-(2,6-dichlorobenzoyl)-4-[1-(4-cyanobenzyl)-1,2,3,6-tetrahydropyridin-4-yl]-L-phenylalanine;
N-(2,6-dichlorobenzoyl)-4-{1-[(5-methyl isoxazol-3-yl)methyl]-1,2,3,6-tetrahydropyridin-4-yl}-L-phenylalanine;
N-(2,6-dichlorobenzoyl)-4-{1-[(3,5-dimethylisoxazol-4-yl)methyl]-1,2,3,6-tetrahydropyridin-4-yl}-L-phenylalanine;
N-(2,6-dichlorobenzoyl)-4-[1-(3-cyanobenzyl)-1,2,3,6-tetrahydropyridin-4-yl]-L-phenylalanine;
N-(2,6-dichlorobenzoyl)-4-[1-(3-chloro-4-fluorobenzyl)-1,2,3,6-tetrahydropyridin-4-yl]-L-phenylalanine;
N-(2,6-dichlorobenzoyl)-4-[1-(2,5-difluorobenzyl)-1,2,3,6-tetrahydropyridin-4-yl]-L-phenylalanine;
N-(2,6-dichlorobenzoyl)-4-[1-(2,4-difluorobenzyl)-1,2,3,6-tetrahydropyridin-4-yl]-L-phenylalanine;
N-(2,6-dichlorobenzoyl)-4-[1-(2,3-difluorobenzyl)-1,2,3,6-tetrahydropyridin-4-yl]-L-phenylalanine;
4-[1-(1,3-benzodioxol-5-ylmethyl)-1,2,3,6-tetrahydropyridin-4-yl]-N-(2,6-dichlorobenzoyl)-L-phenylalanine;
N-(2,6-dichlorobenzoyl)-4-[1-(3,4-difluorobenzyl)-1,2,3,6-tetrahydropyridin-4-yl]-L-phenylalanine;
4-{1-[4-(acetylamino)benzyl]-1,2,3,6-tetrahydropyridin-4-yl}-N-(2,6-dichlorobenzoyl)-L-phenylalanine;
N-(2,6-dichlorobenzoyl)-4-[1-(2,5-dimethoxybenzyl)-1,2,3,6-tetrahydropyridin-4-yl]-L-phenylalanine;
N-(2,6-dichlorobenzoyl)-4-{1-[4-(methylsulfonyl)benzyl]-1,2,3,6-tetrahydropyridin-4-yl}-L-phenylalanine;
N-(2,6-dichlorobenzoyl)-4-(1-phenyl-1,2,3,6-tetrahydropyridin-4-yl)-L-phenylalanine; 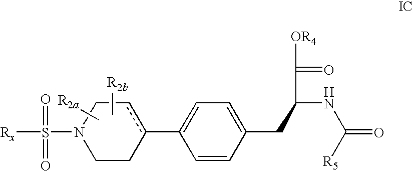
N-(2,6-dimethylbenzoyl)-4-[1-(methylsulfonyl)-1,2,3,6-tetrahydropyridin-4-yl]-L-phenylalanine;
N-(2,6-dichlorobenzoyl)-4-[1-(methylsulfonyl)-1,2,3,6-tetrahydropyridin-4-yl]-L-phenylalanine;
N-(2-chloro-4-fluorobenzoyl)-4-[1-(methylsulfonyl)-1,2,3,6-tetrahydropyridin-4-yl]-L-phenylalanine;
N-(4-bromo-2-chlorobenzoyl)-4-[1-(methylsulfonyl)-1,2,3,6-tetrahydropyridin-4-yl]-L-phenylalanine;
N-(2-chloro-4-pyrrolidin-1-ylbenzoyl)-4-[1-(methylsulfonyl)-1,2,3,6-tetrahydropyridin-4-yl]-L-phenylalanine;
N-(2,6-dichlorobenzoyl)-4-[1-(methylsulfonyl)piperidin-4-yl]-L-phenylalanine;
N-(2-chlorobenzoyl)-4-[1-(methylsulfonyl)piperidin-4-yl]-L-phenylalanine;
N-(2-chloro-6-methylbenzoyl)-4-[1-(pyridin-3-ylsulfonyl)-1,2,3,6-tetrahydropyridin-4-yl]-L-phenylalanine;
4-[1-(butylsulfonyl)piperidin-4-yl]-N-(2,6-dichlorobenzoyl)-L-phenylalanine;
N-(2,6-dichlorobenzoyl)-4-[1-(pyridin-3-ylsulfonyl)piperidin-4-yl]-L-phenylalanine;
N-(2,6-dichlorobenzoyl)-4-[1-(2-thienylsulfonyl)piperidin-4-yl]-L-phenylalanine;
N-(2,6-dichlorobenzoyl)-4-[1-(phenylsulfonyl)-1,2,3,6-tetrahydropyridin-4-yl]-L-phenylalanine;
N-(2,6-dichlorobenzoyl)-4-{1-[(4-fluorophenyl)sulfonyl]-1,2,3,6-tetrahydropyridin-4-yl}-L-phenylalanine;
N-(2,6-dichlorobenzoyl)-4-[1-(propylsulfonyl)-1,2,3,6-tetrahydropyridin-4-yl]-L-phenylalanine;
N-(2,6-dichlorobenzoyl)-4-{1-[(3-fluorophenyl)sulfonyl]-1,2,3,6-tetrahydropyridin-4-yl}-L-phenylalanine;
N-(2,6-dichlorobenzoyl)-4-{1-[(1,3,5-trimethyl-1H-pyrazol-4-yl)sulfonyl]-1,2,3,6-tetrahydropyridin-4-yl}-L-phenylalanine;
N-(2,6-dichlorobenzoyl)-4-[1-(2-thienylsulfonyl)-1,2,3,6-tetrahydropyridin-4-yl}-L-phenylalanine;
N-(2,6-dichlorobenzoyl)-4-{1-[(1,2-dimethyl-1H-imidazol-4-yl)sulfonyl]-1,2,3,6-tetrahydropyridin-4-yl}-L-phenylalanine;
N-(2,6-dichlorobenzoyl)-4-{1-[(3-chlorophenyl)sulfonyl]-1,2,3,6-tetrahydropyridin-4-yl}-L-phenylalanine;
N-(2,6-dichlorobenzoyl)-4-{1-[(4-methoxyphenyl)sulfonyl]-1,2,3,6-tetrahydropyridin-4-yl}-L-phenylalanine;
4-[1-(cyclopropylsulfonyl)-1,2,3,6-tetrahydropyridin-4-yl]-N-(2,6-dichlorobenzoyl)-L-phenylalanine;
N-(2,6-dichlorobenzoyl)-4-{1-[(2,4-dimethyl-1,3-thiazol-5-yl)sulfonyl]-1,2,3,6-tetrahydropyridin-4-yl}-L-phenylalanine; 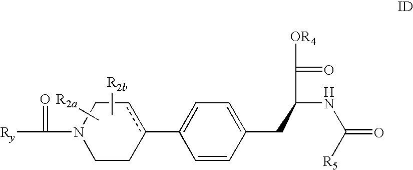
N-(2-chloro-4,5-dimethoxybenzoyl)-4-[1-(4-fluorobenzoyl)-1,2,3,6-tetrahydropyridin-4-yl]-L-phenylalanine;
N-(2-chloro-4,5-dimethoxybenzoyl)-4-[1-(3-fluorobenzoyl)-1,2,3,6-tetrahydropyridin-4-yl]-L-phenylalanine;
N-(2-chloro-6-methylbenzoyl)-4-[1-(3-fluorobenzoyl)-1,2,3,6-tetrahydropyridin-4-yl]-L-phenylalanine;
4-[1-(4-cyanobenzoyl)-1,2,3,6-tetrahydropyridin-4-yl]-N-(2,6-dimethylbenzoyl)-L-phenylalanine;
4-[1-(1H-benzimidazol-2-ylcarbonyl)-1,2,3,6-tetrahydropyridin-4-yl]-N-(2-chloro-3-fluorobenzoyl)-L-phenylalanine;
N-(2,6-dimethylbenzoyl)-4-[1-(3-methoxybenzoyl)-1,2,3,6-tetrahydropyridin-4-yl]-L-phenylalanine;
N-(2-chloro-4,5-dimethoxybenzoyl)-4-[1-(3-methoxybenzoyl)-1,2,3,6-tetrahydropyridin-4-yl]-L-phenylalanine;
N-(2-chloro-6-methyl benzoyl)-4-[1-(3-methoxybenzoyl)-1,2,3,6-tetrahydropyridin-4-yl]-L-phenylalanine;
N-(2,6-dimethylbenzoyl)-4-{1-[(2-methyl-1,3-thiazol-4-yl)carbonyl]-1,2,3,6-tetrahydropyridin-4-yl}-L-phenylalanine;
4-[1-(1H-benzimidazol-2-ylcarbonyl)-1,2,3,6-tetrahydropyridin-4-yl]-N-(4-cyano-2-methoxybenzoyl)-L-phenylalanine;
4-[1-(1H-benzimidazol-2-ylcarbonyl)-1,2,3,6-tetrahydropyridin-4-yl]-N-(2-chloro-4,5-dimethoxybenzoyl)-L-phenylalanine;
N-(2-chloro-4,5-dimethoxybenzoyl)-4-{1-[(2-methyl-1,3-thiazol-4-yl)carbonyl]-1,2,3,6-tetrahydropyridin-4-yl}-L-phenylalanine;
N-(2-chloro-6-methylbenzoyl)-4-[1-(3-cyanobenzoyl)-1,2,3,6-tetrahydropyridin-4-yl]-L-phenylalanine;
4-[1-(1H-benzimidazol-2-ylcarbonyl)-1,2,3,6-tetrahydropyridin-4-yl]-N-(2-chloro-6-methylbenzoyl)-L-phenylalanine;
N-(2-chloro-3-fluorobenzoyl)-4-[1-(4-fluorobenzyl)-1,2,3,6-tetrahydropyridin-4-yl]-L-phenylalanine;
N-(2,6-dimethylbenzoyl)-4-[1-(4-fluorobenzyl)-1,2,3,6-tetrahydropyridin-4-yl]-L-phenylalanine;
4-[1-(cinnolin-4-ylcarbonyl)-1,2,3,6-tetrahydropyridin-4-yl]-N-(2,6-dimethylbenzoyl)-L-phenylalanine;
4-(1-acetyl-1,2,3,6-tetrahydropyridin-4-yl)-N-(2-chloro-6-methylbenzoyl)-L-phenylalanine;
N-(2-chloro-6-methylbenzoyl)-4-[1-(cyclopropylcarbonyl)-1,2,3,6-tetrahydropyridin-4-yl]-L-phenylalanine;
N-(2-chloro-6-methylbenzoyl)-4-[1-(pyridin-2-ylcarbonyl)-1,2,3,6-tetrahydropyridin-4-yl]-L-phenylalanine;
N-(2-chloro-4-fluorobenzoyl)-4-[1-(cinnolin-4-ylcarbonyl)-1,2,3,6-tetrahydropyridin-4-yl]-L-phenylalanine;
N-(2-chloro-4-fluorobenzoyl)-4-[1-(4-fluorobenzoyl)-1,2,3,6-tetrahydropyridin-4-yl]-L-phenylalanine;
N-(2,6-dimethylphenyl)-4-[1-(4-fluorobenzyl)-1,2,3,6-tetrahydropyridin-4-yl]-L-phenylalanine;
N-(2,6-dichlorobenzoyl)-4-[1-(pyrazin-2-ylcarbonyl)piperidin-4-yl]-L-phenylalanine;
N-(2,6-dichlorobenzoyl)-4-[1-(1,3-thiazol-4-ylcarbonyl)piperidin-4-yl]-L-phenylalanine;
N-(2,6-dichlorobenzoyl)-4-[1-(1,3-thiazol-5-ylcarbonyl)piperidin-4-yl]-L-phenylalanine;
N-(2,6-dichlorobenzoyl)-4-(1-isonicotinoylpiperidin-4-yl)-L-phenylalanine;
N-(2,6-dichlorobenzoyl)-4-[1-(1,2,5-thiadiazol-3-ylcarbonyl)piperidin-4-yl]-L-phenylalanine;
4-[1-(cinnolin-4-ylcarbonyl)piperidin-4-yl]-N-(2,6-dichlorobenzoyl)-L-phenylalanine;
N-(2,6-dichlorobenzoyl)-4-{1-[(1-methyl-1H-pyrrol-2-yl)carbonyl]piperidin-4-yl}-L-phenylalanine;
4-[1-(1H-benzimidazol-2-ylcarbonyl)piperidin-4-yl]-N-(2,6-dichlorobenzoyl)-L-phenylalanine;
N-(2,6-dichlorobenzoyl)-4-[1-(1H-indazol-3-ylcarbonyl)piperidin-4-yl]-L-phenylalanine;
N-(2,6-dichlorobenzoyl)-4-[1-(methoxyacetyl)piperidin-4-yl]-L-phenylalanine;
N-(2,6-dichlorobenzoyl)-4-[1-(3-methylbutanoyl)piperidin-4-yl]-L-phenylalanine;
4-{1-[(1-cyanocyclopropyl)carbonyl]piperidin-4-yl}-N-(2,6-dichlorobenzoyl)-L-phenylalanine;
N-(2,6-dichlorobenzoyl)-4-[1-(2-fluorobenzoyl)piperidin-4-yl]-L-phenylalanine;
N-(2,6-dichlorobenzoyl)-4-[1-(3-fluorobenzoyl)piperidin-4-yl]-L-phenylalanine;
N-(2,6-dichlorobenzoyl)-4-[1-(4-fluorobenzoyl)piperidin-4-yl]-L-phenylalanine;
4-[1-(3-cyanobenzoyl)piperidin-4-yl]-N-(2,6-dichlorobenzoyl)-L-phenylalanine;
4-[1-(4-cyanobenzoyl)piperidin-4-yl]-N-(2,6-dichlorobenzoyl)-L-phenylalanine;
N-(2,6-dichlorobenzoyl)-4-[1-(2-methoxybenzoyl)piperidin-4-yl]-L-phenylalanine;
N-(2,6-dichlorobenzoyl)-4-[1-(3-methoxybenzoyl)piperidin-4-yl]-L-phenylalanine;
N-(2,6-dichlorobenzoyl)-4-[1-(4-methoxybenzoyl)piperidin-4-yl]-L-phenylalanine;
4-[1-(2-chlorobenzoyl)piperidin-4-yl]-N-(2,6-dichlorobenzoyl)-L-phenylalanine;
4-[1-(4-chlorobenzoyl)piperidin-4-yl]-N-(2,6-dichlorobenzoyl)-L-phenylalanine;
4-[1-(1,3-benzodioxol-4-ylcarbonyl)piperidin-4-yl]-N-(2,6-dichlorobenzoyl)-L-phenylalanine;
N-(2,6-dichlorobenzoyl)-4-[1-(3-phenylpropanoyl)-1,2,3,6-tetrahydropyridin-4-yl]-L-phenylalanine;
4-(1-acetyl-1,2,3,6-tetrahydropyridin-4-yl)-N-(2,6-dichlorobenzoyl)-L-phenylalanine;
N-(2,6-dichlorobenzoyl)-4-[1-(benzoyl)-1,2,3,6-tetrahydropyridin-4-yl]-L-phenylalanine;
N-(2,6-dichlorobenzoyl)-4-[1-(2-methylbenzoyl)-1,2,3,6-tetrahydropyridin-4-yl]-L-phenylalanine;
N-(2,6-dichlorobenzoyl)-4-[1-(3-cyanobenzoyl)-1,2,3,6-tetrahydropyridin-4-yl]-L-phenylalanine;
N-(2,6-dichlorobenzoyl)-4-[1-(4-cyanobenzoyl)-1,2,3,6-tetrahydropyridin-4-yl]-L-phenylalanine;
N-(2,6-dichlorobenzoyl)-4-[1-(4-methylbenzoyl)-1,2,3,6-tetrahydropyridin-4-yl]-L-phenylalanine;
N-(2,6-dichlorobenzoyl)-4-[1-(2-fluorobenzoyl)-1,2,3,6-tetrahydropyridin-4-yl]-L-phenylalanine;
4-[1-(cyclopropylcarbonyl)-1,2,3,6-tetrahydropyridin-4-yl]-N-(2,6-dichlorobenzoyl)-L-phenylalanine;
N-(2,6-dichlorobenzoyl)-4-[1-(3-fluorobenzoyl)-1,2,3,6-tetrahydropyridin-4-yl]-L-phenylalanine;
N-(2,6-dichlorobenzoyl)-4-[1-(3-methoxybenzoyl)-1,2,3,6-tetrahydropyridin-4-yl]-L-phenylalanine;
N-(2,6-dichlorobenzoyl)-4-[1-(hydroxyacetyl)-1,2,3,6-tetrahydropyridin-4-yl]-L-phenylalanine;
N-(2,6-dichlorobenzoyl)-4-[1-(2-cyanobenzoyl)-1,2,3,6-tetrahydropyridin-4-yl]-L-phenylalanine;
N-(2,6-dichlorobenzoyl)-4-{1-[(1-methyl-1H-imidazol-2-yl)carbonyl]-1,2,3,6-tetrahydropyridin-4-yl}-L-phenylalanine;
N-(2,6-dichlorobenzoyl)-4-{1-[(1,5-dimethyl-1H-pyrrol-2-yl)carbonyl]-1,2,3,6-tetrahydropyridin-4-yl}-L-phenylalanine;
N-(2,6-dichlorobenzoyl)-4-{1-[(1-methyl-1H-pyrazol-3-yl)carbonyl]-1,2,3,6-tetrahydropyridin-4-yl}-L-phenylalanine;
N-(2,6-dichlorobenzoyl)-4-{1-[(5-methylisoxazol-3-yl)carbonyl]-1,2,3,6-tetrahydropyridin-4-yl}-L-phenylalanine;
N-(2,6-dichlorobenzoyl)-4-{1-[(5-methylpyridin-3-yl)carbonyl]-1,2,3,6-tetrahydropyridin-4-yl}-L-phenylalanine;
N-(2,6-dichlorobenzoyl)-4-[1-(1,3-thiazol-4-ylcarbonyl)-1,2,3,6-tetrahydropyridin-4-yl]-L-phenylalanine;
N-(2,6-dichlorobenzoyl)-4-{1-[(4-methyl-1,3-thiazol-5-yl)carbonyl]-1,2,3,6-tetrahydropyridin-4-yl}-L-phenylalanine;
N-(2,6-dichlorobenzoyl)-4-[1-(5-methyl-2-furoyl)-1,2,3,6-tetrahydropyridin-4-yl]-L-phenylalanine;
N-(2,6-dichlorobenzoyl)-4-{1-[(3-methylisoxazol-4-yl)carbonyl]-1,2,3,6-tetrahydropyridin-4-yl}-L-phenylalanine;
4-[1-(1-benzofuran-2-ylcarbonyl)-1,2,3,6-tetrahydropyridin-4-yl]-N-(2,6-dichlorobenzoyl)-L-phenylalanine;
4-[1-(cinnolin-4-ylcarbonyl)-1,2,3,6-tetrahydropyridin-4-yl]-N-(2,6-dichlorobenzoyl)-L-phenylalanine;
N-(2,6-dichlorobenzoyl)-4-[1-(isoquinolin-1-ylcarbonyl)-1,2,3,6-tetrahydropyridin-4-yl]-L-phenylalanine;
N-(2,6-dichlorobenzoyl)-4-{1-[(1,5-dimethyl-1H-pyrazol-3-yl)carbonyl]-1,2,3,6-tetrahydropyridin-4-yl}-L-phenylalanine;
N-(2,6-dichlorobenzoyl)-4-[1-(1H-indazol-3-ylcarbonyl)-1,2,3,6-tetrahydropyridin-4-yl]-L-phenylalanine;
N-(2,6-dichlorobenzoyl)-4-[1-(3-thienylcarbonyl)-1,2,3,6-tetrahydropyridin-4-yl]-L-phenylalanine;
N-(2,6-dichlorobenzoyl)-4-[1-(pyridin-2-ylcarbonyl)-1,2,3,6-tetrahydropyridin-4-yl]-L-phenylalanine;
N-(2,6-dichlorobenzoyl)-4-[1-(2-thienylcarbonyl)-1,2,3,6-tetrahydropyridin-4-yl]-L-phenylalanine;
N-(2,6-dichlorobenzoyl)-4-[1-(quinolin-4-ylcarbonyl)-1,2,3,6-tetrahydropyridin-4-yl]-L-phenylalanine;
N-(2,6-dichlorobenzoyl)-4-{1-[(5-methylpyrazin-2-yl)carbonyl]-1,2,3,6-tetrahydropyridin-4-yl}-L-phenylalanine;
4-[1-(1H-benzimidazol-2-ylcarbonyl)-1,2,3,6-tetrahydropyridin-4-yl]-N-(2,6-dichlorobenzoyl)-L-phenylalanine;
N-(2,6-dichlorobenzoyl)-4-{1-[4-(trifluoromethyl)benzoyl]-1,2,3,6-tetrahydropyridin-4-yl}-L-phenylalanine;
N-(2,6-dichlorobenzoyl)-4-{1-[3-(trifluoromethyl)benzoyl]-1,2,3,6-tetrahydropyridin-4-yl}-L-phenylalanine;
N-(2,6-dichlorobenzoyl)-4-{1-[2-chlorobenzoyl]-1,2,3,6-tetrahydropyridin-4-yl}-L-phenylalanine;
N-(2,6-dichlorobenzoyl)-4-{1-[3-chlorobenzoyl]-1,2,3,6-tetrahydropyridin-4-yl}-L-phenylalanine;
N-(2,6-dichlorobenzoyl)-4-{1-[4-(hydroxymethyl)benzoyl]-1,2,3,6-tetrahydropyridin-4-yl}-L-phenylalanine;
N-(2,6-dichlorobenzoyl)-4-{1-[2-(trifluoromethyl)benzoyl]-1,2,3,6-tetrahydropyridin-4-yl}-L-phenylalanine;
N-(2,6-dichlorobenzoyl)-4-{1-[2-methoxybenzoyl]-1,2,3,6-tetrahydropyridin-4-yl}-L-phenylalanine;
N-(2,6-dichlorobenzoyl)-4-{1-[(1,1-dioxidotetrahydro-2H-thiopyran-4-yl)carbonyl]-1,2,3,6-tetrahydropyridin-4-yl}-L-phenylalanine;
N-(2,6-dichlorobenzoyl)-4-{1-[(3,5-dimethyl-1H-pyrazol-1-yl)acetyl]-1,2,3,6-tetrahydropyridin-4-yl}-L-phenylalanine;
N-(2,6-dichlorobenzoyl)-4-{1-[(2,5-dimethyl-1,3-thiazol-4-yl)acetyl]-1,2,3,6-tetrahydropyridin-4-yl}-L-phenylalanine;
4-[1-(2,1,3-benzoxadiazol-5-ylcarbonyl)-1,2,3,6-tetrahydropyridin-4-yl]-N-(2,6-dichlorobenzoyl)-L-phenylalanine;
N-(2,6-dichlorobenzoyl)-4-[1-(3-hydroxy-2,2-dimethylpropanoyl)-1,2,3,6-tetrahydropyridin-4-yl]-L-phenylalanine;
N-(2,6-dichlorobenzoyl)-4-{1-[(2R)-3,3,3-trifluoro-2-hydroxy-2-methylpropanoyl]-1,2,3,6-tetrahydropyridin-4-yl}-L-phenylalanine;
4-[1-(1,2-benzisoxazol-3-ylacetyl)-1,2,3,6-tetrahydropyridin-4-yl]-N-(2,6-dichlorobenzoyl)-L-phenylalanine;
N-(2,6-dichlorobenzoyl)-4-[1-(1H-1,2,4-triazol-1-ylacetyl)-1,2,3,6-tetrahydropyridin-4-yl]-L-phenylalanine;
N-(2,6-dichlorobenzoyl)-4-[1-(tetrahydro-2H-pyran-4-ylacetyl)-1,2,3,6-tetrahydropyridin-4-yl]-L-phenylalanine;
N-(2,6-dichlorobenzoyl)-4-[1-(tetrahydro-2H-pyran-4-ylcarbonyl)-1,2,3,6-tetrahydropyridin-4-yl]-L-phenylalanine;
4-[1-(N-acetylglycyl)-1,2,3,6-tetrahydropyridin-4-yl]-N-(2,6-dichlorobenzoyl)-L-phenylalanine;
N-(2,6-dichlorobenzoyl)-4-{1-[(3-methoxyphenyl)acetyl]-1,2,3,6-tetrahydropyridin-4-yl}-L-phenylalanine;
4-[1-(cyclohexylacetyl)-1,2,3,6-tetrahydropyridin-4-yl]-N-(2,6-dichlorobenzoyl)-L-phenylalanine;
4-[1-(cyclobutylcarbonyl)-1,2,3,6-tetrahydropyridin-4-yl]-N-(2,6-dichlorobenzoyl)-L-phenylalanine;
4-[1-(cyclohexylcarbonyl)-1,2,3,6-tetrahydropyridin-4-yl]-N-(2,6-dichlorobenzoyl)-L-phenylalanine;
N-(2,6-dichlorobenzoyl)-4-[1-(pyridin-3-ylacetyl)-1,2,3,6-tetrahydropyridin-4-yl]-L-phenylalanine;
N-(2,6-dichlorobenzoyl)-4-[1-(3-pyrrolidin-1-ylbenzoyl)-1,2,3,6-tetrahydropyridin-4-yl]-L-phenylalanine;
4-[1-(4-cyano-2-methoxybenzoyl)-1,2,3,6-tetrahydropyridin-4-yl]-N-(2,6-dichlorobenzoyl)-L-phenylalanine;
N-(2,6-dichlorobenzoyl)-4-[1-(3-fluoro-2-methoxybenzoyl)-1,2,3,6-tetrahydropyridin-4-yl]-L-phenylalanine;
4-[1-(4-chloro-2-methoxybenzoyl)-1,2,3,6-tetrahydropyridin-4-yl]-N-(2,6-dichlorobenzoyl)-L-phenylalanine;
4-{1-[3-(acetylamino)benzoyl]-1,2,3,6-tetrahydropyridin-4-yl}-N-(2,6-dichlorobenzoyl)-L-phenylalanine;
N-(2,6-dichlorobenzoyl)-4-[1-(4-fluoro-3-methylbenzoyl)-1,2,3,6-tetrahydropyridin-4-yl]-L-phenylalanine;
4-[1-(2-chloro-5-fluorobenzoyl)-1,2,3,6-tetrahydropyridin-4-yl]-N-(2,6-dichlorobenzoyl)-L-phenylalanine;
N-(2,6-dichlorobenzoyl)-4-{1-[(3-methyl isoxazol-5-yl)acetyl]-1,2,3,6-tetrahydropyridin-4-yl}-L-phenylalanine;
N-(2,6-dichlorobenzoyl)-4-[1-(2,3-dihydro-1H-inden-2-ylacetyl)-1,2,3,6-tetrahydropyridin-4-yl]-L-phenylalanine;
N-(2,6-dichlorobenzoyl)-4-{1-[(1,1-dioxidotetrahydro-3-thienyl)acetyl]-1,2,3,6-tetrahydropyridin-4-yl}-L-phenylalanine;
N-(2,6-dichlorobenzoyl)-4-[1-(1,2,5-thiadiazol-3-ylcarbonyl)-1,2,3,6-tetrahydropyridin-4-yl]-L-phenylalanine;
N-(2,6-dichlorobenzoyl)-4-{1-[(4-isopropyl-1,2,3-thiadiazol-5-yl)carbonyl]-1,2,3,6-tetrahydropyridin-4-yl}-L-phenylalanine;
4-[1-(2,1-benzisoxazol-3-ylcarbonyl)-1,2,3,6-tetrahydropyridin-4-yl]-N-(2,6-dichlorobenzoyl)-L-phenylalanine;
N-(2,6-dichlorobenzoyl)-4-[1-(quinolin-3-ylcarbonyl)-1,2,3,6-tetrahydropyridin-4-yl]-L-phenylalanine;
N-(2,6-dichlorobenzoyl)-4-[1-(1-methylpiperidin-4-ylcarbonyl)-1,2,3,6-tetrahydropyridin-4-yl]-L-phenylalanine;
N-(2,6-dichlorobenzoyl)-4-(1-{[(2-fluorophenyl)amino]carbonyl}-1,2,3,6-tetrahydropyridin-4-yl)-L-phenylalanine;
N-(2,6-dichlorobenzoyl)-4-(1-{[(2-methoxyphenyl)amino]carbonyl}-1,2,3,6-tetrahydropyridin-4-yl)-L-phenylalanine;
N-(2,6-dichlorobenzoyl)-4-{1-[(propylamino)carbonyl]-1,2,3,6-tetrahydropyridin-4-yl}-L-phenylalanine;
4-{1-[(benzylamino)carbonyl]-1,2,3,6-tetrahydropyridin-4-yl}-N-(2,6-dichlorobenzoyl)-L-phenylalanine;
N-(2,6-dichlorobenzoyl)-4-(1-{[(2-fluorobenzyl)amino]carbonyl}-1,2,3,6-tetrahydropyridin-4-yl)-L-phenylalanine;
4-[1-(aminocarbonyl)-1,2,3,6-tetrahydropyridin-4-yl]-N-(2,6-dichlorobenzoyl)-L-phenylalanine;
N-(2,6-dichlorobenzoyl)-4-{1-[(1-methyl-1H-indol-2-yl)carbonyl]-1,2,3,6-tetrahydropyridin-4-yl}-L-phenylalanine; and
4-[1-(1H-benzimidazol-1-ylacetyl)-1,2,3,6-tetrahydropyridin-4-yl]-N-(2,6-dichlorobenzoyl)-L-phenylalanine;
or a pharmaceutically acceptable salt, prodrug, or hydrate thereof.

54. A pharmaceutical composition comprising a compound of the formula I according to claim 32 or a pharmaceutically acceptable salt, prodrug or hydrate thereof in association with a pharmaceutically acceptable carrier, diluent or excipient.

55. A pharmaceutical product which comprises a compound of the formula I according to claim 32 or a pharmaceutically acceptable salt, prodrug or hydrate thereof, and an additional anti-tumour agent for the conjoint treatment of cancer.

56. A compound of formula I according to claim 32 or a pharmaceutically acceptable salt, prodrug or hydrate thereof, which is an integrin inhibitor useful for the treatment of pathologically angiogenic diseases, thrombosis, coronary heart diseases, arteriosclerosis, atherosclerosis, tumours, osteoporosis, inflammations, autoimmune diseases, or infections.

57. A method of treating a disease or condition mediated by a5b1 which comprises administering to a patient in need of such treatment a compound of formula (I) as defined in claim 32 or a pharmaceutically acceptable salt, prodrug or hydrate thereof.

58. A method for the treatment of cancer in a warm-blooded animal in need of such treatment, which comprises administering to said animal an effective amount of a compound of formula (I) as defined in claim 32 or a pharmaceutically acceptable salt, prodrug or hydrate thereof.

59. A process for the preparation of a compound of formula (I) as defined in claim 32 which comprises:
Process (a) for the preparation of those compounds of formula I “----” is a bond, the coupling in the presence of a suitable catalyst of a compound of the formula VI or an ester thereof: 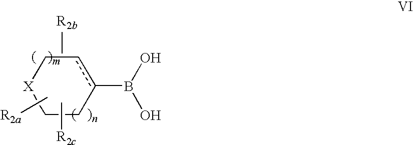
wherein X, R₂ₐ, R_{2b}, R_{2c}, m and n are as defined in claim 32, except any functional group is protected if necessary,
with a compound of the formula VII: 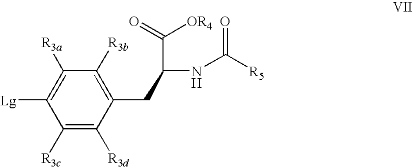
wherein R₃ₐ, R_{3b}, R_{3c}, R_{3d}, R₄ and R₅ are as defined in claim 32, except any functional group is protected if necessary,
and Lg is a leaving group; or
Process (b) for the preparation of those compounds of formula I wherein X is NR₁ and R₁ is a group of the formula RₓS(O)₂—, the reaction, in the presence of a suitable base, of a compound of the formula I of the formula Ia: 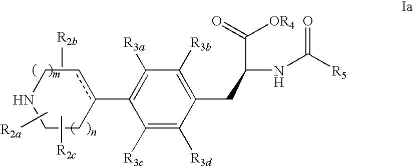
wherein R₂ₐ, R_{2b}, R_{2c}, R₃ₐ, R_{3b}, R_{3c}, R_{3d}, R₄, R₅, X, m and n are as defined in claim 32, except any functional group is protected if necessary,
with a compound of the formula VIII: 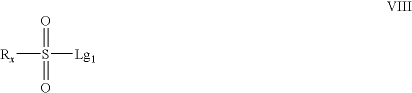
wherein Rₓ is as defined in claim 32, except any functional group is protected if necessary, and Lg₁ is a leaving group; or
Process (c) for the preparation of those compounds of formula I wherein X is NR₁ and R₁ is a group of the formula R_{y}C(O)—, the coupling, in the presence of a suitable base of a compound of the formula I of the formula Ia as defined in relation to Process (b) with a compound of the formula IX or a reactive derivative thereof:
R_{y}COOH IX
wherein R_{y} is as defined in claim 32, except any functional group is protected if necessary; or
Process (d) for the preparation of those compounds of formula I wherein “-----” in the compounds of formula I is absent, the reduction of a compound of the formula I wherein “----” is a bond; or
Process (e) the coupling of a compound of the formula X: 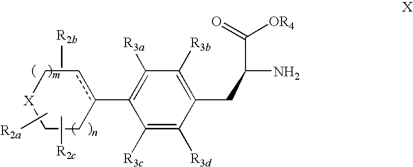
wherein R₂ₐ, R_{2b}, R_{2c}, R₃ₐ, R_{3b}, R_{3c}, R_{3d}, R₄, X, m and n are as defined in claim 32, except any functional group is protected if necessary,
with a compound of the formula XI or a reactive derivative thereof:
R₅COOH XI
wherein R₅ is as defined in claim 32, except any functional group is protected if necessary; or
Process (f) for the preparation of those compounds of formula I wherein X is NR₁ and R₁ is optionally substituted (C₁-C₆)alkyl, (C₃-C₆)cycloalkyl, heterocycloalkyl, (C₃-C₆)cycloalkyl(C₁-C₆)alkyl, heterocycloalkyl(C₁-C₆)alkyl, aralkyl or heteroaralkyl, the reaction, in the presence of a suitable base, of a compound of the formula I of the formula Ia as hereinbefore defined in relation to Process (b), with a compound of the formula XII:
R₁-Lg₂ XII
wherein R₁ is optionally substituted (C₁-C₆)alkyl, (C₃-C₆)cycloalkyl, heterocycloalkyl, (C₃-C₆)cycloalkyl(C₁-C₆)alkyl, heterocycloalkyl(C₁-C₆)alkyl, aralkyl or heteroaralkyl and
Lg₂ is a suitable leaving group; or
Process (g) for the preparation of those compounds of formula I wherein X is NR₁ and R₁ is a group of the formula R′HNC(O)—, the reaction of a compound of the formula I of the formula Ia as hereinbefore defined in relation to Process (b) with an isocyanate of the formula XIII:
R′N═C(O) XIII
wherein R′ is as defined in claim 32, except any functional group is protected if necessary; or
Process (h) for the preparation of those compounds of formula I wherein X is NR₁ and R₁ is aryl or heteroaryl, the coupling in the presence of a suitable catalyst, of a compound of the formula I of the formula Ia as hereinbefore defined in relation to Process (b) with an aryl or heteroaryl boronic acid, or an ester thereof; or
Process (i) for the preparation of those compounds of formula I wherein “----” is a bond, the coupling in the presence of a suitable catalyst of a compound of the formula XIV: 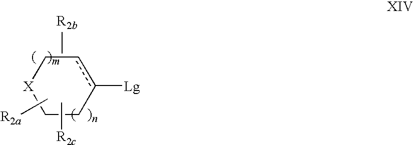
wherein X, R₂ₐ, R_{2b}, R_{2c}, m and n are as defined in claim 32, except any functional group is protected if necessary, and Lg is a leaving group,
with a compound of the formula XV or an ester thereof: 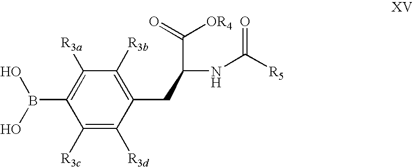
wherein R₃ₐ, R_{3b}, R_{3c}, R_{3d}, R₄ and R₅ are as defined in claim 32, except any functional group is protected if necessary,
and Lg is a leaving group;
and thereafter, if necessary (in any order):
(i) converting a compound of the formula I into another compound of the formula I;
(ii) removing any protecting groups; and
(iii) forming a pharmaceutically acceptable salt of the compound of formula I.

60. A compound selected from a compound of the formula VII, X and XV as defined in claim 59, or a salt thereof.
